(19) United States
(12) Patent Application Publication
(10) Pub. No.: US 2024/0417741 A1
(43) Pub. Date: Dec. 19, 2024

(54) C2 CARBON SOURCE-RESPONSIVE PROMOTERS

(71) Applicant: DANSTAR FERMENT AG, Zug (CH)

(72) Inventors: Alexandra-Elena Panaitiu, Lebanon, NH (US); Carlos Calcines-Cruz, Lebanon, NH (US); Britney Rose Privett, Lebanon, NH (US); David Aaron Argyros, Lebanon, NH (US)

(21) Appl. No.: 18/740,964

(22) Filed: Jun. 12, 2024

Related U.S. Application Data
(60) Provisional application No. 63/508,378, filed on Jun. 15, 2023.

(51) Int. Cl. C12N 15/63; C12N 9/04

(52) U.S. Cl. CPC C12N 15/635; C12N 9/0006; C12Y 101/01001

(57) ABSTRACT

The present disclosure concerns promoters engineered to increase their responsiveness to a C2 carbon source. The engineered promoters include at least one external carbon source-responsive element (CSRE) located upstream and proximal to the transcription start site of a gene operatively associated thereto. The engineered promoters can be used in a heterologous nucleic acid molecule, a vector, or an expression cassette to promote the expression of a gene in a microbe. The present disclosure also concerns a method for generating the engineered promoters as well as a method for expressing a gene in a recombination microbial host cell using the engineered promoter(s).

### CROSS-REFERENCE TO RELATED APPLICATION(S) AND DOCUMENT(S)

This patent application claims priority from U.S. provisional patent application 63/508,378 filed on Jun. 15, 2023, and herewith incorporated in its entirety.

### REFERENCE TO AN ELECTRONIC SEQUENCE LISTING

The contents of the electronic sequence listing (240141_413_Sequence_Listing.xml; Size: 67,315 bytes; and Date of Creation: Jun. 11, 2024) is herein incorporated by reference in its entirety.

### TECHNOLOGICAL FIELD

The present disclosure concerns promoters capable of inducing the expression of a gene in the presence of a C2 carbon source (like ethanol) in a recombinant microbial host.

### BACKGROUND

Microbes are used as platforms for expressing heterologous genes (which may encode heterologous polypeptides). However, expressions systems of heterologous genes should preferably be controlled to limit the metabolic burden on the microbial host. It is why inducible expression systems are usually preferred.

Komagataella phaffii (formerly Pichia pastoris) is a versatile expression system for recombinant polypeptides, allowing post-translational modifications and secretion in a manner similar to Saccharomyces cerevisiae. What distinguishes K. phaffii among recombinant expression hosts is its ability to achieve high density and high protein content, resulting in higher yields of expressed recombinant polypeptides. Moreover, the low levels of endogenous secreted polypeptides result in high purity recombinant polypeptides in the extracellular fraction. K. phaffii is additionally distinguished by its ability to efficiently utilize non-fermentable carbon sources such as glycerol and, particularly, methanol through the action of the alcohol oxidase (AOX) enzymes. In this regard, the AOX1 expression system is well-established in the K. phaffii field and widely employed for heterologous polypeptide expression. Methanol oxidation pathways are tightly regulated such that expression of genes required in the metabolism of methanol are induced only in the presence of methanol (such as the AOX1 alcohol oxidase) and repressed by various carbon sources, including glucose. This tight control of methanol-related genes has been leveraged in biotechnology for the time-controlled production of heterologous proteins: under the control of the AOX1 promoter, heterologous polypeptide expression will be induced only when methanol is added to the system. This presents great advantages for polypeptides that are cytotoxic and it also allows for bioprocess control of protein production. Specifically, in aerobic fermentation processes, the feed is switched from other carbon sources to methanol, thus inducing polypeptide expression. However, methanol presents technical, environmental, and safety risks owing to its flammability and toxicity.

There is thus a need to develop a microbial expression system, and particularly microbial promoters, which would be inducible in the presence of another inducing agent than methanol. In some embodiments, the inducing agent should be compatible with large scale commercial operations (e.g., having a lower flammability and/or toxicity than methanol).

### SUMMARY

The present disclosure concerns promoters which have been engineered to increase the expression strength in the presence of a C2 carbon source like ethanol. The engineered promoters include external carbon source-responsive elements.

According to a first aspect, the present disclosure concerns an engineered promoter (i) derived from a parental promoter having a transcription start site and (ii) for expressing a gene. The engineered promoter has at least one external carbon source-responsive element (CSRE). The at least one external CSRE has the nucleic acid sequence of formula (I):

N₁N₂CCN₃N₄TN₅N₆N₇CCGN₈ (I)

wherein N₁ is any nucleic acid residue; N₂ is any nucleic acid residue, preferably C or T; N₃ is any nucleic acid residue, preferably A, G or T; N₄ is any nucleic acid residue, preferably C or T; N₅ is any nucleic acid residue, preferably A, C or G; N₆ is any nucleic acid residue, preferably A or G; N₇ is any nucleic acid residue, preferably G or T; and N₈ is any nucleic acid residue, preferably A or G. The at least one external CSRE comprises a first external CSRE located upstream of and being proximal to the transcription start site. In an embodiment, the gene comprises an open reading frame having a start codon. In another embodiment, the first external CSRE is located at most 390 base pairs upstream (−390) of the start codon. In still another embodiment, the engineered promoter comprises a TATA box. In still a further embodiment, in the presence of a C2 carbon source like ethanol, the engineered promoter is capable of inducing transcription of the gene at a higher level than the parental promoter. In yet another embodiment, the at least one external CSRE comprises the nucleic acid sequence of any one of SEQ ID NO: 26 to 35. In still yet another embodiment, the engineered promoter of any one of claims 1 to 6 comprising at least two, three, four, five, six, seven, eight, nine, or ten external CSREs. In some embodiments, the parental promoter is an ethanol responsive promoter, such as, for example, the promoter of the adh2 gene (adh2p). In some specific embodiments, the engineered promoter has the nucleic acid sequence of SEQ ID NO: 6, 7, 8, 9, 10, 11, 19, 20, 21, 22, or 23. In some embodiments, the parental promoter is a constitutive promoter, such as, for example, the promoter of the sti1 gene (sti1p). In some specific embodiments, the engineered promoter has the nucleic acid sequence of SEQ ID NO: 12, 13, 14, 15, 16, 17, or 18.

According to a second aspect, the present disclosure provides a heterologous nucleic acid molecule having the engineered promoter described herein operably associated with a gene. In some embodiments, the gene encodes a polypeptide.

According to a third aspect, the present disclosure provides a vector comprising the engineered promoter described herein or the heterologous nucleic acid molecule described herein.

According to a fourth aspect, the present disclosure provides an expression cassette comprising the engineered promoter described herein or the heterologous nucleic acid molecule described herein.

According to a fifth aspect, the present disclosure provides a recombinant microbial host cell comprising the engineered promoter described herein, the heterologous nucleic acid molecule described herein, the vector described herein or the expression cassette described herein. In an embodiment, the recombinant microbial host cell has native alcohol dehydrogenase activity. In another embodiment, the recombinant microbial host is a yeast. In still a further embodiment, the recombinant microbial host cell is from Komagataella sp., and in yet further embodiments, from Komagataella phaffii.

According to a sixth aspect, the present disclosure concerns a method for increasing the responsiveness to a C2 carbon source of an engineered promoter for expressing a gene. The method comprises introducing, in a parental promoter having a transcription start site, upstream and proximal to the transcription start site, a first external carbon source-responsive element (CSRE). The first external CSRE has the nucleic acid sequence of formula (I):

N₁N₂CCN₃N₄TN₅N₆N₇CCGN₈ (I)

wherein N₁ is any nucleic acid residue; N₂ is any nucleic acid residue, preferably C or T; N₃ is any nucleic acid residue, preferably A, G or T; N₄ is any nucleic acid residue, preferably C or T; N₅ is any nucleic acid residue, preferably A, C or G; N₆ is any nucleic acid residue, preferably A or G; N₇ is any nucleic acid residue, preferably G or T; and N₈ is any nucleic acid residue, preferably A or G. In an embodiment, the gene comprises an open reading frame having a start codon. In another embodiment, the method comprises introducing the first external CSRE at most 390 base pairs upstream (−390) of the start codon. In a further embodiment, the parental promoter comprises a TATA box. In still another embodiment, the first external CSRE comprises the nucleic acid sequence of any one of SEQ ID NO: 26 to 35. In some embodiments, the method comprises introducing two, three, four, five, six, seven, eight, nine, or ten external CSREs in the parental promoter. In further embodiments, each of the external CSRE have a nucleic acid sequence independently selected from any one of SEQ ID NO: 26 to 35. In an embodiment, the parental promoter is an ethanol responsive promoter. In another embodiment, the parental promoter is a constitutive promoter.

According to a seventh aspect, the present disclosure provides a method for expressing a gene in the recombinant microbial host cell described herein. The method comprises (i) contacting the recombinant microbial host cell with a C2 carbon source, like ethanol, so as to allow the expression of the gene. In an embodiment, the method further comprises, before the step (i), (i′) propagating the recombinant microbial host cell with an alternative carbon source different from the C2 carbon source. In an embodiment, the alternative carbon source comprises glucose, fructose, and/or glycerol. In another embodiment, the gene encodes a polypeptide. In a further embodiment, the polypeptide is an intracellular polypeptide or a secreted polypeptide. In still another embodiment, the secreted polypeptide is in a free form or is associated to the surface of the recombinant yeast host cell. In still another embodiment, the polypeptide associated to the surface of the recombinant yeast host cell is a tethered polypeptide. In a further embodiment, the polypeptide is an enzyme. In some embodiments, the method further comprises, after step (i), (ii) substantially separating the polypeptide from the recombinant microbial host cell.

### DETAILED DESCRIPTION OF THE DRAWINGS

Having thus generally described the nature of the invention, reference will now be made to the accompanying drawings, showing by way of illustration, a preferred embodiment thereof, and in which:

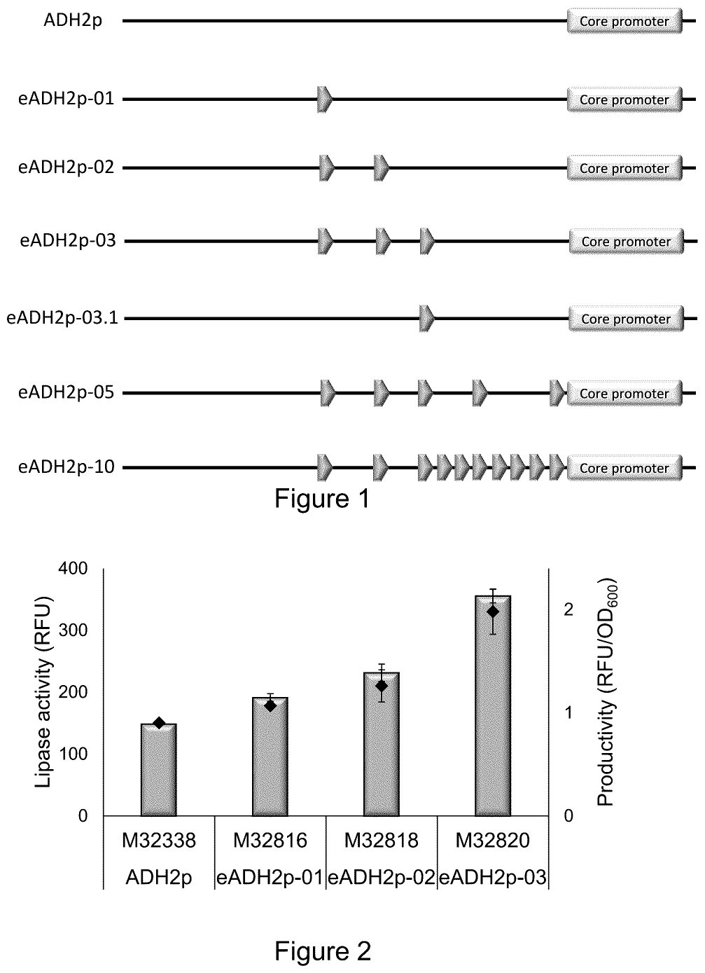

FIG. 1 provides a schematic depiction of one of the engineering schemes applied to the alcohol dehydrogenase 2 (ADH2) promoter. The triangles symbolically depict engineered carbon source-responsive elements (CSREs) and their relative distance on the nucleotide strand from the functional core promoter region proximal to the adh2 gene (not depicted). In the engineered promoter variants denoted eADH2p-01, eADH2p-02, eADH2p-03, eADH2p-03.1, eADH2p-05, and eADH2p-10 additional CSREs were introduced according to the illustration, gradually more proximal to the core promoter. The relative positions of the additional CSREs are not drawn to scale.

FIG. 2 provides the results of a reporter enzymatic activity assay on supernatant fractions from shake flasks of cultures of strains M32338, M32816, M32818, and M32820. Ethanol was used as the carbon source for all strains. Reporter enzymatic activity (bars) is reported as relative fluorescence units (RFU) compared to a negative control. Productivity (ratios of the reporter enzymatic activity and OD₆₀₀, rhombi) is also shown. Error bars represent standard deviation of the mean.

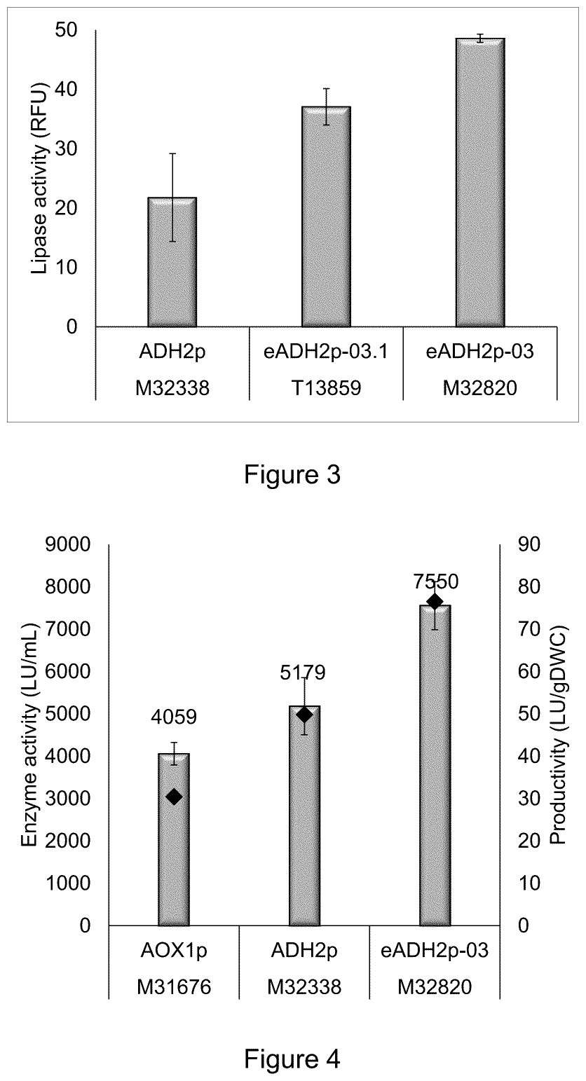

FIG. 3 provides the results of a reporter enzymatic activity assay on supernatant fractions from 96 well culture plates of cultures of strain M32338, isolate T13859, and strain M32820. Ethanol was used as the carbon source for all strains. Reporter enzymatic activity is reported as relative fluorescence units (RFU) compared to a negative control. Error bars represent standard deviation of the mean.

FIG. 4 provides the results of a reporter enzymatic activity assay on supernatant fractions from bioreactor fermentations with strains M31676, M32338, and M32820. Methanol was used as the carbon source for strain M31676 and ethanol for strains M32338 and M32820. Reporter enzymatic activity units (bars) were computed by comparing against a standard curve of a commercial lipase sample. Productivity (ratios of the reporter enzymatic activity and dry cell weight, rhombi) is also shown. Error bars represent error propagated according to established uncertainty propagation rules.

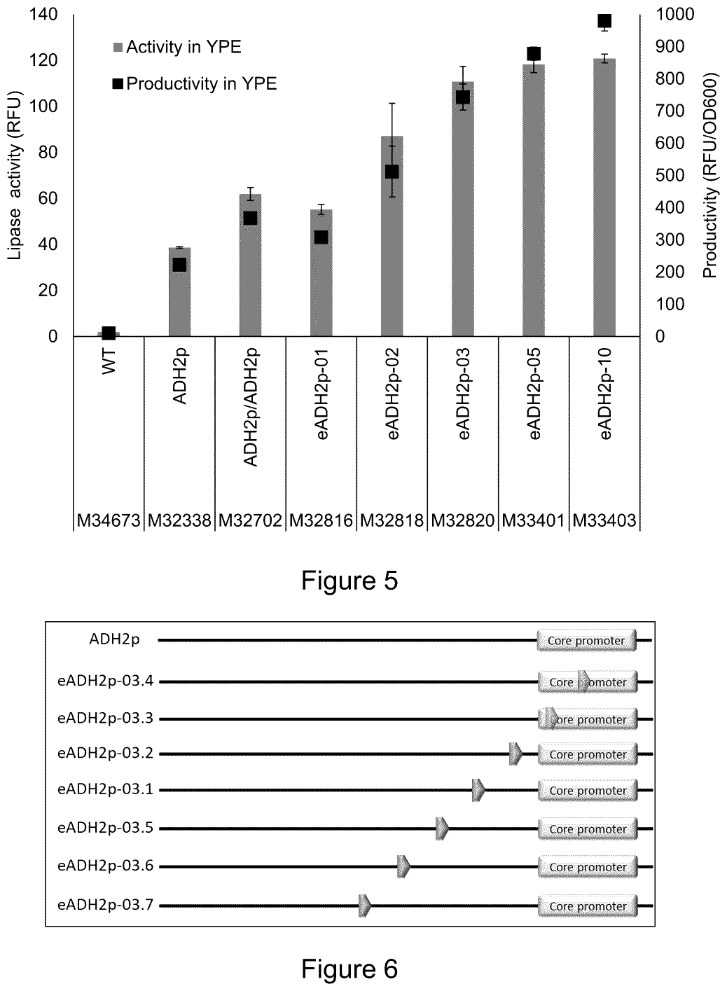

FIG. 5 provides the results of the reporter enzymatic activity assay on supernatant fractions from 96 well culture plates from strains M34673, M32338, M32702, M32816, M32818, M32820, M33401, and M33403. Ethanol was used as the carbon source for all strains. Reporter enzymatic activity (bars) is reported as relative fluorescence units (RFU) compared to a negative control. Productivity (ratios of the reporter enzymatic activity and OD₆₀₀, squares) is also shown. Error bars represent standard deviation of the mean.

FIG. 6 provides a schematic depiction of another engineering scheme applied to the ADH2 promoter. The triangles symbolically depict engineered CSREs and their relative distance on the nucleotide strand from the functional core promoter region proximal to the adh2 gene (not depicted). In the engineered promoter variants denoted eADH2p-03.1 through eADH2p-03.7, one CSRE was introduced according to the illustration, gradually more proximal to the core promoter. The relative positions of the CSREs are not drawn to scale.

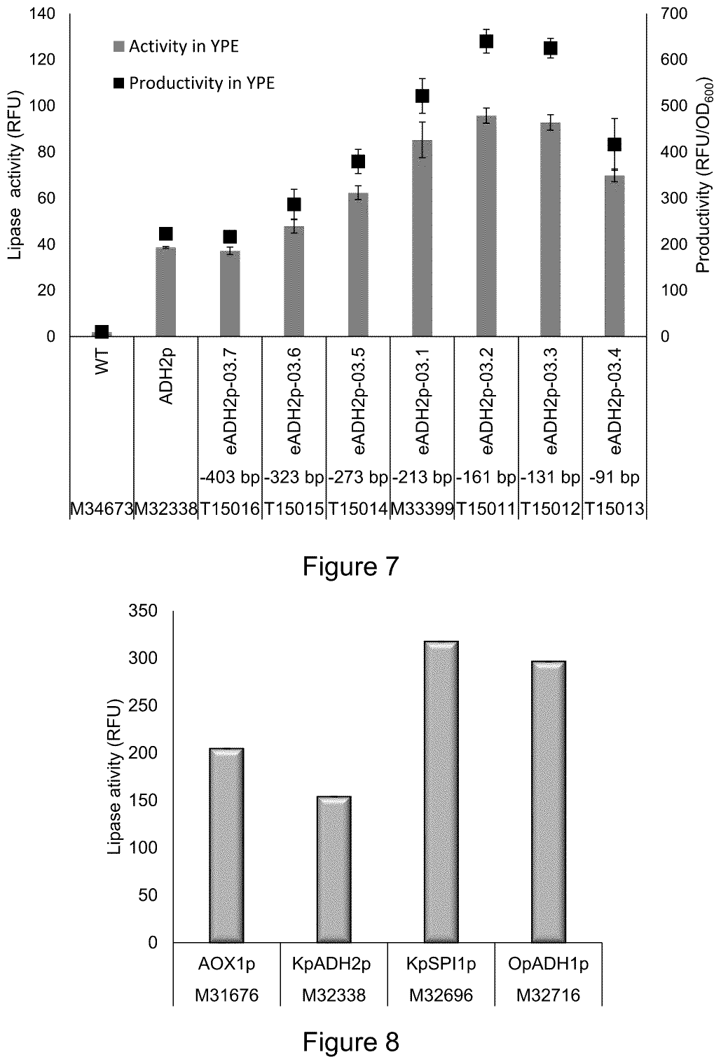

FIG. 7 provides the reporter enzymatic activity assay on supernatant fractions from 96 well culture plates of strains M34673, M32388, M33399, and isolates T15016, T15015, T15014, T15011, T15012, and T15013. The distance between the open reading frame (ORF) and the added CSRE is included for each engineered promoter on the x axis label. Ethanol was used as the carbon source for all strains, and isolates. Reporter enzymatic activity (bars) is reported as relative fluorescence units (RFU) compared to a negative control. Productivity (ratios of the reporter enzyme activity and OD₆₀₀, squares) is also shown. Error bars represent standard deviation of the mean.

FIG. 8 provides the reporter enzymatic activity assay on supernatant fractions from shake flasks of cultures of strains M31676, M32338, M32696, and M32716. Methanol was used as the carbon source for strain M31676 and glucose for strains M32338, M32696, and M32716. Reporter enzymatic activity (bars) is reported as relative fluorescence units (RFU) compared to a negative control. Error bars represent standard deviation of the mean.

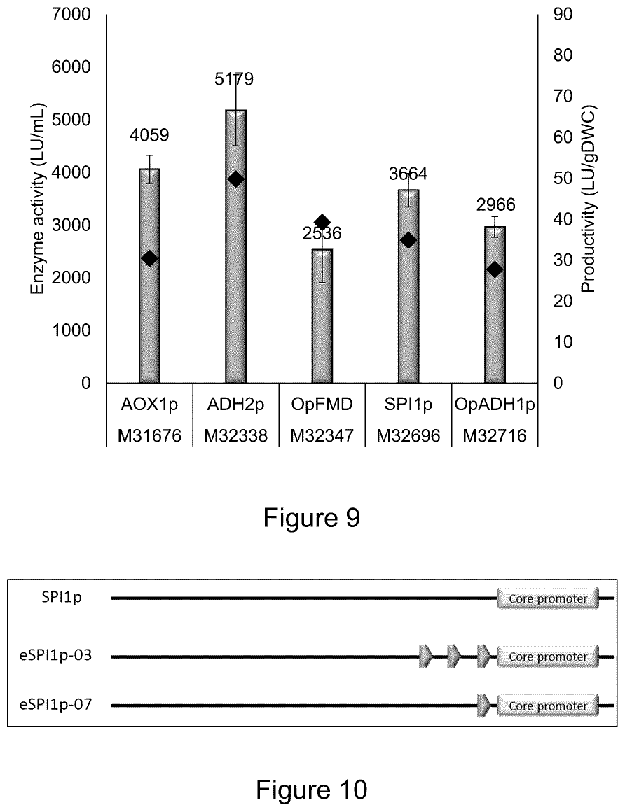

FIG. 9 provides the reporter enzymatic activity assay on supernatant fractions from bioreactor fermentations of strains M31676, M32388, M32347, M32696, and M32716. Methanol was used as the carbon source for strain M31676 and ethanol for strains M32338, M32347, M32696, and M32716. Reporter enzymatic activity units (bars) were computed by comparing against a standard curve of a commercial lipase sample. Productivity (ratios of the reporter enzymatic activity and dry cell weight, rhombi) is also shown. Error bars represent error propagated according to established uncertainty propagation rules.

FIG. 10 provides a schematic depiction of one of the engineering schemes applied to the constitutive stationary phase induced 1 (SPI1) promoter. The triangles symbolically depict engineered CSREs and their relative distance on the nucleotide strand from the functional core promoter region proximal to the spi1 gene (not depicted). In the engineered promoter variant denoted eSPI1p-03 additional CSREs were introduced according to the illustration, gradually more proximal to the core promoter.

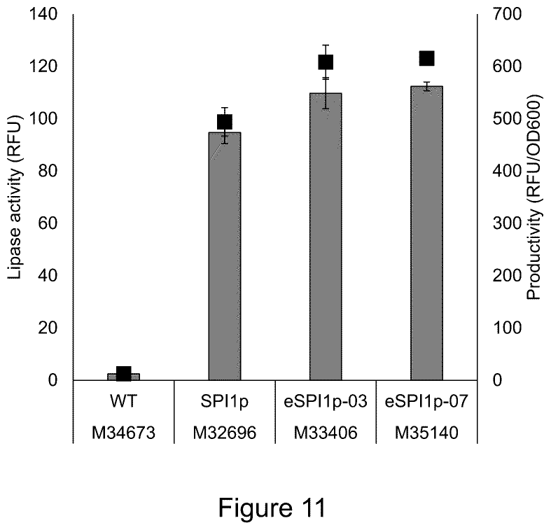

FIG. 11 provides the reporter enzymatic activity assay on supernatant fractions from 96-well culture plates of cultures of strains M34673, M32696, M33406, and M35140. Ethanol was used as the carbon source for all strains. Reporter enzymatic activity (bars) is reported as relative fluorescence units (RFU) compared to a negative control. Productivity (ratios of the reporter enzymatic activity and OD₆₀₀, squares) is also shown. Error bars represent standard deviation of the mean.

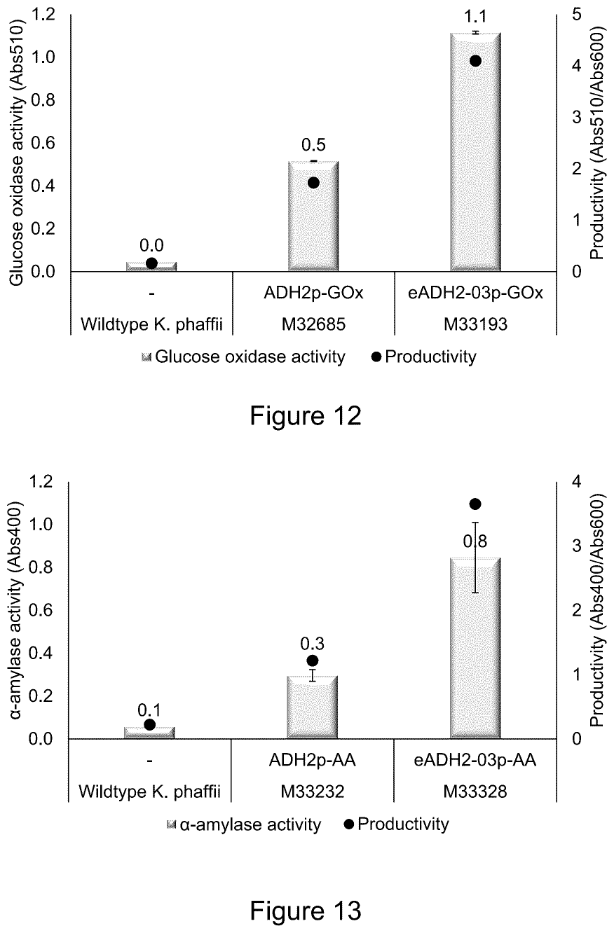

FIG. 12 provides the reporter enzymatic activity assay on supernatant fractions from 96-well culture plates of cultures of strains M17500 (wild type), M32685, and M33193. Ethanol was used as the carbon source for all strains. Reporter enzymatic activity (bars) is reported as the absorbance at 510 nm. Productivity (ratios of the reporter enzymatic activity and OD₆₀₀, black circles) is also shown. Error bars represent standard deviation of the mean.

FIG. 13 provides the reporter enzymatic activity assay on supernatant fractions from 96-well culture plates of cultures of strains M17500 (wild type), M33232, and M33328. Ethanol was used as the carbon source for all strains. Reporter enzymatic activity (bars) is reported as the absorbance at 400 nm. Productivity (ratios of the reporter enzymatic activity and OD₆₀₀, black circles) is also shown. Error bars represent standard deviation of the mean.

### DETAILED DESCRIPTION

The present disclosure concerns promoters for expressing genes (which can be native or heterologous) in a recombinant microbial host cell using a C2 carbon source as an inducer (e.g., C2 carbon source-responsive promoters). In the context of the present disclosure, an inducer is a chemical or biological entity which, when placed in contact with the recombinant microbial host cell, increases the ability of the engineered promoter to promote the expression of a downstream gene operatively linked to the engineered promoter. In some embodiments, more than one inducer can influence the engineered promoter's ability to express a downstream gene. In some embodiments, the promoters can be used in a methanol-free expression system, e.g., an expression system that does not use methanol as an inducer and can be used without the addition of methanol in the medium. The present disclosure further provides leveraging ethanol-responsive promoters and coupling this expression system with an aerobic fermentation process in which ethanol or another C2 carbon source is the carbon source. In some embodiments, the use of an expression system based on ethanol-responsive promoters has the added benefit, in aerobic fermentations, that the presence of ethanol may aid in microbial contamination control.

Still in the context of the present disclosure, the expression “C2 carbon source” refers to a carbon source which is assimilable by the recombinant microbial host and which comprises two (2) carbon atoms. Embodiments of C2 carbon sources include, but are not limited to, ethanol, acetate, and combinations thereof.

### Engineered Promoters

The engineered promoters of the present disclosure exhibit increased expression strength in the presence of C2 carbon sources like, for example, ethanol and/or acetate. This increased expression strength in the presence of a C2 carbon source like ethanol is observed in the absence of methanol. As used in the present disclosure “increased expression strength in the presence of a C2 carbon source like ethanol” refers to an increase, in the recombinant microbial host cell and in the presence of the C2 carbon source, in the expression of a gene which is operatively linked to one or more of the engineered promoters. This increase in gene expression can be observed when compared to the parental promoter's expression strength in the presence of the C2 carbon source.

In some embodiments, the engineered promoters of the present disclosure exhibit derepression in the presence of a non-C2 carbon source. In the context of the present disclosure, a non-C2 carbon source refers to a carbon source that is assimilable by the recombinant microbial host cell and comprises more than two (2) carbon atoms. Embodiments of non-C2 carbon sources include, but are not limited too, glucose, fructose, glycerol and combinations thereof. In such embodiments, the level of derepression in the context of non-C2 carbon sources (such as, for example, glucose, fructose, and/or glycerol) of the engineered promoters is higher than the corresponding level of derepression of the parental promoter.

In embodiments in which the gene encodes a polypeptide having enzymatic activity, the modulation in expression strength associated with the engineered promoters can be reflected by an increase in the enzymatic activity of the polypeptide of at least 10% (when compared to the enzymatic activity of the same polypeptide under the control of the parental promoter).

In some embodiments, the engineered promoters of the present disclosure exhibit increased expression strength in the presence of glucose. This increased expression strength in the presence of glucose is observed in the absence of methanol. As used in the present disclosure “increased expression strength in the presence of glucose” refers to an increase, in the recombinant microbial host cell and in the presence of glucose, in the expression of a gene which is operatively linked to one or more of the engineered promoters. This increase in gene expression is observed when compared to the parental promoter's expression strength in the presence of glucose. As indicated above, once glucose has been consumed, the level of expression of the engineered promoter is further increased (in view of the derepression).

In some embodiments, the engineered promoters of the present disclosure exhibit increased expression strength in the presence of fructose. This increased expression strength in the presence of fructose is observed in the absence of methanol. As used in the present disclosure “increased expression strength in the presence of fructose” refers to an increase, in the recombinant microbial host cell and in the presence of fructose, in the expression of a gene which is operatively linked to one or more of the engineered promoters. This increase in gene expression is observed when compared to the parental promoter's expression strength in the presence of fructose. As indicated above, once fructose has been consumed, the level of expression of the engineered promoter is further increased (in view of the derepression).

In some embodiments, the engineered promoters of the present disclosure exhibit increased expression strength in the presence of glycerol. This increased expression strength in the presence of glycerol is observed in the absence of methanol. As used in the present disclosure “increased expression strength in the presence of glycerol refers to an increase, in the recombinant microbial host cell and in the presence of glycerol, in the expression of a gene which is operatively linked to one or more of the engineered promoters. This increase in gene expression is observed when compared to the parental promoter's expression strength in the presence of glycerol. As indicated above, once glycerol has been consumed, the level of expression of the engineered promoter can be further increased (in view of the derepression).

The engineered promoters of the present disclosure comprise at least one external carbon-source responsive element (CSRE). Carbon source-dependent regulation of promoter activation, where it exists, is mediated by specific nucleotide motifs in the promoter sequence, where transcription factors such as Adr1, Cat8 (also referred to as Cat8-1), Sip4 (also referred to as Cat8-2), or Mig1 bind. Termed “carbon source-responsive elements” (CSREs), these motifs mediate repression, de-repression, or activation of genes downstream of the respective promoter. Cat8 and Sip4 have been described in yeasts, including S. cerevisiae, to have conserved DNA binding domains and to be implicated in gene de-repression in the context of non-fermentable carbon sources.

In the context of the present disclosure, a carbon-source responsive element (CSRE) refers to a nucleic acid motif which can be represented by Formula (I):

N₁N₂CCN₃N₄TN₅N₆N₇CCGN₈ (I)

The consensus sequence for the external CSRE has been obtained by comparing the nucleic acid sequences of the external CSREs used in the example. Table 1 provides an alignment of the external CSREs used in the example.

TABLE 1
Alignment of the external CSREs used in the examples and consensus sequence
derived therefrom. In the consensus sequence, N₁ is any nucleic acid residue; N₂ is
any nucleic acid residue, preferably C or T; N₃ is any nucleic acid residue, preferably A, G or
T; N₄ is any nucleic acid residue, preferably C or T; N₅ is any nucleic acid residue, preferably
A, C or G; N₆ is any nucleic acid residue, preferably A or G; N₇ is any nucleic
acid residue, preferably G or T; and N₈ is any nucleic acid residue, preferably
A or G.

| CSRE | SEQ ID NO: | Nucleic acid sequence | | | | | | | | | | | | | |
|---|---|---|---|---|---|---|---|---|---|---|---|---|---|---|---|
| CSRE#1 | 26 | T | T | C | C | G | T | T | C | G | T | C | C | G | G |
| CSRE#2 | 27 | C | T | C | C | G | C | T | C | A | G | C | C | G | A |
| CSRE#3 | 28 | T | C | C | C | G | T | T | G | G | T | C | C | G | A |
| CSRE#4 | 29 | A | C | C | C | G | T | T | C | A | G | C | C | G | A |
| CSRE#5 | 30 | C | T | C | C | A | T | T | C | G | G | C | C | G | A |
| CSRE#6 | 31 | T | C | C | C | T | T | T | A | A | T | C | C | G | A |
| CSRE#7 | 32 | G | C | C | C | G | T | T | A | G | T | C | C | G | A |
| CSRE#8 | 33 | T | T | C | C | A | T | T | A | A | G | C | C | G | G |
| CSRE#9 | 34 | C | C | C | C | T | T | T | C | G | G | C | C | G | G |
| CSRE#10 | 35 | A | T | C | C | A | T | T | C | A | T | C | C | G | G |
| CSRE | N.A. | N₁ | N₂ | C | C | N₃ | N₄ | T | N₅ | N₆ | N₇ | C | C | G | N₈ |
| consensus | | | | | | | | | | | | | | | |

In Formula (I), N₁ refers to any naturally occurring nucleic acid residue. In some embodiments, N₁ is T. In such embodiments, the CSRE can have the nucleic acid sequence of SEQ ID NO: 26, 28, 31, or 33. In some embodiments, N₁ is C. In such embodiments, the CSRE can have the nucleic acid sequence of SEQ ID NO: 27, 30, or 34. In some embodiments, N₁ is A. In such embodiments, the CSRE can have the nucleic acid sequence of SEQ ID NO: 29, or 35. In some embodiments, N₁ is G. In such embodiments, the CSRE can have the nucleic acid sequence of SEQ ID NO: 32.

In Formula (I), N₂ refers to any naturally occurring nucleic acid residue. In some embodiments, N₂ refers to C or T. In some specific embodiments, N₂ refers to C. In such embodiments, the CSRE can have the nucleic acid sequence of SEQ ID NO: 28, 29, 31, 32, or 34. In some specific embodiments, N₂ refers to T. In such embodiments, the CSRE can have the nucleic acid sequence of SEQ ID NO: 26, 27, 30, 33, or 35.

In Formula (I), N₃ refers to any naturally occurring nucleic acid residue. In some embodiments, N₃ refers to A, G, or T. In some specific embodiments, N₃ refers to A. In such embodiments, the CSRE can have the nucleic acid sequence of SEQ ID NO: 30, 33, or 35. In some specific embodiments, N₃ refers to G. In such embodiments, the CSRE can have the nucleic acid sequence of SEQ ID NO: 26, 27, 28, 29, or 32. In some specific embodiments, N₃ refers to T. In such embodiments, the CSRE can have the nucleic acid sequence of SEQ ID NO: 31 or 34.

In Formula (I), N₄ refers to any naturally occurring nucleic acid residue. In some embodiments, N₄ refers to C or T. In some specific embodiments, N₄ refers to C. In such embodiments, the CSRE can have the nucleic acid sequence of SEQ ID NO: 27. In some specific embodiments, N₄ refers to T. In such embodiments, the CSRE can have the nucleic acid sequence of SEQ ID NO: 26, 28, 29, 30, 31, 32, 33, 34, or 35.

In Formula (I), N₅ refers to any naturally occurring nucleic acid residue. In some embodiments, N₅ refers to A, C, or G. In some specific embodiments, N₅ refers to A. In such embodiments, the CSRE can have the nucleic acid sequence of SEQ ID NO: 31, 32, or 33. In some specific embodiments, N₅ refers to C. In such embodiments, the CSRE can have the nucleic acid sequence of SEQ ID NO: 26, 27, 29, 30, 34, or 35. In some specific embodiments, N₅ refers to G. In such embodiments, the CSRE can have the nucleic acid sequence of SEQ ID NO: 28.

In Formula (I), N₆ refers to any naturally occurring nucleic acid residue. In some embodiments, N₆ refers to A or G. In some specific embodiments, N₆ refers to A. In such embodiments, the CSRE can have the nucleic acid sequence of SEQ ID NO: 27, 29, 31, 33, or 35. In some specific embodiments, N₆ refers to G. In such embodiments, the CSRE can have the nucleic acid sequence of SEQ ID NO: 26, 28, 30, 32, or 34.

In Formula (I), N₇ refers to any naturally occurring nucleic acid residue. In some embodiments, N₇ refers to G or T. In some specific embodiments, N₇ refers to G. In such embodiments, the CSRE can have the nucleic acid sequence of SEQ ID NO: 27, 29, 30, 33, or 34. In some specific embodiments, N₇ refers to T. In such embodiments, the CSRE can have the nucleic acid sequence of SEQ ID NO: 26, 28, 31, 32, or 35.

In Formula (I), Nₑ refers to any naturally occurring nucleic acid residue. In some embodiments, Nₑ to A or G. In some specific embodiments, Nₑ refers to A. In such embodiments, the CSRE can have the nucleic acid sequence of SEQ ID NO: 27, 28, 29, 30, 31, or 32. In some specific embodiments, Nₑ refers to G. In such embodiments, the CSRE can have the nucleic acid sequence of SEQ ID NO: 26, 33, 34, or 35.

The engineered promoters of the present disclosure comprise an external CSRE. In the context of the present disclosure, the term “external” when used in connection with the expression “CSRE”, refers to the fact a CSRE has been added to the parental promoter to generate the engineered promoter(s). A native CSRE which may be present in the parental promoter is not considered to be an external CSRE. A native CSRE which has been modified or replaced is also not considered to be an external CSRE.

In an embodiment, the engineered promoters comprise a single external CSRE. This single external CSRE can have, in some embodiments, the nucleic acid sequence of SEQ ID NO: 26. This single external CSRE can have, in some embodiments, the nucleic acid sequence of SEQ ID NO: 27. This single external CSRE can have, in some embodiments, the nucleic acid sequence of SEQ ID NO: 28. This single external CSRE can have, in some embodiments, the nucleic acid sequence of SEQ ID NO: 29. This single external CSRE can have, in some embodiments, the nucleic acid sequence of SEQ ID NO: 30. This single external CSRE can have, in some embodiments, the nucleic acid sequence of SEQ ID NO: 31. This single external CSRE can have, in some embodiments, the nucleic acid sequence of SEQ ID NO: 32. This single external CSRE can have, in some embodiments, the nucleic acid sequence of SEQ ID NO: 33. This single external CSRE can have, in some embodiments, the nucleic acid sequence of SEQ ID NO: 34. This single external CSRE can have, in some embodiments, the nucleic acid sequence of SEQ ID NO: 35. In some embodiments, the engineered promoter comprising a single external CSRE can have the nucleic acid sequence of SEQ ID NO: 6, 11, 12, 14, 15, 16, 18, 19, 20, 21, 22, 23, 24.

In an embodiment, the engineered promoters comprise at least two external carbon-source responsive elements (CSREs). In an embodiment, the engineered promoters comprise two external carbon-source responsive elements (CSREs). In embodiments, the two external CSREs can have the same nucleic acid sequence or different nucleic acid sequences. In embodiments in which the CSREs have the same nucleic acid sequence, the two external CSREs can have the nucleic acid sequence of SEQ ID NO: 26, 27, 28, 29, 30, 31, 32, 33, 34, or 35. In embodiments in which the CSREs have different nucleic acid sequences, the two external CSREs can be a selection of any two one of the CSREs having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 26 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 27 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 28 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 29 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 30 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 31 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 32 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 33 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 34 and another CSRE having the nucleic acid sequence of SEQ ID NO: 26, 27, 28, 29, 30, 31, 32, 33, 34, or 35. In embodiments, the two external CSREs can comprise one CSRE having the nucleic acid sequence of SEQ ID NO: 35 and another CSRE having the nucleic acid sequence of SEQ ID NO: 26, 27, 28, 29, 30, 31, 32, 33, 34, or 35. In some embodiments, the engineered promoter comprising two external CSREs can have the nucleic acid sequence of SEQ ID NO: 7.

In an embodiment, the engineered promoters comprise at least three external carbon-source responsive elements (CSREs). In an embodiment, the engineered promoters comprise three external carbon-source responsive elements (CSREs). In embodiments, the three external CSREs can have the same nucleic acid sequence or different nucleic acid sequences. In embodiments in which the CSREs have the same nucleic acid sequence, the three external CSREs can have the nucleic acid sequence of SEQ ID NO: 26, 27, 28, 29, 30, 31, 32, 33, 34, or 35. In embodiments, two of the three external CSREs can have the same nucleic acid sequence and a third external CSRE can have a different nucleic acid sequence. For example, two of the external CSREs can have the nucleic acid sequence of SEQ ID NO: 26 and the third external CSRE can have the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the third external CSRE can have the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the third external CSRE can have the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the third external CSRE can have the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the third external CSRE can have the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the third external CSRE can have the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the third external CSRE can have the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the third external CSRE can have the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the third external CSRE can have the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the third external CSRE can have the nucleic acid sequence of any one of SEQ ID NO: 26, 27, 28, 29, 30, 31, 32, 33, or 34. In embodiments, the three external CSREs can have different nucleic acid sequences. In embodiments in which the CSREs have different nucleic acid sequences, the three external CSREs can have a nucleic acid sequence independently selected SEQ ID NO: 26, 27, 28, 29, 30, 31, 32, 33, 34, or 35. In a specific embodiment, the three external CSREs can have a nucleic acid sequence independently selected SEQ ID NO: 26, 27, or 28. In yet another embodiment, the three external CSREs can have a distinct nucleic acid sequence, one CSRE having the nucleic acid sequence of SEQ ID NO: 26, another CSRE having the nucleic acid sequence of SEQ ID NO: 27, and a further CSRE having the nucleic acid sequence of SEQ ID NO: 28. In some embodiments, the engineered promoter comprising three external CSREs can have the nucleic acid sequence of SEQ ID NO: 8 or 13.

In an embodiment, the engineered promoters comprise at least four external carbon-source responsive elements (CSREs). In an embodiment, the engineered promoters comprise four external carbon-source responsive elements (CSREs). In embodiments, the four external CSREs can have the same nucleic acid sequence or different nucleic acid sequences. In embodiments in which the CSREs have the same nucleic acid sequence, the four external CSREs can have the nucleic acid sequence of SEQ ID NO: 26, 27, 28, 29, 30, 31, 32, 33, 34, or 35. In embodiments, at least two of the four external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the four external CSREs can have the same nucleic acid sequence and the remaining external CSRE can have a different nucleic acid sequence. For example, three of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the four external CSREs can have different nucleic acid sequences. In embodiments, the four external CSREs can have a nucleic acid sequence independently selected from SEQ ID NO: 26, 27, 28, 29, 30, 31, 32, 33, 34, or 35. In a specific embodiment, the four external CSREs can have a nucleic acid sequence independently selected SEQ ID NO: 26, 27, 28, or 29. In yet another embodiment, the four external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, and yet another CSRE having the nucleic acid sequence of SEQ ID NO: 29. In some embodiments, the engineered promoter comprising four external CSREs can have the nucleic acid sequence of SEQ ID NO: 14.

In an embodiment, the engineered promoters comprise at least five external carbon-source responsive elements (CSREs). In an embodiment, the engineered promoters comprise five external carbon-source responsive elements (CSREs). In embodiments, the five external CSREs can have the same nucleic acid sequence or different nucleic acid sequences. In embodiments in which the CSREs have the same nucleic acid sequence, the five external CSREs can have the nucleic acid sequence of SEQ ID NO: 26, 27, 28, 29, 30, 31, 32, 33, 34, or 35. In embodiments, at least two of the five external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the five external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the five external CSREs can have the same nucleic acid sequence and the remaining external CSRE can have a different nucleic acid sequence. For example, four of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the five external CSREs can have different nucleic acid sequences. In embodiments, the five external CSREs can have a nucleic acid sequence independently selected from SEQ ID NO: 26, 27, 28, 29, 30, 31, 32, 33, 34, or 35. In a specific embodiment, the five external CSREs can have a nucleic acid sequence independently selected SEQ ID NO: 26, 27, 28, 29, or 30. In yet another embodiment, the five external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, and still further CSRE having the nucleic acid sequence of SEQ ID NO: 30. In some embodiments, the engineered promoter comprising five external CSREs can have the nucleic acid sequence of SEQ ID NO: 9 or 15.

In an embodiment, the engineered promoters comprise at least six external carbon-source responsive elements (CSREs). In an embodiment, the engineered promoters comprise six external carbon-source responsive elements (CSREs). In embodiments, the six external CSREs can have the same nucleic acid sequence or different nucleic acid sequences. In embodiments in which the CSREs have the same nucleic acid sequence, the six external CSREs can have the nucleic acid sequence of SEQ ID NO: 26, 27, 28, 29, 30, 31, 32, 33, 34, or 35. In embodiments, at least two of the six external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the six external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the six external CSREs can have the same nucleic acid sequence and the remaining external CSREs can have different nucleic acid sequences. For example, four of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least five of the six external CSREs can have the same nucleic acid sequence and the remaining external CSRE can have a different nucleic acid sequence. For example, five of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, five of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, five of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, five of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, five of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, five of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, five of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, five of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, five of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, five of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the six external CSREs can have different nucleic acid sequences. In embodiments, the six external CSREs can have a nucleic acid sequence independently selected from SEQ ID NO: 26, 27, 28, 29, 30, 31, 32, 33, 34, or 35. In a specific embodiment, the six external CSREs can have a nucleic acid sequence independently selected SEQ ID NO: 26, 27, 28, 29, 30, or 31. In yet another embodiment, the six external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, still further CSRE having the nucleic acid sequence of SEQ ID NO: 30, and yet further CSRE having the nucleic acid sequence of SEQ ID NO: 31. In some embodiments, the engineered promoter comprising six external CSREs can have the nucleic acid sequence of SEQ ID NO: 16.

In an embodiment, the engineered promoters comprise at least seven external carbon-source responsive elements (CSREs). In an embodiment, the engineered promoters comprise seven external carbon-source responsive elements (CSREs). In embodiments, the seven external CSREs can have the same nucleic acid sequence or different nucleic acid sequences. In embodiments in which the CSREs have the same nucleic acid sequence, the seven external CSREs can have the nucleic acid sequence of SEQ ID NO: 26, 27, 28, 29, 30, 31, 32, 33, 34, or 35. In embodiments, at least two of the seven external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the seven external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the seven external CSREs can have the same nucleic acid sequence and the remaining external CSREs can have different nucleic acid sequences. For example, four of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least five of the seven external CSREs can have the same nucleic acid sequence and the remaining external CSREs can have different nucleic acid sequences. For example, five of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, five of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, five of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, five of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, five of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, five of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, five of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, five of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, five of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, five of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least six of the seven external CSREs can have the same nucleic acid sequence and the remaining external CSRE can have a different nucleic acid sequence. For example, six of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, six of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, six of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, six of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, six of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, six of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, six of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, six of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, six of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, six of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the seven external CSREs can have different nucleic acid sequences. In embodiments, the seven external CSREs can have a nucleic acid sequence independently selected from SEQ ID NO: 26, 27, 28, 29, 30, 31, 32, 33, 34, or 35. In a specific embodiment, the seven external CSREs can have a nucleic acid sequence independently selected SEQ ID NO: 26, 27, 28, 29, 30, 31, or 32. In yet another embodiment, the seven external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, still further CSRE having the nucleic acid sequence of SEQ ID NO: 30, yet further CSRE having the nucleic acid sequence of SEQ ID NO: 31, and still another CSRE having the nucleic acid of SEQ ID NO: 32.

In an embodiment, the engineered promoters comprise at least eight external carbon-source responsive elements (CSREs). In an embodiment, the engineered promoters comprise eight external carbon-source responsive elements (CSREs). In embodiments, the eight external CSREs can have the same nucleic acid sequence or different nucleic acid sequences. In embodiments in which the CSREs have the same nucleic acid sequence, the eight external CSREs can have the nucleic acid sequence of SEQ ID NO: 26, 27, 28, 29, 30, 31, 32, 33, 34, or 35. In embodiments, at least two of the eight external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the eight external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the eight external CSREs can have the same nucleic acid sequence and the remaining external CSREs can have different nucleic acid sequences. For example, four of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least five of the eight external CSREs can have the same nucleic acid sequence and the remaining external CSREs can have different nucleic acid sequences. For example, five of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, five of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, five of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, five of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, five of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, five of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, five of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, five of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, five of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, five of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least six of the eight external CSREs can have the same nucleic acid sequence and the remaining external CSREs can have different nucleic acid sequences. For example, six of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, six of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, six of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, six of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, six of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, six of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, six of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, six of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, six of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, six of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least seven of the eight external CSREs can have the same nucleic acid sequence and the remaining external CSRE can have a different nucleic acid sequence. For example, seven of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, seven of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, seven of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, seven of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, seven of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, seven of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, seven of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, seven of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, seven of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, seven of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the eight external CSREs can have different nucleic acid sequences. In embodiments, the eight external CSREs can have a nucleic acid sequence independently selected from SEQ ID NO: 26, 27, 28, 29, 30, 31, 32, 33, 34, or 35. In a specific embodiment, the eight external CSREs can have a nucleic acid sequence independently selected SEQ ID NO: 26, 27, 28, 29, 30, 31, 32, or 33. In yet another embodiment, the eight external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, still further CSRE having the nucleic acid sequence of SEQ ID NO: 30, yet further CSRE having the nucleic acid sequence of SEQ ID NO: 31, still another CSRE having the nucleic acid of SEQ ID NO: 32, another CSRE having the nucleic acid sequence of SEQ ID NO: 33.

In an embodiment, the engineered promoters comprise at least nine external carbon-source responsive elements (CSREs). In an embodiment, the engineered promoters comprise nine external carbon-source responsive elements (CSREs). In embodiments, the nine external CSREs can have the same nucleic acid sequence or different nucleic acid sequences. In embodiments in which the CSREs have the same nucleic acid sequence, the nine external CSREs can have the nucleic acid sequence of SEQ ID NO: 26, 27, 28, 29, 30, 31, 32, 33, 34, or 35. In embodiments, at least two of the nine external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the nine external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the nine external CSREs can have the same nucleic acid sequence and the remaining external CSREs can have different nucleic acid sequences. For example, four of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least five of the nine external CSREs can have the same nucleic acid sequence and the remaining external CSREs can have different nucleic acid sequences. For example, five of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, five of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, five of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, five of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, five of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, five of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, five of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, five of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, five of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, five of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least six of the nine external CSREs can have the same nucleic acid sequence and the remaining external CSREs can have different nucleic acid sequences. For example, six of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, six of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, six of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, six of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, six of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, six of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, six of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, six of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, six of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, six of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least seven of the nine external CSREs can have the same nucleic acid sequence and the remaining external CSREs can have different nucleic acid sequences. For example, seven of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, seven of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, seven of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, seven of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, seven of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, seven of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, seven of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, seven of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, seven of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, seven of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least eight of the nine external CSREs can have the same nucleic acid sequence and the remaining external CSRE can have a different nucleic acid sequence. For example, eight of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, eight of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, eight of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, eight of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, eight of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, eight of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, eight of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, eight of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, eight of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, eight of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the nine external CSREs can have different nucleic acid sequences. In embodiments, the nine external CSREs can have a nucleic acid sequence independently selected from SEQ ID NO: 26, 27, 28, 29, 30, 31, 32, 33, 34, or 35. In a specific embodiment, the nine external CSREs can have a nucleic acid sequence independently selected SEQ ID NO: 26, 27, 28, 29, 30, 31, 32, 33, or 34. In yet another embodiment, the nine external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, still further CSRE having the nucleic acid sequence of SEQ ID NO: 30, yet further CSRE having the nucleic acid sequence of SEQ ID NO: 31, still another CSRE having the nucleic acid of SEQ ID NO: 32, another CSRE having the nucleic acid sequence of SEQ ID NO: 33; and still another CSRE having the nucleic acid sequence of SEQ ID NO: 34.

In an embodiment, the engineered promoters comprise at least ten external carbon-source responsive elements (CSREs). In an embodiment, the engineered promoters comprise ten external carbon-source responsive elements (CSREs). In an embodiment, the engineered promoters comprise more than ten external carbon-source responsive elements (CSREs). In embodiments, the ten external CSREs can have the same nucleic acid sequence or different nucleic acid sequences. In embodiments in which the CSREs have the same nucleic acid sequence, the ten external CSREs can have the nucleic acid sequence of SEQ ID NO: 26, 27, 28, 29, 30, 31, 32, 33, 34, or 35. In embodiments, at least two of the ten external CSREs can have the same nucleic acid sequence and the remaining external CSREs can have different nucleic acid sequences. For example, two of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, two of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, two of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, two of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, two of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, two of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, two of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, two of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, two of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, two of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least three of the ten external CSREs can have the same nucleic acid sequence and the remaining external CSREs can have different nucleic acid sequences. For example, three of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, three of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, three of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, three of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, three of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, three of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, three of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, three of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, three of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, three of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least four of the ten external CSREs can have the same nucleic acid sequence and the remaining external CSREs can have different nucleic acid sequences. For example, four of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, four of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, four of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, four of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, four of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, four of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, four of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, four of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, four of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, four of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least five of the ten external CSREs can have the same nucleic acid sequence and the remaining external CSREs can have different nucleic acid sequences. For example, five of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, five of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, five of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, five of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, five of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, five of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, five of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, five of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, five of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, five of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least six of the ten external CSREs can have the same nucleic acid sequence and the remaining external CSREs can have different nucleic acid sequences. For example, six of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, six of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, six of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, six of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, six of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, six of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, six of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, six of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, six of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, six of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least seven of the ten external CSREs can have the same nucleic acid sequence and the remaining external CSREs can have different nucleic acid sequences. For example, seven of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, seven of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, seven of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, seven of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, seven of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, seven of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, seven of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, seven of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, seven of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, seven of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least eight of the ten external CSREs can have the same nucleic acid sequence and the remaining external CSREs can have different nucleic acid sequences. For example, eight of the external CSREs can have the nucleic acid sequence of SEQ ID NO: 26 and the remaining external CSREs are independently selected from the nucleic acid sequence of any one of SEQ ID NO: 27, 28, 29, 30, 31, 32, 33, 34, or 35. In another example, eight of the external CSREs can have the nucleic acid sequence of SEQ ID NO: 27 and the remaining external CSREs are independently selected from the nucleic acid sequence of any one of SEQ ID NO: 26, 28, 29, 30, 31, 32, 33, 34, or 35. In still another example, eight of the external CSREs can have the nucleic acid sequence of SEQ ID NO: 28 and the remaining external CSREs are independently selected from the nucleic acid sequence of any one of SEQ ID NO: 26, 27, 29, 30, 31, 32, 33, 34, or 35. In yet another example, eight of the external CSREs can have the nucleic acid sequence of SEQ ID NO: 29 and the remaining external CSREs are independently selected from the nucleic acid sequence of any one of SEQ ID NO: 26, 27, 28, 30, 31, 32, 33, 34, or 35. In an example, eight of the external CSREs can have the nucleic acid sequence of SEQ ID NO: 30 and the remaining external CSREs are independently selected from the nucleic acid sequence of any one of SEQ ID NO: 26, 27, 28, 29, 31, 32, 33, 34, or 35. In an example, eight of the external CSREs can have the nucleic acid sequence of SEQ ID NO: 31 and the remaining external CSREs are independently selected from the nucleic acid sequence of any one of SEQ ID NO: 26, 27, 28, 29, 30, 32, 33, 34, or 35. In another example, eight of the external CSREs can have the nucleic acid sequence of SEQ ID NO: 32 and the remaining external CSREs are independently selected from the nucleic acid sequence of any one of SEQ ID NO: 26, 27, 28, 29, 30, 31, 33, 34, or 35. In an example, eight of the external CSREs can have the nucleic acid sequence of SEQ ID NO: 33 and the remaining external CSREs are independently selected from the nucleic acid sequence of any one of SEQ ID NO: 26, 27, 28, 29, 30, 31, 32, 34, or 35. In an example, eight of the external CSREs can have the nucleic acid sequence of SEQ ID NO: 34 and the remaining external CSREs are independently selected from the nucleic acid sequence of any one of SEQ ID NO: 26, 27, 28, 29, 30, 31, 32, 33, or 35. In an example, eight of the external CSREs can have the nucleic acid sequence of SEQ ID NO: 35 and the remaining external CSREs are independently selected from the nucleic acid sequence of any one of SEQ ID NO: 26, 27, 28, 29, 30, 31, 32, 33, or 34. In embodiments, at least nine of the ten external CSREs can have the same nucleic acid sequence and the remaining external CSREs can have different nucleic acid sequences. For example, nine of the external CSREs can have the nucleic acid sequence of SEQ ID NO: 26 and the remaining external CSRE is independently selected from the nucleic acid sequence of any one of SEQ ID NO: 27, 28, 29, 30, 31, 32, 33, 34, or 35. In another example, nine of the external CSREs can have the nucleic acid sequence of SEQ ID NO: 27 and the remaining external CSRE is independently selected from the nucleic acid sequence of any one of SEQ ID NO: 26, 28, 29, 30, 31, 32, 33, 34, or 35. In still another example, nine of the external CSREs can have the nucleic acid sequence of SEQ ID NO: 28 and the remaining external CSRE is independently selected from the nucleic acid sequence of any one of SEQ ID NO: 26, 27, 29, 30, 31, 32, 33, 34, or 35. In yet another example, nine of the external CSREs can have the nucleic acid sequence of SEQ ID NO: 29 and the remaining external CSRE is independently selected from the nucleic acid sequence of any one of SEQ ID NO: 26, 27, 28, 30, 31, 32, 33, 34, or 35. In an example, nine of the external CSREs can have the nucleic acid sequence of SEQ ID NO: 30 and the remaining external CSRE is independently selected from the nucleic acid sequence of any one of SEQ ID NO: 26, 27, 28, 29, 31, 32, 33, 34, or 35. In an example, nine of the external CSREs can have the nucleic acid sequence of SEQ ID NO: 31 and the remaining external CSRE is independently selected from the nucleic acid sequence of any one of SEQ ID NO: 26, 27, 28, 29, 30, 32, 33, 34, or 35. In another example, nine of the external CSREs can have the nucleic acid sequence of SEQ ID NO: 32 and the remaining external CSRE is independently selected from the nucleic acid sequence of any one of SEQ ID NO: 26, 27, 28, 29, 30, 31, 33, 34, or 35. In an example, nine of the external CSREs can have the nucleic acid sequence of SEQ ID NO: 33 and the remaining external CSRE is independently selected from the nucleic acid sequence of any one of SEQ ID NO: 26, 27, 28, 29, 30, 31, 32, 34, or 35. In an example, nine of the external CSREs can have the nucleic acid sequence of SEQ ID NO: 34 and the remaining external CSRE is independently selected from the nucleic acid sequence of any one of SEQ ID NO: 26, 27, 28, 29, 30, 31, 32, 33, or 35. In an example, nine of the external CSREs can have the nucleic acid sequence of SEQ ID NO: 35 and the remaining external CSRE is independently selected from the nucleic acid sequence of any one of SEQ ID NO: 26, 27, 28, 29, 30, 31, 32, 33, or 34. In embodiments, the ten external CSREs can have different nucleic acid sequences. In embodiments, the ten external CSREs can have a nucleic acid sequence independently selected from SEQ ID NO: 26, 27, 28, 29, 30, 31, 32, 33, 34, or 35. In a specific embodiment, the ten external CSREs can have a nucleic acid sequence independently selected SEQ ID NO: 26, 27, 28, 29, 30, 31, 32, 33, 34, or 35. In yet another embodiment, the ten external CSREs can have a distinct nucleic acid sequence, one CSRE having the nucleic acid sequence of SEQ ID NO: 26, another CSRE having the nucleic acid sequence of SEQ ID NO: 27, a further CSRE having the nucleic acid sequence of SEQ ID NO: 28, yet another CSRE having the nucleic acid sequence of SEQ ID NO: 29, still further CSRE having the nucleic acid sequence of SEQ ID NO: 30, yet further CSRE having the nucleic acid sequence of SEQ ID NO: 31, still another CSRE having the nucleic acid of SEQ ID NO: 32, another CSRE having the nucleic acid sequence of SEQ ID NO: 33; still another CSRE having the nucleic acid sequence of SEQ ID NO: 34, and still further CSRE having the nucleic acid sequence of SEQ ID NO: 35. In some embodiments, the engineered promoter comprising ten external CSREs can have the nucleic acid sequence of SEQ ID NO: 10.

The engineered promoters of the present disclosure are derived from a parental promoter. In embodiments, the parental promoter is not repressed in aerobic conditions and/or in the presence of a C2 carbon source like ethanol. The parental promoter may be a promoter found in native form in any living organism or can be a synthetic promoter. The parental promoter may already include one or more carbon-source responsive element (CSRE) as described herein. The parental promoter may lack any CSRE. In some specific embodiments, the parental promoter is an inducible promoter, e.g., the expression of the gene to which it is operatively linked is increased when the recombinant yeast host cell is placed in contact with an inducer or a combination of inducers. In some specific embodiments, the parental promoter is a C2 carbon source inducible promoter (like an ethanol inducible promoter), e.g., the expression of the gene to which it is operatively linked is increased when the recombinant yeast host cell is placed in contact with the inducer (a C2 carbon source like ethanol). In some embodiments, the parental promoter is not an ethanol inducible promoter, e.g., the expression of the gene to which it is operatively linked is not increased (e.g., remains substantially the same or is decreased) when the recombinant yeast host cell is placed in contact with the inducer. In some specific embodiments, the parental promoter is a glucose inducible promoter, e.g., the expression of the gene to which it is operatively linked is increased when the recombinant yeast host cell is placed in contact with glucose (e.g., the inducer). In some specific embodiments, the parental promoter is a fructose inducible promoter, e.g., the expression of the gene to which it is operatively linked is increased when the recombinant yeast host cell is placed in contact with fructose (e.g., the inducer). In some specific embodiments, the parental promoter is a glycerol inducible promoter, e.g., the expression of the gene to which is operatively linked is increased when the recombinant yeast host cell is placed in contact with glycerol (e.g., the inducer). In some specific embodiments, the parental promoter is a constitutive promoter, e.g. a promoter whose expression strength remains substantially the same irrespective of the presence or absence of an inducer (e.g., a C2 carbon source like ethanol) or a non-C2 carbon source carbon source (like glucose, fructose, or glycerol for example).

In embodiments in which the recombinant microbial host cell is from Saccharomyces cerevisiae, the parental promoter can be an inducible or a constitutive promoter. In such embodiments, the parental promoter can be obtained or derived from a native promoter present in Saccharomyces cerevisiae. Inducible promoters include, but are not limited to glucose-regulated promoters (e.g., the promoter of the hxt7 gene (referred to as hxt7p); the promoter of the ctt1 gene (referred to as ctt1p); the promoter of the glo1 gene (referred to as glo1p); the promoter of the ygp1 gene (referred to as ygp1p); the promoter of the gsy2 gene (referred to as gsy2p); the promoter of the gpm1 gene (referred to as gpm1p), the promoter of the pgk1 gene (referred to as pgk1p)), molasses-regulated promoters (e.g., the promoter of the mol1 gene (referred to as mol1p)), heat shock-regulated promoters (e.g., the promoter of the glo1 gene (referred to as glo1p); the promoter of the sti1 gene (referred to as sti1p); the promoter of the ygp1 gene (referred to as ygp1p); the promoter of the gsy2 gene (referred to as gsy2p)), oxidative stress response promoters (e.g., the promoter of the cup1 gene (referred to as cup1p); the promoter of the ctt1 gene (referred to as ctt1p); the promoter of the trx2 gene (referred to as trx2p); the promoter of the gpd1 gene (referred to as gpd1p); the promoter of the hsp12 gene (referred to as hsp12p); the promoter of the hsp150 gene (referred to as hsp150p); the promoter of the ssc1 gene (referred to as ssc1p)), osmotic stress response promoters (e.g., the promoter of the ctt1 gene (referred to as ctt1p); the promoter of the glo1 gene (referred to as glo1p); the promoter of the gpd1 gene (referred to as gpd1p); the promoter of the ygp1 gene (referred to as ygp1p); the promoter of the hor7 gene (referred to as hor7p); the promoter of the stl1 gene (referred to as stl1p)), nitrogen-regulated promoters (e.g., the promoter of the ygp1 gene (referred to as ygp1p); the promoter of the adh1 gene (referred to as adh1p)); anaerobic-regulated promoters (e.g., the promoter of the tir1 gene (referred to as tir1p), the promoter of the pau5 gene (referred to as pau5p), the promoter of the dan1 gene (referred to as dan1p), the promoter of the tdh1 gene (referred to as tdh1p), the promoter of the spi1 gene (referred to as spi1p), the promoter of the hxk1 gene (referred to as hxk1p), the promoter of the anb1 gene (referred to as anb1p), the promoter of the hxt6 gene (referred to as hxt6p), the promoter of the trx1 gene (referred to as trx1p), the promoter of the aac3 gene (referred to as aac3p), the promoter of the hor7 gene (referred to as hor7p), the promoter of the adh1 gene (referred to as adh1p), the promoter of the tdh2 gene (referred to as tdh2p), the promoter of the tdh3 gene (referred to as tdh3p), the promoter of the gdp1 gene (referred to as gpd1p), the promoter of the cdc19 gene (referred to as cdc19p), the promoter of the eno2 gene (referred to as eno2p), the promoter of the pdc1 gene (referred to as pdc1p), the promoter of the hxt3 gene (referred to as hxt3p), or the promoter of the tpi1 gene (referred to tpi1p)); ethanol-regulated promoters (including ethanol responsive promoters); redox-regulated promoters (including, but not limited to the promoter of the gpd2 gene (referred as gpd2p)); sulfite-regulated promoters (including, but not limited to the promoter of the fzf1 gene (referred to as the fzf1p), the promoter of the ssu1 gene (referred to as ssu1p), and the promoter of the ssu1-r gene (referred to as the ssu1-rp)); and stress-response promoters (including, but not limited to the promoter of the yap1 gene (referred to as yap1p), the promoter of the ssa3 gene (referred to as ssa3p), and the promoter of the hsp104 gene (referred to as hsp104p)). Constitutive promoters include, but are not limited to the promoter of the tef2 gene (referred to as tef2p), the promoter of the cwp2 gene (referred to as cwp2p), the promoter of the ssa1 gene (referred to as ssa1p), the promoter of the eno1 gene (referred to as eno1p), the promoter of the hxk1 gene (referred to as hxk1p), the promoter of the pgk1 gene (referred to as pgk1p), the promoter of the adh1 gene (referred to as adh1p), the promoter of the rev1 gene (referred to as rev1p), the promoter of the cyc1 gene (referred to as cyc1p), and the promoter of the ste5 gene (referred to as ste5p).

In embodiments in which the recombinant microbial host cell is a methylotrophic yeast (like Komagataella phaffii or Ogataea polymorpha), the parental promoter can be an inducible or a constitutive promoter. In such embodiments, the parental promoter can be obtained or derived from a native promoter present in Komagataella phaffii. Inducible promoters include, but are not limited to glucose-regulated promoters, fructose-regulated promoters, glycerol-regulated promoters, heat shock-regulated promoters, oxidative stress response promoters, osmotic stress response promoters, nitrogen-regulated promoters, and ethanol-regulated promoters. In an embodiment, ethanol-regulated promoters include, without limitation, the promoter from the adh2 gene, which is also known as the adh3 gene (referred to as adh2p). Constitutive promoters include, without limitation, the promoter from the spi1 gene (referred to as spi1p). In an embodiment, the parental promoter is a promoter from the gap1 gene (referred to as gap1p). In an embodiment, the parental promoter is a promoter from the hgt1 gene (referred to as hgt1p). In an embodiment, the parental promoter is a promoter from the glc3 gene (referred to as glc3p). In an embodiment, the parental promoter is a promoter from the acb2 gene (referred to as acb2p). In an embodiment, the parental promoter is a promoter from the pex8 gene (referred to as pex8p). In an embodiment, the parental promoter is a promoter from the urc1 gene (referred to as urc1p). In an embodiment, the parental promoter is a promoter from the tpo3 gene (referred to as top3p). In an embodiment, the parental promoter is a promoter from the bio2 gene (referred to as bio2p). In an embodiment, the parental promoter is a promoter from the gut1 gene (referred to as gut1p). In an embodiment, the parental promoter is a promoter from the cat1 gene (referred to as cat1p). In an embodiment, the parental promoter is a promoter from the icl1 gene (referred to as icl1p). In an embodiment, the parental promoter is a promoter from the gcw14 gene (referred to as gcw14p). In an embodiment, the parental promoter is a promoter from the sor1 gene (referred to as sor1p), the O. polymorpha methanol oxidase mox1 gene (referred to as mox1p), the O. polymorpha promoter from the gap1 gene (referred to as OpGAP1p), the O. polymorpha promoter from the gapdh gene (referred to as OpGAPDHp), the O. polymorpha promoter from the gcw14 gene (referred to as OpGCW14p), the O. Polymorpha promoter from the adh1 gene (referred to as OpADH1p), the O. polymorpha promoter from the icl1 gene (referred to as OpICL1p), or the O. polymorpha promoter from the tef1 gene (referred to as OpTEF1p).

The engineered promoters of the present disclosure are intended to be operatively linked (or associated) with a gene to increase/drive its expression in the presence of an inducer (a C2 carbon source like ethanol), and/or derepressed by a non-C2 carbon source like glucose, fructose or glycerol. In the heterologous nucleic acid molecule described herein, the promoter and the nucleic acid molecule comprising the gene are operatively linked to one another. In the context of the present disclosure, the expressions “operatively linked” or “operatively associated” refers to fact that the engineered promoter is physically associated (e.g., in a cis orientation) to the gene in a manner that allows or increases, in the presence of a C2 carbon source like ethanol, for expression of the gene. The engineered promoter(s) is/are usually located upstream (5′) of the gene. As such, the gene operatively linked to the promoter is usually located downstream (3′) of the engineered promoter. In the context of the present disclosure, one or more than one engineered promoter can be used for expressing the operatively linked gene. When more than one promoter is included, each of the promoters is operatively linked to the gene. In the context of the present disclosure, one or more than one genes (e.g., an operon) can be operatively linked to the engineered promoter(s).

The gene(s) operatively linked to the engineered promoter(s) can encode a polypeptide (which can be, in some embodiments, an enzyme) or an RNA molecule (a transfer RNA (tRNA), a ribosomal RNA (rRNA), a guide RNA (gRNA), a small nuclear RNA (snRNA) or a ribozyme for example).

The engineered promoters of the present disclosure have an upstream boundary and a downstream boundary. The upstream boundary is located in the 5′ direction of the engineered promoter. The engineered promoters extend upstream so as to include the elements necessary to initiate/drive transcription in the presence of the inducer (a C2 carbon source like ethanol) or a non-C2 carbon source (glucose, fructose or glycerol for example). The downstream boundary is located in the 3′ direction of the promoter and is intended to be operatively linked to the upstream boundary (located in the 5′ direction) of the gene intended to be expressed. Promoters usually include a core promoter defined as the minimal region required to direct initiation of transcription. Within the core promoter will be found polypeptide binding domains (consensus sequences) responsible for the binding of the RNA polymerase, the transcription start site (TSS), as well as a 5′ untranslated region (5′ UTR, which can also be referred to as a leader sequence). In some embodiments of eukaryotic and archaeal promoters, the core promoter includes a TATA box (which may have been previously validated or is putative) which defines a binding site of the TATA-binding proteins and, ultimately, of the RNA polymerase. The TATA box can be located, as it is known in the art, by determining the presence of a TATA consensus sequence in the core promoter. In yeasts, the TATA consensus sequence (which has been derived from the S. cerevisiae consensus sequence) has the nucleic acid sequence of Formula (II):

TATANₐAN_{b}N_{c} (II)
wherein Nₐ is A or T
   N_{b} is A or T
   N_{c} is A or G

When the gene to be expressed under the control of the engineered promoters encodes a polypeptide, it includes an open reading frame (ORF) as well as a start site (e.g., a start codon). In the promoter from the K. phaffii adh2 gene, and the most 5′ nucleotide of the TATA box is located 82 base pairs upstream (−82) of the ORF's start site. In the promoter from the K. phaffii spi1 gene, the most 5′ nucleotide of the TATA box is located 93 base pairs upstream (−93) of the ORF start site, and the TSS is located 45 base pairs upstream (−45) of the ORF's start site. In the promoter from the K. phaffii gap1 gene, the most 5′ nucleotide of the TATA box is located 69 base pairs upstream (−69) of the ORF's start site. In the promoter from the K. phaffii hgt1 gene, the most 5′ nucleotide of the TATA box is located 64 base pairs upstream (−64) of the ORF's start site. In the promoter from the K. phaffii glc3 gene, the most 5′ nucleotide of the TATA box is located 57 base pairs upstream (−57) of the ORF's start site. In the promoter from the K. phaffii acb2 gene, the most 5′ nucleotide of the TATA box is located 60 base pairs upstream (−60) of the ORF's start site. In the promoter from the K. phaffii pex8 gene, the most 5′ nucleotide of the TATA box is located 73 base pairs upstream (−73) of the ORF's start site. In the promoter from the K. phaffii urc1 gene, the most 5′ nucleotide of the TATA box is located 90 base pairs upstream (−90) of the ORF's start site. In the promoter from the K. phaffii tpo3 gene, the most 5′ nucleotide of the TATA box is located 70 base pairs upstream (−70) of the ORF's start site. In the promoter from the K. phaffii bio2 gene, the most 5′ nucleotide of the TATA box is located 68 base pairs upstream (−68) of the ORF's start site. In the promoter from the K. phaffii gut1 gene, the most 5′ nucleotide of the TATA box is located 60 base pairs upstream (−60) of the ORF's start site. In the promoter from the K. phaffii cat1 gene, the most 5′ nucleotide of the TATA box is located 78 base pairs upstream (−78) of the ORF's start site. In the promoter from the K. phaffii icl1 gene, the most 5′ nucleotide of the TATA box is located 96 base pairs upstream (−96) of the ORF's start site. In the promoter from the K. phaffii gcw14 gene, the most 5′ nucleotide of the TATA box is located 93 base pairs upstream (−93) of the ORF's start site. In embodiments of bacterial promoters, the core promoter includes a Pribnow box, which defines the region in which the RNA polymerase will initially bind.

As indicated above, promoters (engineered and parental) have, close to their 3′ boundary, a transcription start site (TSS). The TSS of a promoter can be determined, as known in the art, by mapping with nuclease S1. Promoters also include, a 5′ UTR is located downstream the TSS. While the 5′ UTR is transcribed into a coding strand of a mRNA, it is usually not transcribed into a polypeptide. In such embodiments, the 5′ UTR is located between the TSS and the gene to be expressed.

In the context of the present disclosure, the engineered promoter comprises a first external CSRE and such first external CSRE(s) is located upstream (5′ direction) of the transcription start site. Consequently, the first external CSRE is not located in the 5′UTR region of the parental promoter. In some embodiments, none of the external CSREs that may be present in the engineered promoters are located within the core region of the engineered promoter. In some embodiments, only one of the external CSREs is located within the core region of the engineered promoter. In other embodiments, all of the external CSRE is located outside and upstream (5′ direction) of the core promoter of the engineered promoters.

Still in the context of the present disclosure, the first external CSRE is located proximal to the transcription start site of the parental promoter. As it will be shown herein, the location of the first external CSRE, with respect to the transcription start site, has an influence with respect to the ability of the resulting engineered promoter to promote the transcription of the gene operatively linked thereto. In the context of the present disclosure the term “proximal” indicates that the location of the first external CSRE is close enough to the transcription start site (and by extension to the TATA box or the start codon) to increase, in the presence of an inducer (a C2 carbon source like ethanol), or a non-C2 carbon source (glucose, glycerol or fructose for example), the transcription of the operatively linked gene and/or the stability of the RNA molecules being transcribed. This increase in transcription can be determined, in some embodiments, by measuring the level of transcription of the gene operatively linked to the engineered promoters. In addition, this increase in transcription can be determined by measuring the amount/activity of the polypeptide encoded by the gene operatively linked to the engineered promoter. In instances in which the gene encodes an enzyme, this increase in transcription can be determined by measuring the enzymatic activity of the polypeptide encoded by the gene operatively linked to the engineered promoter.

In the context of the present disclosure, the insertion position of the at least one external CSRE will be defined by either providing its most upstream position with respect to a transcription start site, a TATA box, or a start codon or by referring to a specific region upstream of a transcription start site, a TATA box, or a start codon. For example, the expression “the at least one external CSRE is located at most XX base pairs upstream of the transcription start site/TATA box/start codon” indicates that the position of insertion of the at least one CSRE is located between the position immediately upstream of the transcription start site/TATA box/start codon and the XX^{th} base pairs upstream of the transcription start site/TATA box/start codon. In another example, the expression “the at least one external CSRE is located between about position AA upstream of the transcription start site/TATA box/start codon and about position BB upstream of the transcription start site/TATA box/start codon” indicates that the position of insertion of the at least one CSRE is located between the AA^{th} base pairs upstream of the transcription start site/TATA box/start codon and the BB^{th} position with respect to the transcription start site/TATA box/start codon.

In some embodiments, the engineered promoter of the present disclosure comprises a first external CSRE located at most 397 base pairs upstream (e.g., −397) of the transcription start site. In some instances, the engineered promoter can include one or more external CSREs which can be located more than 397 base pairs upstream (e.g., −397) of the transcription start site (provided that it includes at least one external CSRE at most 397 base pair upstream of the transcription start site). In some additional embodiments, the engineered promoter comprises a first external CSRE located at most 350 base pairs upstream (e.g., −350) of the transcription start site. In some instances, the engineered promoter can include one or more external CSREs which can be located more than 350 base pairs upstream (e.g., −350) of the transcription start site (provided that it includes at least one external CSRE at most 350 base pair upstream of the transcription start site).

In some embodiments, the first external CSRE is located between the transcription start site and about 397 base pairs upstream (e.g., −397) of the transcription start site. In some alternative embodiments, the first external CSRE is located between about 7 base pairs upstream (e.g., −7) of the transcription start site and about 350 base pairs upstream (e.g., −397) of the transcription start site. In some specific embodiments, the first external CSRE is located between about 7 base pairs upstream (e.g., −7) of the transcription start site and about 86 base pairs upstream (e.g., −86) of the transcription start site. In some additional embodiments, the first external CSRE is located between 46 base pairs upstream (e.g., −46) of the transcription start site and 47 base pairs upstream (e.g., −47) of the transcription start site. In some specific embodiments, the first external CSRE is located between about 37 base pairs upstream (e.g., −37) of the transcription start site and about 116 base pairs upstream (e.g., −116) of the transcription start site. In some additional embodiments, the first external CSRE is located between 76 base pairs upstream (e.g., −76) of the transcription start site and 77 base pairs upstream (e.g., −77) of the transcription start site. In some specific embodiments, the first external CSRE is located between about 47 base pairs upstream (e.g., −47) of the transcription start site and about 126 base pairs upstream (e.g., −126) of the transcription start site. In some additional embodiments, the first external CSRE is located between 86 base pairs upstream (e.g., −86) of the transcription start site and 87 base pairs upstream (e.g., −87) of the transcription start site. In some specific embodiments, the first external CSRE is located between about 55 base pairs upstream (e.g., −55) of the transcription start site and about 134 base pairs upstream (e.g., −134) of the transcription start site. In some additional embodiments, the first external CSRE is located between 94 base pairs upstream (e.g., −94) of the transcription start site and 95 base pairs upstream (e.g., −95) of the transcription start site. In some specific embodiments, the first external CSRE is located between about 77 base pairs upstream (e.g., −77) of the transcription start site and about 156 base pairs upstream (e.g., −156) of the transcription start site. In some additional embodiments, the first external CSRE is located between 116 base pairs upstream (e.g., −116) of the transcription start site and 117 base pairs upstream (e.g., −117) of the transcription start site. In some specific embodiments, the first external CSRE is located between about 81 base pairs upstream (e.g., −81) of the transcription start site and about 160 base pairs upstream (e.g., −160) of the transcription start site. In some additional embodiments, the first external CSRE is located between 120 base pairs upstream (e.g., −120) of the transcription start site and 121 base pairs upstream (e.g., −121) of the transcription start site. In some specific embodiments, the first external CSRE is located between about 87 base pairs upstream (e.g., −87) of the transcription start site and about 166 base pairs upstream (e.g., −166) of the transcription start site. In some additional embodiments, the first external CSRE is located between 126 base pairs upstream (e.g., −126) of the transcription start site and 127 base pairs upstream (e.g., −127) of the transcription start site. In some specific embodiments, the first external CSRE is located between about 93 base pairs upstream (e.g., −93) of the transcription start site and about 172 base pairs upstream (e.g., −172) of the transcription start site. In some additional embodiments, the first external CSRE is located between 132 base pairs upstream (e.g., −132) of the transcription start site and 133 base pairs upstream (e.g., −133) of the transcription start site. In some specific embodiments, the first external CSRE is located between about 94 base pairs upstream (e.g., −94) of the transcription start site and about 173 base pairs upstream (e.g., −173) of the transcription start site. In some additional embodiments, the first external CSRE is located between 133 base pairs upstream (e.g., −133) of the transcription start site and 134 base pairs upstream (e.g., −134) of the transcription start site. In some specific embodiments, the first external CSRE is located between about 99 base pairs upstream (e.g., −99) of the transcription start site and about 178 base pairs upstream (e.g., −178) of the transcription start site. In some additional embodiments, the first external CSRE is located between 138 base pairs upstream (e.g., −138) of the transcription start site and 139 base pairs upstream (e.g., −139) of the transcription start site. In some specific embodiments, the first external CSRE is located between about 104 base pairs upstream (e.g., −104) of the transcription start site and about 183 base pairs upstream (e.g., −183) of the transcription start site. In some additional embodiments, the first external CSRE is located between 143 base pairs upstream (e.g., −143) of the transcription start site and 144 base pairs upstream (e.g., −144) of the transcription start site. In some specific embodiments, the first external CSRE is located between about 109 base pairs upstream (e.g., −109) of the transcription start site and about 188 base pairs upstream (e.g., −188) of the transcription start site. In some additional embodiments, the first external CSRE is located between 148 base pairs upstream (e.g., −148) of the transcription start site and 149 base pairs upstream (e.g., −149) of the transcription start site. In some specific embodiments, the first external CSRE is located between about 111 base pairs upstream (e.g., −111) of the transcription start site and about 190 base pairs upstream (e.g., −190) of the transcription start site. In some additional embodiments, the first external CSRE is located between 150 base pairs upstream (e.g., −150) of the transcription start site and 151 base pairs upstream (e.g., −151) of the transcription start site. In some specific embodiments, the first external CSRE is located between about 114 base pairs upstream (e.g., −114) of the transcription start site and about 193 base pairs upstream (e.g., −193) of the transcription start site. In some additional embodiments, the first external CSRE is located between 153 base pairs upstream (e.g., −153) of the transcription start site and 154 base pairs upstream (e.g., −154) of the transcription start site. In some specific embodiments, the first external CSRE is located between about 119 base pairs upstream (e.g., −119) of the transcription start site and about 198 base pairs upstream (e.g., −198) of the transcription start site. In some additional embodiments, the first external CSRE is located between 158 base pairs upstream (e.g., −158) of the transcription start site and 159 base pairs upstream (e.g., −159) of the transcription start site. In some specific embodiments, the first external CSRE is located between about 124 base pairs upstream (e.g., −124) of the transcription start site and about 203 base pairs upstream (e.g., −203) of the transcription start site. In some additional embodiments, the first external CSRE is located between 163 base pairs upstream (e.g., −163) of the transcription start site and 164 base pairs upstream (e.g., −164) of the transcription start site. In some specific embodiments, the first external CSRE is located between about 129 base pairs upstream (e.g., −129) of the transcription start site and about 208 base pairs upstream (e.g., −208) of the transcription start site. In some additional embodiments, the first external CSRE is located between 168 base pairs upstream (e.g., −168) of the transcription start site and 169 base pairs upstream (e.g., −169) of the transcription start site. In some specific embodiments, the first external CSRE is located between about 189 base pairs upstream (e.g., −189) of the transcription start site and about 268 base pairs upstream (e.g., −268) of the transcription start site. In some additional embodiments, the first external CSRE is located between 228 base pairs upstream (e.g., −228) of the transcription start site and 229 base pairs upstream (e.g., −229) of the transcription start site. In some specific embodiments, the first external CSRE is located between about 198 base pairs upstream (e.g., −198) of the transcription start site and about 277 base pairs upstream (e.g., −277) of the transcription start site. In some additional embodiments, the first external CSRE is located between 237 base pairs upstream (e.g., −237) of the transcription start site and 238 base pairs upstream (e.g., −238) of the transcription start site. In some specific embodiments, the first external CSRE is located between about 239 base pairs upstream (e.g., −239) of the transcription start site and about 318 base pairs upstream (e.g., −318) of the transcription start site. In some additional embodiments, the first external CSRE is located between 278 base pairs upstream (e.g., −278) of the transcription start site and 279 base pairs upstream (e.g., −279) of the transcription start site. In some specific embodiments, the first external CSRE is located between about 318 base pairs upstream (e.g., −318) of the transcription start site and about 397 base pairs upstream (e.g., −397) of the transcription start site. In some additional embodiments, the first external CSRE is located between 357 base pairs upstream (e.g., −357) of the transcription start site and 358 base pairs upstream (e.g., −358) of the transcription start site.

In some embodiments, the engineered promoter of the present disclosure comprises at least two external CSREs. Embodiments of the location and the nucleic acid sequence of the first external CSRE are provided herein and can be used in an engineered promoter comprising two or more external CSREs. When a plurality of CSREs is included in the engineered promoter, the CSREs can be independently located at the same position and be contiguous or be provided at different positions and be non-contiguous. In some embodiments, the second external CSRE is located between the transcription start site and about 397 base pairs upstream (e.g., −397) of the transcription start site. In some alternative embodiments, the second external CSRE is located between about 7 base pairs upstream (e.g., −7) of the transcription start site and about 350 base pairs upstream (e.g., −397) of the transcription start site. In some specific embodiments, the second external CSRE is located between about 7 base pairs upstream (e.g., −7) of the transcription start site and about 86 base pairs upstream (e.g., −86) of the transcription start site. In some additional embodiments, the second external CSRE is located between 46 base pairs upstream (e.g., −46) of the transcription start site and 47 base pairs upstream (e.g., −47) of the transcription start site. In some specific embodiments, the second external CSRE is located between about 37 base pairs upstream (e.g., −37) of the transcription start site and about 116 base pairs upstream (e.g., −116) of the transcription start site. In some additional embodiments, the second external CSRE is located between 76 base pairs upstream (e.g., −76) of the transcription start site and 77 base pairs upstream (e.g., −77) of the transcription start site. In some specific embodiments, the second external CSRE is located between about 47 base pairs upstream (e.g., −47) of the transcription start site and about 126 base pairs upstream (e.g., −126) of the transcription start site. In some additional embodiments, the second external CSRE is located between 86 base pairs upstream (e.g., −86) of the transcription start site and 87 base pairs upstream (e.g., −87) of the transcription start site. In some specific embodiments, the second external CSRE is located between about 55 base pairs upstream (e.g., −55) of the transcription start site and about 134 base pairs upstream (e.g., −134) of the transcription start site. In some additional embodiments, the second external CSRE is located between 94 base pairs upstream (e.g., −94) of the transcription start site and 95 base pairs upstream (e.g., −95) of the transcription start site. In some specific embodiments, the second external CSRE is located between about 77 base pairs upstream (e.g., −77) of the transcription start site and about 156 base pairs upstream (e.g., −156) of the transcription start site. In some additional embodiments, the second external CSRE is located between 116 base pairs upstream (e.g., −116) of the transcription start site and 117 base pairs upstream (e.g., −117) of the transcription start site. In some specific embodiments, the second external CSRE is located between about 81 base pairs upstream (e.g., −81) of the transcription start site and about 160 base pairs upstream (e.g., −160) of the transcription start site. In some additional embodiments, the second external CSRE is located between 120 base pairs upstream (e.g., −120) of the transcription start site and 121 base pairs upstream (e.g., −121) of the transcription start site. In some specific embodiments, the second external CSRE is located between about 87 base pairs upstream (e.g., −87) of the transcription start site and about 166 base pairs upstream (e.g., −166) of the transcription start site. In some additional embodiments, the second external CSRE is located between 126 base pairs upstream (e.g., −126) of the transcription start site and 127 base pairs upstream (e.g., −127) of the transcription start site. In some specific embodiments, the second external CSRE is located between about 93 base pairs upstream (e.g., −93) of the transcription start site and about 172 base pairs upstream (e.g., −172) of the transcription start site. In some additional embodiments, the second external CSRE is located between 132 base pairs upstream (e.g., −132) of the transcription start site and 133 base pairs upstream (e.g., −133) of the transcription start site. In some specific embodiments, the second external CSRE is located between about 94 base pairs upstream (e.g., −94) of the transcription start site and about 173 base pairs upstream (e.g., −173) of the transcription start site. In some additional embodiments, the second external CSRE is located between 133 base pairs upstream (e.g., −133) of the transcription start site and 134 base pairs upstream (e.g., −134) of the transcription start site. In some specific embodiments, the second external CSRE is located between about 99 base pairs upstream (e.g., −99) of the transcription start site and about 178 base pairs upstream (e.g., −178) of the transcription start site. In some additional embodiments, the second external CSRE is located between 138 base pairs upstream (e.g., −138) of the transcription start site and 139 base pairs upstream (e.g., −139) of the transcription start site. In some specific embodiments, the second external CSRE is located between about 104 base pairs upstream (e.g., −104) of the transcription start site and about 183 base pairs upstream (e.g., −183) of the transcription start site. In some additional embodiments, the second external CSRE is located between 143 base pairs upstream (e.g., −143) of the transcription start site and 144 base pairs upstream (e.g., −144) of the transcription start site. In some specific embodiments, the second external CSRE is located between about 109 base pairs upstream (e.g., −109) of the transcription start site and about 188 base pairs upstream (e.g., −188) of the transcription start site. In some additional embodiments, the second external CSRE is located between 148 base pairs upstream (e.g., −148) of the transcription start site and 149 base pairs upstream (e.g., −149) of the transcription start site. In some specific embodiments, the second external CSRE is located between about 111 base pairs upstream (e.g., −111) of the transcription start site and about 190 base pairs upstream (e.g., −190) of the transcription start site. In some additional embodiments, the second external CSRE is located between 150 base pairs upstream (e.g., −150) of the transcription start site and 151 base pairs upstream (e.g., −151) of the transcription start site. In some specific embodiments, the second external CSRE is located between about 114 base pairs upstream (e.g., −114) of the transcription start site and about 193 base pairs upstream (e.g., −193) of the transcription start site. In some additional embodiments, the second external CSRE is located between 153 base pairs upstream (e.g., −153) of the transcription start site and 154 base pairs upstream (e.g., −154) of the transcription start site. In some specific embodiments, the second external CSRE is located between about 119 base pairs upstream (e.g., −119) of the transcription start site and about 198 base pairs upstream (e.g., −198) of the transcription start site. In some additional embodiments, the second external CSRE is located between 158 base pairs upstream (e.g., −158) of the transcription start site and 159 base pairs upstream (e.g., −159) of the transcription start site. In some specific embodiments, the second external CSRE is located between about 124 base pairs upstream (e.g., −124) of the transcription start site and about 203 base pairs upstream (e.g., −203) of the transcription start site. In some additional embodiments, the second external CSRE is located between 163 base pairs upstream (e.g., −163) of the transcription start site and 164 base pairs upstream (e.g., −164) of the transcription start site. In some specific embodiments, the second external CSRE is located between about 129 base pairs upstream (e.g., −129) of the transcription start site and about 208 base pairs upstream (e.g., −208) of the transcription start site. In some additional embodiments, the second external CSRE is located between 168 base pairs upstream (e.g., −168) of the transcription start site and 169 base pairs upstream (e.g., −169) of the transcription start site. In some specific embodiments, the second external CSRE is located between about 189 base pairs upstream (e.g., −189) of the transcription start site and about 268 base pairs upstream (e.g., −268) of the transcription start site. In some additional embodiments, the second external CSRE is located between 228 base pairs upstream (e.g., −228) of the transcription start site and 229 base pairs upstream (e.g., −229) of the transcription start site. In some specific embodiments, the second external CSRE is located between about 198 base pairs upstream (e.g., −198) of the transcription start site and about 277 base pairs upstream (e.g., −277) of the transcription start site. In some additional embodiments, the second external CSRE is located between 237 base pairs upstream (e.g., −237) of the transcription start site and 238 base pairs upstream (e.g., −238) of the transcription start site. In some specific embodiments, the second external CSRE is located between about 239 base pairs upstream (e.g., −239) of the transcription start site and about 318 base pairs upstream (e.g., −318) of the transcription start site. In some additional embodiments, the second external CSRE is located between 278 base pairs upstream (e.g., −278) of the transcription start site and 279 base pairs upstream (e.g., −279) of the transcription start site. In some specific embodiments, the second external CSRE is located between about 318 base pairs upstream (e.g., −318) of the transcription start site and about 397 base pairs upstream (e.g., −397) of the transcription start site. In some additional embodiments, the second external CSRE is located between 357 base pairs upstream (e.g., −357) of the transcription start site and 358 base pairs upstream (e.g., −358) of the transcription start site. In yet another specific embodiment, the engineered promoter with at least two external CSREs comprises a first external CSRE located between 129 base pairs upstream (e.g., −129) of the transcription start site and 208 base pairs upstream (e.g., −208) of the transcription start site; and a second external CSRE located between 198 base pairs upstream (e.g., −198) of the transcription start site and 277 base pairs upstream (e.g., −277) of the transcription start site. For example, the engineered promoter with at least two external CSREs comprises a first external CSRE located between 168 base pairs upstream (e.g., −168) of the transcription start site and 169 base pairs upstream (e.g., −169) of the transcription start site; and a second external CSRE located between 237 base pairs upstream (e.g., −237) of the transcription start site and 238 base pairs upstream (e.g., −238) of the transcription start site.

In some embodiments, the engineered promoter of the present disclosure comprises at least three external CSREs. Embodiments of the location and the nucleic acid sequence of the first, and the second external CSREs are provided herein and can be used in an engineered promoter comprising three or more external CSREs. When a plurality of CSREs is included in the engineered promoter, the CSREs can be independently located at the same position and be contiguous or be provided at different positions and be non-contiguous. In some embodiments, the third external CSRE is located between the transcription start site and about 397 base pairs upstream (e.g., −397) of the transcription start site. In some alternative embodiments, the third external CSRE is located between about 7 base pairs upstream (e.g., −7) of the transcription start site and about 350 base pairs upstream (e.g., −397) of the transcription start site. In some specific embodiments, the third external CSRE is located between about 7 base pairs upstream (e.g., −7) of the transcription start site and about 86 base pairs upstream (e.g., −86) of the transcription start site. In some additional embodiments, the third external CSRE is located between 46 base pairs upstream (e.g., −46) of the transcription start site and 47 base pairs upstream (e.g., −47) of the transcription start site. In some specific embodiments, the third external CSRE is located between about 37 base pairs upstream (e.g., −37) of the transcription start site and about 116 base pairs upstream (e.g., −116) of the transcription start site. In some additional embodiments, the third external CSRE is located between 76 base pairs upstream (e.g., −76) of the transcription start site and 77 base pairs upstream (e.g., −77) of the transcription start site. In some specific embodiments, the third external CSRE is located between about 47 base pairs upstream (e.g., −47) of the transcription start site and about 126 base pairs upstream (e.g., −126) of the transcription start site. In some additional embodiments, the third external CSRE is located between 86 base pairs upstream (e.g., −86) of the transcription start site and 87 base pairs upstream (e.g., −87) of the transcription start site. In some specific embodiments, the third external CSRE is located between about 55 base pairs upstream (e.g., −55) of the transcription start site and about 134 base pairs upstream (e.g., −134) of the transcription start site. In some additional embodiments, the third external CSRE is located between 94 base pairs upstream (e.g., −94) of the transcription start site and 95 base pairs upstream (e.g., −95) of the transcription start site. In some specific embodiments, the third external CSRE is located between about 77 base pairs upstream (e.g., −77) of the transcription start site and about 156 base pairs upstream (e.g., −156) of the transcription start site. In some additional embodiments, the third external CSRE is located between 116 base pairs upstream (e.g., −116) of the transcription start site and 117 base pairs upstream (e.g., −117) of the transcription start site. In some specific embodiments, the third external CSRE is located between about 81 base pairs upstream (e.g., −81) of the transcription start site and about 160 base pairs upstream (e.g., −160) of the transcription start site. In some additional embodiments, the third external CSRE is located between 120 base pairs upstream (e.g., −120) of the transcription start site and 121 base pairs upstream (e.g., −121) of the transcription start site. In some specific embodiments, the third external CSRE is located between about 87 base pairs upstream (e.g., −87) of the transcription start site and about 166 base pairs upstream (e.g., −166) of the transcription start site. In some additional embodiments, the third external CSRE is located between 126 base pairs upstream (e.g., −126) of the transcription start site and 127 base pairs upstream (e.g., −127) of the transcription start site. In some specific embodiments, the third external CSRE is located between about 93 base pairs upstream (e.g., −93) of the transcription start site and about 172 base pairs upstream (e.g., −172) of the transcription start site. In some additional embodiments, the third external CSRE is located between 132 base pairs upstream (e.g., −132) of the transcription start site and 133 base pairs upstream (e.g., −133) of the transcription start site. In some specific embodiments, the third external CSRE is located between about 94 base pairs upstream (e.g., −94) of the transcription start site and about 173 base pairs upstream (e.g., −173) of the transcription start site. In some additional embodiments, the third external CSRE is located between 133 base pairs upstream (e.g., −133) of the transcription start site and 134 base pairs upstream (e.g., −134) of the transcription start site. In some specific embodiments, the third external CSRE is located between about 99 base pairs upstream (e.g., −99) of the transcription start site and about 178 base pairs upstream (e.g., −178) of the transcription start site. In some additional embodiments, the third external CSRE is located between 138 base pairs upstream (e.g., −138) of the transcription start site and 139 base pairs upstream (e.g., −139) of the transcription start site. In some specific embodiments, the third external CSRE is located between about 104 base pairs upstream (e.g., −104) of the transcription start site and about 183 base pairs upstream (e.g., −183) of the transcription start site. In some additional embodiments, the third external CSRE is located between 143 base pairs upstream (e.g., −143) of the transcription start site and 144 base pairs upstream (e.g., −144) of the transcription start site. In some specific embodiments, the third external CSRE is located between about 109 base pairs upstream (e.g., −109) of the transcription start site and about 188 base pairs upstream (e.g., −188) of the transcription start site. In some additional embodiments, the third external CSRE is located between 148 base pairs upstream (e.g., −148) of the transcription start site and 149 base pairs upstream (e.g., −149) of the transcription start site. In some specific embodiments, the third external CSRE is located between about 111 base pairs upstream (e.g., −111) of the transcription start site and about 190 base pairs upstream (e.g., −190) of the transcription start site. In some additional embodiments, the third external CSRE is located between 150 base pairs upstream (e.g., −150) of the transcription start site and 151 base pairs upstream (e.g., −151) of the transcription start site. In some specific embodiments, the third external CSRE is located between about 114 base pairs upstream (e.g., −114) of the transcription start site and about 193 base pairs upstream (e.g., −193) of the transcription start site. In some additional embodiments, the third external CSRE is located between 153 base pairs upstream (e.g., −153) of the transcription start site and 154 base pairs upstream (e.g., −154) of the transcription start site. In some specific embodiments, the third external CSRE is located between about 119 base pairs upstream (e.g., −119) of the transcription start site and about 198 base pairs upstream (e.g., −198) of the transcription start site. In some additional embodiments, the third external CSRE is located between 158 base pairs upstream (e.g., −158) of the transcription start site and 159 base pairs upstream (e.g., −159) of the transcription start site. In some specific embodiments, the third external CSRE is located between about 124 base pairs upstream (e.g., −124) of the transcription start site and about 203 base pairs upstream (e.g., −203) of the transcription start site. In some additional embodiments, the third external CSRE is located between 163 base pairs upstream (e.g., −163) of the transcription start site and 164 base pairs upstream (e.g., −164) of the transcription start site. In some specific embodiments, the third external CSRE is located between about 129 base pairs upstream (e.g., −129) of the transcription start site and about 208 base pairs upstream (e.g., −208) of the transcription start site. In some additional embodiments, the third external CSRE is located between 168 base pairs upstream (e.g., −168) of the transcription start site and 169 base pairs upstream (e.g., −169) of the transcription start site. In some specific embodiments, the third external CSRE is located between about 189 base pairs upstream (e.g., −189) of the transcription start site and about 268 base pairs upstream (e.g., −268) of the transcription start site. In some additional embodiments, the third external CSRE is located between 228 base pairs upstream (e.g., −228) of the transcription start site and 229 base pairs upstream (e.g., −229) of the transcription start site. In some specific embodiments, the third external CSRE is located between about 198 base pairs upstream (e.g., −198) of the transcription start site and about 277 base pairs upstream (e.g., −277) of the transcription start site. In some additional embodiments, the third external CSRE is located between 237 base pairs upstream (e.g., −237) of the transcription start site and 238 base pairs upstream (e.g., −238) of the transcription start site. In some specific embodiments, the third external CSRE is located between about 239 base pairs upstream (e.g., −239) of the transcription start site and about 318 base pairs upstream (e.g., −318) of the transcription start site. In some additional embodiments, the third external CSRE is located between 278 base pairs upstream (e.g., −278) of the transcription start site and 279 base pairs upstream (e.g., −279) of the transcription start site. In some specific embodiments, the third external CSRE is located between about 318 base pairs upstream (e.g., −318) of the transcription start site and about 397 base pairs upstream (e.g., −397) of the transcription start site. In some additional embodiments, the third external CSRE is located between 357 base pairs upstream (e.g., −357) of the transcription start site and 358 base pairs upstream (e.g., −358) of the transcription start site. In yet another specific embodiment, the engineered promoter with at least three external CSREs comprises a first external CSRE located between 129 base pairs upstream (e.g., −129) of the transcription start site and 208 base pairs upstream (e.g., −208) of the transcription start site; a second external CSRE located between 198 base pairs upstream (e.g., −198) of the transcription start site and 277 base pairs upstream (e.g., −277) of the transcription start site; and a third external CSRE located between 318 base pairs upstream (e.g., −318) of the transcription start site and 397 base pairs upstream (e.g., −397) of the transcription start site. For example, the engineered promoter with at least three external CSREs comprises a first external CSRE located between 168 base pairs upstream (e.g., −168) of the transcription start site and 169 base pairs upstream (e.g., −169) of the transcription start site; a second external CSRE located between 237 base pairs upstream (e.g., −237) of the transcription start site and 238 base pairs upstream (e.g., −238) of the transcription start site; and a third external CSRE located between 357 base pairs upstream (e.g., −357) of the transcription start site and 358 base pairs upstream (e.g., −358) of the transcription start site. In yet another specific embodiment, the engineered promoter with at least three external CSREs comprises a first external CSRE located between 55 base pairs upstream (e.g., −55) of the transcription start site and 134 base pairs upstream (e.g., −134) of the transcription start site; a second external CSRE located between 81 base pairs upstream (e.g., −81) of the transcription start site and 160 base pairs upstream (e.g., −160) of the transcription start site; and a third external CSRE located between 87 base pairs upstream (e.g., −87) of the transcription start site and 166 base pairs upstream (e.g., −166) of the transcription start site. For example, the engineered promoter with at least three external CSREs comprises a first external CSRE located between 94 base pairs upstream (e.g., −94) of the transcription start site and 95 base pairs upstream (e.g., −95) of the transcription start site; a second external CSRE located between 120 base pairs upstream (e.g., −120) of the transcription start site and 121 base pairs upstream (e.g., −121) of the transcription start site; and a third external CSRE located between 126 base pairs upstream (e.g., −126) of the transcription start site and 127 base pairs upstream (e.g., −127) of the transcription start site.

In some embodiments, the engineered promoter of the present disclosure comprises at least four external CSREs. Embodiments of the location and the nucleic acid sequence of the first, second, and third external CSREs are provided herein and can be used in an engineered promoter comprising four or more external CSREs. When a plurality of CSREs is included in the engineered promoter, the CSREs can be independently located at the same position and be contiguous or be provided at different positions and be non-contiguous. In some embodiments, the fourth external CSRE is located between the transcription start site and about 397 base pairs upstream (e.g., −397) of the transcription start site. In some alternative embodiments, the fourth external CSRE is located between about 7 base pairs upstream (e.g., −7) of the transcription start site and about 350 base pairs upstream (e.g., −397) of the transcription start site. In some specific embodiments, the fourth external CSRE is located between about 7 base pairs upstream (e.g., −7) of the transcription start site and about 86 base pairs upstream (e.g., −86) of the transcription start site. In some additional embodiments, the fourth external CSRE is located between 46 base pairs upstream (e.g., −46) of the transcription start site and 47 base pairs upstream (e.g., −47) of the transcription start site. In some specific embodiments, the fourth external CSRE is located between about 37 base pairs upstream (e.g., −37) of the transcription start site and about 116 base pairs upstream (e.g., −116) of the transcription start site. In some additional embodiments, the fourth external CSRE is located between 76 base pairs upstream (e.g., −76) of the transcription start site and 77 base pairs upstream (e.g., −77) of the transcription start site. In some specific embodiments, the fourth external CSRE is located between about 47 base pairs upstream (e.g., −47) of the transcription start site and about 126 base pairs upstream (e.g., −126) of the transcription start site. In some additional embodiments, the fourth external CSRE is located between 86 base pairs upstream (e.g., −86) of the transcription start site and 87 base pairs upstream (e.g., −87) of the transcription start site. In some specific embodiments, the fourth external CSRE is located between about 55 base pairs upstream (e.g., −55) of the transcription start site and about 134 base pairs upstream (e.g., −134) of the transcription start site. In some additional embodiments, the fourth external CSRE is located between 94 base pairs upstream (e.g., −94) of the transcription start site and 95 base pairs upstream (e.g., −95) of the transcription start site. In some specific embodiments, the fourth external CSRE is located between about 77 base pairs upstream (e.g., −77) of the transcription start site and about 156 base pairs upstream (e.g., −156) of the transcription start site. In some additional embodiments, the fourth external CSRE is located between 116 base pairs upstream (e.g., −116) of the transcription start site and 117 base pairs upstream (e.g., −117) of the transcription start site. In some specific embodiments, the fourth external CSRE is located between about 81 base pairs upstream (e.g., −81) of the transcription start site and about 160 base pairs upstream (e.g., −160) of the transcription start site. In some additional embodiments, the fourth external CSRE is located between 120 base pairs upstream (e.g., −120) of the transcription start site and 121 base pairs upstream (e.g., −121) of the transcription start site. In some specific embodiments, the fourth external CSRE is located between about 87 base pairs upstream (e.g., −87) of the transcription start site and about 166 base pairs upstream (e.g., −166) of the transcription start site. In some additional embodiments, the fourth external CSRE is located between 126 base pairs upstream (e.g., −126) of the transcription start site and 127 base pairs upstream (e.g., −127) of the transcription start site. In some specific embodiments, the fourth external CSRE is located between about 93 base pairs upstream (e.g., −93) of the transcription start site and about 172 base pairs upstream (e.g., −172) of the transcription start site. In some additional embodiments, the fourth external CSRE is located between 132 base pairs upstream (e.g., −132) of the transcription start site and 133 base pairs upstream (e.g., −133) of the transcription start site. In some specific embodiments, the fourth external CSRE is located between about 94 base pairs upstream (e.g., −94) of the transcription start site and about 173 base pairs upstream (e.g., −173) of the transcription start site. In some additional embodiments, the fourth external CSRE is located between 133 base pairs upstream (e.g., −133) of the transcription start site and 134 base pairs upstream (e.g., −134) of the transcription start site. In some specific embodiments, the fourth external CSRE is located between about 99 base pairs upstream (e.g., −99) of the transcription start site and about 178 base pairs upstream (e.g., −178) of the transcription start site. In some additional embodiments, the fourth external CSRE is located between 138 base pairs upstream (e.g., −138) of the transcription start site and 139 base pairs upstream (e.g., −139) of the transcription start site. In some specific embodiments, the fourth external CSRE is located between about 104 base pairs upstream (e.g., −104) of the transcription start site and about 183 base pairs upstream (e.g., −183) of the transcription start site. In some additional embodiments, the fourth external CSRE is located between 143 base pairs upstream (e.g., −143) of the transcription start site and 144 base pairs upstream (e.g., −144) of the transcription start site. In some specific embodiments, the fourth external CSRE is located between about 109 base pairs upstream (e.g., −109) of the transcription start site and about 188 base pairs upstream (e.g., −188) of the transcription start site. In some additional embodiments, the fourth external CSRE is located between 148 base pairs upstream (e.g., −148) of the transcription start site and 149 base pairs upstream (e.g., −149) of the transcription start site. In some specific embodiments, the fourth external CSRE is located between about 111 base pairs upstream (e.g., −111) of the transcription start site and about 190 base pairs upstream (e.g., −190) of the transcription start site. In some additional embodiments, the fourth external CSRE is located between 150 base pairs upstream (e.g., −150) of the transcription start site and 151 base pairs upstream (e.g., −151) of the transcription start site. In some specific embodiments, the fourth external CSRE is located between about 114 base pairs upstream (e.g., −114) of the transcription start site and about 193 base pairs upstream (e.g., −193) of the transcription start site. In some additional embodiments, the fourth external CSRE is located between 153 base pairs upstream (e.g., −153) of the transcription start site and 154 base pairs upstream (e.g., −154) of the transcription start site. In some specific embodiments, the fourth external CSRE is located between about 119 base pairs upstream (e.g., −119) of the transcription start site and about 198 base pairs upstream (e.g., −198) of the transcription start site. In some additional embodiments, the fourth external CSRE is located between 158 base pairs upstream (e.g., −158) of the transcription start site and 159 base pairs upstream (e.g., −159) of the transcription start site. In some specific embodiments, the fourth external CSRE is located between about 124 base pairs upstream (e.g., −124) of the transcription start site and about 203 base pairs upstream (e.g., −203) of the transcription start site. In some additional embodiments, the fourth external CSRE is located between 163 base pairs upstream (e.g., −163) of the transcription start site and 164 base pairs upstream (e.g., −164) of the transcription start site. In some specific embodiments, the fourth external CSRE is located between about 129 base pairs upstream (e.g., −129) of the transcription start site and about 208 base pairs upstream (e.g., −208) of the transcription start site. In some additional embodiments, the fourth external CSRE is located between 168 base pairs upstream (e.g., −168) of the transcription start site and 169 base pairs upstream (e.g., −169) of the transcription start site. In some specific embodiments, the fourth external CSRE is located between about 189 base pairs upstream (e.g., −189) of the transcription start site and about 268 base pairs upstream (e.g., −268) of the transcription start site. In some additional embodiments, the fourth external CSRE is located between 228 base pairs upstream (e.g., −228) of the transcription start site and 229 base pairs upstream (e.g., −229) of the transcription start site. In some specific embodiments, the fourth external CSRE is located between about 198 base pairs upstream (e.g., −198) of the transcription start site and about 277 base pairs upstream (e.g., −277) of the transcription start site. In some additional embodiments, the fourth external CSRE is located between 237 base pairs upstream (e.g., −237) of the transcription start site and 238 base pairs upstream (e.g., −238) of the transcription start site. In some specific embodiments, the fourth external CSRE is located between about 239 base pairs upstream (e.g., −239) of the transcription start site and about 318 base pairs upstream (e.g., −318) of the transcription start site. In some additional embodiments, the fourth external CSRE is located between 278 base pairs upstream (e.g., −278) of the transcription start site and 279 base pairs upstream (e.g., −279) of the transcription start site. In some specific embodiments, the fourth external CSRE is located between about 318 base pairs upstream (e.g., −318) of the transcription start site and about 397 base pairs upstream (e.g., −397) of the transcription start site. In some additional embodiments, the fourth external CSRE is located between 357 base pairs upstream (e.g., −357) of the transcription start site and 358 base pairs upstream (e.g., −358) of the transcription start site.

In some embodiments, the engineered promoter of the present disclosure comprises at least five external CSREs. Embodiments of the location and the nucleic acid sequence of the first, second, third, and fourth external CSREs are provided herein and can be used in an engineered promoter comprising five or more external CSREs. When a plurality of CSREs is included in the engineered promoter, the CSREs can be independently located at the same position and be contiguous or be provided at different positions and be non-contiguous. In some embodiments, the fifth external CSRE is located between the transcription start site and about 397 base pairs upstream (e.g., −397) of the transcription start site. In some alternative embodiments, the fifth external CSRE is located between about 7 base pairs upstream (e.g., −7) of the transcription start site and about 350 base pairs upstream (e.g., −397) of the transcription start site. In some specific embodiments, the fifth external CSRE is located between about 7 base pairs upstream (e.g., −7) of the transcription start site and about 86 base pairs upstream (e.g., −86) of the transcription start site. In some additional embodiments, the fifth external CSRE is located between 46 base pairs upstream (e.g., −46) of the transcription start site and 47 base pairs upstream (e.g., −47) of the transcription start site. In some specific embodiments, the fifth external CSRE is located between about 37 base pairs upstream (e.g., −37) of the transcription start site and about 116 base pairs upstream (e.g., −116) of the transcription start site. In some additional embodiments, the fifth external CSRE is located between 76 base pairs upstream (e.g., −76) of the transcription start site and 77 base pairs upstream (e.g., −77) of the transcription start site. In some specific embodiments, the fifth external CSRE is located between about 47 base pairs upstream (e.g., −47) of the transcription start site and about 126 base pairs upstream (e.g., −126) of the transcription start site. In some additional embodiments, the fifth external CSRE is located between 86 base pairs upstream (e.g., −86) of the transcription start site and 87 base pairs upstream (e.g., −87) of the transcription start site. In some specific embodiments, the fifth external CSRE is located between about 55 base pairs upstream (e.g., −55) of the transcription start site and about 134 base pairs upstream (e.g., −134) of the transcription start site. In some additional embodiments, the fifth external CSRE is located between 94 base pairs upstream (e.g., −94) of the transcription start site and 95 base pairs upstream (e.g., −95) of the transcription start site. In some specific embodiments, the fifth external CSRE is located between about 77 base pairs upstream (e.g., −77) of the transcription start site and about 156 base pairs upstream (e.g., −156) of the transcription start site. In some additional embodiments, the fifth external CSRE is located between 116 base pairs upstream (e.g., −116) of the transcription start site and 117 base pairs upstream (e.g., −117) of the transcription start site. In some specific embodiments, the fifth external CSRE is located between about 81 base pairs upstream (e.g., −81) of the transcription start site and about 160 base pairs upstream (e.g., −160) of the transcription start site. In some additional embodiments, the fifth external CSRE is located between 120 base pairs upstream (e.g., −120) of the transcription start site and 121 base pairs upstream (e.g., −121) of the transcription start site. In some specific embodiments, the fifth external CSRE is located between about 87 base pairs upstream (e.g., −87) of the transcription start site and about 166 base pairs upstream (e.g., −166) of the transcription start site. In some additional embodiments, the fifth external CSRE is located between 126 base pairs upstream (e.g., −126) of the transcription start site and 127 base pairs upstream (e.g., −127) of the transcription start site. In some specific embodiments, the fifth external CSRE is located between about 93 base pairs upstream (e.g., −93) of the transcription start site and about 172 base pairs upstream (e.g., −172) of the transcription start site. In some additional embodiments, the fifth external CSRE is located between 132 base pairs upstream (e.g., −132) of the transcription start site and 133 base pairs upstream (e.g., −133) of the transcription start site. In some specific embodiments, the fifth external CSRE is located between about 94 base pairs upstream (e.g., −94) of the transcription start site and about 173 base pairs upstream (e.g., −173) of the transcription start site. In some additional embodiments, the fifth external CSRE is located between 133 base pairs upstream (e.g., −133) of the transcription start site and 134 base pairs upstream (e.g., −134) of the transcription start site. In some specific embodiments, the fifth external CSRE is located between about 99 base pairs upstream (e.g., −99) of the transcription start site and about 178 base pairs upstream (e.g., −178) of the transcription start site. In some additional embodiments, the fifth external CSRE is located between 138 base pairs upstream (e.g., −138) of the transcription start site and 139 base pairs upstream (e.g., −139) of the transcription start site. In some specific embodiments, the fifth external CSRE is located between about 104 base pairs upstream (e.g., −104) of the transcription start site and about 183 base pairs upstream (e.g., −183) of the transcription start site. In some additional embodiments, the fifth external CSRE is located between 143 base pairs upstream (e.g., −143) of the transcription start site and 144 base pairs upstream (e.g., −144) of the transcription start site. In some specific embodiments, the fifth external CSRE is located between about 109 base pairs upstream (e.g., −109) of the transcription start site and about 188 base pairs upstream (e.g., −188) of the transcription start site. In some additional embodiments, the fifth external CSRE is located between 148 base pairs upstream (e.g., −148) of the transcription start site and 149 base pairs upstream (e.g., −149) of the transcription start site. In some specific embodiments, the fifth external CSRE is located between about 111 base pairs upstream (e.g., −111) of the transcription start site and about 190 base pairs upstream (e.g., −190) of the transcription start site. In some additional embodiments, the fifth external CSRE is located between 150 base pairs upstream (e.g., −150) of the transcription start site and 151 base pairs upstream (e.g., −151) of the transcription start site. In some specific embodiments, the fifth external CSRE is located between about 114 base pairs upstream (e.g., −114) of the transcription start site and about 193 base pairs upstream (e.g., −193) of the transcription start site. In some additional embodiments, the fifth external CSRE is located between 153 base pairs upstream (e.g., −153) of the transcription start site and 154 base pairs upstream (e.g., −154) of the transcription start site. In some specific embodiments, the fifth external CSRE is located between about 119 base pairs upstream (e.g., −119) of the transcription start site and about 198 base pairs upstream (e.g., −198) of the transcription start site. In some additional embodiments, the fifth external CSRE is located between 158 base pairs upstream (e.g., −158) of the transcription start site and 159 base pairs upstream (e.g., −159) of the transcription start site. In some specific embodiments, the fifth external CSRE is located between about 124 base pairs upstream (e.g., −124) of the transcription start site and about 203 base pairs upstream (e.g., −203) of the transcription start site. In some additional embodiments, the fifth external CSRE is located between 163 base pairs upstream (e.g., −163) of the transcription start site and 164 base pairs upstream (e.g., −164) of the transcription start site. In some specific embodiments, the fifth external CSRE is located between about 129 base pairs upstream (e.g., −129) of the transcription start site and about 208 base pairs upstream (e.g., −208) of the transcription start site. In some additional embodiments, the fifth external CSRE is located between 168 base pairs upstream (e.g., −168) of the transcription start site and 169 base pairs upstream (e.g., −169) of the transcription start site. In some specific embodiments, the fifth external CSRE is located between about 189 base pairs upstream (e.g., −189) of the transcription start site and about 268 base pairs upstream (e.g., −268) of the transcription start site. In some additional embodiments, the fifth external CSRE is located between 228 base pairs upstream (e.g., −228) of the transcription start site and 229 base pairs upstream (e.g., −229) of the transcription start site. In some specific embodiments, the fifth external CSRE is located between about 198 base pairs upstream (e.g., −198) of the transcription start site and about 277 base pairs upstream (e.g., −277) of the transcription start site. In some additional embodiments, the fifth external CSRE is located between 237 base pairs upstream (e.g., −237) of the transcription start site and 238 base pairs upstream (e.g., −238) of the transcription start site. In some specific embodiments, the fifth external CSRE is located between about 239 base pairs upstream (e.g., −239) of the transcription start site and about 318 base pairs upstream (e.g., −318) of the transcription start site. In some additional embodiments, the fifth external CSRE is located between 278 base pairs upstream (e.g., −278) of the transcription start site and 279 base pairs upstream (e.g., −279) of the transcription start site. In some specific embodiments, the fifth external CSRE is located between about 318 base pairs upstream (e.g., −318) of the transcription start site and about 397 base pairs upstream (e.g., −397) of the transcription start site. In some additional embodiments, the fifth external CSRE is located between 357 base pairs upstream (e.g., −357) of the transcription start site and 358 base pairs upstream (e.g., −358) of the transcription start site. In yet another specific embodiment, the engineered promoter with at least five external CSREs comprises a first external CSRE located between 93 base pairs upstream (e.g., −93) of the transcription start site and 172 base pairs upstream (e.g., −172) of the transcription start site; a second external CSRE located between 111 base pairs upstream (e.g., −111) of the transcription start site and 190 base pairs upstream (e.g., −190) of the transcription start site; a third external CSRE located between 129 base pairs upstream (e.g., −129) of the transcription start site and 208 base pairs upstream (e.g., −208) of the transcription start site; a fourth external CSRE located between 198 base pairs upstream (e.g., −198) of the transcription start site and 277 base pairs upstream (e.g., −277) of the transcription start site; and a fifth external CSRE located between 318 base pairs upstream (e.g., −318) of the transcription start site and 397 base pairs upstream (e.g., −397) of the transcription start site. For example, the engineered promoter with at least five external CSREs comprises a first external CSRE located between 132 base pairs upstream (e.g., −132) of the transcription start site and 133 base pairs upstream (e.g., −133) of the transcription start site; a second external CSRE located between 150 base pairs upstream (e.g., −150) of the transcription start site and 151 base pairs upstream (e.g., −151) of the transcription start site; a third external CSRE located between 168 base pairs upstream (e.g., −168) of the transcription start site and 169 base pairs upstream (e.g., −169) of the transcription start site; a fourth external CSRE located between 237 base pairs upstream (e.g., −237) of the transcription start site and 238 base pairs upstream (e.g., −238) of the transcription start site; and a fifth external CSRE located between 357 base pairs upstream (e.g., −357) of the transcription start site and 358 base pairs upstream (e.g., −358) of the transcription start site.

In some embodiments, the engineered promoter of the present disclosure comprises at least six external CSREs. Embodiments of the location and the nucleic acid sequence of the first, second, third, fourth, and fifth external CSREs are provided herein and can be used in an engineered promoter comprising six or more external CSREs. When a plurality of CSREs is included in the engineered promoter, the CSREs can be independently located at the same position and be contiguous or be provided at different positions and be non-contiguous. In some embodiments, the sixth external CSRE is located between the transcription start site and about 397 base pairs upstream (e.g., −397) of the transcription start site. In some alternative embodiments, the sixth external CSRE is located between about 7 base pairs upstream (e.g., −7) of the transcription start site and about 350 base pairs upstream (e.g., −397) of the transcription start site. In some specific embodiments, the sixth external CSRE is located between about 7 base pairs upstream (e.g., −7) of the transcription start site and about 86 base pairs upstream (e.g., −86) of the transcription start site. In some additional embodiments, the sixth external CSRE is located between 46 base pairs upstream (e.g., −46) of the transcription start site and 47 base pairs upstream (e.g., −47) of the transcription start site. In some specific embodiments, the sixth external CSRE is located between about 37 base pairs upstream (e.g., −37) of the transcription start site and about 116 base pairs upstream (e.g., −116) of the transcription start site. In some additional embodiments, the sixth external CSRE is located between 76 base pairs upstream (e.g., −76) of the transcription start site and 77 base pairs upstream (e.g., −77) of the transcription start site. In some specific embodiments, the sixth external CSRE is located between about 47 base pairs upstream (e.g., −47) of the transcription start site and about 126 base pairs upstream (e.g., −126) of the transcription start site. In some additional embodiments, the sixth external CSRE is located between 86 base pairs upstream (e.g., −86) of the transcription start site and 87 base pairs upstream (e.g., −87) of the transcription start site. In some specific embodiments, the sixth external CSRE is located between about 55 base pairs upstream (e.g., −55) of the transcription start site and about 134 base pairs upstream (e.g., −134) of the transcription start site. In some additional embodiments, the sixth external CSRE is located between 94 base pairs upstream (e.g., −94) of the transcription start site and 95 base pairs upstream (e.g., −95) of the transcription start site. In some specific embodiments, the sixth external CSRE is located between about 77 base pairs upstream (e.g., −77) of the transcription start site and about 156 base pairs upstream (e.g., −156) of the transcription start site. In some additional embodiments, the sixth external CSRE is located between 116 base pairs upstream (e.g., −116) of the transcription start site and 117 base pairs upstream (e.g., −117) of the transcription start site. In some specific embodiments, the sixth external CSRE is located between about 81 base pairs upstream (e.g., −81) of the transcription start site and about 160 base pairs upstream (e.g., −160) of the transcription start site. In some additional embodiments, the sixth external CSRE is located between 120 base pairs upstream (e.g., −120) of the transcription start site and 121 base pairs upstream (e.g., −121) of the transcription start site. In some specific embodiments, the sixth external CSRE is located between about 87 base pairs upstream (e.g., −87) of the transcription start site and about 166 base pairs upstream (e.g., −166) of the transcription start site. In some additional embodiments, the sixth external CSRE is located between 126 base pairs upstream (e.g., −126) of the transcription start site and 127 base pairs upstream (e.g., −127) of the transcription start site. In some specific embodiments, the sixth external CSRE is located between about 93 base pairs upstream (e.g., −93) of the transcription start site and about 172 base pairs upstream (e.g., −172) of the transcription start site. In some additional embodiments, the sixth external CSRE is located between 132 base pairs upstream (e.g., −132) of the transcription start site and 133 base pairs upstream (e.g., −133) of the transcription start site. In some specific embodiments, the sixth external CSRE is located between about 94 base pairs upstream (e.g., −94) of the transcription start site and about 173 base pairs upstream (e.g., −173) of the transcription start site. In some additional embodiments, the sixth external CSRE is located between 133 base pairs upstream (e.g., −133) of the transcription start site and 134 base pairs upstream (e.g., −134) of the transcription start site. In some specific embodiments, the sixth external CSRE is located between about 99 base pairs upstream (e.g., −99) of the transcription start site and about 178 base pairs upstream (e.g., −178) of the transcription start site. In some additional embodiments, the sixth external CSRE is located between 138 base pairs upstream (e.g., −138) of the transcription start site and 139 base pairs upstream (e.g., −139) of the transcription start site. In some specific embodiments, the sixth external CSRE is located between about 104 base pairs upstream (e.g., −104) of the transcription start site and about 183 base pairs upstream (e.g., −183) of the transcription start site. In some additional embodiments, the sixth external CSRE is located between 143 base pairs upstream (e.g., −143) of the transcription start site and 144 base pairs upstream (e.g., −144) of the transcription start site. In some specific embodiments, the sixth external CSRE is located between about 109 base pairs upstream (e.g., −109) of the transcription start site and about 188 base pairs upstream (e.g., −188) of the transcription start site. In some additional embodiments, the sixth external CSRE is located between 148 base pairs upstream (e.g., −148) of the transcription start site and 149 base pairs upstream (e.g., −149) of the transcription start site. In some specific embodiments, the sixth external CSRE is located between about 111 base pairs upstream (e.g., −111) of the transcription start site and about 190 base pairs upstream (e.g., −190) of the transcription start site. In some additional embodiments, the sixth external CSRE is located between 150 base pairs upstream (e.g., −150) of the transcription start site and 151 base pairs upstream (e.g., −151) of the transcription start site. In some specific embodiments, the sixth external CSRE is located between about 114 base pairs upstream (e.g., −114) of the transcription start site and about 193 base pairs upstream (e.g., −193) of the transcription start site. In some additional embodiments, the sixth external CSRE is located between 153 base pairs upstream (e.g., −153) of the transcription start site and 154 base pairs upstream (e.g., −154) of the transcription start site. In some specific embodiments, the sixth external CSRE is located between about 119 base pairs upstream (e.g., −119) of the transcription start site and about 198 base pairs upstream (e.g., −198) of the transcription start site. In some additional embodiments, the sixth external CSRE is located between 158 base pairs upstream (e.g., −158) of the transcription start site and 159 base pairs upstream (e.g., −159) of the transcription start site. In some specific embodiments, the sixth external CSRE is located between about 124 base pairs upstream (e.g., −124) of the transcription start site and about 203 base pairs upstream (e.g., −203) of the transcription start site. In some additional embodiments, the sixth external CSRE is located between 163 base pairs upstream (e.g., −163) of the transcription start site and 164 base pairs upstream (e.g., −164) of the transcription start site. In some specific embodiments, the sixth external CSRE is located between about 129 base pairs upstream (e.g., −129) of the transcription start site and about 208 base pairs upstream (e.g., −208) of the transcription start site. In some additional embodiments, the sixth external CSRE is located between 168 base pairs upstream (e.g., −168) of the transcription start site and 169 base pairs upstream (e.g., −169) of the transcription start site. In some specific embodiments, the sixth external CSRE is located between about 189 base pairs upstream (e.g., −189) of the transcription start site and about 268 base pairs upstream (e.g., −268) of the transcription start site. In some additional embodiments, the sixth external CSRE is located between 228 base pairs upstream (e.g., −228) of the transcription start site and 229 base pairs upstream (e.g., −229) of the transcription start site. In some specific embodiments, the sixth external CSRE is located between about 198 base pairs upstream (e.g., −198) of the transcription start site and about 277 base pairs upstream (e.g., −277) of the transcription start site. In some additional embodiments, the sixth external CSRE is located between 237 base pairs upstream (e.g., −237) of the transcription start site and 238 base pairs upstream (e.g., −238) of the transcription start site. In some specific embodiments, the sixth external CSRE is located between about 239 base pairs upstream (e.g., −239) of the transcription start site and about 318 base pairs upstream (e.g., −318) of the transcription start site. In some additional embodiments, the sixth external CSRE is located between 278 base pairs upstream (e.g., −278) of the transcription start site and 279 base pairs upstream (e.g., −279) of the transcription start site. In some specific embodiments, the sixth external CSRE is located between about 318 base pairs upstream (e.g., −318) of the transcription start site and about 397 base pairs upstream (e.g., −397) of the transcription start site. In some additional embodiments, the sixth external CSRE is located between 357 base pairs upstream (e.g., −357) of the transcription start site and 358 base pairs upstream (e.g., −358) of the transcription start site.

In some embodiments, the engineered promoter of the present disclosure comprises at least seven external CSREs. Embodiments of the location and the nucleic acid sequence of the first, second, third, fourth, fifth, and sixth external CSREs are provided herein and can be used in an engineered promoter comprising seven or more external CSREs. When a plurality of CSREs is included in the engineered promoter, the CSREs can be independently located at the same position and be contiguous or be provided at different positions and be non-contiguous. In some embodiments, the seventh external CSRE is located between the transcription start site and about 397 base pairs upstream (e.g., −397) of the transcription start site. In some alternative embodiments, the seventh external CSRE is located between about 7 base pairs upstream (e.g., −7) of the transcription start site and about 350 base pairs upstream (e.g., −397) of the transcription start site. In some specific embodiments, the seventh external CSRE is located between about 7 base pairs upstream (e.g., −7) of the transcription start site and about 86 base pairs upstream (e.g., −86) of the transcription start site. In some additional embodiments, the seventh external CSRE is located between 46 base pairs upstream (e.g., −46) of the transcription start site and 47 base pairs upstream (e.g., −47) of the transcription start site. In some specific embodiments, the seventh external CSRE is located between about 37 base pairs upstream (e.g., −37) of the transcription start site and about 116 base pairs upstream (e.g., −116) of the transcription start site. In some additional embodiments, the seventh external CSRE is located between 76 base pairs upstream (e.g., −76) of the transcription start site and 77 base pairs upstream (e.g., −77) of the transcription start site. In some specific embodiments, the seventh external CSRE is located between about 47 base pairs upstream (e.g., −47) of the transcription start site and about 126 base pairs upstream (e.g., −126) of the transcription start site. In some additional embodiments, the seventh external CSRE is located between 86 base pairs upstream (e.g., −86) of the transcription start site and 87 base pairs upstream (e.g., −87) of the transcription start site. In some specific embodiments, the seventh external CSRE is located between about 55 base pairs upstream (e.g., −55) of the transcription start site and about 134 base pairs upstream (e.g., −134) of the transcription start site. In some additional embodiments, the seventh external CSRE is located between 94 base pairs upstream (e.g., −94) of the transcription start site and 95 base pairs upstream (e.g., −95) of the transcription start site. In some specific embodiments, the seventh external CSRE is located between about 77 base pairs upstream (e.g., −77) of the transcription start site and about 156 base pairs upstream (e.g., −156) of the transcription start site. In some additional embodiments, the seventh external CSRE is located between 116 base pairs upstream (e.g., −116) of the transcription start site and 117 base pairs upstream (e.g., −117) of the transcription start site. In some specific embodiments, the seventh external CSRE is located between about 81 base pairs upstream (e.g., −81) of the transcription start site and about 160 base pairs upstream (e.g., −160) of the transcription start site. In some additional embodiments, the seventh external CSRE is located between 120 base pairs upstream (e.g., −120) of the transcription start site and 121 base pairs upstream (e.g., −121) of the transcription start site. In some specific embodiments, the seventh external CSRE is located between about 87 base pairs upstream (e.g., −87) of the transcription start site and about 166 base pairs upstream (e.g., −166) of the transcription start site. In some additional embodiments, the seventh external CSRE is located between 126 base pairs upstream (e.g., −126) of the transcription start site and 127 base pairs upstream (e.g., −127) of the transcription start site. In some specific embodiments, the seventh external CSRE is located between about 93 base pairs upstream (e.g., −93) of the transcription start site and about 172 base pairs upstream (e.g., −172) of the transcription start site. In some additional embodiments, the seventh external CSRE is located between 132 base pairs upstream (e.g., −132) of the transcription start site and 133 base pairs upstream (e.g., −133) of the transcription start site. In some specific embodiments, the seventh external CSRE is located between about 94 base pairs upstream (e.g., −94) of the transcription start site and about 173 base pairs upstream (e.g., −173) of the transcription start site. In some additional embodiments, the seventh external CSRE is located between 133 base pairs upstream (e.g., −133) of the transcription start site and 134 base pairs upstream (e.g., −134) of the transcription start site. In some specific embodiments, the seventh external CSRE is located between about 99 base pairs upstream (e.g., −99) of the transcription start site and about 178 base pairs upstream (e.g., −178) of the transcription start site. In some additional embodiments, the seventh external CSRE is located between 138 base pairs upstream (e.g., −138) of the transcription start site and 139 base pairs upstream (e.g., −139) of the transcription start site. In some specific embodiments, the seventh external CSRE is located between about 104 base pairs upstream (e.g., −104) of the transcription start site and about 183 base pairs upstream (e.g., −183) of the transcription start site. In some additional embodiments, the seventh external CSRE is located between 143 base pairs upstream (e.g., −143) of the transcription start site and 144 base pairs upstream (e.g., −144) of the transcription start site. In some specific embodiments, the seventh external CSRE is located between about 109 base pairs upstream (e.g., −109) of the transcription start site and about 188 base pairs upstream (e.g., −188) of the transcription start site. In some additional embodiments, the seventh external CSRE is located between 148 base pairs upstream (e.g., −148) of the transcription start site and 149 base pairs upstream (e.g., −149) of the transcription start site. In some specific embodiments, the seventh external CSRE is located between about 111 base pairs upstream (e.g., −111) of the transcription start site and about 190 base pairs upstream (e.g., −190) of the transcription start site. In some additional embodiments, the seventh external CSRE is located between 150 base pairs upstream (e.g., −150) of the transcription start site and 151 base pairs upstream (e.g., −151) of the transcription start site. In some specific embodiments, the seventh external CSRE is located between about 114 base pairs upstream (e.g., −114) of the transcription start site and about 193 base pairs upstream (e.g., −193) of the transcription start site. In some additional embodiments, the seventh external CSRE is located between 153 base pairs upstream (e.g., −153) of the transcription start site and 154 base pairs upstream (e.g., −154) of the transcription start site. In some specific embodiments, the seventh external CSRE is located between about 119 base pairs upstream (e.g., −119) of the transcription start site and about 198 base pairs upstream (e.g., −198) of the transcription start site. In some additional embodiments, the seventh external CSRE is located between 158 base pairs upstream (e.g., −158) of the transcription start site and 159 base pairs upstream (e.g., −159) of the transcription start site. In some specific embodiments, the seventh external CSRE is located between about 124 base pairs upstream (e.g., −124) of the transcription start site and about 203 base pairs upstream (e.g., −203) of the transcription start site. In some additional embodiments, the seventh external CSRE is located between 163 base pairs upstream (e.g., −163) of the transcription start site and 164 base pairs upstream (e.g., −164) of the transcription start site. In some specific embodiments, the seventh external CSRE is located between about 129 base pairs upstream (e.g., −129) of the transcription start site and about 208 base pairs upstream (e.g., −208) of the transcription start site. In some additional embodiments, the seventh external CSRE is located between 168 base pairs upstream (e.g., −168) of the transcription start site and 169 base pairs upstream (e.g., −169) of the transcription start site. In some specific embodiments, the seventh external CSRE is located between about 189 base pairs upstream (e.g., −189) of the transcription start site and about 268 base pairs upstream (e.g., −268) of the transcription start site. In some additional embodiments, the seventh external CSRE is located between 228 base pairs upstream (e.g., −228) of the transcription start site and 229 base pairs upstream (e.g., −229) of the transcription start site. In some specific embodiments, the seventh external CSRE is located between about 198 base pairs upstream (e.g., −198) of the transcription start site and about 277 base pairs upstream (e.g., −277) of the transcription start site. In some additional embodiments, the seventh external CSRE is located between 237 base pairs upstream (e.g., −237) of the transcription start site and 238 base pairs upstream (e.g., −238) of the transcription start site. In some specific embodiments, the seventh external CSRE is located between about 239 base pairs upstream (e.g., −239) of the transcription start site and about 318 base pairs upstream (e.g., −318) of the transcription start site. In some additional embodiments, the seventh external CSRE is located between 278 base pairs upstream (e.g., −278) of the transcription start site and 279 base pairs upstream (e.g., −279) of the transcription start site. In some specific embodiments, the seventh external CSRE is located between about 318 base pairs upstream (e.g., −318) of the transcription start site and about 397 base pairs upstream (e.g., −397) of the transcription start site. In some additional embodiments, the seventh external CSRE is located between 357 base pairs upstream (e.g., −357) of the transcription start site and 358 base pairs upstream (e.g., −358) of the transcription start site.

In some embodiments, the engineered promoter of the present disclosure comprises at least eight external CSREs. Embodiments of the location and the nucleic acid sequence of the first, second, third, fourth, fifth, sixth, and seventh external CSREs are provided herein and can be used in an engineered promoter comprising eight or more external CSREs. When a plurality of CSREs is included in the engineered promoter, the CSREs can be independently located at the same position and be contiguous or be provided at different positions and be non-contiguous. In some embodiments, the eighth external CSRE is located between the transcription start site and about 397 base pairs upstream (e.g., −397) of the transcription start site. In some alternative embodiments, the eighth external CSRE is located between about 7 base pairs upstream (e.g., −7) of the transcription start site and about 350 base pairs upstream (e.g., −397) of the transcription start site. In some specific embodiments, the eighth external CSRE is located between about 7 base pairs upstream (e.g., −7) of the transcription start site and about 86 base pairs upstream (e.g., −86) of the transcription start site. In some additional embodiments, the eighth external CSRE is located between 46 base pairs upstream (e.g., −46) of the transcription start site and 47 base pairs upstream (e.g., −47) of the transcription start site. In some specific embodiments, the eighth external CSRE is located between about 37 base pairs upstream (e.g., −37) of the transcription start site and about 116 base pairs upstream (e.g., −116) of the transcription start site. In some additional embodiments, the eighth external CSRE is located between 76 base pairs upstream (e.g., −76) of the transcription start site and 77 base pairs upstream (e.g., −77) of the transcription start site. In some specific embodiments, the eighth external CSRE is located between about 47 base pairs upstream (e.g., −47) of the transcription start site and about 126 base pairs upstream (e.g., −126) of the transcription start site. In some additional embodiments, the eighth external CSRE is located between 86 base pairs upstream (e.g., −86) of the transcription start site and 87 base pairs upstream (e.g., −87) of the transcription start site. In some specific embodiments, the eighth external CSRE is located between about 55 base pairs upstream (e.g., −55) of the transcription start site and about 134 base pairs upstream (e.g., −134) of the transcription start site. In some additional embodiments, the eighth external CSRE is located between 94 base pairs upstream (e.g., −94) of the transcription start site and 95 base pairs upstream (e.g., −95) of the transcription start site. In some specific embodiments, the eighth external CSRE is located between about 77 base pairs upstream (e.g., −77) of the transcription start site and about 156 base pairs upstream (e.g., −156) of the transcription start site. In some additional embodiments, the eighth external CSRE is located between 116 base pairs upstream (e.g., −116) of the transcription start site and 117 base pairs upstream (e.g., −117) of the transcription start site. In some specific embodiments, the eighth external CSRE is located between about 81 base pairs upstream (e.g., −81) of the transcription start site and about 160 base pairs upstream (e.g., −160) of the transcription start site. In some additional embodiments, the eighth external CSRE is located between 120 base pairs upstream (e.g., −120) of the transcription start site and 121 base pairs upstream (e.g., −121) of the transcription start site. In some specific embodiments, the eighth external CSRE is located between about 87 base pairs upstream (e.g., −87) of the transcription start site and about 166 base pairs upstream (e.g., −166) of the transcription start site. In some additional embodiments, the eighth external CSRE is located between 126 base pairs upstream (e.g., −126) of the transcription start site and 127 base pairs upstream (e.g., −127) of the transcription start site. In some specific embodiments, the eighth external CSRE is located between about 93 base pairs upstream (e.g., −93) of the transcription start site and about 172 base pairs upstream (e.g., −172) of the transcription start site. In some additional embodiments, the eighth external CSRE is located between 132 base pairs upstream (e.g., −132) of the transcription start site and 133 base pairs upstream (e.g., −133) of the transcription start site. In some specific embodiments, the eighth external CSRE is located between about 94 base pairs upstream (e.g., −94) of the transcription start site and about 173 base pairs upstream (e.g., −173) of the transcription start site. In some additional embodiments, the eighth external CSRE is located between 133 base pairs upstream (e.g., −133) of the transcription start site and 134 base pairs upstream (e.g., −134) of the transcription start site. In some specific embodiments, the eighth external CSRE is located between about 99 base pairs upstream (e.g., −99) of the transcription start site and about 178 base pairs upstream (e.g., −178) of the transcription start site. In some additional embodiments, the eighth external CSRE is located between 138 base pairs upstream (e.g., −138) of the transcription start site and 139 base pairs upstream (e.g., −139) of the transcription start site. In some specific embodiments, the eighth external CSRE is located between about 104 base pairs upstream (e.g., −104) of the transcription start site and about 183 base pairs upstream (e.g., −183) of the transcription start site. In some additional embodiments, the eighth external CSRE is located between 143 base pairs upstream (e.g., −143) of the transcription start site and 144 base pairs upstream (e.g., −144) of the transcription start site. In some specific embodiments, the eighth external CSRE is located between about 109 base pairs upstream (e.g., −109) of the transcription start site and about 188 base pairs upstream (e.g., −188) of the transcription start site. In some additional embodiments, the eighth external CSRE is located between 148 base pairs upstream (e.g., −148) of the transcription start site and 149 base pairs upstream (e.g., −149) of the transcription start site. In some specific embodiments, the eighth external CSRE is located between about 111 base pairs upstream (e.g., −111) of the transcription start site and about 190 base pairs upstream (e.g., −190) of the transcription start site. In some additional embodiments, the eighth external CSRE is located between 150 base pairs upstream (e.g., −150) of the transcription start site and 151 base pairs upstream (e.g., −151) of the transcription start site. In some specific embodiments, the eighth external CSRE is located between about 114 base pairs upstream (e.g., −114) of the transcription start site and about 193 base pairs upstream (e.g., −193) of the transcription start site. In some additional embodiments, the eighth external CSRE is located between 153 base pairs upstream (e.g., −153) of the transcription start site and 154 base pairs upstream (e.g., −154) of the transcription start site. In some specific embodiments, the eighth external CSRE is located between about 119 base pairs upstream (e.g., −119) of the transcription start site and about 198 base pairs upstream (e.g., −198) of the transcription start site. In some additional embodiments, the eighth external CSRE is located between 158 base pairs upstream (e.g., −158) of the transcription start site and 159 base pairs upstream (e.g., −159) of the transcription start site. In some specific embodiments, the eighth external CSRE is located between about 124 base pairs upstream (e.g., −124) of the transcription start site and about 203 base pairs upstream (e.g., −203) of the transcription start site. In some additional embodiments, the eighth external CSRE is located between 163 base pairs upstream (e.g., −163) of the transcription start site and 164 base pairs upstream (e.g., −164) of the transcription start site. In some specific embodiments, the eighth external CSRE is located between about 129 base pairs upstream (e.g., −129) of the transcription start site and about 208 base pairs upstream (e.g., −208) of the transcription start site. In some additional embodiments, the eighth external CSRE is located between 168 base pairs upstream (e.g., −168) of the transcription start site and 169 base pairs upstream (e.g., −169) of the transcription start site. In some specific embodiments, the eighth external CSRE is located between about 189 base pairs upstream (e.g., −189) of the transcription start site and about 268 base pairs upstream (e.g., −268) of the transcription start site. In some additional embodiments, the eighth external CSRE is located between 228 base pairs upstream (e.g., −228) of the transcription start site and 229 base pairs upstream (e.g., −229) of the transcription start site. In some specific embodiments, the eighth external CSRE is located between about 198 base pairs upstream (e.g., −198) of the transcription start site and about 277 base pairs upstream (e.g., −277) of the transcription start site. In some additional embodiments, the eighth external CSRE is located between 237 base pairs upstream (e.g., −237) of the transcription start site and 238 base pairs upstream (e.g., −238) of the transcription start site. In some specific embodiments, the eighth external CSRE is located between about 239 base pairs upstream (e.g., −239) of the transcription start site and about 318 base pairs upstream (e.g., −318) of the transcription start site. In some additional embodiments, the eighth external CSRE is located between 278 base pairs upstream (e.g., −278) of the transcription start site and 279 base pairs upstream (e.g., −279) of the transcription start site. In some specific embodiments, the eighth external CSRE is located between about 318 base pairs upstream (e.g., −318) of the transcription start site and about 397 base pairs upstream (e.g., −397) of the transcription start site. In some additional embodiments, the eighth external CSRE is located between 357 base pairs upstream (e.g., −357) of the transcription start site and 358 base pairs upstream (e.g., −358) of the transcription start site.

In some embodiments, the engineered promoter of the present disclosure comprises at least nine external CSREs. Embodiments of the location and the nucleic acid sequence of the first, second, third, fourth, fifth, sixth, seventh, and eighth external CSREs are provided herein and can be used in an engineered promoter comprising nine or more external CSREs. When a plurality of CSREs is included in the engineered promoter, the CSREs can be independently located at the same position and be contiguous or be provided at different positions and be non-contiguous. In some embodiments, the ninth external CSRE is located between the transcription start site and about 397 base pairs upstream (e.g., −397) of the transcription start site. In some alternative embodiments, the ninth external CSRE is located between about 7 base pairs upstream (e.g., −7) of the transcription start site and about 350 base pairs upstream (e.g., −397) of the transcription start site. In some specific embodiments, the ninth external CSRE is located between about 7 base pairs upstream (e.g., −7) of the transcription start site and about 86 base pairs upstream (e.g., −86) of the transcription start site. In some additional embodiments, the ninth external CSRE is located between 46 base pairs upstream (e.g., −46) of the transcription start site and 47 base pairs upstream (e.g., −47) of the transcription start site. In some specific embodiments, the ninth external CSRE is located between about 37 base pairs upstream (e.g., −37) of the transcription start site and about 116 base pairs upstream (e.g., −116) of the transcription start site. In some additional embodiments, the ninth external CSRE is located between 76 base pairs upstream (e.g., −76) of the transcription start site and 77 base pairs upstream (e.g., −77) of the transcription start site. In some specific embodiments, the ninth external CSRE is located between about 47 base pairs upstream (e.g., −47) of the transcription start site and about 126 base pairs upstream (e.g., −126) of the transcription start site. In some additional embodiments, the ninth external CSRE is located between 86 base pairs upstream (e.g., −86) of the transcription start site and 87 base pairs upstream (e.g., −87) of the transcription start site. In some specific embodiments, the ninth external CSRE is located between about 55 base pairs upstream (e.g., −55) of the transcription start site and about 134 base pairs upstream (e.g., −134) of the transcription start site. In some additional embodiments, the ninth external CSRE is located between 94 base pairs upstream (e.g., −94) of the transcription start site and 95 base pairs upstream (e.g., −95) of the transcription start site. In some specific embodiments, the ninth external CSRE is located between about 77 base pairs upstream (e.g., −77) of the transcription start site and about 156 base pairs upstream (e.g., −156) of the transcription start site. In some additional embodiments, the ninth external CSRE is located between 116 base pairs upstream (e.g., −116) of the transcription start site and 117 base pairs upstream (e.g., −117) of the transcription start site. In some specific embodiments, the ninth external CSRE is located between about 81 base pairs upstream (e.g., −81) of the transcription start site and about 160 base pairs upstream (e.g., −160) of the transcription start site. In some additional embodiments, the ninth external CSRE is located between 120 base pairs upstream (e.g., −120) of the transcription start site and 121 base pairs upstream (e.g., −121) of the transcription start site. In some specific embodiments, the ninth external CSRE is located between about 87 base pairs upstream (e.g., −87) of the transcription start site and about 166 base pairs upstream (e.g., −166) of the transcription start site. In some additional embodiments, the ninth external CSRE is located between 126 base pairs upstream (e.g., −126) of the transcription start site and 127 base pairs upstream (e.g., −127) of the transcription start site. In some specific embodiments, the ninth external CSRE is located between about 93 base pairs upstream (e.g., −93) of the transcription start site and about 172 base pairs upstream (e.g., −172) of the transcription start site. In some additional embodiments, the ninth external CSRE is located between 132 base pairs upstream (e.g., −132) of the transcription start site and 133 base pairs upstream (e.g., −133) of the transcription start site. In some specific embodiments, the ninth external CSRE is located between about 94 base pairs upstream (e.g., −94) of the transcription start site and about 173 base pairs upstream (e.g., −173) of the transcription start site. In some additional embodiments, the ninth external CSRE is located between 133 base pairs upstream (e.g., −133) of the transcription start site and 134 base pairs upstream (e.g., −134) of the transcription start site. In some specific embodiments, the ninth external CSRE is located between about 99 base pairs upstream (e.g., −99) of the transcription start site and about 178 base pairs upstream (e.g., −178) of the transcription start site. In some additional embodiments, the ninth external CSRE is located between 138 base pairs upstream (e.g., −138) of the transcription start site and 139 base pairs upstream (e.g., −139) of the transcription start site. In some specific embodiments, the ninth external CSRE is located between about 104 base pairs upstream (e.g., −104) of the transcription start site and about 183 base pairs upstream (e.g., −183) of the transcription start site. In some additional embodiments, the ninth external CSRE is located between 143 base pairs upstream (e.g., −143) of the transcription start site and 144 base pairs upstream (e.g., −144) of the transcription start site. In some specific embodiments, the ninth external CSRE is located between about 109 base pairs upstream (e.g., −109) of the transcription start site and about 188 base pairs upstream (e.g., −188) of the transcription start site. In some additional embodiments, the ninth external CSRE is located between 148 base pairs upstream (e.g., −148) of the transcription start site and 149 base pairs upstream (e.g., −149) of the transcription start site. In some specific embodiments, the ninth external CSRE is located between about 111 base pairs upstream (e.g., −111) of the transcription start site and about 190 base pairs upstream (e.g., −190) of the transcription start site. In some additional embodiments, the ninth external CSRE is located between 150 base pairs upstream (e.g., −150) of the transcription start site and 151 base pairs upstream (e.g., −151) of the transcription start site. In some specific embodiments, the ninth external CSRE is located between about 114 base pairs upstream (e.g., −114) of the transcription start site and about 193 base pairs upstream (e.g., −193) of the transcription start site. In some additional embodiments, the ninth external CSRE is located between 153 base pairs upstream (e.g., −153) of the transcription start site and 154 base pairs upstream (e.g., −154) of the transcription start site. In some specific embodiments, the ninth external CSRE is located between about 119 base pairs upstream (e.g., −119) of the transcription start site and about 198 base pairs upstream (e.g., −198) of the transcription start site. In some additional embodiments, the ninth external CSRE is located between 158 base pairs upstream (e.g., −158) of the transcription start site and 159 base pairs upstream (e.g., −159) of the transcription start site. In some specific embodiments, the ninth external CSRE is located between about 124 base pairs upstream (e.g., −124) of the transcription start site and about 203 base pairs upstream (e.g., −203) of the transcription start site. In some additional embodiments, the ninth external CSRE is located between 163 base pairs upstream (e.g., −163) of the transcription start site and 164 base pairs upstream (e.g., −164) of the transcription start site. In some specific embodiments, the ninth external CSRE is located between about 129 base pairs upstream (e.g., −129) of the transcription start site and about 208 base pairs upstream (e.g., −208) of the transcription start site. In some additional embodiments, the ninth external CSRE is located between 168 base pairs upstream (e.g., −168) of the transcription start site and 169 base pairs upstream (e.g., −169) of the transcription start site. In some specific embodiments, the ninth external CSRE is located between about 189 base pairs upstream (e.g., −189) of the transcription start site and about 268 base pairs upstream (e.g., −268) of the transcription start site. In some additional embodiments, the ninth external CSRE is located between 228 base pairs upstream (e.g., −228) of the transcription start site and 229 base pairs upstream (e.g., −229) of the transcription start site. In some specific embodiments, the ninth external CSRE is located between about 198 base pairs upstream (e.g., −198) of the transcription start site and about 277 base pairs upstream (e.g., −277) of the transcription start site. In some additional embodiments, the ninth external CSRE is located between 237 base pairs upstream (e.g., −237) of the transcription start site and 238 base pairs upstream (e.g., −238) of the transcription start site. In some specific embodiments, the ninth external CSRE is located between about 239 base pairs upstream (e.g., −239) of the transcription start site and about 318 base pairs upstream (e.g., −318) of the transcription start site. In some additional embodiments, the ninth external CSRE is located between 278 base pairs upstream (e.g., −278) of the transcription start site and 279 base pairs upstream (e.g., −279) of the transcription start site. In some specific embodiments, the ninth external CSRE is located between about 318 base pairs upstream (e.g., −318) of the transcription start site and about 397 base pairs upstream (e.g., −397) of the transcription start site. In some additional embodiments, the ninth external CSRE is located between 357 base pairs upstream (e.g., −357) of the transcription start site and 358 base pairs upstream (e.g., −358) of the transcription start site.

In some embodiments, the engineered promoter of the present disclosure comprises at least ten external CSREs. Embodiments of the location and the nucleic acid sequence of the first, second, third, fourth, fifth, sixth, seventh, eighth, and ninth external CSREs are provided herein and can be used in an engineered promoter comprising ten or more external CSREs. When a plurality of CSREs is included in the engineered promoter, the CSREs can be independently located at the same position and be contiguous or be provided at different positions and be non-contiguous. In some embodiments, the tenth external CSRE is located between the transcription start site and about 397 base pairs upstream (e.g., −397) of the transcription start site. In some alternative embodiments, the tenth external CSRE is located between about 7 base pairs upstream (e.g., −7) of the transcription start site and about 350 base pairs upstream (e.g., −397) of the transcription start site. In some specific embodiments, the tenth external CSRE is located between about 7 base pairs upstream (e.g., −7) of the transcription start site and about 86 base pairs upstream (e.g., −86) of the transcription start site. In some additional embodiments, the tenth external CSRE is located between 46 base pairs upstream (e.g., −46) of the transcription start site and 47 base pairs upstream (e.g., −47) of the transcription start site. In some specific embodiments, the tenth external CSRE is located between about 37 base pairs upstream (e.g., −37) of the transcription start site and about 116 base pairs upstream (e.g., −116) of the transcription start site. In some additional embodiments, the tenth external CSRE is located between 76 base pairs upstream (e.g., −76) of the transcription start site and 77 base pairs upstream (e.g., −77) of the transcription start site. In some specific embodiments, the tenth external CSRE is located between about 47 base pairs upstream (e.g., −47) of the transcription start site and about 126 base pairs upstream (e.g., −126) of the transcription start site. In some additional embodiments, the tenth external CSRE is located between 86 base pairs upstream (e.g., −86) of the transcription start site and 87 base pairs upstream (e.g., −87) of the transcription start site. In some specific embodiments, the tenth external CSRE is located between about 55 base pairs upstream (e.g., −55) of the transcription start site and about 134 base pairs upstream (e.g., −134) of the transcription start site. In some additional embodiments, the tenth external CSRE is located between 94 base pairs upstream (e.g., −94) of the transcription start site and 95 base pairs upstream (e.g., −95) of the transcription start site. In some specific embodiments, the tenth external CSRE is located between about 77 base pairs upstream (e.g., −77) of the transcription start site and about 156 base pairs upstream (e.g., −156) of the transcription start site. In some additional embodiments, the tenth external CSRE is located between 116 base pairs upstream (e.g., −116) of the transcription start site and 117 base pairs upstream (e.g., −117) of the transcription start site. In some specific embodiments, the tenth external CSRE is located between about 81 base pairs upstream (e.g., −81) of the transcription start site and about 160 base pairs upstream (e.g., −160) of the transcription start site. In some additional embodiments, the tenth external CSRE is located between 120 base pairs upstream (e.g., −120) of the transcription start site and 121 base pairs upstream (e.g., −121) of the transcription start site. In some specific embodiments, the tenth external CSRE is located between about 87 base pairs upstream (e.g., −87) of the transcription start site and about 166 base pairs upstream (e.g., −166) of the transcription start site. In some additional embodiments, the tenth external CSRE is located between 126 base pairs upstream (e.g., −126) of the transcription start site and 127 base pairs upstream (e.g., −127) of the transcription start site. In some specific embodiments, the tenth external CSRE is located between about 93 base pairs upstream (e.g., −93) of the transcription start site and about 172 base pairs upstream (e.g., −172) of the transcription start site. In some additional embodiments, the tenth external CSRE is located between 132 base pairs upstream (e.g., −132) of the transcription start site and 133 base pairs upstream (e.g., −133) of the transcription start site. In some specific embodiments, the tenth external CSRE is located between about 94 base pairs upstream (e.g., −94) of the transcription start site and about 173 base pairs upstream (e.g., −173) of the transcription start site. In some additional embodiments, the tenth external CSRE is located between 133 base pairs upstream (e.g., −133) of the transcription start site and 134 base pairs upstream (e.g., −134) of the transcription start site. In some specific embodiments, the tenth external CSRE is located between about 99 base pairs upstream (e.g., −99) of the transcription start site and about 178 base pairs upstream (e.g., −178) of the transcription start site. In some additional embodiments, the tenth external CSRE is located between 138 base pairs upstream (e.g., −138) of the transcription start site and 139 base pairs upstream (e.g., −139) of the transcription start site. In some specific embodiments, the tenth external CSRE is located between about 104 base pairs upstream (e.g., −104) of the transcription start site and about 183 base pairs upstream (e.g., −183) of the transcription start site. In some additional embodiments, the tenth external CSRE is located between 143 base pairs upstream (e.g., −143) of the transcription start site and 144 base pairs upstream (e.g., −144) of the transcription start site. In some specific embodiments, the tenth external CSRE is located between about 109 base pairs upstream (e.g., −109) of the transcription start site and about 188 base pairs upstream (e.g., −188) of the transcription start site. In some additional embodiments, the tenth external CSRE is located between 148 base pairs upstream (e.g., −148) of the transcription start site and 149 base pairs upstream (e.g., −149) of the transcription start site. In some specific embodiments, the tenth external CSRE is located between about 111 base pairs upstream (e.g., −111) of the transcription start site and about 190 base pairs upstream (e.g., −190) of the transcription start site. In some additional embodiments, the tenth external CSRE is located between 150 base pairs upstream (e.g., −150) of the transcription start site and 151 base pairs upstream (e.g., −151) of the transcription start site. In some specific embodiments, the tenth external CSRE is located between about 114 base pairs upstream (e.g., −114) of the transcription start site and about 193 base pairs upstream (e.g., −193) of the transcription start site. In some additional embodiments, the tenth external CSRE is located between 153 base pairs upstream (e.g., −153) of the transcription start site and 154 base pairs upstream (e.g., −154) of the transcription start site. In some specific embodiments, the tenth external CSRE is located between about 119 base pairs upstream (e.g., −119) of the transcription start site and about 198 base pairs upstream (e.g., −198) of the transcription start site. In some additional embodiments, the tenth external CSRE is located between 158 base pairs upstream (e.g., −158) of the transcription start site and 159 base pairs upstream (e.g., −159) of the transcription start site. In some specific embodiments, the tenth external CSRE is located between about 124 base pairs upstream (e.g., −124) of the transcription start site and about 203 base pairs upstream (e.g., −203) of the transcription start site. In some additional embodiments, the tenth external CSRE is located between 163 base pairs upstream (e.g., −163) of the transcription start site and 164 base pairs upstream (e.g., −164) of the transcription start site. In some specific embodiments, the tenth external CSRE is located between about 129 base pairs upstream (e.g., −129) of the transcription start site and about 208 base pairs upstream (e.g., −208) of the transcription start site. In some additional embodiments, the tenth external CSRE is located between 168 base pairs upstream (e.g., −168) of the transcription start site and 169 base pairs upstream (e.g., −169) of the transcription start site. In some specific embodiments, the tenth external CSRE is located between about 189 base pairs upstream (e.g., −189) of the transcription start site and about 268 base pairs upstream (e.g., −268) of the transcription start site. In some additional embodiments, the tenth external CSRE is located between 228 base pairs upstream (e.g., −228) of the transcription start site and 229 base pairs upstream (e.g., −229) of the transcription start site. In some specific embodiments, the tenth external CSRE is located between about 198 base pairs upstream (e.g., −198) of the transcription start site and about 277 base pairs upstream (e.g., −277) of the transcription start site. In some additional embodiments, the tenth external CSRE is located between 237 base pairs upstream (e.g., −237) of the transcription start site and 238 base pairs upstream (e.g., −238) of the transcription start site. In some specific embodiments, the tenth external CSRE is located between about 239 base pairs upstream (e.g., −239) of the transcription start site and about 318 base pairs upstream (e.g., −318) of the transcription start site. In some additional embodiments, the tenth external CSRE is located between 278 base pairs upstream (e.g., −278) of the transcription start site and 279 base pairs upstream (e.g., −279) of the transcription start site. In some specific embodiments, the tenth external CSRE is located between about 318 base pairs upstream (e.g., −318) of the transcription start site and about 397 base pairs upstream (e.g., −397) of the transcription start site. In some additional embodiments, the tenth external CSRE is located between 357 base pairs upstream (e.g., −357) of the transcription start site and 358 base pairs upstream (e.g., −358) of the transcription start site. In yet another specific embodiment, the engineered promoter with at least ten external CSREs comprises a first external CSRE located between 94 base pairs upstream (e.g., −94) of the transcription start site and 173 base pairs upstream (e.g., −173) of the transcription start site; a second external CSRE located between 99 base pairs upstream (e.g., −99) of the transcription start site and 178 base pairs upstream (e.g., −178) of the transcription start site; a third external CSRE located between 104 base pairs upstream (e.g., −104) of the transcription start site and 183 base pairs upstream (e.g., −183) of the transcription start site; a fourth external CSRE located between 109 base pairs upstream (e.g., −109) of the transcription start site and 188 base pairs upstream (e.g., −188) of the transcription start site; a fifth external CSRE located between 114 base pairs upstream (e.g., −114) of the transcription start site and 193 base pairs upstream (e.g., −193) of the transcription start site; a sixth external CSRE located between 119 base pairs upstream (e.g., −119) of the transcription start site and 198 base pairs upstream (e.g., −198) of the transcription start site; a seventh external CSRE located between 124 base pairs upstream (e.g., −124) of the transcription start site and 203 base pairs upstream (e.g., −203) of the transcription start site; an eighth external CSRE located between 129 base pairs upstream (e.g., −129) of the transcription start site and 208 base pairs upstream (e.g., −208) of the transcription start site; a ninth external CSRE located between 198 base pairs upstream (e.g., −198) of the transcription start site and 277 base pairs upstream (e.g., −277) of the transcription start site; and a tenth external CSRE located between 318 base pairs upstream (e.g., −318) of the transcription start site and 397 base pairs upstream (e.g., −397) of the transcription start site. For example, the engineered promoter with at least ten external CSREs comprises a first external CSRE located between 133 base pairs upstream (e.g., −133) of the transcription start site and 134 base pairs upstream (e.g., −134) of the transcription start site; a second external CSRE located between 138 base pairs upstream (e.g., −138) of the transcription start site and 139 base pairs upstream (e.g., −139) of the transcription start site; a third external CSRE located between 143 base pairs upstream (e.g., −143) of the transcription start site and 144 base pairs upstream (e.g., −144) of the transcription start site; a fourth external CSRE located between 148 base pairs upstream (e.g., −148) of the transcription start site and 149 base pairs upstream (e.g., −149) of the transcription start site; a fifth external CSRE located between 153 base pairs upstream (e.g., −153) of the transcription start site and 154 base pairs upstream (e.g., −154) of the transcription start site; a sixth external CSRE located between 158 base pairs upstream (e.g., −158) of the transcription start site and 159 base pairs upstream (e.g., −159) of the transcription start site; a seventh external CSRE located between 163 base pairs upstream (e.g., −163) of the transcription start site and 164 base pairs upstream (e.g., −164) of the transcription start site; an eighth external CSRE located between 168 base pairs upstream (e.g., −168) of the transcription start site and 169 base pairs upstream (e.g., −169) of the transcription start site; a ninth external CSRE located between 237 base pairs upstream (e.g., −237) of the transcription start site and 238 base pairs upstream (e.g., −238) of the transcription start site; and a tenth external CSRE located between 357 base pairs upstream (e.g., −357) of the transcription start site and 358 base pairs upstream (e.g., −358) of the transcription start site.

Some of the parental/engineered promoters include a TATA box and, in some embodiments, the CSREs are located with respect to the position of the TATA box. In some embodiments, the engineered promoter of the present disclosure comprises a first external CSRE located between about 38 base pairs downstream (e.g., +38) of the TATA box and at most 363 base pairs upstream (e.g., −363) of the TATA box. In some instances, the engineered promoter can include one or more external CSREs which can be located more than 363 base pairs upstream (e.g., −363) of the TATA box (provided that it includes at least one external CSRE at most 363 base pairs upstream of the TATA box). In some embodiments, the engineered promoter of the present disclosure comprises a first external CSRE located at most 275 base pairs upstream (e.g., −275) of the TATA box. In some instances, the engineered promoter can include one or more external CSREs which can be located more than 275 base pairs upstream (e.g., −275) of the TATA box (provided that it includes at least one external CSRE at most 275 base pairs upstream of the TATA box).

In some alternative embodiments, the first external CSRE is located between about 38 base pairs downstream (e.g., +38) of the TATA box and about 363 base pairs upstream (e.g., −363) of the TATA box. In some specific embodiments, the first external CSRE is located between about 38 base pairs downstream (e.g., +38) of the TATA box and about 41 base pairs upstream (e.g., −41) of the TATA box. In some additional embodiments, the first external CSRE is located between 1 base pair upstream (e.g., −1) of the TATA box and 2 base pairs upstream (e.g., −2) of the TATA box. In some specific embodiments, the first external CSRE is located between about 8 base pairs downstream (e.g., +8) of the TATA box and about 71 base pairs upstream (e.g., −71) of the TATA box. In some additional embodiments, the first external CSRE is located between 31 base pairs upstream (e.g., −31) of the TATA box and 32 base pairs upstream (e.g., −32) of the TATA box. In some specific embodiments, the first external CSRE is located between about 2 base pairs upstream (e.g., −2) of the TATA box and about 81 base pairs upstream (e.g., −81) of the TATA box. In some additional embodiments, the first external CSRE is located between 41 base pairs upstream (e.g., −41) of the TATA box and 42 base pairs upstream (e.g., −42) of the TATA box. In some specific embodiments, the first external CSRE is located between about 10 base pairs upstream (e.g., −10) of the TATA box and about 89 base pairs upstream (e.g., −89) of the TATA box. In some additional embodiments, the first external CSRE is located between 49 base pairs upstream (e.g., −49) of the TATA box and 50 base pairs upstream (e.g., −50) of the TATA box. In some specific embodiments, the first external CSRE is located between about 32 base pairs upstream (e.g., −32) of the TATA box and about 111 base pairs upstream (e.g., −111) of the TATA box. In some additional embodiments, the first external CSRE is located between 71 base pairs upstream (e.g., −71) of the TATA box and 72 base pairs upstream (e.g., −72) of the TATA box. In some specific embodiments, the first external CSRE is located between about 36 base pairs upstream (e.g., −36) of the TATA box and about 115 base pairs upstream (e.g., −115) of the TATA box. In some additional embodiments, the first external CSRE is located between 75 base pairs upstream (e.g., −75) of the TATA box and 76 base pairs upstream (e.g., −76) of the TATA box. In some specific embodiments, the first external CSRE is located between about 42 base pairs upstream (e.g., −42) of the TATA box and about 121 base pairs upstream (e.g., −121) of the TATA box. In some additional embodiments, the first external CSRE is located between 81 base pairs upstream (e.g., −81) of the TATA box and 82 base pairs upstream (e.g., −82) of the TATA box. In some specific embodiments, the first external CSRE is located between about 59 base pairs upstream (e.g., −59) of the TATA box and about 138 base pairs upstream (e.g., −138) of the TATA box. In some additional embodiments, the first external CSRE is located between 98 base pairs upstream (e.g., −98) of the TATA box and 99 base pairs upstream (e.g., −99) of the TATA box. In some specific embodiments, the first external CSRE is located between about 60 base pairs upstream (e.g., −60) of the TATA box and about 139 base pairs upstream (e.g., −139) of the TATA box. In some additional embodiments, the first external CSRE is located between 99 base pairs upstream (e.g., −99) of the TATA box and 100 base pairs upstream (e.g., −100) of the TATA box. In some specific embodiments, the first external CSRE is located between about 65 base pairs upstream (e.g., −65) of the TATA box and about 144 base pairs upstream (e.g., −144) of the TATA box. In some additional embodiments, the first external CSRE is located between 104 base pairs upstream (e.g., −104) of the TATA box and 105 base pairs upstream (e.g., −105) of the TATA box. In some specific embodiments, the first external CSRE is located between about 70 base pairs upstream (e.g., −70) of the TATA box and about 149 base pairs upstream (e.g., −149) of the TATA box. In some additional embodiments, the first external CSRE is located between 109 base pairs upstream (e.g., −109) of the TATA box and 110 base pairs upstream (e.g., −110) of the TATA box. In some specific embodiments, the first external CSRE is located between about 75 base pairs upstream (e.g., −75) of the TATA box and about 154 base pairs upstream (e.g., −154) of the TATA box. In some additional embodiments, the first external CSRE is located between 114 base pairs upstream (e.g., −114) of the TATA box and 115 base pairs upstream (e.g., −115) of the TATA box. In some specific embodiments, the first external CSRE is located between about 77 base pairs upstream (e.g., −77) of the TATA box and about 156 base pairs upstream (e.g., −156) of the TATA box. In some additional embodiments, the first external CSRE is located between 116 base pairs upstream (e.g., −116) of the TATA box and 117 base pairs upstream (e.g., −117) of the TATA box. In some specific embodiments, the first external CSRE is located between about 80 base pairs upstream (e.g., −80) of the TATA box and about 159 base pairs upstream (e.g., −159) of the TATA box. In some additional embodiments, the first external CSRE is located between 119 base pairs upstream (e.g., −119) of the TATA box and 120 base pairs upstream (e.g., −120) of the TATA box. In some specific embodiments, the first external CSRE is located between about 85 base pairs upstream (e.g., −85) of the TATA box and about 164 base pairs upstream (e.g., −164) of the TATA box. In some additional embodiments, the first external CSRE is located between 124 base pairs upstream (e.g., −124) of the TATA box and 125 base pairs upstream (e.g., −125) of the TATA box. In some specific embodiments, the first external CSRE is located between about 90 base pairs upstream (e.g., −90) of the TATA box and about 169 base pairs upstream (e.g., −169) of the TATA box. In some additional embodiments, the first external CSRE is located between 129 base pairs upstream (e.g., −129) of the TATA box and 130 base pairs upstream (e.g., −130) of the TATA box. In some specific embodiments, the first external CSRE is located between about 95 base pairs upstream (e.g., −95) of the TATA box and about 174 base pairs upstream (e.g., −174) of the TATA box. In some additional embodiments, the first external CSRE is located between 134 base pairs upstream (e.g., −134) of the TATA box and 135 base pairs upstream (e.g., −135) of the TATA box. In some specific embodiments, the first external CSRE is located between about 155 base pairs upstream (e.g., −155) of the TATA box and about 234 base pairs upstream (e.g., −234) of the TATA box. In some additional embodiments, the first external CSRE is located between 194 base pairs upstream (e.g., −194) of the TATA box and 195 base pairs upstream (e.g., −195) of the TATA box. In some specific embodiments, the first external CSRE is located between about 164 base pairs upstream (e.g., −164) of the TATA box and about 243 base pairs upstream (e.g., −243) of the TATA box. In some additional embodiments, the first external CSRE is located between 203 base pairs upstream (e.g., −203) of the TATA box and 204 base pairs upstream (e.g., −204) of the TATA box. In some specific embodiments, the first external CSRE is located between about 205 base pairs upstream (e.g., −205) of the TATA box and about 284 base pairs upstream (e.g., −284) of the TATA box. In some additional embodiments, the first external CSRE is located between 244 base pairs upstream (e.g., −244) of the TATA box and 245 base pairs upstream (e.g., −245) of the TATA box. In some specific embodiments, the first external CSRE is located between about 284 base pairs upstream (e.g., −284) of the TATA box and about 363 base pairs upstream (e.g., −363) of the TATA box. In some additional embodiments, the first external CSRE is located between 323 base pairs upstream (e.g., −323) of the TATA box and 324 base pairs upstream (e.g., −324) of the TATA box.

In some embodiments, the engineered promoter of the present disclosure comprises at least two external CSREs. Embodiments of the location and the nucleic acid sequence of the first external CSRE are provided herein and can be used in an engineered promoter comprising two or more external CSREs. When a plurality of CSREs is included in the engineered promoter, the CSREs can be independently located at the same position and be contiguous or be provided at different positions and be non-contiguous. In some alternative embodiments, the second external CSRE is located between about 38 base pairs downstream (e.g., +38) of the TATA box and about 363 base pairs upstream (e.g., −363) of the TATA box. In some specific embodiments, the second external CSRE is located between about 38 base pairs downstream (e.g., +38) of the TATA box and about 41 base pairs upstream (e.g., −41) of the TATA box. In some additional embodiments, the second external CSRE is located between 1 base pair upstream (e.g., −1) of the TATA box and 2 base pairs upstream (e.g., −2) of the TATA box. In some specific embodiments, the second external CSRE is located between about 8 base pairs downstream (e.g., +8) of the TATA box and about 71 base pairs upstream (e.g., −71) of the TATA box. In some additional embodiments, the second external CSRE is located between 31 base pairs upstream (e.g., −31) of the TATA box and 32 base pairs upstream (e.g., −32) of the TATA box. In some specific embodiments, the second external CSRE is located between about 2 base pairs upstream (e.g., −2) of the TATA box and about 81 base pairs upstream (e.g., −81) of the TATA box. In some additional embodiments, the second external CSRE is located between 41 base pairs upstream (e.g., −41) of the TATA box and 42 base pairs upstream (e.g., −42) of the TATA box. In some specific embodiments, the second external CSRE is located between about 10 base pairs upstream (e.g., −10) of the TATA box and about 89 base pairs upstream (e.g., −89) of the TATA box. In some additional embodiments, the second external CSRE is located between 49 base pairs upstream (e.g., −49) of the TATA box and 50 base pairs upstream (e.g., −50) of the TATA box. In some specific embodiments, the second external CSRE is located between about 32 base pairs upstream (e.g., −32) of the TATA box and about 111 base pairs upstream (e.g., −111) of the TATA box. In some additional embodiments, the second external CSRE is located between 71 base pairs upstream (e.g., −71) of the TATA box and 72 base pairs upstream (e.g., −72) of the TATA box. In some specific embodiments, the second external CSRE is located between about 36 base pairs upstream (e.g., −36) of the TATA box and about 115 base pairs upstream (e.g., −115) of the TATA box. In some additional embodiments, the second external CSRE is located between 75 base pairs upstream (e.g., −75) of the TATA box and 76 base pairs upstream (e.g., −76) of the TATA box. In some specific embodiments, the second external CSRE is located between about 42 base pairs upstream (e.g., −42) of the TATA box and about 121 base pairs upstream (e.g., −121) of the TATA box. In some additional embodiments, the second external CSRE is located between 81 base pairs upstream (e.g., −81) of the TATA box and 82 base pairs upstream (e.g., −82) of the TATA box. In some specific embodiments, the second external CSRE is located between about 59 base pairs upstream (e.g., −59) of the TATA box and about 138 base pairs upstream (e.g., −138) of the TATA box. In some additional embodiments, the second external CSRE is located between 98 base pairs upstream (e.g., −98) of the TATA box and 99 base pairs upstream (e.g., −99) of the TATA box. In some specific embodiments, the second external CSRE is located between about 60 base pairs upstream (e.g., −60) of the TATA box and about 139 base pairs upstream (e.g., −139) of the TATA box. In some additional embodiments, the second external CSRE is located between 99 base pairs upstream (e.g., −99) of the TATA box and 100 base pairs upstream (e.g., −100) of the TATA box. In some specific embodiments, the second external CSRE is located between about 65 base pairs upstream (e.g., −65) of the TATA box and about 144 base pairs upstream (e.g., −144) of the TATA box. In some additional embodiments, the second external CSRE is located between 104 base pairs upstream (e.g., −104) of the TATA box and 105 base pairs upstream (e.g., −105) of the TATA box. In some specific embodiments, the second external CSRE is located between about 70 base pairs upstream (e.g., −70) of the TATA box and about 149 base pairs upstream (e.g., −149) of the TATA box. In some additional embodiments, the second external CSRE is located between 109 base pairs upstream (e.g., −109) of the TATA box and 110 base pairs upstream (e.g., −110) of the TATA box. In some specific embodiments, the second external CSRE is located between about 75 base pairs upstream (e.g., −75) of the TATA box and about 154 base pairs upstream (e.g., −154) of the TATA box. In some additional embodiments, the second external CSRE is located between 114 base pairs upstream (e.g., −114) of the TATA box and 115 base pairs upstream (e.g., −115) of the TATA box. In some specific embodiments, the second external CSRE is located between about 77 base pairs upstream (e.g., −77) of the TATA box and about 156 base pairs upstream (e.g., −156) of the TATA box. In some additional embodiments, the second external CSRE is located between 116 base pairs upstream (e.g., −116) of the TATA box and 117 base pairs upstream (e.g., −117) of the TATA box. In some specific embodiments, the second external CSRE is located between about 80 base pairs upstream (e.g., −80) of the TATA box and about 159 base pairs upstream (e.g., −159) of the TATA box. In some additional embodiments, the second external CSRE is located between 119 base pairs upstream (e.g., −119) of the TATA box and 120 base pairs upstream (e.g., −120) of the TATA box. In some specific embodiments, the second external CSRE is located between about 85 base pairs upstream (e.g., −85) of the TATA box and about 164 base pairs upstream (e.g., −164) of the TATA box. In some additional embodiments, the second external CSRE is located between 124 base pairs upstream (e.g., −124) of the TATA box and 125 base pairs upstream (e.g., −125) of the TATA box. In some specific embodiments, the second external CSRE is located between about 90 base pairs upstream (e.g., −90) of the TATA box and about 169 base pairs upstream (e.g., −169) of the TATA box. In some additional embodiments, the second external CSRE is located between 129 base pairs upstream (e.g., −129) of the TATA box and 130 base pairs upstream (e.g., −130) of the TATA box. In some specific embodiments, the second external CSRE is located between about 95 base pairs upstream (e.g., −95) of the TATA box and about 174 base pairs upstream (e.g., −174) of the TATA box. In some additional embodiments, the second external CSRE is located between 134 base pairs upstream (e.g., −134) of the TATA box and 135 base pairs upstream (e.g., −135) of the TATA box. In some specific embodiments, the second external CSRE is located between about 155 base pairs upstream (e.g., −155) of the TATA box and about 234 base pairs upstream (e.g., −234) of the TATA box. In some additional embodiments, the second external CSRE is located between 194 base pairs upstream (e.g., −194) of the TATA box and 195 base pairs upstream (e.g., −195) of the TATA box. In some specific embodiments, the second external CSRE is located between about 164 base pairs upstream (e.g., −164) of the TATA box and about 243 base pairs upstream (e.g., −243) of the TATA box. In some additional embodiments, the second external CSRE is located between 203 base pairs upstream (e.g., −203) of the TATA box and 204 base pairs upstream (e.g., −204) of the TATA box. In some specific embodiments, the second external CSRE is located between about 205 base pairs upstream (e.g., −205) of the TATA box and about 284 base pairs upstream (e.g., −284) of the TATA box. In some additional embodiments, the second external CSRE is located between 244 base pairs upstream (e.g., −244) of the TATA box and 245 base pairs upstream (e.g., −245) of the TATA box. In some specific embodiments, the second external CSRE is located between about 284 base pairs upstream (e.g., −284) of the TATA box and about 363 base pairs upstream (e.g., −363) of the TATA box. In some additional embodiments, the second external CSRE is located between 323 base pairs upstream (e.g., −323) of the TATA box and 324 base pairs upstream (e.g., −324) of the TATA box. In still another embodiment, the engineered promoter with two or more external CSREs comprises a first external CSRE located between about 95 base pairs upstream (e.g., −95) of the TATA box and about 174 base pairs upstream (e.g., −174) of the TATA box; and a second external CSRE located between about 164 base pairs upstream (e.g., −164) of the TATA box and about 243 base pairs upstream (e.g., −243) of the TATA box. In yet still another embodiment, the engineered promoter with two or more external CSREs comprises a first external CSRE located between 134 base pairs upstream (e.g., −134) of the TATA box and 135 base pairs upstream (e.g., −135) of the TATA box; and a second external CSRE located between 203 base pairs upstream (e.g., −203) of the TATA box and 204 base pairs upstream (e.g., −204) of the TATA box.

In some embodiments, the engineered promoter of the present disclosure comprises at least three external CSREs. Embodiments of the location and the nucleic acid sequence of the first and second external CSREs are provided herein and can be used in an engineered promoter comprising three or more external CSREs. When a plurality of CSREs is included in the engineered promoter, the CSREs can be independently located at the same position and be contiguous or be provided at different positions and be non-contiguous. In some alternative embodiments, the third external CSRE is located between about 38 base pairs downstream (e.g., +38) of the TATA box and about 363 base pairs upstream (e.g., −363) of the TATA box. In some specific embodiments, the third external CSRE is located between about 38 base pairs downstream (e.g., +38) of the TATA box and about 41 base pairs upstream (e.g., −41) of the TATA box. In some additional embodiments, the third external CSRE is located between 1 base pair upstream (e.g., −1) of the TATA box and 2 base pairs upstream (e.g., −2) of the TATA box. In some specific embodiments, the third external CSRE is located between about 8 base pairs downstream (e.g., +8) of the TATA box and about 71 base pairs upstream (e.g., −71) of the TATA box. In some additional embodiments, the third external CSRE is located between 31 base pairs upstream (e.g., −31) of the TATA box and 32 base pairs upstream (e.g., −32) of the TATA box. In some specific embodiments, the third external CSRE is located between about 2 base pairs upstream (e.g., −2) of the TATA box and about 81 base pairs upstream (e.g., −81) of the TATA box. In some additional embodiments, the third external CSRE is located between 41 base pairs upstream (e.g., −41) of the TATA box and 42 base pairs upstream (e.g., −42) of the TATA box. In some specific embodiments, the third external CSRE is located between about 10 base pairs upstream (e.g., −10) of the TATA box and about 89 base pairs upstream (e.g., −89) of the TATA box. In some additional embodiments, the third external CSRE is located between 49 base pairs upstream (e.g., −49) of the TATA box and 50 base pairs upstream (e.g., −50) of the TATA box. In some specific embodiments, the third external CSRE is located between about 32 base pairs upstream (e.g., −32) of the TATA box and about 111 base pairs upstream (e.g., −111) of the TATA box. In some additional embodiments, the third external CSRE is located between 71 base pairs upstream (e.g., −71) of the TATA box and 72 base pairs upstream (e.g., −72) of the TATA box. In some specific embodiments, the third external CSRE is located between about 36 base pairs upstream (e.g., −36) of the TATA box and about 115 base pairs upstream (e.g., −115) of the TATA box. In some additional embodiments, the third external CSRE is located between 75 base pairs upstream (e.g., −75) of the TATA box and 76 base pairs upstream (e.g., −76) of the TATA box. In some specific embodiments, the third external CSRE is located between about 42 base pairs upstream (e.g., −42) of the TATA box and about 121 base pairs upstream (e.g., −121) of the TATA box. In some additional embodiments, the third external CSRE is located between 81 base pairs upstream (e.g., −81) of the TATA box and 82 base pairs upstream (e.g., −82) of the TATA box. In some specific embodiments, the third external CSRE is located between about 59 base pairs upstream (e.g., −59) of the TATA box and about 138 base pairs upstream (e.g., −138) of the TATA box. In some additional embodiments, the third external CSRE is located between 98 base pairs upstream (e.g., −98) of the TATA box and 99 base pairs upstream (e.g., −99) of the TATA box. In some specific embodiments, the third external CSRE is located between about 60 base pairs upstream (e.g., −60) of the TATA box and about 139 base pairs upstream (e.g., −139) of the TATA box. In some additional embodiments, the third external CSRE is located between 99 base pairs upstream (e.g., −99) of the TATA box and 100 base pairs upstream (e.g., −100) of the TATA box. In some specific embodiments, the third external CSRE is located between about 65 base pairs upstream (e.g., −65) of the TATA box and about 144 base pairs upstream (e.g., −144) of the TATA box. In some additional embodiments, the third external CSRE is located between 104 base pairs upstream (e.g., −104) of the TATA box and 105 base pairs upstream (e.g., −105) of the TATA box. In some specific embodiments, the third external CSRE is located between about 70 base pairs upstream (e.g., −70) of the TATA box and about 149 base pairs upstream (e.g., −149) of the TATA box. In some additional embodiments, the third external CSRE is located between 109 base pairs upstream (e.g., −109) of the TATA box and 110 base pairs upstream (e.g., −110) of the TATA box. In some specific embodiments, the third external CSRE is located between about 75 base pairs upstream (e.g., −75) of the TATA box and about 154 base pairs upstream (e.g., −154) of the TATA box. In some additional embodiments, the third external CSRE is located between 114 base pairs upstream (e.g., −114) of the TATA box and 115 base pairs upstream (e.g., −115) of the TATA box. In some specific embodiments, the third external CSRE is located between about 77 base pairs upstream (e.g., −77) of the TATA box and about 156 base pairs upstream (e.g., −156) of the TATA box. In some additional embodiments, the third external CSRE is located between 116 base pairs upstream (e.g., −116) of the TATA box and 117 base pairs upstream (e.g., −117) of the TATA box. In some specific embodiments, the third external CSRE is located between about 80 base pairs upstream (e.g., −80) of the TATA box and about 159 base pairs upstream (e.g., −159) of the TATA box. In some additional embodiments, the third external CSRE is located between 119 base pairs upstream (e.g., −119) of the TATA box and 120 base pairs upstream (e.g., −120) of the TATA box. In some specific embodiments, the third external CSRE is located between about 85 base pairs upstream (e.g., −85) of the TATA box and about 164 base pairs upstream (e.g., −164) of the TATA box. In some additional embodiments, the third external CSRE is located between 124 base pairs upstream (e.g., −124) of the TATA box and 125 base pairs upstream (e.g., −125) of the TATA box. In some specific embodiments, the third external CSRE is located between about 90 base pairs upstream (e.g., −90) of the TATA box and about 169 base pairs upstream (e.g., −169) of the TATA box. In some additional embodiments, the third external CSRE is located between 129 base pairs upstream (e.g., −129) of the TATA box and 130 base pairs upstream (e.g., −130) of the TATA box. In some specific embodiments, the third external CSRE is located between about 95 base pairs upstream (e.g., −95) of the TATA box and about 174 base pairs upstream (e.g., −174) of the TATA box. In some additional embodiments, the third external CSRE is located between 134 base pairs upstream (e.g., −134) of the TATA box and 135 base pairs upstream (e.g., −135) of the TATA box. In some specific embodiments, the third external CSRE is located between about 155 base pairs upstream (e.g., −155) of the TATA box and about 234 base pairs upstream (e.g., −234) of the TATA box. In some additional embodiments, the third external CSRE is located between 194 base pairs upstream (e.g., −194) of the TATA box and 195 base pairs upstream (e.g., −195) of the TATA box. In some specific embodiments, the third external CSRE is located between about 164 base pairs upstream (e.g., −164) of the TATA box and about 243 base pairs upstream (e.g., −243) of the TATA box. In some additional embodiments, the third external CSRE is located between 203 base pairs upstream (e.g., −203) of the TATA box and 204 base pairs upstream (e.g., −204) of the TATA box. In some specific embodiments, the third external CSRE is located between about 205 base pairs upstream (e.g., −205) of the TATA box and about 284 base pairs upstream (e.g., −284) of the TATA box. In some additional embodiments, the third external CSRE is located between 244 base pairs upstream (e.g., −244) of the TATA box and 245 base pairs upstream (e.g., −245) of the TATA box. In some specific embodiments, the third external CSRE is located between about 284 base pairs upstream (e.g., −284) of the TATA box and about 363 base pairs upstream (e.g., −363) of the TATA box. In some additional embodiments, the third external CSRE is located between 323 base pairs upstream (e.g., −323) of the TATA box and 324 base pairs upstream (e.g., −324) of the TATA box. In still another embodiment, the engineered promoter with three or more external CSREs comprises a first external CSRE located between about 95 base pairs upstream (e.g., −95) of the TATA box and about 174 base pairs upstream (e.g., −174) of the TATA box; a second external CSRE located between about 164 base pairs upstream (e.g., −164) of the TATA box and about 243 base pairs upstream (e.g., −243) of the TATA box; and a third external CSRE located between about 284 base pairs upstream (e.g., −284) of the TATA box and about 363 base pairs upstream (e.g., −363) of the TATA box. In yet still another embodiment, the engineered promoter with three or more external CSREs comprises a first external CSRE located between 134 base pairs upstream (e.g., −134) of the TATA box and 135 base pairs upstream (e.g., −135) of the TATA box; a second external CSRE located between 203 base pairs upstream (e.g., −203) of the TATA box and 204 base pairs upstream (e.g., −204) of the TATA box; and a third external CSRE located between 323 base pairs upstream (e.g., −323) of the TATA box and 324 base pairs upstream (e.g., −324) of the TATA box.

In some embodiments, the engineered promoter of the present disclosure comprises at least four external CSREs. Embodiments of the location and the nucleic acid sequence of the first, second, and third external CSREs are provided herein and can be used in an engineered promoter comprising four or more external CSREs. When a plurality of CSREs is included in the engineered promoter, the CSREs can be independently located at the same position and be contiguous or be provided at different positions and be non-contiguous. In some alternative embodiments, the fourth external CSRE is located between about 38 base pairs downstream (e.g., +38) of the TATA box and about 363 base pairs upstream (e.g., −363) of the TATA box. In some specific embodiments, the fourth external CSRE is located between about 38 base pairs downstream (e.g., +38) of the TATA box and about 41 base pairs upstream (e.g., −41) of the TATA box. In some additional embodiments, the fourth external CSRE is located between 1 base pair upstream (e.g., −1) of the TATA box and 2 base pairs upstream (e.g., −2) of the TATA box. In some specific embodiments, the fourth external CSRE is located between about 8 base pairs downstream (e.g., +8) of the TATA box and about 71 base pairs upstream (e.g., −71) of the TATA box. In some additional embodiments, the fourth external CSRE is located between 31 base pairs upstream (e.g., −31) of the TATA box and 32 base pairs upstream (e.g., −32) of the TATA box. In some specific embodiments, the fourth external CSRE is located between about 2 base pairs upstream (e.g., −2) of the TATA box and about 81 base pairs upstream (e.g., −81) of the TATA box. In some additional embodiments, the fourth external CSRE is located between 41 base pairs upstream (e.g., −41) of the TATA box and 42 base pairs upstream (e.g., −42) of the TATA box. In some specific embodiments, the fourth external CSRE is located between about 10 base pairs upstream (e.g., −10) of the TATA box and about 89 base pairs upstream (e.g., −89) of the TATA box. In some additional embodiments, the fourth external CSRE is located between 49 base pairs upstream (e.g., −49) of the TATA box and 50 base pairs upstream (e.g., −50) of the TATA box. In some specific embodiments, the fourth external CSRE is located between about 32 base pairs upstream (e.g., −32) of the TATA box and about 111 base pairs upstream (e.g., −111) of the TATA box. In some additional embodiments, the fourth external CSRE is located between 71 base pairs upstream (e.g., −71) of the TATA box and 72 base pairs upstream (e.g., −72) of the TATA box. In some specific embodiments, the fourth external CSRE is located between about 36 base pairs upstream (e.g., −36) of the TATA box and about 115 base pairs upstream (e.g., −115) of the TATA box. In some additional embodiments, the fourth external CSRE is located between 75 base pairs upstream (e.g., −75) of the TATA box and 76 base pairs upstream (e.g., −76) of the TATA box. In some specific embodiments, the fourth external CSRE is located between about 42 base pairs upstream (e.g., −42) of the TATA box and about 121 base pairs upstream (e.g., −121) of the TATA box. In some additional embodiments, the fourth external CSRE is located between 81 base pairs upstream (e.g., −81) of the TATA box and 82 base pairs upstream (e.g., −82) of the TATA box. In some specific embodiments, the fourth external CSRE is located between about 59 base pairs upstream (e.g., −59) of the TATA box and about 138 base pairs upstream (e.g., −138) of the TATA box. In some additional embodiments, the fourth external CSRE is located between 98 base pairs upstream (e.g., −98) of the TATA box and 99 base pairs upstream (e.g., −99) of the TATA box. In some specific embodiments, the fourth external CSRE is located between about 60 base pairs upstream (e.g., −60) of the TATA box and about 139 base pairs upstream (e.g., −139) of the TATA box. In some additional embodiments, the fourth external CSRE is located between 99 base pairs upstream (e.g., −99) of the TATA box and 100 base pairs upstream (e.g., −100) of the TATA box. In some specific embodiments, the fourth external CSRE is located between about 65 base pairs upstream (e.g., −65) of the TATA box and about 144 base pairs upstream (e.g., −144) of the TATA box. In some additional embodiments, the fourth external CSRE is located between 104 base pairs upstream (e.g., −104) of the TATA box and 105 base pairs upstream (e.g., −105) of the TATA box. In some specific embodiments, the fourth external CSRE is located between about 70 base pairs upstream (e.g., −70) of the TATA box and about 149 base pairs upstream (e.g., −149) of the TATA box. In some additional embodiments, the fourth external CSRE is located between 109 base pairs upstream (e.g., −109) of the TATA box and 110 base pairs upstream (e.g., −110) of the TATA box. In some specific embodiments, the fourth external CSRE is located between about 75 base pairs upstream (e.g., −75) of the TATA box and about 154 base pairs upstream (e.g., −154) of the TATA box. In some additional embodiments, the fourth external CSRE is located between 114 base pairs upstream (e.g., −114) of the TATA box and 115 base pairs upstream (e.g., −115) of the TATA box. In some specific embodiments, the fourth external CSRE is located between about 77 base pairs upstream (e.g., −77) of the TATA box and about 156 base pairs upstream (e.g., −156) of the TATA box. In some additional embodiments, the fourth external CSRE is located between 116 base pairs upstream (e.g., −116) of the TATA box and 117 base pairs upstream (e.g., −117) of the TATA box. In some specific embodiments, the fourth external CSRE is located between about 80 base pairs upstream (e.g., −80) of the TATA box and about 159 base pairs upstream (e.g., −159) of the TATA box. In some additional embodiments, the fourth external CSRE is located between 119 base pairs upstream (e.g., −119) of the TATA box and 120 base pairs upstream (e.g., −120) of the TATA box. In some specific embodiments, the fourth external CSRE is located between about 85 base pairs upstream (e.g., −85) of the TATA box and about 164 base pairs upstream (e.g., −164) of the TATA box. In some additional embodiments, the fourth external CSRE is located between 124 base pairs upstream (e.g., −124) of the TATA box and 125 base pairs upstream (e.g., −125) of the TATA box. In some specific embodiments, the fourth external CSRE is located between about 90 base pairs upstream (e.g., −90) of the TATA box and about 169 base pairs upstream (e.g., −169) of the TATA box. In some additional embodiments, the fourth external CSRE is located between 129 base pairs upstream (e.g., −129) of the TATA box and 130 base pairs upstream (e.g., −130) of the TATA box. In some specific embodiments, the fourth external CSRE is located between about 95 base pairs upstream (e.g., −95) of the TATA box and about 174 base pairs upstream (e.g., −174) of the TATA box. In some additional embodiments, the fourth external CSRE is located between 134 base pairs upstream (e.g., −134) of the TATA box and 135 base pairs upstream (e.g., −135) of the TATA box. In some specific embodiments, the fourth external CSRE is located between about 155 base pairs upstream (e.g., −155) of the TATA box and about 234 base pairs upstream (e.g., −234) of the TATA box. In some additional embodiments, the fourth external CSRE is located between 194 base pairs upstream (e.g., −194) of the TATA box and 195 base pairs upstream (e.g., −195) of the TATA box. In some specific embodiments, the fourth external CSRE is located between about 164 base pairs upstream (e.g., −164) of the TATA box and about 243 base pairs upstream (e.g., −243) of the TATA box. In some additional embodiments, the fourth external CSRE is located between 203 base pairs upstream (e.g., −203) of the TATA box and 204 base pairs upstream (e.g., −204) of the TATA box. In some specific embodiments, the fourth external CSRE is located between about 205 base pairs upstream (e.g., −205) of the TATA box and about 284 base pairs upstream (e.g., −284) of the TATA box. In some additional embodiments, the fourth external CSRE is located between 244 base pairs upstream (e.g., −244) of the TATA box and 245 base pairs upstream (e.g., −245) of the TATA box. In some specific embodiments, the fourth external CSRE is located between about 284 base pairs upstream (e.g., −284) of the TATA box and about 363 base pairs upstream (e.g., −363) of the TATA box. In some additional embodiments, the fourth external CSRE is located between 323 base pairs upstream (e.g., −323) of the TATA box and 324 base pairs upstream (e.g., −324) of the TATA box.

In some embodiments, the engineered promoter of the present disclosure comprises at least five external CSREs. Embodiments of the location and the nucleic acid sequence of the first, second, third, and fourth external CSREs are provided herein and can be used in an engineered promoter comprising five or more external CSREs. When a plurality of CSREs is included in the engineered promoter, the CSREs can be independently located at the same position and be contiguous or be provided at different positions and be non-contiguous. In some alternative embodiments, the fifth external CSRE is located between about 38 base pairs downstream (e.g., +38) of the TATA box and about 363 base pairs upstream (e.g., −363) of the TATA box. In some specific embodiments, the fifth external CSRE is located between about 38 base pairs downstream (e.g., +38) of the TATA box and about 41 base pairs upstream (e.g., −41) of the TATA box. In some additional embodiments, the fifth external CSRE is located between 1 base pair upstream (e.g., −1) of the TATA box and 2 base pairs upstream (e.g., −2) of the TATA box. In some specific embodiments, the fifth external CSRE is located between about 8 base pairs downstream (e.g., +8) of the TATA box and about 71 base pairs upstream (e.g., −71) of the TATA box. In some additional embodiments, the fifth external CSRE is located between 31 base pairs upstream (e.g., −31) of the TATA box and 32 base pairs upstream (e.g., −32) of the TATA box. In some specific embodiments, the fifth external CSRE is located between about 2 base pairs upstream (e.g., −2) of the TATA box and about 81 base pairs upstream (e.g., −81) of the TATA box. In some additional embodiments, the fifth external CSRE is located between 41 base pairs upstream (e.g., −41) of the TATA box and 42 base pairs upstream (e.g., −42) of the TATA box. In some specific embodiments, the fifth external CSRE is located between about 10 base pairs upstream (e.g., −10) of the TATA box and about 89 base pairs upstream (e.g., −89) of the TATA box. In some additional embodiments, the fifth external CSRE is located between 49 base pairs upstream (e.g., −49) of the TATA box and 50 base pairs upstream (e.g., −50) of the TATA box. In some specific embodiments, the fifth external CSRE is located between about 32 base pairs upstream (e.g., −32) of the TATA box and about 111 base pairs upstream (e.g., −111) of the TATA box. In some additional embodiments, the fifth external CSRE is located between 71 base pairs upstream (e.g., −71) of the TATA box and 72 base pairs upstream (e.g., −72) of the TATA box. In some specific embodiments, the fifth external CSRE is located between about 36 base pairs upstream (e.g., −36) of the TATA box and about 115 base pairs upstream (e.g., −115) of the TATA box. In some additional embodiments, the fifth external CSRE is located between 75 base pairs upstream (e.g., −75) of the TATA box and 76 base pairs upstream (e.g., −76) of the TATA box. In some specific embodiments, the fifth external CSRE is located between about 42 base pairs upstream (e.g., −42) of the TATA box and about 121 base pairs upstream (e.g., −121) of the TATA box. In some additional embodiments, the fifth external CSRE is located between 81 base pairs upstream (e.g., −81) of the TATA box and 82 base pairs upstream (e.g., −82) of the TATA box. In some specific embodiments, the fifth external CSRE is located between about 59 base pairs upstream (e.g., −59) of the TATA box and about 138 base pairs upstream (e.g., −138) of the TATA box. In some additional embodiments, the fifth external CSRE is located between 98 base pairs upstream (e.g., −98) of the TATA box and 99 base pairs upstream (e.g., −99) of the TATA box. In some specific embodiments, the fifth external CSRE is located between about 60 base pairs upstream (e.g., −60) of the TATA box and about 139 base pairs upstream (e.g., −139) of the TATA box. In some additional embodiments, the fifth external CSRE is located between 99 base pairs upstream (e.g., −99) of the TATA box and 100 base pairs upstream (e.g., −100) of the TATA box. In some specific embodiments, the fifth external CSRE is located between about 65 base pairs upstream (e.g., −65) of the TATA box and about 144 base pairs upstream (e.g., −144) of the TATA box. In some additional embodiments, the fifth external CSRE is located between 104 base pairs upstream (e.g., −104) of the TATA box and 105 base pairs upstream (e.g., −105) of the TATA box. In some specific embodiments, the fifth external CSRE is located between about 70 base pairs upstream (e.g., −70) of the TATA box and about 149 base pairs upstream (e.g., −149) of the TATA box. In some additional embodiments, the fifth external CSRE is located between 109 base pairs upstream (e.g., −109) of the TATA box and 110 base pairs upstream (e.g., −110) of the TATA box. In some specific embodiments, the fifth external CSRE is located between about 75 base pairs upstream (e.g., −75) of the TATA box and about 154 base pairs upstream (e.g., −154) of the TATA box. In some additional embodiments, the fifth external CSRE is located between 114 base pairs upstream (e.g., −114) of the TATA box and 115 base pairs upstream (e.g., −115) of the TATA box. In some specific embodiments, the fifth external CSRE is located between about 77 base pairs upstream (e.g., −77) of the TATA box and about 156 base pairs upstream (e.g., −156) of the TATA box. In some additional embodiments, the fifth external CSRE is located between 116 base pairs upstream (e.g., −116) of the TATA box and about 117 base pairs upstream (e.g., −117) of the TATA box. In some specific embodiments, the fifth external CSRE is located between about 80 base pairs upstream (e.g., −80) of the TATA box and about 159 base pairs upstream (e.g., −159) of the TATA box. In some additional embodiments, the fifth external CSRE is located between 119 base pairs upstream (e.g., −119) of the TATA box and 120 base pairs upstream (e.g., −120) of the TATA box. In some specific embodiments, the fifth external CSRE is located between about 85 base pairs upstream (e.g., −85) of the TATA box and about 164 base pairs upstream (e.g., −164) of the TATA box. In some additional embodiments, the fifth external CSRE is located between 124 base pairs upstream (e.g., −124) of the TATA box and 125 base pairs upstream (e.g., −125) of the TATA box. In some specific embodiments, the fifth external CSRE is located between about 90 base pairs upstream (e.g., −90) of the TATA box and about 169 base pairs upstream (e.g., −169) of the TATA box. In some additional embodiments, the fifth external CSRE is located between 129 base pairs upstream (e.g., −129) of the TATA box and 130 base pairs upstream (e.g., −130) of the TATA box. In some specific embodiments, the fifth external CSRE is located between about 95 base pairs upstream (e.g., −95) of the TATA box and about 174 base pairs upstream (e.g., −174) of the TATA box. In some additional embodiments, the fifth external CSRE is located between 134 base pairs upstream (e.g., −134) of the TATA box and 135 base pairs upstream (e.g., −135) of the TATA box. In some specific embodiments, the fifth external CSRE is located between about 155 base pairs upstream (e.g., −155) of the TATA box and about 234 base pairs upstream (e.g., −234) of the TATA box. In some additional embodiments, the fifth external CSRE is located between 194 base pairs upstream (e.g., −194) of the TATA box and 195 base pairs upstream (e.g., −195) of the TATA box. In some specific embodiments, the fifth external CSRE is located between about 164 base pairs upstream (e.g., −164) of the TATA box and about 243 base pairs upstream (e.g., −243) of the TATA box. In some additional embodiments, the fifth external CSRE is located between 203 base pairs upstream (e.g., −203) of the TATA box and 204 base pairs upstream (e.g., −204) of the TATA box. In some specific embodiments, the fifth external CSRE is located between about 205 base pairs upstream (e.g., −205) of the TATA box and about 284 base pairs upstream (e.g., −284) of the TATA box. In some additional embodiments, the fifth external CSRE is located between 244 base pairs upstream (e.g., −244) of the TATA box and 245 base pairs upstream (e.g., −245) of the TATA box. In some specific embodiments, the fifth external CSRE is located between about 284 base pairs upstream (e.g., −284) of the TATA box and about 363 base pairs upstream (e.g., −363) of the TATA box. In some additional embodiments, the fifth external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a fifth external CSRE located between about 284 base pairs upstream (e.g., −284) of the TATA box and about 363 base pairs upstream (e.g., −363) of the TATA box. In yet still another embodiment, the engineered promoter with five or more external CSREs comprises a first external CSRE located between 98 base pairs upstream (e.g., −98) of the TATA box and 99 base pairs upstream (e.g., −99) of the TATA box; a second external CSRE located between 116 base pairs upstream (e.g., −116) of the TATA box and about 117 base pairs upstream (e.g., −117) of the TATA box; a third external CSRE located between 134 base pairs upstream (e.g., −134) of the TATA box and 135 base pairs upstream (e.g., −135) of the TATA box; a fourth external CSRE located between 203 base pairs upstream (e.g., −203) of the TATA box and 204 base pairs upstream (e.g., −204) of the TATA box; and a third external CSRE located between 323 base pairs upstream (e.g., −323) of the TATA box and 324 base pairs upstream (e.g., −324) of the TATA box.

In some embodiments, the engineered promoter of the present disclosure comprises at least six external CSREs. Embodiments of the location and the nucleic acid sequence of the first, second, third, fourth, and fifth external CSREs are provided herein and can be used in an engineered promoter comprising six or more external CSREs. When a plurality of CSREs is included in the engineered promoter, the CSREs can be independently located at the same position and be contiguous or be provided at different positions and be non-contiguous. In some alternative embodiments, the sixth external CSRE is located between about 38 base pairs downstream (e.g., +38) of the TATA box and about 363 base pairs upstream (e.g., −363) of the TATA box. In some specific embodiments, the sixth external CSRE is located between about 38 base pairs downstream (e.g., +38) of the TATA box and about 41 base pairs upstream (e.g., −41) of the TATA box. In some additional embodiments, the sixth external CSRE is located between 1 base pair upstream (e.g., −1) of the TATA box and 2 base pairs upstream (e.g., −2) of the TATA box. In some specific embodiments, the sixth external CSRE is located between about 8 base pairs downstream (e.g., +8) of the TATA box and about 71 base pairs upstream (e.g., −71) of the TATA box. In some additional embodiments, the sixth external CSRE is located between 31 base pairs upstream (e.g., −31) of the TATA box and 32 base pairs upstream (e.g., −32) of the TATA box. In some specific embodiments, the sixth external CSRE is located between about 2 base pairs upstream (e.g., −2) of the TATA box and about 81 base pairs upstream (e.g., −81) of the TATA box. In some additional embodiments, the sixth external CSRE is located between 41 base pairs upstream (e.g., −41) of the TATA box and 42 base pairs upstream (e.g., −42) of the TATA box. In some specific embodiments, the sixth external CSRE is located between about 10 base pairs upstream (e.g., −10) of the TATA box and about 89 base pairs upstream (e.g., −89) of the TATA box. In some additional embodiments, the sixth external CSRE is located between 49 base pairs upstream (e.g., −49) of the TATA box and 50 base pairs upstream (e.g., −50) of the TATA box. In some specific embodiments, the sixth external CSRE is located between about 32 base pairs upstream (e.g., −32) of the TATA box and about 111 base pairs upstream (e.g., −111) of the TATA box. In some additional embodiments, the sixth external CSRE is located between 71 base pairs upstream (e.g., −71) of the TATA box and 72 base pairs upstream (e.g., −72) of the TATA box. In some specific embodiments, the sixth external CSRE is located between about 36 base pairs upstream (e.g., −36) of the TATA box and about 115 base pairs upstream (e.g., −115) of the TATA box. In some additional embodiments, the sixth external CSRE is located between 75 base pairs upstream (e.g., −75) of the TATA box and 76 base pairs upstream (e.g., −76) of the TATA box. In some specific embodiments, the sixth external CSRE is located between about 42 base pairs upstream (e.g., −42) of the TATA box and about 121 base pairs upstream (e.g., −121) of the TATA box. In some additional embodiments, the sixth external CSRE is located between 81 base pairs upstream (e.g., −81) of the TATA box and 82 base pairs upstream (e.g., −82) of the TATA box. In some specific embodiments, the sixth external CSRE is located between about 59 base pairs upstream (e.g., −59) of the TATA box and about 138 base pairs upstream (e.g., −138) of the TATA box. In some additional embodiments, the sixth external CSRE is located between 98 base pairs upstream (e.g., −98) of the TATA box and 99 base pairs upstream (e.g., −99) of the TATA box. In some specific embodiments, the sixth external CSRE is located between about 60 base pairs upstream (e.g., −60) of the TATA box and about 139 base pairs upstream (e.g., −139) of the TATA box. In some additional embodiments, the sixth external CSRE is located between 99 base pairs upstream (e.g., −99) of the TATA box and 100 base pairs upstream (e.g., −100) of the TATA box. In some specific embodiments, the sixth external CSRE is located between about 65 base pairs upstream (e.g., −65) of the TATA box and about 144 base pairs upstream (e.g., −144) of the TATA box. In some additional embodiments, the sixth external CSRE is located between 104 base pairs upstream (e.g., −104) of the TATA box and 105 base pairs upstream (e.g., −105) of the TATA box. In some specific embodiments, the sixth external CSRE is located between about 70 base pairs upstream (e.g., −70) of the TATA box and about 149 base pairs upstream (e.g., −149) of the TATA box. In some additional embodiments, the sixth external CSRE is located between 109 base pairs upstream (e.g., −109) of the TATA box and 110 base pairs upstream (e.g., −110) of the TATA box. In some specific embodiments, the sixth external CSRE is located between about 75 base pairs upstream (e.g., −75) of the TATA box and about 154 base pairs upstream (e.g., −154) of the TATA box. In some additional embodiments, the sixth external CSRE is located between 114 base pairs upstream (e.g., −114) of the TATA box and 115 base pairs upstream (e.g., −115) of the TATA box. In some specific embodiments, the sixth external CSRE is located between about 77 base pairs upstream (e.g., −77) of the TATA box and about 156 base pairs upstream (e.g., −156) of the TATA box. In some additional embodiments, the sixth external CSRE is located between 116 base pairs upstream (e.g., −116) of the TATA box and about 117 base pairs upstream (e.g., −117) of the TATA box. In some specific embodiments, the sixth external CSRE is located between about 80 base pairs upstream (e.g., −80) of the TATA box and about 159 base pairs upstream (e.g., −159) of the TATA box. In some additional embodiments, the sixth external CSRE is located between 119 base pairs upstream (e.g., −119) of the TATA box and 120 base pairs upstream (e.g., −120) of the TATA box. In some specific embodiments, the sixth external CSRE is located between about 85 base pairs upstream (e.g., −85) of the TATA box and about 164 base pairs upstream (e.g., −164) of the TATA box. In some additional embodiments, the sixth external CSRE is located between 124 base pairs upstream (e.g., −124) of the TATA box and 125 base pairs upstream (e.g., −125) of the TATA box. In some specific embodiments, the sixth external CSRE is located between about 90 base pairs upstream (e.g., −90) of the TATA box and about 169 base pairs upstream (e.g., −169) of the TATA box. In some additional embodiments, the sixth external CSRE is located between 129 base pairs upstream (e.g., −129) of the TATA box and 130 base pairs upstream (e.g., −130) of the TATA box. In some specific embodiments, the sixth external CSRE is located between about 95 base pairs upstream (e.g., −95) of the TATA box and about 174 base pairs upstream (e.g., −174) of the TATA box. In some additional embodiments, the sixth external CSRE is located between 134 base pairs upstream (e.g., −134) of the TATA box and 135 base pairs upstream (e.g., −135) of the TATA box. In some specific embodiments, the sixth external CSRE is located between about 155 base pairs upstream (e.g., −155) of the TATA box and about 234 base pairs upstream (e.g., −234) of the TATA box. In some additional embodiments, the sixth external CSRE is located between 194 base pairs upstream (e.g., −194) of the TATA box and 195 base pairs upstream (e.g., −195) of the TATA box. In some specific embodiments, the sixth external CSRE is located between about 164 base pairs upstream (e.g., −164) of the TATA box and about 243 base pairs upstream (e.g., −243) of the TATA box. In some additional embodiments, the sixth external CSRE is located between 203 base pairs upstream (e.g., −203) of the TATA box and 204 base pairs upstream (e.g., −204) of the TATA box. In some specific embodiments, the sixth external CSRE is located between about 205 base pairs upstream (e.g., −205) of the TATA box and about 284 base pairs upstream (e.g., −284) of the TATA box. In some additional embodiments, the sixth external CSRE is located between 244 base pairs upstream (e.g., −244) of the TATA box and 245 base pairs upstream (e.g., −245) of the TATA box. In some specific embodiments, the sixth external CSRE is located between about 284 base pairs upstream (e.g., −284) of the TATA box and about 363 base pairs upstream (e.g., −363) of the TATA box. In some additional embodiments, the sixth external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a sixth external CSRE located between about 284 base pairs upstream (e.g., −284) of the TATA box and about 363 base pairs upstream (e.g., −363) of the TATA box.

In some embodiments, the engineered promoter of the present disclosure comprises at least seven external CSREs. Embodiments of the location and the nucleic acid sequence of the first, second, third, fourth, fifth, and sixth external CSREs are provided herein and can be used in an engineered promoter comprising seven or more external CSREs. When a plurality of CSREs is included in the engineered promoter, the CSREs can be independently located at the same position and be contiguous or be provided at different positions and be non-contiguous. In some alternative embodiments, the seventh external CSRE is located between about 38 base pairs downstream (e.g., +38) of the TATA box and about 363 base pairs upstream (e.g., −363) of the TATA box. In some specific embodiments, the seventh external CSRE is located between about 38 base pairs downstream (e.g., +38) of the TATA box and about 41 base pairs upstream (e.g., −41) of the TATA box. In some additional embodiments, the seventh external CSRE is located between 1 base pair upstream (e.g., −1) of the TATA box and 2 base pairs upstream (e.g., −2) of the TATA box. In some specific embodiments, the seventh external CSRE is located between about 8 base pairs downstream (e.g., +8) of the TATA box and about 71 base pairs upstream (e.g., −71) of the TATA box. In some additional embodiments, the seventh external CSRE is located between 31 base pairs upstream (e.g., −31) of the TATA box and 32 base pairs upstream (e.g., −32) of the TATA box. In some specific embodiments, the seventh external CSRE is located between about 2 base pairs upstream (e.g., −2) of the TATA box and about 81 base pairs upstream (e.g., −81) of the TATA box. In some additional embodiments, the seventh external CSRE is located between 41 base pairs upstream (e.g., −41) of the TATA box and 42 base pairs upstream (e.g., −42) of the TATA box. In some specific embodiments, the seventh external CSRE is located between about 10 base pairs upstream (e.g., −10) of the TATA box and about 89 base pairs upstream (e.g., −89) of the TATA box. In some additional embodiments, the seventh external CSRE is located between 49 base pairs upstream (e.g., −49) of the TATA box and 50 base pairs upstream (e.g., −50) of the TATA box. In some specific embodiments, the seventh external CSRE is located between about 32 base pairs upstream (e.g., −32) of the TATA box and about 111 base pairs upstream (e.g., −111) of the TATA box. In some additional embodiments, the seventh external CSRE is located between 71 base pairs upstream (e.g., −71) of the TATA box and 72 base pairs upstream (e.g., −72) of the TATA box. In some specific embodiments, the seventh external CSRE is located between about 36 base pairs upstream (e.g., −36) of the TATA box and about 115 base pairs upstream (e.g., −115) of the TATA box. In some additional embodiments, the seventh external CSRE is located between 75 base pairs upstream (e.g., −75) of the TATA box and 76 base pairs upstream (e.g., −76) of the TATA box. In some specific embodiments, the seventh external CSRE is located between about 42 base pairs upstream (e.g., −42) of the TATA box and about 121 base pairs upstream (e.g., −121) of the TATA box. In some additional embodiments, the seventh external CSRE is located between 81 base pairs upstream (e.g., −81) of the TATA box and 82 base pairs upstream (e.g., −82) of the TATA box. In some specific embodiments, the seventh external CSRE is located between about 59 base pairs upstream (e.g., −59) of the TATA box and about 138 base pairs upstream (e.g., −138) of the TATA box. In some additional embodiments, the seventh external CSRE is located between 98 base pairs upstream (e.g., −98) of the TATA box and 99 base pairs upstream (e.g., −99) of the TATA box. In some specific embodiments, the seventh external CSRE is located between about 60 base pairs upstream (e.g., −60) of the TATA box and about 139 base pairs upstream (e.g., −139) of the TATA box. In some additional embodiments, the seventh external CSRE is located between 99 base pairs upstream (e.g., −99) of the TATA box and 100 base pairs upstream (e.g., −100) of the TATA box. In some specific embodiments, the seventh external CSRE is located between about 65 base pairs upstream (e.g., −65) of the TATA box and about 144 base pairs upstream (e.g., −144) of the TATA box. In some additional embodiments, the seventh external CSRE is located between 104 base pairs upstream (e.g., −104) of the TATA box and 105 base pairs upstream (e.g., −105) of the TATA box. In some specific embodiments, the seventh external CSRE is located between about 70 base pairs upstream (e.g., −70) of the TATA box and about 149 base pairs upstream (e.g., −149) of the TATA box. In some additional embodiments, the seventh external CSRE is located between 109 base pairs upstream (e.g., −109) of the TATA box and 110 base pairs upstream (e.g., −110) of the TATA box. In some specific embodiments, the seventh external CSRE is located between about 75 base pairs upstream (e.g., −75) of the TATA box and about 154 base pairs upstream (e.g., −154) of the TATA box. In some additional embodiments, the seventh external CSRE is located between 114 base pairs upstream (e.g., −114) of the TATA box and 115 base pairs upstream (e.g., −115) of the TATA box. In some specific embodiments, the seventh external CSRE is located between about 77 base pairs upstream (e.g., −77) of the TATA box and about 156 base pairs upstream (e.g., −156) of the TATA box. In some additional embodiments, the seventh external CSRE is located between 116 base pairs upstream (e.g., −116) of the TATA box and about 117 base pairs upstream (e.g., −117) of the TATA box. In some specific embodiments, the seventh external CSRE is located between about 80 base pairs upstream (e.g., −80) of the TATA box and about 159 base pairs upstream (e.g., −159) of the TATA box. In some additional embodiments, the seventh external CSRE is located between 119 base pairs upstream (e.g., −119) of the TATA box and 120 base pairs upstream (e.g., −120) of the TATA box. In some specific embodiments, the seventh external CSRE is located between about 85 base pairs upstream (e.g., −85) of the TATA box and about 164 base pairs upstream (e.g., −164) of the TATA box. In some additional embodiments, the seventh external CSRE is located between 124 base pairs upstream (e.g., −124) of the TATA box and 125 base pairs upstream (e.g., −125) of the TATA box. In some specific embodiments, the seventh external CSRE is located between about 90 base pairs upstream (e.g., −90) of the TATA box and about 169 base pairs upstream (e.g., −169) of the TATA box. In some additional embodiments, the seventh external CSRE is located between 129 base pairs upstream (e.g., −129) of the TATA box and 130 base pairs upstream (e.g., −130) of the TATA box. In some specific embodiments, the seventh external CSRE is located between about 95 base pairs upstream (e.g., −95) of the TATA box and about 174 base pairs upstream (e.g., −174) of the TATA box. In some additional embodiments, the seventh external CSRE is located between 134 base pairs upstream (e.g., −134) of the TATA box and 135 base pairs upstream (e.g., −135) of the TATA box. In some specific embodiments, the seventh external CSRE is located between about 155 base pairs upstream (e.g., −155) of the TATA box and about 234 base pairs upstream (e.g., −234) of the TATA box. In some additional embodiments, the seventh external CSRE is located between 194 base pairs upstream (e.g., −194) of the TATA box and 195 base pairs upstream (e.g., −195) of the TATA box. In some specific embodiments, the seventh external CSRE is located between about 164 base pairs upstream (e.g., −164) of the TATA box and about 243 base pairs upstream (e.g., −243) of the TATA box. In some additional embodiments, the seventh external CSRE is located between 203 base pairs upstream (e.g., −203) of the TATA box and 204 base pairs upstream (e.g., −204) of the TATA box. In some specific embodiments, the seventh external CSRE is located between about 205 base pairs upstream (e.g., −205) of the TATA box and about 284 base pairs upstream (e.g., −284) of the TATA box. In some additional embodiments, the seventh external CSRE is located between 244 base pairs upstream (e.g., −244) of the TATA box and 245 base pairs upstream (e.g., −245) of the TATA box. In some specific embodiments, the seventh external CSRE is located between about 284 base pairs upstream (e.g., −284) of the TATA box and about 363 base pairs upstream (e.g., −363) of the TATA box. In some additional embodiments, the seventh external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a seventh external CSRE located between about 284 base pairs upstream (e.g., −284) of the TATA box and about 363 base pairs upstream (e.g., −363) of the TATA box.

In some embodiments, the engineered promoter of the present disclosure comprises at least eight external CSREs. Embodiments of the location and the nucleic acid sequence of the first, second, third, fourth, fifth, sixth, and seventh external CSREs are provided herein and can be used in an engineered promoter comprising eight or more external CSREs. When a plurality of CSREs is included in the engineered promoter, the CSREs can be independently located at the same position and be contiguous or be provided at different positions and be non-contiguous. In some alternative embodiments, the eighth external CSRE is located between about 38 base pairs downstream (e.g., +38) of the TATA box and about 363 base pairs upstream (e.g., −363) of the TATA box. In some specific embodiments, the eighth external CSRE is located between about 38 base pairs downstream (e.g., +38) of the TATA box and about 41 base pairs upstream (e.g., −41) of the TATA box. In some additional embodiments, the eighth external CSRE is located between 1 base pair upstream (e.g., −1) of the TATA box and 2 base pairs upstream (e.g., −2) of the TATA box. In some specific embodiments, the eighth external CSRE is located between about 8 base pairs downstream (e.g., +8) of the TATA box and about 71 base pairs upstream (e.g., −71) of the TATA box. In some additional embodiments, the eighth external CSRE is located between 31 base pairs upstream (e.g., −31) of the TATA box and 32 base pairs upstream (e.g., −32) of the TATA box. In some specific embodiments, the eighth external CSRE is located between about 2 base pairs upstream (e.g., −2) of the TATA box and about 81 base pairs upstream (e.g., −81) of the TATA box. In some additional embodiments, the eighth external CSRE is located between 41 base pairs upstream (e.g., −41) of the TATA box and 42 base pairs upstream (e.g., −42) of the TATA box. In some specific embodiments, the eighth external CSRE is located between about 10 base pairs upstream (e.g., −10) of the TATA box and about 89 base pairs upstream (e.g., −89) of the TATA box. In some additional embodiments, the eighth external CSRE is located between 49 base pairs upstream (e.g., −49) of the TATA box and 50 base pairs upstream (e.g., −50) of the TATA box. In some specific embodiments, the eighth external CSRE is located between about 32 base pairs upstream (e.g., −32) of the TATA box and about 111 base pairs upstream (e.g., −111) of the TATA box. In some additional embodiments, the eighth external CSRE is located between 71 base pairs upstream (e.g., −71) of the TATA box and 72 base pairs upstream (e.g., −72) of the TATA box. In some specific embodiments, the eighth external CSRE is located between about 36 base pairs upstream (e.g., −36) of the TATA box and about 115 base pairs upstream (e.g., −115) of the TATA box. In some additional embodiments, the eighth external CSRE is located between 75 base pairs upstream (e.g., −75) of the TATA box and 76 base pairs upstream (e.g., −76) of the TATA box. In some specific embodiments, the eighth external CSRE is located between about 42 base pairs upstream (e.g., −42) of the TATA box and about 121 base pairs upstream (e.g., −121) of the TATA box. In some additional embodiments, the eighth external CSRE is located between 81 base pairs upstream (e.g., −81) of the TATA box and 82 base pairs upstream (e.g., −82) of the TATA box. In some specific embodiments, the eighth external CSRE is located between about 59 base pairs upstream (e.g., −59) of the TATA box and about 138 base pairs upstream (e.g., −138) of the TATA box. In some additional embodiments, the eighth external CSRE is located between 98 base pairs upstream (e.g., −98) of the TATA box and 99 base pairs upstream (e.g., −99) of the TATA box. In some specific embodiments, the eighth external CSRE is located between about 60 base pairs upstream (e.g., −60) of the TATA box and about 139 base pairs upstream (e.g., −139) of the TATA box. In some additional embodiments, the eighth external CSRE is located between 99 base pairs upstream (e.g., −99) of the TATA box and 100 base pairs upstream (e.g., −100) of the TATA box. In some specific embodiments, the eighth external CSRE is located between about 65 base pairs upstream (e.g., −65) of the TATA box and about 144 base pairs upstream (e.g., −144) of the TATA box. In some additional embodiments, the eighth external CSRE is located between 104 base pairs upstream (e.g., −104) of the TATA box and 105 base pairs upstream (e.g., −105) of the TATA box. In some specific embodiments, the eighth external CSRE is located between about 70 base pairs upstream (e.g., −70) of the TATA box and about 149 base pairs upstream (e.g., −149) of the TATA box. In some additional embodiments, the eighth external CSRE is located between 109 base pairs upstream (e.g., −109) of the TATA box and 110 base pairs upstream (e.g., −110) of the TATA box. In some specific embodiments, the eighth external CSRE is located between about 75 base pairs upstream (e.g., −75) of the TATA box and about 154 base pairs upstream (e.g., −154) of the TATA box. In some additional embodiments, the eighth external CSRE is located between 114 base pairs upstream (e.g., −114) of the TATA box and 115 base pairs upstream (e.g., −115) of the TATA box. In some specific embodiments, the eighth external CSRE is located between about 77 base pairs upstream (e.g., −77) of the TATA box and about 156 base pairs upstream (e.g., −156) of the TATA box. In some additional embodiments, the eighth external CSRE is located between 116 base pairs upstream (e.g., −116) of the TATA box and about 117 base pairs upstream (e.g., −117) of the TATA box. In some specific embodiments, the eighth external CSRE is located between about 80 base pairs upstream (e.g., −80) of the TATA box and about 159 base pairs upstream (e.g., −159) of the TATA box. In some additional embodiments, the eighth external CSRE is located between 119 base pairs upstream (e.g., −119) of the TATA box and 120 base pairs upstream (e.g., −120) of the TATA box. In some specific embodiments, the eighth external CSRE is located between about 85 base pairs upstream (e.g., −85) of the TATA box and about 164 base pairs upstream (e.g., −164) of the TATA box. In some additional embodiments, the eighth external CSRE is located between 124 base pairs upstream (e.g., −124) of the TATA box and 125 base pairs upstream (e.g., −125) of the TATA box. In some specific embodiments, the eighth external CSRE is located between about 90 base pairs upstream (e.g., −90) of the TATA box and about 169 base pairs upstream (e.g., −169) of the TATA box. In some additional embodiments, the eighth external CSRE is located between 129 base pairs upstream (e.g., −129) of the TATA box and 130 base pairs upstream (e.g., −130) of the TATA box. In some specific embodiments, the eighth external CSRE is located between about 95 base pairs upstream (e.g., −95) of the TATA box and about 174 base pairs upstream (e.g., −174) of the TATA box. In some additional embodiments, the eighth external CSRE is located between 134 base pairs upstream (e.g., −134) of the TATA box and 135 base pairs upstream (e.g., −135) of the TATA box. In some specific embodiments, the eighth external CSRE is located between about 155 base pairs upstream (e.g., −155) of the TATA box and about 234 base pairs upstream (e.g., −234) of the TATA box. In some additional embodiments, the eighth external CSRE is located between 194 base pairs upstream (e.g., −194) of the TATA box and 195 base pairs upstream (e.g., −195) of the TATA box. In some specific embodiments, the eighth external CSRE is located between about 164 base pairs upstream (e.g., −164) of the TATA box and about 243 base pairs upstream (e.g., −243) of the TATA box. In some additional embodiments, the eighth external CSRE is located between 203 base pairs upstream (e.g., −203) of the TATA box and 204 base pairs upstream (e.g., −204) of the TATA box. In some specific embodiments, the eighth external CSRE is located between about 205 base pairs upstream (e.g., −205) of the TATA box and about 284 base pairs upstream (e.g., −284) of the TATA box. In some additional embodiments, the eighth external CSRE is located between 244 base pairs upstream (e.g., −244) of the TATA box and 245 base pairs upstream (e.g., −245) of the TATA box. In some specific embodiments, the eighth external CSRE is located between about 284 base pairs upstream (e.g., −284) of the TATA box and about 363 base pairs upstream (e.g., −363) of the TATA box. In some additional embodiments, the eighth external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a eighth external CSRE located between about 284 base pairs upstream (e.g., −284) of the TATA box and about 363 base pairs upstream (e.g., −363) of the TATA box.

In some embodiments, the engineered promoter of the present disclosure comprises at least nine external CSREs. Embodiments of the location and the nucleic acid sequence of the first, second, third, fourth, fifth, sixth, seventh, and eighth external CSREs are provided herein and can be used in an engineered promoter comprising nine or more external CSREs. When a plurality of CSREs is included in the engineered promoter, the CSREs can be independently located at the same position and be contiguous or be provided at different positions and be non-contiguous. In some alternative embodiments, the ninth external CSRE is located between about 38 base pairs downstream (e.g., +38) of the TATA box and about 363 base pairs upstream (e.g., −363) of the TATA box. In some specific embodiments, the ninth external CSRE is located between about 38 base pairs downstream (e.g., +38) of the TATA box and about 41 base pairs upstream (e.g., −41) of the TATA box. In some additional embodiments, the ninth external CSRE is located between 1 base pair upstream (e.g., −1) of the TATA box and 2 base pairs upstream (e.g., −2) of the TATA box. In some specific embodiments, the ninth external CSRE is located between about 8 base pairs downstream (e.g., +8) of the TATA box and about 71 base pairs upstream (e.g., −71) of the TATA box. In some additional embodiments, the ninth external CSRE is located between 31 base pairs upstream (e.g., −31) of the TATA box and 32 base pairs upstream (e.g., −32) of the TATA box. In some specific embodiments, the ninth external CSRE is located between about 2 base pairs upstream (e.g., −2) of the TATA box and about 81 base pairs upstream (e.g., −81) of the TATA box. In some additional embodiments, the ninth external CSRE is located between 41 base pairs upstream (e.g., −41) of the TATA box and 42 base pairs upstream (e.g., −42) of the TATA box. In some specific embodiments, the ninth external CSRE is located between about 10 base pairs upstream (e.g., −10) of the TATA box and about 89 base pairs upstream (e.g., −89) of the TATA box. In some additional embodiments, the ninth external CSRE is located between 49 base pairs upstream (e.g., −49) of the TATA box and 50 base pairs upstream (e.g., −50) of the TATA box. In some specific embodiments, the ninth external CSRE is located between about 32 base pairs upstream (e.g., −32) of the TATA box and about 111 base pairs upstream (e.g., −111) of the TATA box. In some additional embodiments, the ninth external CSRE is located between 71 base pairs upstream (e.g., −71) of the TATA box and 72 base pairs upstream (e.g., −72) of the TATA box. In some specific embodiments, the ninth external CSRE is located between about 36 base pairs upstream (e.g., −36) of the TATA box and about 115 base pairs upstream (e.g., −115) of the TATA box. In some additional embodiments, the ninth external CSRE is located between 75 base pairs upstream (e.g., −75) of the TATA box and 76 base pairs upstream (e.g., −76) of the TATA box. In some specific embodiments, the ninth external CSRE is located between about 42 base pairs upstream (e.g., −42) of the TATA box and about 121 base pairs upstream (e.g., −121) of the TATA box. In some additional embodiments, the ninth external CSRE is located between 81 base pairs upstream (e.g., −81) of the TATA box and 82 base pairs upstream (e.g., −82) of the TATA box. In some specific embodiments, the ninth external CSRE is located between about 59 base pairs upstream (e.g., −59) of the TATA box and about 138 base pairs upstream (e.g., −138) of the TATA box. In some additional embodiments, the ninth external CSRE is located between 98 base pairs upstream (e.g., −98) of the TATA box and 99 base pairs upstream (e.g., −99) of the TATA box. In some specific embodiments, the ninth external CSRE is located between about 60 base pairs upstream (e.g., −60) of the TATA box and about 139 base pairs upstream (e.g., −139) of the TATA box. In some additional embodiments, the ninth external CSRE is located between 99 base pairs upstream (e.g., −99) of the TATA box and 100 base pairs upstream (e.g., −100) of the TATA box. In some specific embodiments, the ninth external CSRE is located between about 65 base pairs upstream (e.g., −65) of the TATA box and about 144 base pairs upstream (e.g., −144) of the TATA box. In some additional embodiments, the ninth external CSRE is located between 104 base pairs upstream (e.g., −104) of the TATA box and 105 base pairs upstream (e.g., −105) of the TATA box. In some specific embodiments, the ninth external CSRE is located between about 70 base pairs upstream (e.g., −70) of the TATA box and about 149 base pairs upstream (e.g., −149) of the TATA box. In some additional embodiments, the ninth external CSRE is located between 109 base pairs upstream (e.g., −109) of the TATA box and 110 base pairs upstream (e.g., −110) of the TATA box. In some specific embodiments, the ninth external CSRE is located between about 75 base pairs upstream (e.g., −75) of the TATA box and about 154 base pairs upstream (e.g., −154) of the TATA box. In some additional embodiments, the ninth external CSRE is located between 114 base pairs upstream (e.g., −114) of the TATA box and 115 base pairs upstream (e.g., −115) of the TATA box. In some specific embodiments, the ninth external CSRE is located between about 77 base pairs upstream (e.g., −77) of the TATA box and about 156 base pairs upstream (e.g., −156) of the TATA box. In some additional embodiments, the ninth external CSRE is located between 116 base pairs upstream (e.g., −116) of the TATA box and about 117 base pairs upstream (e.g., −117) of the TATA box. In some specific embodiments, the ninth external CSRE is located between about 80 base pairs upstream (e.g., −80) of the TATA box and about 159 base pairs upstream (e.g., −159) of the TATA box. In some additional embodiments, the ninth external CSRE is located between 119 base pairs upstream (e.g., −119) of the TATA box and 120 base pairs upstream (e.g., −120) of the TATA box. In some specific embodiments, the ninth external CSRE is located between about 85 base pairs upstream (e.g., −85) of the TATA box and about 164 base pairs upstream (e.g., −164) of the TATA box. In some additional embodiments, the ninth external CSRE is located between 124 base pairs upstream (e.g., −124) of the TATA box and 125 base pairs upstream (e.g., −125) of the TATA box. In some specific embodiments, the ninth external CSRE is located between about 90 base pairs upstream (e.g., −90) of the TATA box and about 169 base pairs upstream (e.g., −169) of the TATA box. In some additional embodiments, the ninth external CSRE is located between 129 base pairs upstream (e.g., −129) of the TATA box and 130 base pairs upstream (e.g., −130) of the TATA box. In some specific embodiments, the ninth external CSRE is located between about 95 base pairs upstream (e.g., −95) of the TATA box and about 174 base pairs upstream (e.g., −174) of the TATA box. In some additional embodiments, the ninth external CSRE is located between 134 base pairs upstream (e.g., −134) of the TATA box and 135 base pairs upstream (e.g., −135) of the TATA box. In some specific embodiments, the ninth external CSRE is located between about 155 base pairs upstream (e.g., −155) of the TATA box and about 234 base pairs upstream (e.g., −234) of the TATA box. In some additional embodiments, the ninth external CSRE is located between 194 base pairs upstream (e.g., −194) of the TATA box and 195 base pairs upstream (e.g., −195) of the TATA box. In some specific embodiments, the ninth external CSRE is located between about 164 base pairs upstream (e.g., −164) of the TATA box and about 243 base pairs upstream (e.g., −243) of the TATA box. In some additional embodiments, the ninth external CSRE is located between 203 base pairs upstream (e.g., −203) of the TATA box and 204 base pairs upstream (e.g., −204) of the TATA box. In some specific embodiments, the ninth external CSRE is located between about 205 base pairs upstream (e.g., −205) of the TATA box and about 284 base pairs upstream (e.g., −284) of the TATA box. In some additional embodiments, the ninth external CSRE is located between 244 base pairs upstream (e.g., −244) of the TATA box and 245 base pairs upstream (e.g., −245) of the TATA box. In some specific embodiments, the ninth external CSRE is located between about 284 base pairs upstream (e.g., −284) of the TATA box and about 363 base pairs upstream (e.g., −363) of the TATA box. In some additional embodiments, the ninth external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a ninth external CSRE located between about 284 base pairs upstream (e.g., −284) of the TATA box and about 363 base pairs upstream (e.g., −363) of the TATA box.

In some embodiments, the engineered promoter of the present disclosure comprises at least ten external CSREs. Embodiments of the location and the nucleic acid sequence of the first, second, third, fourth, fifth, sixth, seventh, eighth, and ninth external CSREs are provided herein and can be used in an engineered promoter comprising ten or more external CSREs. When a plurality of CSREs is included in the engineered promoter, the CSREs can be independently located at the same position and be contiguous or be provided at different positions and be non-contiguous. In some alternative embodiments, the tenth external CSRE is located between about 38 base pairs downstream (e.g., +38) of the TATA box and about 363 base pairs upstream (e.g., −363) of the TATA box. In some specific embodiments, the tenth external CSRE is located between about 38 base pairs downstream (e.g., +38) of the TATA box and about 41 base pairs upstream (e.g., −41) of the TATA box. In some additional embodiments, the tenth external CSRE is located between 1 base pair upstream (e.g., −1) of the TATA box and 2 base pairs upstream (e.g., −2) of the TATA box. In some specific embodiments, the tenth external CSRE is located between about 8 base pairs downstream (e.g., +8) of the TATA box and about 71 base pairs upstream (e.g., −71) of the TATA box. In some additional embodiments, the tenth external CSRE is located between 31 base pairs upstream (e.g., −31) of the TATA box and 32 base pairs upstream (e.g., −32) of the TATA box. In some specific embodiments, the tenth external CSRE is located between about 2 base pairs upstream (e.g., −2) of the TATA box and about 81 base pairs upstream (e.g., −81) of the TATA box. In some additional embodiments, the tenth external CSRE is located between 41 base pairs upstream (e.g., −41) of the TATA box and 42 base pairs upstream (e.g., −42) of the TATA box. In some specific embodiments, the tenth external CSRE is located between about 10 base pairs upstream (e.g., −10) of the TATA box and about 89 base pairs upstream (e.g., −89) of the TATA box. In some additional embodiments, the tenth external CSRE is located between 49 base pairs upstream (e.g., −49) of the TATA box and 50 base pairs upstream (e.g., −50) of the TATA box. In some specific embodiments, the tenth external CSRE is located between about 32 base pairs upstream (e.g., −32) of the TATA box and about 111 base pairs upstream (e.g., −111) of the TATA box. In some additional embodiments, the tenth external CSRE is located between 71 base pairs upstream (e.g., −71) of the TATA box and 72 base pairs upstream (e.g., −72) of the TATA box. In some specific embodiments, the tenth external CSRE is located between about 36 base pairs upstream (e.g., −36) of the TATA box and about 115 base pairs upstream (e.g., −115) of the TATA box. In some additional embodiments, the tenth external CSRE is located between 75 base pairs upstream (e.g., −75) of the TATA box and 76 base pairs upstream (e.g., −76) of the TATA box. In some specific embodiments, the tenth external CSRE is located between about 42 base pairs upstream (e.g., −42) of the TATA box and about 121 base pairs upstream (e.g., −121) of the TATA box. In some additional embodiments, the tenth external CSRE is located between 81 base pairs upstream (e.g., −81) of the TATA box and 82 base pairs upstream (e.g., −82) of the TATA box. In some specific embodiments, the tenth external CSRE is located between about 59 base pairs upstream (e.g., −59) of the TATA box and about 138 base pairs upstream (e.g., −138) of the TATA box. In some additional embodiments, the tenth external CSRE is located between 98 base pairs upstream (e.g., −98) of the TATA box and 99 base pairs upstream (e.g., −99) of the TATA box. In some specific embodiments, the tenth external CSRE is located between about 60 base pairs upstream (e.g., −60) of the TATA box and about 139 base pairs upstream (e.g., −139) of the TATA box. In some additional embodiments, the tenth external CSRE is located between 99 base pairs upstream (e.g., −99) of the TATA box and 100 base pairs upstream (e.g., −100) of the TATA box. In some specific embodiments, the tenth external CSRE is located between about 65 base pairs upstream (e.g., −65) of the TATA box and about 144 base pairs upstream (e.g., −144) of the TATA box. In some additional embodiments, the tenth external CSRE is located between 104 base pairs upstream (e.g., −104) of the TATA box and 105 base pairs upstream (e.g., −105) of the TATA box. In some specific embodiments, the tenth external CSRE is located between about 70 base pairs upstream (e.g., −70) of the TATA box and about 149 base pairs upstream (e.g., −149) of the TATA box. In some additional embodiments, the tenth external CSRE is located between 109 base pairs upstream (e.g., −109) of the TATA box and 110 base pairs upstream (e.g., −110) of the TATA box. In some specific embodiments, the tenth external CSRE is located between about 75 base pairs upstream (e.g., −75) of the TATA box and about 154 base pairs upstream (e.g., −154) of the TATA box. In some additional embodiments, the tenth external CSRE is located between 114 base pairs upstream (e.g., −114) of the TATA box and 115 base pairs upstream (e.g., −115) of the TATA box. In some specific embodiments, the tenth external CSRE is located between about 77 base pairs upstream (e.g., −77) of the TATA box and about 156 base pairs upstream (e.g., −156) of the TATA box. In some additional embodiments, the tenth external CSRE is located between 116 base pairs upstream (e.g., −116) of the TATA box and about 117 base pairs upstream (e.g., −117) of the TATA box. In some specific embodiments, the tenth external CSRE is located between about 80 base pairs upstream (e.g., −80) of the TATA box and about 159 base pairs upstream (e.g., −159) of the TATA box. In some additional embodiments, the tenth external CSRE is located between 119 base pairs upstream (e.g., −119) of the TATA box and 120 base pairs upstream (e.g., −120) of the TATA box. In some specific embodiments, the tenth external CSRE is located between about 85 base pairs upstream (e.g., −85) of the TATA box and about 164 base pairs upstream (e.g., −164) of the TATA box. In some additional embodiments, the tenth external CSRE is located between 124 base pairs upstream (e.g., −124) of the TATA box and 125 base pairs upstream (e.g., −125) of the TATA box. In some specific embodiments, the tenth external CSRE is located between about 90 base pairs upstream (e.g., −90) of the TATA box and about 169 base pairs upstream (e.g., −169) of the TATA box. In some additional embodiments, the tenth external CSRE is located between 129 base pairs upstream (e.g., −129) of the TATA box and 130 base pairs upstream (e.g., −130) of the TATA box. In some specific embodiments, the tenth external CSRE is located between about 95 base pairs upstream (e.g., −95) of the TATA box and about 174 base pairs upstream (e.g., −174) of the TATA box. In some additional embodiments, the tenth external CSRE is located between 134 base pairs upstream (e.g., −134) of the TATA box and 135 base pairs upstream (e.g., −135) of the TATA box. In some specific embodiments, the tenth external CSRE is located between about 155 base pairs upstream (e.g., −155) of the TATA box and about 234 base pairs upstream (e.g., −234) of the TATA box. In some additional embodiments, the tenth external CSRE is located between 194 base pairs upstream (e.g., −194) of the TATA box and 195 base pairs upstream (e.g., −195) of the TATA box. In some specific embodiments, the tenth external CSRE is located between about 164 base pairs upstream (e.g., −164) of the TATA box and about 243 base pairs upstream (e.g., −243) of the TATA box. In some additional embodiments, the tenth external CSRE is located between 203 base pairs upstream (e.g., −203) of the TATA box and 204 base pairs upstream (e.g., −204) of the TATA box. In some specific embodiments, the tenth external CSRE is located between about 205 base pairs upstream (e.g., −205) of the TATA box and about 284 base pairs upstream (e.g., −284) of the TATA box. In some additional embodiments, the tenth external CSRE is located between 244 base pairs upstream (e.g., −244) of the TATA box and 245 base pairs upstream (e.g., −245) of the TATA box. In some specific embodiments, the tenth external CSRE is located between about 284 base pairs upstream (e.g., −284) of the TATA box and about 363 base pairs upstream (e.g., −363) of the TATA box. In some additional embodiments, the tenth external CSRE is located between 323 base pairs upstream (e.g., −323) of the TATA box and 324 base pairs upstream (e.g., −324) of the TATA box. In still another embodiment, the engineered promoter with five or more external CSREs comprises a first external CSRE located between about 59 base pairs upstream (e.g., −59) of the TATA box and about 138 base pairs upstream (e.g., −138) of the TATA box; a second external CSRE located between about 77 base pairs upstream (e.g., −77) of the TATA box and about 156 base pairs upstream (e.g., −156) of the TATA box; a third external CSRE located between about 95 base pairs upstream (e.g., −95) of the TATA box and about 174 base pairs upstream (e.g., −174) of the TATA box; a fourth external CSRE located between about 164 base pairs upstream (e.g., −164) of the TATA box and about 243 base pairs upstream (e.g., −243) of the TATA box; and a tenth external CSRE located between about 284 base pairs upstream (e.g., −284) of the TATA box and about 363 base pairs upstream (e.g., −363) of the TATA box. In still another embodiment, the engineered promoter with ten or more external CSREs comprises a first external CSRE located between about 60 base pairs upstream (e.g., −60) of the TATA box and about 139 base pairs upstream (e.g., −139) of the TATA box; a second external CSRE located between about 65 base pairs upstream (e.g., −65) of the TATA box and about 144 base pairs upstream (e.g., −144) of the TATA box; a third external CSRE located between about 70 base pairs upstream (e.g., −70) of the TATA box and about 149 base pairs upstream (e.g., −149) of the TATA box; a fourth external CSRE located between about 75 base pairs upstream (e.g., −75) of the TATA box and about 154 base pairs upstream (e.g., −154) of the TATA box; a fifth external CSRE located between about 80 base pairs upstream (e.g., −80) of the TATA box and about 159 base pairs upstream (e.g., −159) of the TATA box; a sixth external CSRE located between about 85 base pairs upstream (e.g., −85) of the TATA box and about 164 base pairs upstream (e.g., −164) of the TATA box; a seventh external CSRE located between about 90 base pairs upstream (e.g., −90) of the TATA box and about 169 base pairs upstream (e.g., −169) of the TATA box; an eighth external CSRE located between about 95 base pairs upstream (e.g., −95) of the TATA box and about 174 base pairs upstream (e.g., −174) of the TATA box; a ninth external CSRE located between about 164 base pairs upstream (e.g., −164) of the TATA box and about 243 base pairs upstream (e.g., −243) of the TATA box; and a tenth external CSRE located between about 284 base pairs upstream (e.g., −284) of the TATA box and about 363 base pairs upstream (e.g., −363) of the TATA box. In yet still another embodiment, the engineered promoter with ten or more external CSREs comprises a first external CSRE located between 99 base pairs upstream (e.g., −99) of the TATA box and 100 base pairs upstream (e.g., −100) of the TATA box; a second external CSRE located between 104 base pairs upstream (e.g., −104) of the TATA box and 105 base pairs upstream (e.g., −105) of the TATA box; a third external CSRE located between 109 base pairs upstream (e.g., −109) of the TATA box and 110 base pairs upstream (e.g., −110) of the TATA box; a fourth external CSRE located between 114 base pairs upstream (e.g., −114) of the TATA box and 115 base pairs upstream (e.g., −115) of the TATA box; a fifth external CSRE located between 119 base pairs upstream (e.g., −119) of the TATA box and 120 base pairs upstream (e.g., −120) of the TATA box; a sixth external CSRE located between 124 base pairs upstream (e.g., −124) of the TATA box and 125 base pairs upstream (e.g., −125) of the TATA box; a seventh external CSRE located between 129 base pairs upstream (e.g., −129) of the TATA box and 130 base pairs upstream (e.g., −130) of the TATA box; an eighth external CSRE located between 134 base pairs upstream (e.g., −134) of the TATA box and 135 base pairs upstream (e.g., −135) of the TATA box; a ninth external CSRE located between 203 base pairs upstream (e.g., −203) of the TATA box and 204 base pairs upstream (e.g., −204) of the TATA box; and a tenth external CSRE located between 323 base pairs upstream (e.g., −323) of the TATA box and 324 base pairs upstream (e.g., −324) of the TATA box.

In some embodiments the engineered promoter is intended to be used with a gene comprising an open reading frame and a start codon, and as such, the first external CSRE can be located in a region defined from the start codon. In some embodiments, the first external CSRE is located at most 442 base pairs upstream (e.g., −442) of the start codon. In some instances, the engineered promoter can include one or more external CSREs which can be located more than 442 base pairs upstream (e.g., −442) of the start codon (provided that it includes at least one external CSRE at most 442 base pairs upstream of the start codon). In some embodiments, the engineered promoter of the present disclosure comprises a first external CSRE located between about 52 base pairs upstream (e.g., −52) of the start codon and at most about 442 base pairs upstream (e.g., −442) of the start codon. In some embodiments, the first external CSRE is located at most 390 base pairs upstream (e.g., −390) of the start codon. The first external CSRE can be located between the transcription start site and about 390 base pairs upstream (e.g., −390) of the start codon. In some instances, the engineered promoter can include one or more external CSREs which can be located more than 390 base pairs upstream (e.g., −390) of the start codon (provided that it includes at least one external CSRE at most 390 base pairs upstream of the start codon).

In some specific embodiments, the first external CSRE is located between about 52 base pairs upstream (e.g., −52) of the start codon and about 131 base pairs upstream (e.g., −131) of the start codon. In some additional embodiments, the first external CSRE is located between 91 base pairs upstream (e.g., −91) of the start codon and 92 base pairs upstream (e.g., −92) of the start codon. In some specific embodiments, the first external CSRE is located between about 82 base pairs upstream (e.g., −82) of the start codon and about 161 base pairs upstream (e.g., −161) of the start codon. In some additional embodiments, the first external CSRE is located between 121 base pairs upstream (e.g., −121) of the start codon and 122 base pairs upstream (e.g., −122) of the start codon. In some specific embodiments, the first external CSRE is located between about 92 base pairs upstream (e.g., −92) of the start codon and about 171 base pairs upstream (e.g., −171) of the start codon. In some additional embodiments, the first external CSRE is located between 131 base pairs upstream (e.g., −131) of the start codon and 132 base pairs upstream (e.g., −132) of the start codon. In some specific embodiments, the first external CSRE is located between about 100 base pairs upstream (e.g., −100) of the start codon and about 179 base pairs upstream (e.g., −179) of the start codon. In some additional embodiments, the first external CSRE is located between 139 base pairs upstream (e.g., −139) of the start codon and 140 base pairs upstream (e.g., −140) of the start codon. In some specific embodiments, the first external CSRE is located between about 122 base pairs upstream (e.g., −122) of the start codon and about 201 base pairs upstream (e.g., −201) of the start codon. In some additional embodiments, the first external CSRE is located between 161 base pairs upstream (e.g., −161) of the start codon and 162 base pairs upstream (e.g., −162) of the start codon. In some specific embodiments, the first external CSRE is located between about 126 base pairs upstream (e.g., −126) of the start codon and about 205 base pairs upstream (e.g., −205) of the start codon. In some additional embodiments, the first external CSRE is located between 165 base pairs upstream (e.g., −165) of the start codon and 166 base pairs upstream (e.g., −166) of the start codon. In some specific embodiments, the first external CSRE is located between about 132 base pairs upstream (e.g., −132) of the start codon and about 211 base pairs upstream (e.g., −211) of the start codon. In some additional embodiments, the first external CSRE is located between 171 base pairs upstream (e.g., −171) of the start codon and 172 base pairs upstream (e.g., −172) of the start codon. In some specific embodiments, the first external CSRE is located between about 138 base pairs upstream (e.g., −138) of the start codon and about 217 base pairs upstream (e.g., −217) of the start codon. In some additional embodiments, the first external CSRE is located between 177 base pairs upstream (e.g., −177) of the start codon and 178 base pairs upstream (e.g., −178) of the start codon. In some specific embodiments, the first external CSRE is located between about 139 base pairs upstream (e.g., −139) of the start codon and about 218 base pairs upstream (e.g., −218) of the start codon. In some additional embodiments, the first external CSRE is located between 178 base pairs upstream (e.g., −178) of the start codon and 179 base pairs upstream (e.g., −179) of the start codon. In some specific embodiments, the first external CSRE is located between about 144 base pairs upstream (e.g., −144) of the start codon and about 223 base pairs upstream (e.g., −223) of the start codon. In some additional embodiments, the first external CSRE is located between 183 base pairs upstream (e.g., −183) of the start codon and 184 base pairs upstream (e.g., −184) of the start codon. In some specific embodiments, the first external CSRE is located between about 149 base pairs upstream (e.g., −149) of the start codon and about 228 base pairs upstream (e.g., −228) of the start codon. In some additional embodiments, the first external CSRE is located between 188 base pairs upstream (e.g., −188) of the start codon and 189 base pairs upstream (e.g., −189) of the start codon. In some specific embodiments, the first external CSRE is located between about 154 base pairs upstream (e.g., −154) of the start codon and about 233 base pairs upstream (e.g., −233) of the start codon. In some additional embodiments, the first external CSRE is located between 193 base pairs upstream (e.g., −193) of the start codon and 194 base pairs upstream (e.g., −194) of the start codon. In some specific embodiments, the first external CSRE is located between about 156 base pairs upstream (e.g., −156) of the start codon and about 235 base pairs upstream (e.g., −235) of the start codon. In some additional embodiments, the first external CSRE is located between 195 base pairs upstream (e.g., −195) of the start codon and 196 base pairs upstream (e.g., −196) of the start codon. In some specific embodiments, the first external CSRE is located between about 159 base pairs upstream (e.g., −159) of the start codon and about 238 base pairs upstream (e.g., −238) of the start codon. In some additional embodiments, the first external CSRE is located between 198 base pairs upstream (e.g., −198) of the start codon and 199 base pairs upstream (e.g., −199) of the start codon. In some specific embodiments, the first external CSRE is located between about 164 base pairs upstream (e.g., −164) of the start codon and about 243 base pairs upstream (e.g., −243) of the start codon. In some additional embodiments, the first external CSRE is located between 203 base pairs upstream (e.g., −203) of the start codon and 204 base pairs upstream (e.g., −204) of the start codon. In some specific embodiments, the first external CSRE is located between about 169 base pairs upstream (e.g., −169) of the start codon and about 248 base pairs upstream (e.g., −248) of the start codon. In some additional embodiments, the first external CSRE is located between 208 base pairs upstream (e.g., −208) of the start codon and 209 base pairs upstream (e.g., −209) of the start codon. In some specific embodiments, the first external CSRE is located between about 174 base pairs upstream (e.g., −174) of the start codon and about 253 base pairs upstream (e.g., −253) of the start codon. In some additional embodiments, the first external CSRE is located between 213 base pairs upstream (e.g., −213) of the start codon and 214 base pairs upstream (e.g., −214) of the start codon. In some specific embodiments, the first external CSRE is located between about 234 base pairs upstream (e.g., −234) of the start codon and about 313 base pairs upstream (e.g., −313) of the start codon. In some additional embodiments, the first external CSRE is located between 273 base pairs upstream (e.g., −273) of the start codon and 274 base pairs upstream (e.g., −274) of the start codon. In some specific embodiments, the first external CSRE is located between about 243 base pairs upstream (e.g., −243) of the start codon and about 322 base pairs upstream (e.g., −322) of the start codon. In some additional embodiments, the first external CSRE is located between 282 base pairs upstream (e.g., −282) of the start codon and 283 base pairs upstream (e.g., −283) of the start codon. In some specific embodiments, the first external CSRE is located between about 284 base pairs upstream (e.g., −284) of the start codon and about 363 base pairs upstream (e.g., −363) of the start codon. In some additional embodiments, the first external CSRE is located between 323 base pairs upstream (e.g., −323) of the start codon and 324 base pairs upstream (e.g., −324) of the start codon. In some additional embodiments, the first external CSRE is located between 363 base pairs upstream (e.g., −363) of the start codon and 442 base pairs upstream (e.g., −442) of the start codon. In some additional embodiments, the first external CSRE is located between 402 base pairs upstream (e.g., −402) of the start codon and 403 base pairs upstream (e.g., −403) of the start codon.

In some embodiments, the engineered promoter of the present disclosure comprises at least two external CSREs. Embodiments of the location and the nucleic acid sequence of the first external CSRE are provided herein and can be used in an engineered promoter comprising two or more external CSREs. When a plurality of CSREs is included in the engineered promoter, the CSREs can be independently located at the same position and be contiguous or be provided at different positions and be non-contiguous. In some specific embodiments, the second external CSRE is located between about 52 base pairs upstream (e.g., −52) of the start codon and about 131 base pairs upstream (e.g., −131) of the start codon. In some additional embodiments, the second external CSRE is located between 91 base pairs upstream (e.g., −91) of the start codon and 92 base pairs upstream (e.g., −92) of the start codon. In some specific embodiments, the second external CSRE is located between about 82 base pairs upstream (e.g., −82) of the start codon and about 161 base pairs upstream (e.g., −161) of the start codon. In some additional embodiments, the second external CSRE is located between 121 base pairs upstream (e.g., −121) of the start codon and 122 base pairs upstream (e.g., −122) of the start codon. In some specific embodiments, the second external CSRE is located between about 92 base pairs upstream (e.g., −92) of the start codon and about 171 base pairs upstream (e.g., −171) of the start codon. In some additional embodiments, the second external CSRE is located between 131 base pairs upstream (e.g., −131) of the start codon and 132 base pairs upstream (e.g., −132) of the start codon. In some specific embodiments, the second external CSRE is located between about 100 base pairs upstream (e.g., −100) of the start codon and about 179 base pairs upstream (e.g., −179) of the start codon. In some additional embodiments, the second external CSRE is located between 139 base pairs upstream (e.g., −139) of the start codon and 140 base pairs upstream (e.g., −140) of the start codon. In some specific embodiments, the second external CSRE is located between about 122 base pairs upstream (e.g., −122) of the start codon and about 201 base pairs upstream (e.g., −201) of the start codon. In some additional embodiments, the second external CSRE is located between 161 base pairs upstream (e.g., −161) of the start codon and 162 base pairs upstream (e.g., −162) of the start codon. In some specific embodiments, the second external CSRE is located between about 126 base pairs upstream (e.g., −126) of the start codon and about 205 base pairs upstream (e.g., −205) of the start codon. In some additional embodiments, the second external CSRE is located between 165 base pairs upstream (e.g., −165) of the start codon and 166 base pairs upstream (e.g., −166) of the start codon. In some specific embodiments, the second external CSRE is located between about 132 base pairs upstream (e.g., −132) of the start codon and about 211 base pairs upstream (e.g., −211) of the start codon. In some additional embodiments, the second external CSRE is located between 171 base pairs upstream (e.g., −171) of the start codon and 172 base pairs upstream (e.g., −172) of the start codon. In some specific embodiments, the second external CSRE is located between about 138 base pairs upstream (e.g., −138) of the start codon and about 217 base pairs upstream (e.g., −217) of the start codon. In some additional embodiments, the second external CSRE is located between 177 base pairs upstream (e.g., −177) of the start codon and 178 base pairs upstream (e.g., −178) of the start codon. In some specific embodiments, the second external CSRE is located between about 139 base pairs upstream (e.g., −139) of the start codon and about 218 base pairs upstream (e.g., −218) of the start codon. In some additional embodiments, the second external CSRE is located between 178 base pairs upstream (e.g., −178) of the start codon and 179 base pairs upstream (e.g., −179) of the start codon. In some specific embodiments, the second external CSRE is located between about 144 base pairs upstream (e.g., −144) of the start codon and about 223 base pairs upstream (e.g., −223) of the start codon. In some additional embodiments, the second external CSRE is located between 183 base pairs upstream (e.g., −183) of the start codon and 184 base pairs upstream (e.g., −184) of the start codon. In some specific embodiments, the second external CSRE is located between about 149 base pairs upstream (e.g., −149) of the start codon and about 228 base pairs upstream (e.g., −228) of the start codon. In some additional embodiments, the second external CSRE is located between 188 base pairs upstream (e.g., −188) of the start codon and 189 base pairs upstream (e.g., −189) of the start codon. In some specific embodiments, the second external CSRE is located between about 154 base pairs upstream (e.g., −154) of the start codon and about 233 base pairs upstream (e.g., −233) of the start codon. In some additional embodiments, the second external CSRE is located between 193 base pairs upstream (e.g., −193) of the start codon and 194 base pairs upstream (e.g., −194) of the start codon. In some specific embodiments, the second external CSRE is located between about 156 base pairs upstream (e.g., −156) of the start codon and about 235 base pairs upstream (e.g., −235) of the start codon. In some additional embodiments, the second external CSRE is located between 195 base pairs upstream (e.g., −195) of the start codon and 196 base pairs upstream (e.g., −196) of the start codon. In some specific embodiments, the second external CSRE is located between about 159 base pairs upstream (e.g., −159) of the start codon and about 238 base pairs upstream (e.g., −238) of the start codon. In some additional embodiments, the second external CSRE is located between 198 base pairs upstream (e.g., −198) of the start codon and 199 base pairs upstream (e.g., −199) of the start codon. In some specific embodiments, the second external CSRE is located between about 164 base pairs upstream (e.g., −164) of the start codon and about 243 base pairs upstream (e.g., −243) of the start codon. In some additional embodiments, the second external CSRE is located between 203 base pairs upstream (e.g., −203) of the start codon and 204 base pairs upstream (e.g., −204) of the start codon. In some specific embodiments, the second external CSRE is located between about 169 base pairs upstream (e.g., −169) of the start codon and about 248 base pairs upstream (e.g., −248) of the start codon. In some additional embodiments, the second external CSRE is located between 208 base pairs upstream (e.g., −208) of the start codon and 209 base pairs upstream (e.g., −209) of the start codon. In some specific embodiments, the second external CSRE is located between about 174 base pairs upstream (e.g., −174) of the start codon and about 253 base pairs upstream (e.g., −253) of the start codon. In some additional embodiments, the second external CSRE is located between 213 base pairs upstream (e.g., −213) of the start codon and 214 base pairs upstream (e.g., −214) of the start codon. In some specific embodiments, the second external CSRE is located between about 234 base pairs upstream (e.g., −234) of the start codon and about 313 base pairs upstream (e.g., −313) of the start codon. In some additional embodiments, the second external CSRE is located between 273 base pairs upstream (e.g., −273) of the start codon and 274 base pairs upstream (e.g., −274) of the start codon. In some specific embodiments, the second external CSRE is located between about 243 base pairs upstream (e.g., −243) of the start codon and about 322 base pairs upstream (e.g., −322) of the start codon. In some additional embodiments, the second external CSRE is located between 282 base pairs upstream (e.g., −282) of the start codon and 283 base pairs upstream (e.g., −283) of the start codon. In some specific embodiments, the second external CSRE is located between about 284 base pairs upstream (e.g., −284) of the start codon and about 363 base pairs upstream (e.g., −363) of the start codon. In some additional embodiments, the second external CSRE is located between 323 base pairs upstream (e.g., −323) of the start codon and 324 base pairs upstream (e.g., −324) of the start codon. In some additional embodiments, the second external CSRE is located between 363 base pairs upstream (e.g., −363) of the start codon and 442 base pairs upstream (e.g., −442) of the start codon. In some additional embodiments, the second external CSRE is located between 402 base pairs upstream (e.g., −402) of the start codon and 403 base pairs upstream (e.g., −403) of the start codon. In still another embodiment, the engineered promoter with two or more external CSREs comprises a first external CSRE located between about 174 base pairs upstream (e.g., −174) of the start codon and about 253 base pairs upstream (e.g., −253) of the start codon; and a second external CSRE located between about 243 base pairs upstream (e.g., −243) of the start codon and about 322 base pairs upstream (e.g., −322) of the start codon. In yet still another embodiment, the engineered promoter with two or more external CSREs comprises a first external CSRE located between 213 base pairs upstream (e.g., −213) of the start codon and 214 base pairs upstream (e.g., −214) of the start codon; and a second external CSRE located between 282 base pairs upstream (e.g., −282) of the start codon and 283 base pairs upstream (e.g., −283) of the start codon.

In some embodiments, the engineered promoter of the present disclosure comprises at least three external CSREs. Embodiments of the location and the nucleic acid sequence of the first and second external CSREs are provided herein and can be used in an engineered promoter comprising three or more external CSREs. When a plurality of CSREs is included in the engineered promoter, the CSREs can be independently located at the same position and be contiguous or be provided at different positions and be non-contiguous. In some specific embodiments, the third external CSRE is located between about 52 base pairs upstream (e.g., −52) of the start codon and about 131 base pairs upstream (e.g., −131) of the start codon. In some additional embodiments, the third external CSRE is located between 91 base pairs upstream (e.g., −91) of the start codon and 92 base pairs upstream (e.g., −92) of the start codon. In some specific embodiments, the third external CSRE is located between about 82 base pairs upstream (e.g., −82) of the start codon and about 161 base pairs upstream (e.g., −161) of the start codon. In some additional embodiments, the third external CSRE is located between 121 base pairs upstream (e.g., −121) of the start codon and 122 base pairs upstream (e.g., −122) of the start codon. In some specific embodiments, the third external CSRE is located between about 92 base pairs upstream (e.g., −92) of the start codon and about 171 base pairs upstream (e.g., −171) of the start codon. In some additional embodiments, the third external CSRE is located between 131 base pairs upstream (e.g., −131) of the start codon and 132 base pairs upstream (e.g., −132) of the start codon. In some specific embodiments, the third external CSRE is located between about 100 base pairs upstream (e.g., −100) of the start codon and about 179 base pairs upstream (e.g., −179) of the start codon. In some additional embodiments, the third external CSRE is located between 139 base pairs upstream (e.g., −139) of the start codon and 140 base pairs upstream (e.g., −140) of the start codon. In some specific embodiments, the third external CSRE is located between about 122 base pairs upstream (e.g., −122) of the start codon and about 201 base pairs upstream (e.g., −201) of the start codon. In some additional embodiments, the third external CSRE is located between 161 base pairs upstream (e.g., −161) of the start codon and 162 base pairs upstream (e.g., −162) of the start codon. In some specific embodiments, the third external CSRE is located between about 126 base pairs upstream (e.g., −126) of the start codon and about 205 base pairs upstream (e.g., −205) of the start codon. In some additional embodiments, the third external CSRE is located between 165 base pairs upstream (e.g., −165) of the start codon and 166 base pairs upstream (e.g., −166) of the start codon. In some specific embodiments, the third external CSRE is located between about 132 base pairs upstream (e.g., −132) of the start codon and about 211 base pairs upstream (e.g., −211) of the start codon. In some additional embodiments, the third external CSRE is located between 171 base pairs upstream (e.g., −171) of the start codon and 172 base pairs upstream (e.g., −172) of the start codon. In some specific embodiments, the third external CSRE is located between about 138 base pairs upstream (e.g., −138) of the start codon and about 217 base pairs upstream (e.g., −217) of the start codon. In some additional embodiments, the third external CSRE is located between 177 base pairs upstream (e.g., −177) of the start codon and 178 base pairs upstream (e.g., −178) of the start codon. In some specific embodiments, the third external CSRE is located between about 139 base pairs upstream (e.g., −139) of the start codon and about 218 base pairs upstream (e.g., −218) of the start codon. In some additional embodiments, the third external CSRE is located between 178 base pairs upstream (e.g., −178) of the start codon and 179 base pairs upstream (e.g., −179) of the start codon. In some specific embodiments, the third external CSRE is located between about 144 base pairs upstream (e.g., −144) of the start codon and about 223 base pairs upstream (e.g., −223) of the start codon. In some additional embodiments, the third external CSRE is located between 183 base pairs upstream (e.g., −183) of the start codon and 184 base pairs upstream (e.g., −184) of the start codon. In some specific embodiments, the third external CSRE is located between about 149 base pairs upstream (e.g., −149) of the start codon and about 228 base pairs upstream (e.g., −228) of the start codon. In some additional embodiments, the third external CSRE is located between 188 base pairs upstream (e.g., −188) of the start codon and 189 base pairs upstream (e.g., −189) of the start codon. In some specific embodiments, the third external CSRE is located between about 154 base pairs upstream (e.g., −154) of the start codon and about 233 base pairs upstream (e.g., −233) of the start codon. In some additional embodiments, the third external CSRE is located between 193 base pairs upstream (e.g., −193) of the start codon and 194 base pairs upstream (e.g., −194) of the start codon. In some specific embodiments, the third external CSRE is located between about 156 base pairs upstream (e.g., −156) of the start codon and about 235 base pairs upstream (e.g., −235) of the start codon. In some additional embodiments, the third external CSRE is located between 195 base pairs upstream (e.g., −195) of the start codon and 196 base pairs upstream (e.g., −196) of the start codon. In some specific embodiments, the third external CSRE is located between about 159 base pairs upstream (e.g., −159) of the start codon and about 238 base pairs upstream (e.g., −238) of the start codon. In some additional embodiments, the third external CSRE is located between 198 base pairs upstream (e.g., −198) of the start codon and 199 base pairs upstream (e.g., −199) of the start codon. In some specific embodiments, the third external CSRE is located between about 164 base pairs upstream (e.g., −164) of the start codon and about 243 base pairs upstream (e.g., −243) of the start codon. In some additional embodiments, the third external CSRE is located between 203 base pairs upstream (e.g., −203) of the start codon and 204 base pairs upstream (e.g., −204) of the start codon. In some specific embodiments, the third external CSRE is located between about 169 base pairs upstream (e.g., −169) of the start codon and about 248 base pairs upstream (e.g., −248) of the start codon. In some additional embodiments, the third external CSRE is located between 208 base pairs upstream (e.g., −208) of the start codon and 209 base pairs upstream (e.g., −209) of the start codon. In some specific embodiments, the third external CSRE is located between about 174 base pairs upstream (e.g., −174) of the start codon and about 253 base pairs upstream (e.g., −253) of the start codon. In some additional embodiments, the third external CSRE is located between 213 base pairs upstream (e.g., −213) of the start codon and 214 base pairs upstream (e.g., −214) of the start codon. In some specific embodiments, the third external CSRE is located between about 234 base pairs upstream (e.g., −234) of the start codon and about 313 base pairs upstream (e.g., −313) of the start codon. In some additional embodiments, the third external CSRE is located between 273 base pairs upstream (e.g., −273) of the start codon and 274 base pairs upstream (e.g., −274) of the start codon. In some specific embodiments, the third external CSRE is located between about 243 base pairs upstream (e.g., −243) of the start codon and about 322 base pairs upstream (e.g., −322) of the start codon. In some additional embodiments, the third external CSRE is located between 282 base pairs upstream (e.g., −282) of the start codon and 283 base pairs upstream (e.g., −283) of the start codon. In some specific embodiments, the third external CSRE is located between about 284 base pairs upstream (e.g., −284) of the start codon and about 363 base pairs upstream (e.g., −363) of the start codon. In some additional embodiments, the third external CSRE is located between 323 base pairs upstream (e.g., −323) of the start codon and 324 base pairs upstream (e.g., −324) of the start codon. In some additional embodiments, the third external CSRE is located between 363 base pairs upstream (e.g., −363) of the start codon and 442 base pairs upstream (e.g., −442) of the start codon. In some additional embodiments, the third external CSRE is located between 402 base pairs upstream (e.g., −402) of the start codon and 403 base pairs upstream (e.g., −403) of the start codon. In still another embodiment, the engineered promoter with three or more external CSREs comprises a first external CSRE located between about 174 base pairs upstream (e.g., −174) of the start codon and about 253 base pairs upstream (e.g., −253) of the start codon; a second external CSRE located between about 243 base pairs upstream (e.g., −243) of the start codon and about 322 base pairs upstream (e.g., −322) of the start codon; and a third external CSRE located between about 363 base pairs upstream (e.g., −363) of the start codon and about 442 base pairs upstream (e.g., −442) of the start codon. In yet still another embodiment, the engineered promoter with three or more external CSREs comprises a first external CSRE located between 213 base pairs upstream (e.g., −213) of the start codon and 214 base pairs upstream (e.g., −214) of the start codon; a second external CSRE located between 282 base pairs upstream (e.g., −282) of the start codon and 283 base pairs upstream (e.g., −283) of the start codon; and a third external CSRE located between 402 base pairs upstream (e.g., −402) of the start codon and 403 base pairs upstream (e.g., −403) of the start codon.

In some embodiments, the engineered promoter of the present disclosure comprises at least four external CSREs. Embodiments of the location and the nucleic acid sequence of the first, second, and third external CSREs are provided herein and can be used in an engineered promoter comprising four or more external CSREs. When a plurality of CSREs is included in the engineered promoter, the CSREs can be independently located at the same position and be contiguous or be provided at different positions and be non-contiguous. In some specific embodiments, the fourth external CSRE is located between about 52 base pairs upstream (e.g., −52) of the start codon and about 131 base pairs upstream (e.g., −131) of the start codon. In some additional embodiments, the fourth external CSRE is located between 91 base pairs upstream (e.g., −91) of the start codon and 92 base pairs upstream (e.g., −92) of the start codon. In some specific embodiments, the fourth external CSRE is located between about 82 base pairs upstream (e.g., −82) of the start codon and about 161 base pairs upstream (e.g., −161) of the start codon. In some additional embodiments, the fourth external CSRE is located between 121 base pairs upstream (e.g., −121) of the start codon and 122 base pairs upstream (e.g., −122) of the start codon. In some specific embodiments, the fourth external CSRE is located between about 92 base pairs upstream (e.g., −92) of the start codon and about 171 base pairs upstream (e.g., −171) of the start codon. In some additional embodiments, the fourth external CSRE is located between 131 base pairs upstream (e.g., −131) of the start codon and 132 base pairs upstream (e.g., −132) of the start codon. In some specific embodiments, the fourth external CSRE is located between about 100 base pairs upstream (e.g., −100) of the start codon and about 179 base pairs upstream (e.g., −179) of the start codon. In some additional embodiments, the fourth external CSRE is located between 139 base pairs upstream (e.g., −139) of the start codon and 140 base pairs upstream (e.g., −140) of the start codon. In some specific embodiments, the fourth external CSRE is located between about 122 base pairs upstream (e.g., −122) of the start codon and about 201 base pairs upstream (e.g., −201) of the start codon. In some additional embodiments, the fourth external CSRE is located between 161 base pairs upstream (e.g., −161) of the start codon and 162 base pairs upstream (e.g., −162) of the start codon. In some specific embodiments, the fourth external CSRE is located between about 126 base pairs upstream (e.g., −126) of the start codon and about 205 base pairs upstream (e.g., −205) of the start codon. In some additional embodiments, the fourth external CSRE is located between 165 base pairs upstream (e.g., −165) of the start codon and 166 base pairs upstream (e.g., −166) of the start codon. In some specific embodiments, the fourth external CSRE is located between about 132 base pairs upstream (e.g., −132) of the start codon and about 211 base pairs upstream (e.g., −211) of the start codon. In some additional embodiments, the fourth external CSRE is located between 171 base pairs upstream (e.g., −171) of the start codon and 172 base pairs upstream (e.g., −172) of the start codon. In some specific embodiments, the fourth external CSRE is located between about 138 base pairs upstream (e.g., −138) of the start codon and about 217 base pairs upstream (e.g., −217) of the start codon. In some additional embodiments, the fourth external CSRE is located between 177 base pairs upstream (e.g., −177) of the start codon and 178 base pairs upstream (e.g., −178) of the start codon. In some specific embodiments, the fourth external CSRE is located between about 139 base pairs upstream (e.g., −139) of the start codon and about 218 base pairs upstream (e.g., −218) of the start codon. In some additional embodiments, the fourth external CSRE is located between 178 base pairs upstream (e.g., −178) of the start codon and 179 base pairs upstream (e.g., −179) of the start codon. In some specific embodiments, the fourth external CSRE is located between about 144 base pairs upstream (e.g., −144) of the start codon and about 223 base pairs upstream (e.g., −223) of the start codon. In some additional embodiments, the fourth external CSRE is located between 183 base pairs upstream (e.g., −183) of the start codon and 184 base pairs upstream (e.g., −184) of the start codon. In some specific embodiments, the fourth external CSRE is located between about 149 base pairs upstream (e.g., −149) of the start codon and about 228 base pairs upstream (e.g., −228) of the start codon. In some additional embodiments, the fourth external CSRE is located between 188 base pairs upstream (e.g., −188) of the start codon and 189 base pairs upstream (e.g., −189) of the start codon. In some specific embodiments, the fourth external CSRE is located between about 154 base pairs upstream (e.g., −154) of the start codon and about 233 base pairs upstream (e.g., −233) of the start codon. In some additional embodiments, the fourth external CSRE is located between 193 base pairs upstream (e.g., −193) of the start codon and 194 base pairs upstream (e.g., −194) of the start codon. In some specific embodiments, the fourth external CSRE is located between about 156 base pairs upstream (e.g., −156) of the start codon and about 235 base pairs upstream (e.g., −235) of the start codon. In some additional embodiments, the fourth external CSRE is located between 195 base pairs upstream (e.g., −195) of the start codon and 196 base pairs upstream (e.g., −196) of the start codon. In some specific embodiments, the fourth external CSRE is located between about 159 base pairs upstream (e.g., −159) of the start codon and about 238 base pairs upstream (e.g., −238) of the start codon. In some additional embodiments, the fourth external CSRE is located between 198 base pairs upstream (e.g., −198) of the start codon and 199 base pairs upstream (e.g., −199) of the start codon. In some specific embodiments, the fourth external CSRE is located between about 164 base pairs upstream (e.g., −164) of the start codon and about 243 base pairs upstream (e.g., −243) of the start codon. In some additional embodiments, the fourth external CSRE is located between 203 base pairs upstream (e.g., −203) of the start codon and 204 base pairs upstream (e.g., −204) of the start codon. In some specific embodiments, the fourth external CSRE is located between about 169 base pairs upstream (e.g., −169) of the start codon and about 248 base pairs upstream (e.g., −248) of the start codon. In some additional embodiments, the fourth external CSRE is located between 208 base pairs upstream (e.g., −208) of the start codon and 209 base pairs upstream (e.g., −209) of the start codon. In some specific embodiments, the fourth external CSRE is located between about 174 base pairs upstream (e.g., −174) of the start codon and about 253 base pairs upstream (e.g., −253) of the start codon. In some additional embodiments, the fourth external CSRE is located between 213 base pairs upstream (e.g., −213) of the start codon and 214 base pairs upstream (e.g., −214) of the start codon. In some specific embodiments, the fourth external CSRE is located between about 234 base pairs upstream (e.g., −234) of the start codon and about 313 base pairs upstream (e.g., −313) of the start codon. In some additional embodiments, the fourth external CSRE is located between 273 base pairs upstream (e.g., −273) of the start codon and 274 base pairs upstream (e.g., −274) of the start codon. In some specific embodiments, the fourth external CSRE is located between about 243 base pairs upstream (e.g., −243) of the start codon and about 322 base pairs upstream (e.g., −322) of the start codon. In some additional embodiments, the fourth external CSRE is located between 282 base pairs upstream (e.g., −282) of the start codon and 283 base pairs upstream (e.g., −283) of the start codon. In some specific embodiments, the fourth external CSRE is located between about 284 base pairs upstream (e.g., −284) of the start codon and about 363 base pairs upstream (e.g., −363) of the start codon. In some additional embodiments, the fourth external CSRE is located between 323 base pairs upstream (e.g., −323) of the start codon and 324 base pairs upstream (e.g., −324) of the start codon. In some additional embodiments, the fourth external CSRE is located between 363 base pairs upstream (e.g., −363) of the start codon and 442 base pairs upstream (e.g., −442) of the start codon. In some additional embodiments, the fourth external CSRE is located between 402 base pairs upstream (e.g., −402) of the start codon and 403 base pairs upstream (e.g., −403) of the start codon.

In some embodiments, the engineered promoter of the present disclosure comprises at least five external CSREs. Embodiments of the location and the nucleic acid sequence of the first, second, third, and fourth external CSREs are provided herein and can be used in an engineered promoter comprising five or more external CSREs. When a plurality of CSREs is included in the engineered promoter, the CSREs can be independently located at the same position and be contiguous or be provided at different positions and be non-contiguous. In some specific embodiments, the fifth external CSRE is located between about 52 base pairs upstream (e.g., −52) of the start codon and about 131 base pairs upstream (e.g., −131) of the start codon. In some additional embodiments, the fifth external CSRE is located between 91 base pairs upstream (e.g., −91) of the start codon and 92 base pairs upstream (e.g., −92) of the start codon. In some specific embodiments, the fifth external CSRE is located between about 82 base pairs upstream (e.g., −82) of the start codon and about 161 base pairs upstream (e.g., −161) of the start codon. In some additional embodiments, the fifth external CSRE is located between 121 base pairs upstream (e.g., −121) of the start codon and 122 base pairs upstream (e.g., −122) of the start codon. In some specific embodiments, the fifth external CSRE is located between about 92 base pairs upstream (e.g., −92) of the start codon and about 171 base pairs upstream (e.g., −171) of the start codon. In some additional embodiments, the fifth external CSRE is located between 131 base pairs upstream (e.g., −131) of the start codon and 132 base pairs upstream (e.g., −132) of the start codon. In some specific embodiments, the fifth external CSRE is located between about 100 base pairs upstream (e.g., −100) of the start codon and about 179 base pairs upstream (e.g., −179) of the start codon. In some additional embodiments, the fifth external CSRE is located between 139 base pairs upstream (e.g., −139) of the start codon and 140 base pairs upstream (e.g., −140) of the start codon. In some specific embodiments, the fifth external CSRE is located between about 122 base pairs upstream (e.g., −122) of the start codon and about 201 base pairs upstream (e.g., −201) of the start codon. In some additional embodiments, the fifth external CSRE is located between 161 base pairs upstream (e.g., −161) of the start codon and 162 base pairs upstream (e.g., −162) of the start codon. In some specific embodiments, the fifth external CSRE is located between about 126 base pairs upstream (e.g., −126) of the start codon and about 205 base pairs upstream (e.g., −205) of the start codon. In some additional embodiments, the fifth external CSRE is located between 165 base pairs upstream (e.g., −165) of the start codon and 166 base pairs upstream (e.g., −166) of the start codon. In some specific embodiments, the fifth external CSRE is located between about 132 base pairs upstream (e.g., −132) of the start codon and about 211 base pairs upstream (e.g., −211) of the start codon. In some additional embodiments, the fifth external CSRE is located between 171 base pairs upstream (e.g., −171) of the start codon and 172 base pairs upstream (e.g., −172) of the start codon. In some specific embodiments, the fifth external CSRE is located between about 138 base pairs upstream (e.g., −138) of the start codon and about 217 base pairs upstream (e.g., −217) of the start codon. In some additional embodiments, the fifth external CSRE is located between 177 base pairs upstream (e.g., −177) of the start codon and 178 base pairs upstream (e.g., −178) of the start codon. In some specific embodiments, the fifth external CSRE is located between about 139 base pairs upstream (e.g., −139) of the start codon and about 218 base pairs upstream (e.g., −218) of the start codon. In some additional embodiments, the fifth external CSRE is located between 178 base pairs upstream (e.g., −178) of the start codon and 179 base pairs upstream (e.g., −179) of the start codon. In some specific embodiments, the fifth external CSRE is located between about 144 base pairs upstream (e.g., −144) of the start codon and about 223 base pairs upstream (e.g., −223) of the start codon. In some additional embodiments, the fifth external CSRE is located between 183 base pairs upstream (e.g., −183) of the start codon and 184 base pairs upstream (e.g., −184) of the start codon. In some specific embodiments, the fifth external CSRE is located between about 149 base pairs upstream (e.g., −149) of the start codon and about 228 base pairs upstream (e.g., −228) of the start codon. In some additional embodiments, the fifth external CSRE is located between 188 base pairs upstream (e.g., −188) of the start codon and 189 base pairs upstream (e.g., −189) of the start codon. In some specific embodiments, the fifth external CSRE is located between about 154 base pairs upstream (e.g., −154) of the start codon and about 233 base pairs upstream (e.g., −233) of the start codon. In some additional embodiments, the fifth external CSRE is located between 193 base pairs upstream (e.g., −193) of the start codon and 194 base pairs upstream (e.g., −194) of the start codon. In some specific embodiments, the fifth external CSRE is located between about 156 base pairs upstream (e.g., −156) of the start codon and about 235 base pairs upstream (e.g., −235) of the start codon. In some additional embodiments, the fifth external CSRE is located between 195 base pairs upstream (e.g., −195) of the start codon and 196 base pairs upstream (e.g., −196) of the start codon. In some specific embodiments, the fifth external CSRE is located between about 159 base pairs upstream (e.g., −159) of the start codon and about 238 base pairs upstream (e.g., −238) of the start codon. In some additional embodiments, the fifth external CSRE is located between 198 base pairs upstream (e.g., −198) of the start codon and 199 base pairs upstream (e.g., −199) of the start codon. In some specific embodiments, the fifth external CSRE is located between about 164 base pairs upstream (e.g., −164) of the start codon and about 243 base pairs upstream (e.g., −243) of the start codon. In some additional embodiments, the fifth external CSRE is located between 203 base pairs upstream (e.g., −203) of the start codon and 204 base pairs upstream (e.g., −204) of the start codon. In some specific embodiments, the fifth external CSRE is located between about 169 base pairs upstream (e.g., −169) of the start codon and about 248 base pairs upstream (e.g., −248) of the start codon. In some additional embodiments, the fifth external CSRE is located between 208 base pairs upstream (e.g., −208) of the start codon and 209 base pairs upstream (e.g., −209) of the start codon. In some specific embodiments, the fifth external CSRE is located between about 174 base pairs upstream (e.g., −174) of the start codon and about 253 base pairs upstream (e.g., −253) of the start codon. In some additional embodiments, the fifth external CSRE is located between 213 base pairs upstream (e.g., −213) of the start codon and 214 base pairs upstream (e.g., −214) of the start codon. In some specific embodiments, the fifth external CSRE is located between about 234 base pairs upstream (e.g., −234) of the start codon and about 313 base pairs upstream (e.g., −313) of the start codon. In some additional embodiments, the fifth external CSRE is located between 273 base pairs upstream (e.g., −273) of the start codon and 274 base pairs upstream (e.g., −274) of the start codon. In some specific embodiments, the fifth external CSRE is located between about 243 base pairs upstream (e.g., −243) of the start codon and about 322 base pairs upstream (e.g., −322) of the start codon. In some additional embodiments, the fifth external CSRE is located between 282 base pairs upstream (e.g., −282) of the start codon and 283 base pairs upstream (e.g., −283) of the start codon. In some specific embodiments, the fifth external CSRE is located between about 284 base pairs upstream (e.g., −284) of the start codon and about 363 base pairs upstream (e.g., −363) of the start codon. In some additional embodiments, the fifth external CSRE is located between 323 base pairs upstream (e.g., −323) of the start codon and 324 base pairs upstream (e.g., −324) of the start codon. In some additional embodiments, the fifth external CSRE is located between 363 base pairs upstream (e.g., −363) of the start codon and 442 base pairs upstream (e.g., −442) of the start codon. some additional embodiments, the fifth external CSRE is located between 402 base pairs upstream (e.g., −402) of the start codon and 403 base pairs upstream (e.g., −403) of the start codon. In still another embodiment, the engineered promoter with five or more external CSREs comprises a first external CSRE located between about 138 base pairs upstream (e.g., −138) of the start codon and about 217 base pairs upstream (e.g., −217) of the start codon; a second external CSRE located between about 156 base pairs upstream (e.g., −156) of the start codon and about 235 base pairs upstream (e.g., −235) of the start codon; a third external CSRE located between about 174 base pairs upstream (e.g., −174) of the start codon and about 253 base pairs upstream (e.g., −253) of the start codon; a fourth external CSRE located between about 243 base pairs upstream (e.g., −243) of the start codon and about 322 base pairs upstream (e.g., −322) of the start codon; and a fifth external CSRE located between about 363 base pairs upstream (e.g., −363) of the start codon and about 442 base pairs upstream (e.g., −442) of the start codon. In yet still another embodiment, the engineered promoter with five or more external CSREs comprises a first external CSRE located between 177 base pairs upstream (e.g., −177) of the start codon and 178 base pairs upstream (e.g., −178) of the start codon; a second external CSRE located between 195 base pairs upstream (e.g., −195) of the start codon and 196 base pairs upstream (e.g., −196) of the start codon; a third external CSRE located between 213 base pairs upstream (e.g., −213) of the start codon and 214 base pairs upstream (e.g., −214) of the start codon; a fourth external CSRE located between 282 base pairs upstream (e.g., −282) of the start codon and 283 base pairs upstream (e.g., −283) of the start codon; and a fifth external CSRE located between 402 base pairs upstream (e.g., −402) of the start codon and 403 base pairs upstream (e.g., −403) of the start codon.

In some embodiments, the engineered promoter of the present disclosure comprises at least six external CSREs. Embodiments of the location and the nucleic acid sequence of the first, second, third, fourth, and fifth external CSREs are provided herein and can be used in an engineered promoter comprising six or more external CSREs. When a plurality of CSREs is included in the engineered promoter, the CSREs can be independently located at the same position and be contiguous or be provided at different positions and be non-contiguous. In some specific embodiments, the sixth external CSRE is located between about 52 base pairs upstream (e.g., −52) of the start codon and about 131 base pairs upstream (e.g., −131) of the start codon. In some additional embodiments, the sixth external CSRE is located between 91 base pairs upstream (e.g., −91) of the start codon and 92 base pairs upstream (e.g., −92) of the start codon. In some specific embodiments, the sixth external CSRE is located between about 82 base pairs upstream (e.g., −82) of the start codon and about 161 base pairs upstream (e.g., −161) of the start codon. In some additional embodiments, the sixth external CSRE is located between 121 base pairs upstream (e.g., −121) of the start codon and 122 base pairs upstream (e.g., −122) of the start codon. In some specific embodiments, the sixth external CSRE is located between about 92 base pairs upstream (e.g., −92) of the start codon and about 171 base pairs upstream (e.g., −171) of the start codon. In some additional embodiments, the sixth external CSRE is located between 131 base pairs upstream (e.g., −131) of the start codon and 132 base pairs upstream (e.g., −132) of the start codon. In some specific embodiments, the sixth external CSRE is located between about 100 base pairs upstream (e.g., −100) of the start codon and about 179 base pairs upstream (e.g., −179) of the start codon. In some additional embodiments, the sixth external CSRE is located between 139 base pairs upstream (e.g., −139) of the start codon and 140 base pairs upstream (e.g., −140) of the start codon. In some specific embodiments, the sixth external CSRE is located between about 122 base pairs upstream (e.g., −122) of the start codon and about 201 base pairs upstream (e.g., −201) of the start codon. In some additional embodiments, the sixth external CSRE is located between 161 base pairs upstream (e.g., −161) of the start codon and 162 base pairs upstream (e.g., −162) of the start codon. In some specific embodiments, the sixth external CSRE is located between about 126 base pairs upstream (e.g., −126) of the start codon and about 205 base pairs upstream (e.g., −205) of the start codon. In some additional embodiments, the sixth external CSRE is located between 165 base pairs upstream (e.g., −165) of the start codon and 166 base pairs upstream (e.g., −166) of the start codon. In some specific embodiments, the sixth external CSRE is located between about 132 base pairs upstream (e.g., −132) of the start codon and about 211 base pairs upstream (e.g., −211) of the start codon. In some additional embodiments, the sixth external CSRE is located between 171 base pairs upstream (e.g., −171) of the start codon and 172 base pairs upstream (e.g., −172) of the start codon. In some specific embodiments, the sixth external CSRE is located between about 138 base pairs upstream (e.g., −138) of the start codon and about 217 base pairs upstream (e.g., −217) of the start codon. In some additional embodiments, the sixth external CSRE is located between 177 base pairs upstream (e.g., −177) of the start codon and 178 base pairs upstream (e.g., −178) of the start codon. In some specific embodiments, the sixth external CSRE is located between about 139 base pairs upstream (e.g., −139) of the start codon and about 218 base pairs upstream (e.g., −218) of the start codon. In some additional embodiments, the sixth external CSRE is located between 178 base pairs upstream (e.g., −178) of the start codon and 179 base pairs upstream (e.g., −179) of the start codon. In some specific embodiments, the sixth external CSRE is located between about 144 base pairs upstream (e.g., −144) of the start codon and about 223 base pairs upstream (e.g., −223) of the start codon. In some additional embodiments, the sixth external CSRE is located between 183 base pairs upstream (e.g., −183) of the start codon and 184 base pairs upstream (e.g., −184) of the start codon. In some specific embodiments, the sixth external CSRE is located between about 149 base pairs upstream (e.g., −149) of the start codon and about 228 base pairs upstream (e.g., −228) of the start codon. In some additional embodiments, the sixth external CSRE is located between 188 base pairs upstream (e.g., −188) of the start codon and 189 base pairs upstream (e.g., −189) of the start codon. In some specific embodiments, the sixth external CSRE is located between about 154 base pairs upstream (e.g., −154) of the start codon and about 233 base pairs upstream (e.g., −233) of the start codon. In some additional embodiments, the sixth external CSRE is located between 193 base pairs upstream (e.g., −193) of the start codon and 194 base pairs upstream (e.g., −194) of the start codon. In some specific embodiments, the sixth external CSRE is located between about 156 base pairs upstream (e.g., −156) of the start codon and about 235 base pairs upstream (e.g., −235) of the start codon. In some additional embodiments, the sixth external CSRE is located between 195 base pairs upstream (e.g., −195) of the start codon and 196 base pairs upstream (e.g., −196) of the start codon. In some specific embodiments, the sixth external CSRE is located between about 159 base pairs upstream (e.g., −159) of the start codon and about 238 base pairs upstream (e.g., −238) of the start codon. In some additional embodiments, the sixth external CSRE is located between 198 base pairs upstream (e.g., −198) of the start codon and 199 base pairs upstream (e.g., −199) of the start codon. In some specific embodiments, the sixth external CSRE is located between about 164 base pairs upstream (e.g., −164) of the start codon and about 243 base pairs upstream (e.g., −243) of the start codon. In some additional embodiments, the sixth external CSRE is located between 203 base pairs upstream (e.g., −203) of the start codon and 204 base pairs upstream (e.g., −204) of the start codon. In some specific embodiments, the sixth external CSRE is located between about 169 base pairs upstream (e.g., −169) of the start codon and about 248 base pairs upstream (e.g., −248) of the start codon. In some additional embodiments, the sixth external CSRE is located between 208 base pairs upstream (e.g., −208) of the start codon and 209 base pairs upstream (e.g., −209) of the start codon. In some specific embodiments, the sixth external CSRE is located between about 174 base pairs upstream (e.g., −174) of the start codon and about 253 base pairs upstream (e.g., −253) of the start codon. In some additional embodiments, the sixth external CSRE is located between 213 base pairs upstream (e.g., −213) of the start codon and 214 base pairs upstream (e.g., −214) of the start codon. In some specific embodiments, the sixth external CSRE is located between about 234 base pairs upstream (e.g., −234) of the start codon and about 313 base pairs upstream (e.g., −313) of the start codon. In some additional embodiments, the sixth external CSRE is located between 273 base pairs upstream (e.g., −273) of the start codon and 274 base pairs upstream (e.g., −274) of the start codon. In some specific embodiments, the sixth external CSRE is located between about 243 base pairs upstream (e.g., −243) of the start codon and about 322 base pairs upstream (e.g., −322) of the start codon. In some additional embodiments, the sixth external CSRE is located between 282 base pairs upstream (e.g., −282) of the start codon and 283 base pairs upstream (e.g., −283) of the start codon. In some specific embodiments, the sixth external CSRE is located between about 284 base pairs upstream (e.g., −284) of the start codon and about 363 base pairs upstream (e.g., −363) of the start codon. In some additional embodiments, the sixth external CSRE is located between 323 base pairs upstream (e.g., −323) of the start codon and 324 base pairs upstream (e.g., −324) of the start codon. In some additional embodiments, the sixth external CSRE is located between 363 base pairs upstream (e.g., −363) of the start codon and 442 base pairs upstream (e.g., −442) of the start codon. In some additional embodiments, the sixth external CSRE is located between 402 base pairs upstream (e.g., −402) of the start codon and 403 base pairs upstream (e.g., −403) of the start codon.

In some embodiments, the engineered promoter of the present disclosure comprises at least seven external CSREs. Embodiments of the location and the nucleic acid sequence of the first, second, third, fourth, fifth, and sixth external CSREs are provided herein and can be used in an engineered promoter comprising seven or more external CSREs. When a plurality of CSREs is included in the engineered promoter, the CSREs can be independently located at the same position and be contiguous or be provided at different positions and be non-contiguous. In some specific embodiments, the seventh external CSRE is located between about 52 base pairs upstream (e.g., −52) of the start codon and about 131 base pairs upstream (e.g., −131) of the start codon. In some additional embodiments, the seventh external CSRE is located between 91 base pairs upstream (e.g., −91) of the start codon and 92 base pairs upstream (e.g., −92) of the start codon. In some specific embodiments, the seventh external CSRE is located between about 82 base pairs upstream (e.g., −82) of the start codon and about 161 base pairs upstream (e.g., −161) of the start codon. In some additional embodiments, the seventh external CSRE is located between 121 base pairs upstream (e.g., −121) of the start codon and 122 base pairs upstream (e.g., −122) of the start codon. In some specific embodiments, the seventh external CSRE is located between about 92 base pairs upstream (e.g., −92) of the start codon and about 171 base pairs upstream (e.g., −171) of the start codon. In some additional embodiments, the seventh external CSRE is located between 131 base pairs upstream (e.g., −131) of the start codon and 132 base pairs upstream (e.g., −132) of the start codon. In some specific embodiments, the seventh external CSRE is located between about 100 base pairs upstream (e.g., −100) of the start codon and about 179 base pairs upstream (e.g., −179) of the start codon. In some additional embodiments, the seventh external CSRE is located between 139 base pairs upstream (e.g., −139) of the start codon and 140 base pairs upstream (e.g., −140) of the start codon. In some specific embodiments, the seventh external CSRE is located between about 122 base pairs upstream (e.g., −122) of the start codon and about 201 base pairs upstream (e.g., −201) of the start codon. In some additional embodiments, the seventh external CSRE is located between 161 base pairs upstream (e.g., −161) of the start codon and 162 base pairs upstream (e.g., −162) of the start codon. In some specific embodiments, the seventh external CSRE is located between about 126 base pairs upstream (e.g., −126) of the start codon and about 205 base pairs upstream (e.g., −205) of the start codon. In some additional embodiments, the seventh external CSRE is located between 165 base pairs upstream (e.g., −165) of the start codon and 166 base pairs upstream (e.g., −166) of the start codon. In some specific embodiments, the seventh external CSRE is located between about 132 base pairs upstream (e.g., −132) of the start codon and about 211 base pairs upstream (e.g., −211) of the start codon. In some additional embodiments, the seventh external CSRE is located between 171 base pairs upstream (e.g., −171) of the start codon and 172 base pairs upstream (e.g., −172) of the start codon. In some specific embodiments, the seventh external CSRE is located between about 138 base pairs upstream (e.g., −138) of the start codon and about 217 base pairs upstream (e.g., −217) of the start codon. In some additional embodiments, the seventh external CSRE is located between 177 base pairs upstream (e.g., −177) of the start codon and 178 base pairs upstream (e.g., −178) of the start codon. In some specific embodiments, the seventh external CSRE is located between about 139 base pairs upstream (e.g., −139) of the start codon and about 218 base pairs upstream (e.g., −218) of the start codon. In some additional embodiments, the seventh external CSRE is located between 178 base pairs upstream (e.g., −178) of the start codon and 179 base pairs upstream (e.g., −179) of the start codon. In some specific embodiments, the seventh external CSRE is located between about 144 base pairs upstream (e.g., −144) of the start codon and about 223 base pairs upstream (e.g., −223) of the start codon. In some additional embodiments, the seventh external CSRE is located between 183 base pairs upstream (e.g., −183) of the start codon and 184 base pairs upstream (e.g., −184) of the start codon. In some specific embodiments, the seventh external CSRE is located between about 149 base pairs upstream (e.g., −149) of the start codon and about 228 base pairs upstream (e.g., −228) of the start codon. In some additional embodiments, the seventh external CSRE is located between 188 base pairs upstream (e.g., −188) of the start codon and 189 base pairs upstream (e.g., −189) of the start codon. In some specific embodiments, the seventh external CSRE is located between about 154 base pairs upstream (e.g., −154) of the start codon and about 233 base pairs upstream (e.g., −233) of the start codon. In some additional embodiments, the seventh external CSRE is located between 193 base pairs upstream (e.g., −193) of the start codon and 194 base pairs upstream (e.g., −194) of the start codon. In some specific embodiments, the seventh external CSRE is located between about 156 base pairs upstream (e.g., −156) of the start codon and about 235 base pairs upstream (e.g., −235) of the start codon. In some additional embodiments, the seventh external CSRE is located between 195 base pairs upstream (e.g., −195) of the start codon and 196 base pairs upstream (e.g., −196) of the start codon. In some specific embodiments, the seventh external CSRE is located between about 159 base pairs upstream (e.g., −159) of the start codon and about 238 base pairs upstream (e.g., −238) of the start codon. In some additional embodiments, the seventh external CSRE is located between 198 base pairs upstream (e.g., −198) of the start codon and 199 base pairs upstream (e.g., −199) of the start codon. In some specific embodiments, the seventh external CSRE is located between about 164 base pairs upstream (e.g., −164) of the start codon and about 243 base pairs upstream (e.g., −243) of the start codon. In some additional embodiments, the seventh external CSRE is located between 203 base pairs upstream (e.g., −203) of the start codon and 204 base pairs upstream (e.g., −204) of the start codon. In some specific embodiments, the seventh external CSRE is located between about 169 base pairs upstream (e.g., −169) of the start codon and about 248 base pairs upstream (e.g., −248) of the start codon. In some additional embodiments, the seventh external CSRE is located between 208 base pairs upstream (e.g., −208) of the start codon and 209 base pairs upstream (e.g., −209) of the start codon. In some specific embodiments, the seventh external CSRE is located between about 174 base pairs upstream (e.g., −174) of the start codon and about 253 base pairs upstream (e.g., −253) of the start codon. In some additional embodiments, the seventh external CSRE is located between 213 base pairs upstream (e.g., −213) of the start codon and 214 base pairs upstream (e.g., −214) of the start codon. In some specific embodiments, the seventh external CSRE is located between about 234 base pairs upstream (e.g., −234) of the start codon and about 313 base pairs upstream (e.g., −313) of the start codon. In some additional embodiments, the seventh external CSRE is located between 273 base pairs upstream (e.g., −273) of the start codon and 274 base pairs upstream (e.g., −274) of the start codon. In some specific embodiments, the seventh external CSRE is located between about 243 base pairs upstream (e.g., −243) of the start codon and about 322 base pairs upstream (e.g., −322) of the start codon. In some additional embodiments, the seventh external CSRE is located between 282 base pairs upstream (e.g., −282) of the start codon and 283 base pairs upstream (e.g., −283) of the start codon. In some specific embodiments, the seventh external CSRE is located between about 284 base pairs upstream (e.g., −284) of the start codon and about 363 base pairs upstream (e.g., −363) of the start codon. In some additional embodiments, the seventh external CSRE is located between 323 base pairs upstream (e.g., −323) of the start codon and 324 base pairs upstream (e.g., −324) of the start codon. In some additional embodiments, the seventh external CSRE is located between 363 base pairs upstream (e.g., −363) of the start codon and 442 base pairs upstream (e.g., −442) of the start codon. In some additional embodiments, the seventh external CSRE is located between 402 base pairs upstream (e.g., −402) of the start codon and 403 base pairs upstream (e.g., −403) of the start codon.

In some embodiments, the engineered promoter of the present disclosure comprises at least eight external CSREs. Embodiments of the location and the nucleic acid sequence of the first, second, third, fourth, fifth, sixth, and seventh external CSREs are provided herein and can be used in an engineered promoter comprising eight or more external CSREs. When a plurality of CSREs is included in the engineered promoter, the CSREs can be independently located at the same position and be contiguous or be provided at different positions and be non-contiguous. In some specific embodiments, the eighth external CSRE is located between about 52 base pairs upstream (e.g., −52) of the start codon and about 131 base pairs upstream (e.g., −131) of the start codon. In some additional embodiments, the eighth external CSRE is located between 91 base pairs upstream (e.g., −91) of the start codon and 92 base pairs upstream (e.g., −92) of the start codon. In some specific embodiments, the eighth external CSRE is located between about 82 base pairs upstream (e.g., −82) of the start codon and about 161 base pairs upstream (e.g., −161) of the start codon. In some additional embodiments, the eighth external CSRE is located between 121 base pairs upstream (e.g., −121) of the start codon and 122 base pairs upstream (e.g., −122) of the start codon. In some specific embodiments, the eighth external CSRE is located between about 92 base pairs upstream (e.g., −92) of the start codon and about 171 base pairs upstream (e.g., −171) of the start codon. In some additional embodiments, the eighth external CSRE is located between 131 base pairs upstream (e.g., −131) of the start codon and 132 base pairs upstream (e.g., −132) of the start codon. In some specific embodiments, the eighth external CSRE is located between about 100 base pairs upstream (e.g., −100) of the start codon and about 179 base pairs upstream (e.g., −179) of the start codon. In some additional embodiments, the eighth external CSRE is located between 139 base pairs upstream (e.g., −139) of the start codon and 140 base pairs upstream (e.g., −140) of the start codon. In some specific embodiments, the eighth external CSRE is located between about 122 base pairs upstream (e.g., −122) of the start codon and about 201 base pairs upstream (e.g., −201) of the start codon. In some additional embodiments, the eighth external CSRE is located between 161 base pairs upstream (e.g., −161) of the start codon and 162 base pairs upstream (e.g., −162) of the start codon. In some specific embodiments, the eighth external CSRE is located between about 126 base pairs upstream (e.g., −126) of the start codon and about 205 base pairs upstream (e.g., −205) of the start codon. In some additional embodiments, the eighth external CSRE is located between 165 base pairs upstream (e.g., −165) of the start codon and 166 base pairs upstream (e.g., −166) of the start codon. In some specific embodiments, the eighth external CSRE is located between about 132 base pairs upstream (e.g., −132) of the start codon and about 211 base pairs upstream (e.g., −211) of the start codon. In some additional embodiments, the eighth external CSRE is located between 171 base pairs upstream (e.g., −171) of the start codon and 172 base pairs upstream (e.g., −172) of the start codon. In some specific embodiments, the eighth external CSRE is located between about 138 base pairs upstream (e.g., −138) of the start codon and about 217 base pairs upstream (e.g., −217) of the start codon. In some additional embodiments, the eighth external CSRE is located between 177 base pairs upstream (e.g., −177) of the start codon and 178 base pairs upstream (e.g., −178) of the start codon. In some specific embodiments, the eighth external CSRE is located between about 139 base pairs upstream (e.g., −139) of the start codon and about 218 base pairs upstream (e.g., −218) of the start codon. In some additional embodiments, the eighth external CSRE is located between 178 base pairs upstream (e.g., −178) of the start codon and 179 base pairs upstream (e.g., −179) of the start codon. In some specific embodiments, the eighth external CSRE is located between about 144 base pairs upstream (e.g., −144) of the start codon and about 223 base pairs upstream (e.g., −223) of the start codon. In some additional embodiments, the eighth external CSRE is located between 183 base pairs upstream (e.g., −183) of the start codon and 184 base pairs upstream (e.g., −184) of the start codon. In some specific embodiments, the eighth external CSRE is located between about 149 base pairs upstream (e.g., −149) of the start codon and about 228 base pairs upstream (e.g., −228) of the start codon. In some additional embodiments, the eighth external CSRE is located between 188 base pairs upstream (e.g., −188) of the start codon and 189 base pairs upstream (e.g., −189) of the start codon. In some specific embodiments, the eighth external CSRE is located between about 154 base pairs upstream (e.g., −154) of the start codon and about 233 base pairs upstream (e.g., −233) of the start codon. In some additional embodiments, the eighth external CSRE is located between 193 base pairs upstream (e.g., −193) of the start codon and 194 base pairs upstream (e.g., −194) of the start codon. In some specific embodiments, the eighth external CSRE is located between about 156 base pairs upstream (e.g., −156) of the start codon and about 235 base pairs upstream (e.g., −235) of the start codon. In some additional embodiments, the eighth external CSRE is located between 195 base pairs upstream (e.g., −195) of the start codon and 196 base pairs upstream (e.g., −196) of the start codon. In some specific embodiments, the eighth external CSRE is located between about 159 base pairs upstream (e.g., −159) of the start codon and about 238 base pairs upstream (e.g., −238) of the start codon. In some additional embodiments, the eighth external CSRE is located between 198 base pairs upstream (e.g., −198) of the start codon and 199 base pairs upstream (e.g., −199) of the start codon. In some specific embodiments, the eighth external CSRE is located between about 164 base pairs upstream (e.g., −164) of the start codon and about 243 base pairs upstream (e.g., −243) of the start codon. In some additional embodiments, the eighth external CSRE is located between 203 base pairs upstream (e.g., −203) of the start codon and 204 base pairs upstream (e.g., −204) of the start codon. In some specific embodiments, the eighth external CSRE is located between about 169 base pairs upstream (e.g., −169) of the start codon and about 248 base pairs upstream (e.g., −248) of the start codon. In some additional embodiments, the eighth external CSRE is located between 208 base pairs upstream (e.g., −208) of the start codon and 209 base pairs upstream (e.g., −209) of the start codon. In some specific embodiments, the eighth external CSRE is located between about 174 base pairs upstream (e.g., −174) of the start codon and about 253 base pairs upstream (e.g., −253) of the start codon. In some additional embodiments, the eighth external CSRE is located between 213 base pairs upstream (e.g., −213) of the start codon and 214 base pairs upstream (e.g., −214) of the start codon. In some specific embodiments, the eighth external CSRE is located between about 234 base pairs upstream (e.g., −234) of the start codon and about 313 base pairs upstream (e.g., −313) of the start codon. In some additional embodiments, the eighth external CSRE is located between 273 base pairs upstream (e.g., −273) of the start codon and 274 base pairs upstream (e.g., −274) of the start codon. In some specific embodiments, the eighth external CSRE is located between about 243 base pairs upstream (e.g., −243) of the start codon and about 322 base pairs upstream (e.g., −322) of the start codon. In some additional embodiments, the eighth external CSRE is located between 282 base pairs upstream (e.g., −282) of the start codon and 283 base pairs upstream (e.g., −283) of the start codon. In some specific embodiments, the eighth external CSRE is located between about 284 base pairs upstream (e.g., −284) of the start codon and about 363 base pairs upstream (e.g., −363) of the start codon. In some additional embodiments, the eighth external CSRE is located between 323 base pairs upstream (e.g., −323) of the start codon and 324 base pairs upstream (e.g., −324) of the start codon. In some additional embodiments, the eighth external CSRE is located between 363 base pairs upstream (e.g., −363) of the start codon and 442 base pairs upstream (e.g., −442) of the start codon. In some additional embodiments, the eighth external CSRE is located between 402 base pairs upstream (e.g., −402) of the start codon and 403 base pairs upstream (e.g., −403) of the start codon.

In some embodiments, the engineered promoter of the present disclosure comprises at least nine external CSREs. Embodiments of the location and the nucleic acid sequence of the first, second, third, fourth, fifth, sixth, seventh, and eighth external CSREs are provided herein and can be used in an engineered promoter comprising nine or more external CSREs. When a plurality of CSREs is included in the engineered promoter, the CSREs can be independently located at the same position and be contiguous or be provided at different positions and be non-contiguous. In some specific embodiments, the ninth external CSRE is located between about 52 base pairs upstream (e.g., −52) of the start codon and about 131 base pairs upstream (e.g., −131) of the start codon. In some additional embodiments, the ninth external CSRE is located between 91 base pairs upstream (e.g., −91) of the start codon and 92 base pairs upstream (e.g., −92) of the start codon. In some specific embodiments, the ninth external CSRE is located between about 82 base pairs upstream (e.g., −82) of the start codon and about 161 base pairs upstream (e.g., −161) of the start codon. In some additional embodiments, the ninth external CSRE is located between 121 base pairs upstream (e.g., −121) of the start codon and 122 base pairs upstream (e.g., −122) of the start codon. In some specific embodiments, the ninth external CSRE is located between about 92 base pairs upstream (e.g., −92) of the start codon and about 171 base pairs upstream (e.g., −171) of the start codon. In some additional embodiments, the ninth external CSRE is located between 131 base pairs upstream (e.g., −131) of the start codon and 132 base pairs upstream (e.g., −132) of the start codon. In some specific embodiments, the ninth external CSRE is located between about 100 base pairs upstream (e.g., −100) of the start codon and about 179 base pairs upstream (e.g., −179) of the start codon. In some additional embodiments, the ninth external CSRE is located between 139 base pairs upstream (e.g., −139) of the start codon and 140 base pairs upstream (e.g., −140) of the start codon. In some specific embodiments, the ninth external CSRE is located between about 122 base pairs upstream (e.g., −122) of the start codon and about 201 base pairs upstream (e.g., −201) of the start codon. In some additional embodiments, the ninth external CSRE is located between 161 base pairs upstream (e.g., −161) of the start codon and 162 base pairs upstream (e.g., −162) of the start codon. In some specific embodiments, the ninth external CSRE is located between about 126 base pairs upstream (e.g., −126) of the start codon and about 205 base pairs upstream (e.g., −205) of the start codon. In some additional embodiments, the ninth external CSRE is located between 165 base pairs upstream (e.g., −165) of the start codon and 166 base pairs upstream (e.g., −166) of the start codon. In some specific embodiments, the ninth external CSRE is located between about 132 base pairs upstream (e.g., −132) of the start codon and about 211 base pairs upstream (e.g., −211) of the start codon. In some additional embodiments, the ninth external CSRE is located between 171 base pairs upstream (e.g., −171) of the start codon and 172 base pairs upstream (e.g., −172) of the start codon. In some specific embodiments, the ninth external CSRE is located between about 138 base pairs upstream (e.g., −138) of the start codon and about 217 base pairs upstream (e.g., −217) of the start codon. In some additional embodiments, the ninth external CSRE is located between 177 base pairs upstream (e.g., −177) of the start codon and 178 base pairs upstream (e.g., −178) of the start codon. In some specific embodiments, the ninth external CSRE is located between about 139 base pairs upstream (e.g., −139) of the start codon and about 218 base pairs upstream (e.g., −218) of the start codon. In some additional embodiments, the ninth external CSRE is located between 178 base pairs upstream (e.g., −178) of the start codon and 179 base pairs upstream (e.g., −179) of the start codon. In some specific embodiments, the ninth external CSRE is located between about 144 base pairs upstream (e.g., −144) of the start codon and about 223 base pairs upstream (e.g., −223) of the start codon. In some additional embodiments, the ninth external CSRE is located between 183 base pairs upstream (e.g., −183) of the start codon and 184 base pairs upstream (e.g., −184) of the start codon. In some specific embodiments, the ninth external CSRE is located between about 149 base pairs upstream (e.g., −149) of the start codon and about 228 base pairs upstream (e.g., −228) of the start codon. In some additional embodiments, the ninth external CSRE is located between 188 base pairs upstream (e.g., −188) of the start codon and 189 base pairs upstream (e.g., −189) of the start codon. In some specific embodiments, the ninth external CSRE is located between about 154 base pairs upstream (e.g., −154) of the start codon and about 233 base pairs upstream (e.g., −233) of the start codon. In some additional embodiments, the ninth external CSRE is located between 193 base pairs upstream (e.g., −193) of the start codon and 194 base pairs upstream (e.g., −194) of the start codon. In some specific embodiments, the ninth external CSRE is located between about 156 base pairs upstream (e.g., −156) of the start codon and about 235 base pairs upstream (e.g., −235) of the start codon. In some additional embodiments, the ninth external CSRE is located between 195 base pairs upstream (e.g., −195) of the start codon and 196 base pairs upstream (e.g., −196) of the start codon. In some specific embodiments, the ninth external CSRE is located between about 159 base pairs upstream (e.g., −159) of the start codon and about 238 base pairs upstream (e.g., −238) of the start codon. In some additional embodiments, the ninth external CSRE is located between 198 base pairs upstream (e.g., −198) of the start codon and 199 base pairs upstream (e.g., −199) of the start codon. In some specific embodiments, the ninth external CSRE is located between about 164 base pairs upstream (e.g., −164) of the start codon and about 243 base pairs upstream (e.g., −243) of the start codon. In some additional embodiments, the ninth external CSRE is located between 203 base pairs upstream (e.g., −203) of the start codon and 204 base pairs upstream (e.g., −204) of the start codon. In some specific embodiments, the ninth external CSRE is located between about 169 base pairs upstream (e.g., −169) of the start codon and about 248 base pairs upstream (e.g., −248) of the start codon. In some additional embodiments, the ninth external CSRE is located between 208 base pairs upstream (e.g., −208) of the start codon and 209 base pairs upstream (e.g., −209) of the start codon. In some specific embodiments, the ninth external CSRE is located between about 174 base pairs upstream (e.g., −174) of the start codon and about 253 base pairs upstream (e.g., −253) of the start codon. In some additional embodiments, the ninth external CSRE is located between 213 base pairs upstream (e.g., −213) of the start codon and 214 base pairs upstream (e.g., −214) of the start codon. In some specific embodiments, the ninth external CSRE is located between about 234 base pairs upstream (e.g., −234) of the start codon and about 313 base pairs upstream (e.g., −313) of the start codon. In some additional embodiments, the ninth external CSRE is located between 273 base pairs upstream (e.g., −273) of the start codon and 274 base pairs upstream (e.g., −274) of the start codon. In some specific embodiments, the ninth external CSRE is located between about 243 base pairs upstream (e.g., −243) of the start codon and about 322 base pairs upstream (e.g., −322) of the start codon. In some additional embodiments, the ninth external CSRE is located between 282 base pairs upstream (e.g., −282) of the start codon and 283 base pairs upstream (e.g., −283) of the start codon. In some specific embodiments, the ninth external CSRE is located between about 284 base pairs upstream (e.g., −284) of the start codon and about 363 base pairs upstream (e.g., −363) of the start codon. In some additional embodiments, the ninth external CSRE is located between 323 base pairs upstream (e.g., −323) of the start codon and 324 base pairs upstream (e.g., −324) of the start codon. In some additional embodiments, the ninth external CSRE is located between 363 base pairs upstream (e.g., −363) of the start codon and 442 base pairs upstream (e.g., −442) of the start codon. In some additional embodiments, the ninth external CSRE is located between 402 base pairs upstream (e.g., −402) of the start codon and 403 base pairs upstream (e.g., −403) of the start codon.

In some embodiments, the engineered promoter of the present disclosure comprises at least ten external CSREs. Embodiments of the location and the nucleic acid sequence of the first, second, third, fourth, fifth, sixth, seventh, eighth, and ninth external CSREs are provided herein and can be used in an engineered promoter comprising ten or more external CSREs. When a plurality of CSREs is included in the engineered promoter, the CSREs can be independently located at the same position and be contiguous or be provided at different positions and be non-contiguous. In some specific embodiments, the tenth external CSRE is located between about 52 base pairs upstream (e.g., −52) of the start codon and about 131 base pairs upstream (e.g., −131) of the start codon. In some additional embodiments, the tenth external CSRE is located between 91 base pairs upstream (e.g., −91) of the start codon and 92 base pairs upstream (e.g., −92) of the start codon. In some specific embodiments, the tenth external CSRE is located between about 82 base pairs upstream (e.g., −82) of the start codon and about 161 base pairs upstream (e.g., −161) of the start codon. In some additional embodiments, the tenth external CSRE is located between 121 base pairs upstream (e.g., −121) of the start codon and 122 base pairs upstream (e.g., −122) of the start codon. In some specific embodiments, the tenth external CSRE is located between about 92 base pairs upstream (e.g., −92) of the start codon and about 171 base pairs upstream (e.g., −171) of the start codon. In some additional embodiments, the tenth external CSRE is located between 131 base pairs upstream (e.g., −131) of the start codon and 132 base pairs upstream (e.g., −132) of the start codon. In some specific embodiments, the tenth external CSRE is located between about 100 base pairs upstream (e.g., −100) of the start codon and about 179 base pairs upstream (e.g., −179) of the start codon. In some additional embodiments, the tenth external CSRE is located between 139 base pairs upstream (e.g., −139) of the start codon and 140 base pairs upstream (e.g., −140) of the start codon. In some specific embodiments, the tenth external CSRE is located between about 122 base pairs upstream (e.g., −122) of the start codon and about 201 base pairs upstream (e.g., −201) of the start codon. In some additional embodiments, the tenth external CSRE is located between 161 base pairs upstream (e.g., −161) of the start codon and 162 base pairs upstream (e.g., −162) of the start codon. In some specific embodiments, the tenth external CSRE is located between about 126 base pairs upstream (e.g., −126) of the start codon and about 205 base pairs upstream (e.g., −205) of the start codon. In some additional embodiments, the tenth external CSRE is located between 165 base pairs upstream (e.g., −165) of the start codon and 166 base pairs upstream (e.g., −166) of the start codon. In some specific embodiments, the tenth external CSRE is located between about 132 base pairs upstream (e.g., −132) of the start codon and about 211 base pairs upstream (e.g., −211) of the start codon. In some additional embodiments, the tenth external CSRE is located between 171 base pairs upstream (e.g., −171) of the start codon and 172 base pairs upstream (e.g., −172) of the start codon. In some specific embodiments, the tenth external CSRE is located between about 138 base pairs upstream (e.g., −138) of the start codon and about 217 base pairs upstream (e.g., −217) of the start codon. In some additional embodiments, the tenth external CSRE is located between 177 base pairs upstream (e.g., −177) of the start codon and 178 base pairs upstream (e.g., −178) of the start codon. In some specific embodiments, the tenth external CSRE is located between about 139 base pairs upstream (e.g., −139) of the start codon and about 218 base pairs upstream (e.g., −218) of the start codon. In some additional embodiments, the tenth external CSRE is located between 178 base pairs upstream (e.g., −178) of the start codon and 179 base pairs upstream (e.g., −179) of the start codon. In some specific embodiments, the tenth external CSRE is located between about 144 base pairs upstream (e.g., −144) of the start codon and about 223 base pairs upstream (e.g., −223) of the start codon. In some additional embodiments, the tenth external CSRE is located between 183 base pairs upstream (e.g., −183) of the start codon and 184 base pairs upstream (e.g., −184) of the start codon. In some specific embodiments, the tenth external CSRE is located between about 149 base pairs upstream (e.g., −149) of the start codon and about 228 base pairs upstream (e.g., −228) of the start codon. In some additional embodiments, the tenth external CSRE is located between 188 base pairs upstream (e.g., −188) of the start codon and 189 base pairs upstream (e.g., −189) of the start codon. In some specific embodiments, the tenth external CSRE is located between about 154 base pairs upstream (e.g., −154) of the start codon and about 233 base pairs upstream (e.g., −233) of the start codon. In some additional embodiments, the tenth external CSRE is located between 193 base pairs upstream (e.g., −193) of the start codon and 194 base pairs upstream (e.g., −194) of the start codon. In some specific embodiments, the tenth external CSRE is located between about 156 base pairs upstream (e.g., −156) of the start codon and about 235 base pairs upstream (e.g., −235) of the start codon. In some additional embodiments, the tenth external CSRE is located between 195 base pairs upstream (e.g., −195) of the start codon and 196 base pairs upstream (e.g., −196) of the start codon. In some specific embodiments, the tenth external CSRE is located between about 159 base pairs upstream (e.g., −159) of the start codon and about 238 base pairs upstream (e.g., −238) of the start codon. In some additional embodiments, the tenth external CSRE is located between 198 base pairs upstream (e.g., −198) of the start codon and 199 base pairs upstream (e.g., −199) of the start codon. In some specific embodiments, the tenth external CSRE is located between about 164 base pairs upstream (e.g., −164) of the start codon and about 243 base pairs upstream (e.g., −243) of the start codon. In some additional embodiments, the tenth external CSRE is located between 203 base pairs upstream (e.g., −203) of the start codon and 204 base pairs upstream (e.g., −204) of the start codon. In some specific embodiments, the tenth external CSRE is located between about 169 base pairs upstream (e.g., −169) of the start codon and about 248 base pairs upstream (e.g., −248) of the start codon. In some additional embodiments, the tenth external CSRE is located between 208 base pairs upstream (e.g., −208) of the start codon and 209 base pairs upstream (e.g., −209) of the start codon. In some specific embodiments, the tenth external CSRE is located between about 174 base pairs upstream (e.g., −174) of the start codon and about 253 base pairs upstream (e.g., −253) of the start codon. In some additional embodiments, the tenth external CSRE is located between 213 base pairs upstream (e.g., −213) of the start codon and 214 base pairs upstream (e.g., −214) of the start codon. In some specific embodiments, the tenth external CSRE is located between about 234 base pairs upstream (e.g., −234) of the start codon and about 313 base pairs upstream (e.g., −313) of the start codon. In some additional embodiments, the tenth external CSRE is located between 273 base pairs upstream (e.g., −273) of the start codon and 274 base pairs upstream (e.g., −274) of the start codon. In some specific embodiments, the tenth external CSRE is located between about 243 base pairs upstream (e.g., −243) of the start codon and about 322 base pairs upstream (e.g., −322) of the start codon. In some additional embodiments, the tenth external CSRE is located between 282 base pairs upstream (e.g., −282) of the start codon and 283 base pairs upstream (e.g., −283) of the start codon. In some specific embodiments, the tenth external CSRE is located between about 284 base pairs upstream (e.g., −284) of the start codon and about 363 base pairs upstream (e.g., −363) of the start codon. In some additional embodiments, the tenth external CSRE is located between 323 base pairs upstream (e.g., −323) of the start codon and 324 base pairs upstream (e.g., −324) of the start codon. In some additional embodiments, the tenth external CSRE is located between 363 base pairs upstream (e.g., −363) of the start codon and 442 base pairs upstream (e.g., −442) of the start codon. some additional embodiments, the tenth external CSRE is located between 402 base pairs upstream (e.g., −402) of the start codon and 403 base pairs upstream (e.g., −403) of the start codon. In still another embodiment, the engineered promoter with ten or more external CSREs comprises a first external CSRE located between about 139 base pairs upstream (e.g., −139) of the start codon and about 218 base pairs upstream (e.g., −218) of the start codon; a second external CSRE located between about 144 base pairs upstream (e.g., −144) of the start codon and about 223 base pairs upstream (e.g., −223) of the start codon; a third external CSRE located between about 149 base pairs upstream (e.g., −149) of the start codon and about 228 base pairs upstream (e.g., −228) of the start codon; a fourth external CSRE located between about 154 base pairs upstream (e.g., −154) of the start codon and about 233 base pairs upstream (e.g., −233) of the start codon; a fifth external CSRE located between about 159 base pairs upstream (e.g., −159) of the start codon and about 238 base pairs upstream (e.g., −238) of the start codon; a sixth external CSRE located between about 164 base pairs upstream (e.g., −164) of the start codon and about 243 base pairs upstream (e.g., −243) of the start codon; a seven external CSRE located between about 169 base pairs upstream (e.g., −169) of the start codon and about 248 base pairs upstream (e.g., −248) of the start codon; an eighth external CSRE located between about 174 base pairs upstream (e.g., −174) of the start codon and about 248 base pairs upstream (e.g., −253) of the start codon; a ninth external CSRE located between about 243 base pairs upstream (e.g., −243) of the start codon and about 322 base pairs upstream (e.g., −322) of the start codon; and a tenth external CSRE located between about 363 base pairs upstream (e.g., −363) of the start codon and about 442 base pairs upstream (e.g., −442) of the start codon. In yet still another embodiment, the engineered promoter with ten or more external CSREs comprises a first external CSRE located between 178 base pairs upstream (e.g., −178) of the start codon and 179 base pairs upstream (e.g., −179) of the start codon; a second external CSRE located between 183 base pairs upstream (e.g., −183) of the start codon and 184 base pairs upstream (e.g., −184) of the start codon; a third external CSRE located between 188 base pairs upstream (e.g., −188) of the start codon and 189 base pairs upstream (e.g., −189) of the start codon; a fourth external CSRE located between 193 base pairs upstream (e.g., −193) of the start codon and 194 base pairs upstream (e.g., −194) of the start codon; a fifth external CSRE located between 198 base pairs upstream (e.g., −198) of the start codon and 199 base pairs upstream (e.g., −199) of the start codon; a sixth external CSRE located between 203 base pairs upstream (e.g., −203) of the start codon and 204 base pairs upstream (e.g., −204) of the start codon; a seven external CSRE located between 208 base pairs upstream (e.g., −208) of the start codon and 209 base pairs upstream (e.g., −209) of the start codon; an eighth external CSRE located between 213 base pairs upstream (e.g., −213) of the start codon and 214 base pairs upstream (e.g., −214) of the start codon; a ninth external CSRE located between 282 base pairs upstream (e.g., −282) of the start codon and 283 base pairs upstream (e.g., −283) of the start codon; and a tenth external CSRE located between 402 base pairs upstream (e.g., −402) of the start codon and 403 base pairs upstream (e.g., −403) of the start codon.

In some embodiments, the engineered promoter comprises a first external CSRE of formula (I). In additional embodiments, the first external CSRE can have the nucleic acid sequence of any one of SEQ ID NO: 26 to 35. The first external CSRE is located anywhere upstream of the transcription start site. In some embodiments, the first external CSRE is located upstream and proximal to the transcription start site. Embodiments of engineered promoters comprising a single external CSRE including, but are not limited to, engineered promoters having the nucleic acid sequence of SEQ ID NO: 6, 11, 18, 19, 20, 21, 22, or 23.

In some embodiments, the engineered promoter comprises a second external CSRE of formula (I). In additional embodiments, the second external CSRE can have the nucleic acid sequence of any one of SEQ ID NO: 26 to 35. The second external CSRE does not necessarily refer to the fact that it has been introduced in the engineered promoter after the first external CSRE. The second external CSRE is located anywhere upstream of the transcription start site. In some embodiments, the second external CSRE is located upstream and proximal to the transcription start site. Embodiments of engineered promoters comprising two external CSREs include, but are not limited to, an engineered promotes having the nucleic acid sequence of SEQ ID NO: 7.

In some embodiments, the engineered promoter comprises a third external CSRE of formula (I). In additional embodiments, the third external CSRE can have the nucleic acid sequence of any one of SEQ ID NO: 26 to 35. The third external CSRE does not necessarily refer to the fact that it has been introduced in the engineered promoter after the first and the second external CSRE. The third external CSRE is located any where upstream of the transcription start site. In some embodiments, the third external CSRE is located upstream and proximal to the transcription start site.

Embodiments of engineered promoters comprising three external CSREs include, but are not limited to, engineered promoters having the nucleic acid sequence of SEQ ID NO: 8, or 13.

In some embodiments, the engineered promoter comprises a fourth external CSRE of formula (I). In additional embodiments, the fourth external CSRE can have the nucleic acid sequence of any one of SEQ ID NO: 26 to 35. The fourth external CSRE does not necessarily refer to the fact that it has been introduced in the engineered promoter after the first, second, and third external CSREs. The fourth external CSRE is located any where upstream of the transcription start site. In some embodiments, the fourth external CSRE is located upstream and proximal to the transcription start site.

In some embodiments, the engineered promoter comprises a fifth external CSRE of formula (I). In additional embodiments, the fifth external CSRE can have the nucleic acid sequence of any one of SEQ ID NO: 26 to 35. The fifth external CSRE does not necessarily refer to the fact that it has been introduced in the engineered promoter after the first, second, third, and fourth external CSREs. The fifth external CSRE is located any where upstream of the transcription start site. In some embodiments, the fifth external CSRE is located upstream and proximal to the transcription start site. In an embodiment, the fifth external CSRE is located at any one of the positions described herein for the position the first external CSRE. Embodiments of engineered promoters comprising five external CSREs include, but are not limited to, an engineered promoter having the nucleic acid sequence of SEQ ID NO: 9.

In some embodiments, the engineered promoter comprises a sixth external CSRE of formula (I). In additional embodiments, the sixth external CSRE can have the nucleic acid sequence of any one of SEQ ID NO: 26 to 35. The sixth external CSRE does not necessarily refer to the fact that it has been introduced in the engineered promoter after the first, second, third, fourth, and fifth external CSREs. The sixth external CSRE is located any where upstream of the transcription start site. In some embodiments, the sixth external CSRE is located upstream and proximal to the transcription start site

In some embodiments, the engineered promoter comprises a seventh external CSRE of formula (I). In additional embodiments, the seventh external CSRE can have the nucleic acid sequence of any one of SEQ ID NO: 26 to 35. The seventh external CSRE does not necessarily refer to the fact that it has been introduced in the engineered promoter after the first, second, third, fourth, fifth, and sixth external CSREs. The seventh external CSRE is located any where upstream of the transcription start site. In some embodiments, the seventh external CSRE is located upstream and proximal to the transcription start site.

In some embodiments, the engineered promoter comprises an eighth external CSRE of formula (I). In additional embodiments, the eight external CSRE can have the nucleic acid sequence of any one of SEQ ID NO: 26 to 35. The eighth external CSRE does not necessarily refer to the fact that it has been introduced in the engineered promoter after the first, second, third, fourth, fifth, sixth, and seventh external CSREs. The eighth external CSRE is located any where upstream of the transcription start site. In some embodiments, the eighth external CSRE is located upstream and proximal to the transcription start site.

In some embodiments, the engineered promoter comprises a ninth external CSRE of formula (I). In additional embodiments, the ninth external CSRE can have the nucleic acid sequence of any one of SEQ ID NO: 26 to 35. The ninth external CSRE does not necessarily refer to the fact that it has been introduced in the engineered promoter after the first, second, third, fourth, fifth, sixth, seventh, and eighth external CSREs. The ninth external CSRE is located any where upstream of the transcription start site. In some embodiments, the ninth external CSRE is located upstream and proximal to the transcription start site.

In some embodiments, the engineered promoter comprises a tenth external CSRE of formula (I). In additional embodiments, the tenth external CSRE can have the nucleic acid sequence of any one of SEQ ID NO: 26 to 35. The tenth external CSRE does not necessarily refer to the fact that it has been introduced in the engineered promoter after the first, second, third, fourth, fifth, sixth, seventh, eighth, and ninth external CSREs. The tenth external CSRE is located any where upstream of the transcription start site. In some embodiments, the tenth external CSRE is located upstream and proximal to the transcription start site. Embodiments of engineered promoters comprising a single external CSRE including, but are not limited to, engineered promoters having the nucleic acid sequence of SEQ ID NO: 10.

The present disclosure provides heterologous nucleic acid molecules comprising the engineered promoters described herein. In some embodiments, the heterologous nucleic acid molecule comprises the engineered promoter(s) and lacks a gene. In such embodiment, the heterologous nucleic acid molecule is intended to be inserted upstream of a native gene in a microbe so as to control the expression of such native gene. In alternative embodiments, the heterologous nucleic acid molecule comprises both an engineered promoter and, directly downstream and operatively linked to, a heterologous gene (or a combination of heterologous genes). In such embodiment, the engineered promoter is intended to control the expression of such heterologous gene in vitro or in the recombinant microbial host cell. As used herein, the term “heterologous” when used in reference to a biological molecule (such as a nucleic acid molecule (such as a promoter, a terminator or a gene) or a polypeptide) refers to a biological molecule that is not natively found in the recombinant microbial host cell or the control recombinant microbial host cell. “Heterologous” also includes a native coding region/promoter/terminator/gene, or portion thereof, that was introduced into the recombinant microbial host cell in a form and/or at a location that is different from the corresponding native gene, e.g., not in its endogenous location in the recombinant microbial host cell's genome. In the context of the present disclosure, heterologous nucleic acid molecule is purposively introduced into the recombinant microbial host cell.

In embodiments in which the engineered promoters are operatively linked to a gene, the nucleic acid sequence of the gene can be codon-optimized with respect to the intended recipient recombinant microbial host cell. As used herein, the term “codon-optimized” means that a nucleic acid region (e.g., gene) that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous genes can correspond to between about 0.8 and 1.0, between about 0.8 and 0.9, or about 1.0.

The heterologous nucleic acid sequences can include, besides the engineered promoter(s) and optional heterologous gene, additional suitable regulatory regions. “Suitable regulatory regions” refer to nucleic acid regions located upstream (5 non-coding sequences), within, or downstream (3′ non-coding sequences) of a gene, and which influence the transcription, RNA processing or stability, or translation of the associated coding region. Regulatory regions may include promoters, translation leader sequences, RNA processing site, effector binding site, and stem-loop structure. The boundaries of the coding region of a gene are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA, or RNA, that comprises a translation start signal or initiation codon, such as an ATG or AUG, and a termination codon and can be potentially translated into a polypeptide sequence.

In embodiments in which the gene encodes a polypeptide, the heterologous nucleic acid molecules include one or a combination of terminator sequence(s) to end the translation of the polypeptide. In some embodiments, one or more terminators can be used. In some embodiments, the one or more terminators used are terminators derived from genes found in yeasts (such as for example Saccharomyces or Komagataella). In some embodiments, the terminator comprises the terminator derived from is from the dit1 gene (dit1t, a functional variant or a functional fragment thereof), from the idp1 gene (idp1t, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), from the ira2 gene (ira2t, a functional variant or a functional fragment thereof), from the rpl3 gene (rpl3t, a functional variant thereof or a functional fragment thereof), from the bna4 gene (bna4t, a functional variant thereof or a functional fragment thereof), from the pgk1 gene (pgk1t, a functional variant thereof or a functional fragment thereof), from the fur4 gene (fur4t, a functional variant thereof or a functional fragment thereof), from the mig2 gene (mig2t, a functional variant thereof or a functional fragment thereof), from the icy2 gene (icy2t, a functional variant thereof or a functional fragment thereof), from the gic1 gene (gic1t, a functional variant thereof or a functional fragment thereof), from the aox1 gene (aox1t, a functional variant thereof or a functional fragment thereof), from the gap1 gene (gap1t, a functional variant thereof or a functional fragment thereof), from the gapdh gene (gapdht, a functional variant thereof or a functional fragment thereof), from the dhas gene (dhast, a functional variant thereof or a functional fragment thereof), from the fdh gene (fdht, a functional variant thereof or a functional fragment thereof), from the fld gene (fldt, a functional variant thereof or a functional fragment thereof), from the act gene (actt, a functional variant thereof or a functional fragment thereof), from the arg4 gene (arg4t, a functional variant thereof or a functional fragment thereof), from the icl1 gene (icl1t, a functional variant thereof or a functional fragment thereof), from the prm9 gene (prm9t, a functional variant thereof or a functional fragment thereof), from the vps13 gene (vps13t, a functional variant thereof or a functional fragment thereof), and/or from the lac4 gene (lac4t, a functional variant thereof or a functional fragment thereof).

The present disclosure provides specific nucleic acid sequences for some embodiments of the engineered promoters. In some embodiments, the engineered promoter comprises the nucleic acid sequence of SEQ ID NO: 6 or a variant thereof. In some embodiments, the engineered promoter comprises the nucleic acid sequence of SEQ ID NO: 7 or a variant thereof. In some embodiments, the engineered promoter comprises the nucleic acid sequence of SEQ ID NO: 8 or a variant thereof. In some embodiments, the engineered promoter comprises the nucleic acid sequence of SEQ ID NO: 9 or a variant thereof. In some embodiments, the engineered promoter comprises the nucleic acid sequence of SEQ ID NO: 10 or a variant thereof. In some embodiments, the engineered promoter comprises the nucleic acid sequence of SEQ ID NO: 11 or a variant thereof. In some embodiments, the engineered promoter comprises the nucleic acid sequence of SEQ ID NO: 12 or a variant thereof. In some embodiments, the engineered promoter comprises the nucleic acid sequence of SEQ ID NO: 13 or a variant thereof. In some embodiments, the engineered promoter comprises the nucleic acid sequence of SEQ ID NO: 14 or a variant thereof. In some embodiments, the engineered promoter comprises the nucleic acid sequence of SEQ ID NO: 15 or a variant thereof. In some embodiments, the engineered promoter comprises the nucleic acid sequence of SEQ ID NO: 16 or a variant thereof. In some embodiments, the engineered promoter comprises the nucleic acid sequence of SEQ ID NO: 17 or a variant thereof. In some embodiments, the engineered promoter comprises the nucleic acid sequence of SEQ ID NO: 18 or a variant thereof. In some embodiments, the engineered promoter comprises the nucleic acid sequence of SEQ ID NO: 19 or a variant thereof. In some embodiments, the engineered promoter comprises the nucleic acid sequence of SEQ ID NO: 20 or a variant thereof. In some embodiments, the engineered promoter comprises the nucleic acid sequence of SEQ ID NO: 21 or a variant thereof. In some embodiments, the engineered promoter comprises the nucleic acid sequence of SEQ ID NO: 22 or a variant thereof. In some embodiments, the engineered promoter comprises the nucleic acid sequence of SEQ ID NO: 23 or a variant thereof. In some embodiments, the engineered promoter comprises the nucleic acid sequence of SEQ ID NO: 24 or a variant thereof.

A “variant” of a nucleic acid sequence of the engineered promoter exhibits at least 70%, 75%, 80%, 85%, 90%, 95%, 96%, 97%, 98%, 99% identity or more to the nucleic acid sequence of any one of SEQ ID NO: 6 to 23. The term “% identity”, as known in the art, is a relationship between two or more nucleic sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.) or Geneious Prime. Multiple alignments of the sequences disclosed herein were performed using the Clustal Omega alignment method, which employs a Hidden Markov Model-based algorithm, using default parameters (including a cluster size=100).

The variants engineered promoters also exhibit a similar expression strength as the engineered promoter having the nucleic acid sequence of any one of SEQ ID NO: 6 to 23. It is well known in the art how to determine if two engineered promoters have a similar expression strength. For example, in order to do so, the putative variant engineered promoter and the engineered promoter are independently operatively associated with a reporter gene (intended to be included in a microbial host cell) or a native gene of a microbial host cell to generate to distinct heterologous nucleic acid molecules in distinct microbial host cells. The first microbial host comprises an engineered promoter described herein (such as, for example, an) engineered promoter having the nucleic acid sequence of any one of SEQ ID NO: 6 to 23) operatively associated with a heterologous or native gene; and the second microbial host cell comprises the putative variant engineered promoter having at least 70% (but less than 100%) identity with the engineered promoter of the first microbial cell. Then, both microbial host cells are placed in substantially similar culture conditions in the absence and in the presence of the inducer (a C2 carbon source like ethanol) or the non-C2 carbon source (glucose, fructose, or glycerol for example). The expression of the heterologous or the native genes under the control of the engineered promoter and of the putative variant engineered promoter is then determined. If the expression of the heterologous or the native gene is modulated in a substantially similar way with the engineered promoter and with the putative variant engineered promoter, then the putative variant engineered promoter is considered to exhibit a similar expression strength as the engineered promoter. As used herein, the expression “a substantially similar way” refers to the ability of the putative engineered promoter to recruit the Adr1, Cat8 (also referred to as Cat8-1), Sip4 (also referred to as Cat8-2), and/or Mig1 transcription factors in the presence of the inducer (a C2 carbon source, like ethanol) or the non-C2 carbon source (like glucose, fructose and/or glycerol). If the expression of the heterologous or the native gene is modulated in a dissimilar way in the engineered promoter and in the putative variant engineered promoter, then the putative variant engineered promoter is not considered to exhibit a similar expression strength as the engineered promoter. As used herein, the expression “a dissimilar way” refers to the lack of the ability of the putative engineered promoter to recruit the Adr1, Cat8 (also referred to as Cat8-1), Sip4 (also referred to as Cat8-2), and/or Mig1 transcription factors in the presence of the inducer (a C2 carbon source like ethanol) or the non-C2 carbon source (like glucose, fructose and/or glycerol).

The variants engineered promoters include the same number of external CSRE(s) at the same location as the corresponding engineered promoters from which they are derived. In some embodiments, the external CSRE(s) present in the variant engineered promoters have the same nucleic acid sequences than the external CSRE(s) present in the corresponding engineered promoters from which they are derived. In alternative embodiments, the external CSRE(s) present in the variant engineered promoters have different nucleic acid sequences than the external CSRE(s) present in the corresponding engineered promoters from which they are derived. The external CSRE(s) of the variant engineered promoters are of Formula (I).

The present disclosure provides vectors comprising the engineered promoters described herein and, in some embodiments, the heterologous nucleic acid molecules also comprise a gene operatively linked thereto. The heterologous nucleic acid molecules of the present disclosure can be introduced in the recombinant microbial host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phages or nucleotide sequences, linear, circular, or supercoiled, of a single- or double-stranded DNA or RNA, derived from any source, in which a number of nucleotide sequences have been joined or recombined into a unique construction which is capable of introducing a promoter and a nucleic acid molecule for a selected gene product along with appropriate 3′ untranslated sequence into a cell. In some embodiments, the vector also comprises a gene coding for a selection marker to allow the section of microbial host cell bearing and expressing the vector. As such, the present disclosure provides vectors comprising the engineered promoters disclosed herein intended to be integrated in the genome, and in some embodiments in the chromosome of the recombinant microbial host cell.

The present disclosure provides expression cassettes comprising the engineered promoters described herein and, in some embodiments, the heterologous nucleic acid molecules comprising the engineered promoters described herein. In the context of the present disclosure, an “expression cassette” refers to a module containing genes to be expressed as well as regulatory elements associated with the genes. Expression cassettes comprise one or more of the engineered promoters described herein operatively linked to one or more genes. In some embodiments, the expression cassettes can also include one or more selection markers to allow the selection of microbial host cell bearing and expressing the expression cassettes. In yet additional embodiments, the expression cassettes also include, at their 5′ and 3′ termini locus-specific targeting sequences to favor specific homologous recombination in the recombinant microbial host cell. As such, the present disclosure provides expression cassettes comprising the engineered promoters disclosed herein intended to be integrated in the chromosome of the recombinant microbial host cell.

The present disclosure provides a method of increasing responsiveness of a parental promoter to a C2 carbon source, like ethanol. The method can be applied to any promoter (inducible or constitutive) to increase the ability of the engineered promoter (when compared to the parental promoter) to augment the transcription (and in some embodiments the translation) of a gene operatively associated thereto. In some embodiments, the method can be applied to the parental promoters described herein. Broadly, the method comprises introducing, upstream and proximal to the transcription start site of the parental promoter, a first external CSRE of formula (I). In some embodiments, the first external CSRE can have the nucleic acid sequence of any one of SEQ ID NO: 26 to 35. The integration site of this first external CSRE (with respect to the transcription start site) can be any one of those described herein. In an embodiment, the engineered promoter is intended to be operatively linked to a gene comprising an open reading frame having a start codon. In such embodiments, the integration site of this first external CSRE (with respect to the start coding) can be any one of those described herein. In some specific embodiments, the first external CSRE is integrated at most 390 base pairs upstream (−390) of the start codon. In another embodiment, the parental promoter (and, by extension, the engineered promoter) comprises a TATA box. In such embodiments, the integration site of this first external CSRE (with respect to the TATA box) can be any one of those described herein.

The method can include, in some embodiments, introducing a second external CSRE of formula (I) in the engineered promoter. In additional embodiments, the second external CSRE can have the nucleic acid sequence of any one of SEQ ID NO: 26 to 35. The second external CSRE can be introduced anywhere in the engineered promoter. In an embodiment, the second external CSRE is located upstream and proximal to the transcription start site. In a further embodiment, the second external CSRE can be integrated at any of the insertion location described herein for the first external CSRE, provided that the second external CSRE does not disrupt the first external CSRE. The method can include, in some embodiments, introducing a third external CSRE of formula (I) in the engineered promoter. In additional embodiments, the third external CSRE can have the nucleic acid sequence of any one of SEQ ID NO: 26 to 35. The third external CSRE can be introduced anywhere in the engineered promoter. In an embodiment, the third external CSRE is located upstream and proximal to the transcription start site. In a further embodiment, the third external CSRE can be integrated at any of the insertion location described herein for the first external CSRE, provided that the third external CSRE does not disrupt the first or the second external CSRE. The method can include, in some embodiments, introducing a fourth external CSRE of formula (I) in the engineered promoter. In additional embodiments, the fourth external CSRE can have the nucleic acid sequence of any one of SEQ ID NO: 26 to 35. The fourth external CSRE can be introduced anywhere in the engineered promoter. In an embodiment, the fourth external CSRE is located upstream and proximal to the transcription start site. In a further embodiment, the fourth external CSRE can be integrated at any of the insertion location described herein for the first external CSRE, provided that the fourth external CSRE does not disrupt the first, second, or third external CSRE. The method can include, in some embodiments, introducing a fifth external CSRE of formula (I) in the engineered promoter. In additional embodiments, the fifth external CSRE can have the nucleic acid sequence of any one of SEQ ID NO: 26 to 35. The fifth external CSRE can be introduced anywhere in the engineered promoter. In an embodiment, the fifth external CSRE is located upstream and proximal to the transcription start site. In a further embodiment, the fifth external CSRE can be integrated at any of the insertion location described herein for the first external CSRE, provided that the fifth external CSRE does not disrupt the first, second, third, or fourth external CSRE. The method can include, in some embodiments, introducing a sixth external CSRE of formula (I) in the engineered promoter. In additional embodiments, the sixth external CSRE can have the nucleic acid sequence of any one of SEQ ID NO: 26 to 35. The sixth external CSRE can be introduced anywhere in the engineered promoter. In an embodiment, the sixth external CSRE is located upstream and proximal to the transcription start site. In a further embodiment, the sixth external CSRE can be integrated at any of the insertion location described herein for the first external CSRE, provided that the sixth external CSRE does not disrupt the first, second, third, fourth, or fifth external CSRE. The method can include, in some embodiments, introducing a seventh external CSRE of formula (I) in the engineered promoter. In additional embodiments, the seventh external CSRE can have the nucleic acid sequence of any one of SEQ ID NO: 26 to 35. The seventh external CSRE can be introduced anywhere in the engineered promoter. In an embodiment, the seventh external CSRE is located upstream and proximal to the transcription start site. In a further embodiment, the seventh external CSRE can be integrated at any of the insertion location described herein for the first external CSRE, provided that the seventh external CSRE does not disrupt the first, second, third, fourth, fifth, or sixth external CSRE. The method can include, in some embodiments, introducing an eighth external CSRE of formula (I) in the engineered promoter. In additional embodiments, the eighth external CSRE can have the nucleic acid sequence of any one of SEQ ID NO: 26 to 35. The eighth external CSRE can be introduced anywhere in the engineered promoter. In an embodiment, the eighth external CSRE is located upstream and proximal to the transcription start site. In a further embodiment, the eighth external CSRE can be integrated at any of the insertion location described herein for the first external CSRE, provided that the eighth external CSRE does not disrupt the first, second, third, fourth, fifth, sixth, or seventh external CSRE. The method can include, in some embodiments, introducing a ninth external CSRE of formula (I) in the engineered promoter. In additional embodiments, the ninth external CSRE can have the nucleic acid sequence of any one of SEQ ID NO: 26 to 35. The ninth external CSRE can be introduced anywhere in the engineered promoter. In an embodiment, the ninth external CSRE is located upstream and proximal to the transcription start site. In a further embodiment, the ninth external CSRE can be integrated at any of the insertion location described herein for the first external CSRE, provided that the ninth external CSRE does not disrupt the first, second, third, fourth, fifth, sixth, seventh, or eighth external CSRE. The method can include, in some embodiments, introducing a tenth external CSRE of formula (I) in the engineered promoter. In additional embodiments, the tenth external CSRE can have the nucleic acid sequence of any one of SEQ ID NO: 26 to 35. The tenth external CSRE can be introduced anywhere in the engineered promoter. In an embodiment, the tenth external CSRE is located upstream and proximal to the transcription start site. In a further embodiment, the tenth external CSRE can be integrated at any of the insertion location described herein for the first external CSRE, provided that the tenth external CSRE does not disrupt the first, second, third, fourth, fifth, sixth, seventh, eighth, or ninth external CSRE.

### Recombinant Microbial Host Cells

The recombinant microbial host cell is obtained from a microbial cell which can be a bacterium, a yeast or a fungus. The recombinant microbial host cell of the present disclosure is capable of metabolizing a C2 carbon source like ethanol (e.g., it exhibits alcohol dehydrogenase activity). In some embodiments, the recombinant microbial host cell is obtained from a microbial cell which natively is capable of metabolizing a C2 carbon source like ethanol (e.g., it exhibits native alcohol dehydrogenase activity). In other embodiments, the recombinant microbial host cell is obtained from a microbial cell which does not have a native capacity of metabolizing a C2 carbon source like ethanol, but it has been genetically engineered to be capable of metabolizing the C2 carbon source (e.g., it exhibits heterologous alcohol dehydrogenase activity). In further embodiments, the recombinant microbial host cell is obtained from a microbial cell which is natively capable of metabolizing a C2 carbon source like ethanol and it has been genetically engineered to be capable of metabolizing even more of the C2 carbon source (e.g., it exhibits native and heterologous alcohol dehydrogenase activity).

In some embodiments, the recombinant microbial host cell of the present disclosure is capable of metabolizing a non-C2 carbon source like glucose, fructose, or glycerol. In some embodiments, the recombinant microbial host cell is obtained from a microbial cell which natively is capable of metabolizing a non-C2 carbon source like glucose, fructose, or glycerol. In other embodiments, the recombinant microbial host cell is obtained from a microbial cell which does not have a native capacity of metabolizing a non-C2 carbon source like glucose, fructose, or glycerol, but it has been genetically engineered to be capable of metabolizing the non-C2 carbon source. In further embodiments, the recombinant microbial host cell is obtained from a microbial cell which is natively capable of metabolizing a non-C2 carbon source like glucose, fructose, or glycerol and it has been genetically engineered to be capable of metabolizing even more of the non-C2 carbon source.

In an embodiment, the recombinant host cell is obtained from a microbial cell which is a bacterium. In some embodiments, the bacterium is a Gram-positive bacterium. In other embodiments, the bacterium is a Gram-negative bacterium. In an embodiment, the recombinant microbial host cell/microbial cell is from Actinoplanes sp. In another embodiment, the recombinant microbial host cell/microbial cell is from Actinoplanes missouriensis. In an embodiment, the recombinant microbial host cell/microbial cell is from Aeribacillus sp. In another embodiment, the recombinant microbial host cell/microbial cell is from Aeribacillus pallidus. In an embodiment, the recombinant microbial host cell/microbial cell is from Anoxybacillus sp. In another embodiment, the recombinant microbial host cell/microbial cell is from Anoxybacillus caldiproteolyticus. In an embodiment, the recombinant microbial host cell/microbial cell is from Bacillus sp. In another embodiment, the recombinant microbial host cell/microbial cell is from Bacillus acidopullulyticus. In another embodiment, the recombinant microbial host cell/microbial cell is from Bacillus amyloliquefaciens. In another embodiment, the recombinant microbial host cell/microbial cell is from Bacillus licheniformis. In another embodiment, the recombinant microbial host cell/microbial cell is from Bacillus pumilus. In another embodiment, the recombinant microbial host cell/microbial cell is from Bacillus subtilis. In an embodiment, the recombinant microbial host cell/microbial cell is from Chryseobacterium sp. In another embodiment, the recombinant microbial host cell/microbial cell is from Chryseobacterium proteolyticum. In an embodiment, the recombinant microbial host cell/microbial cell is from Escherichia sp. In another embodiment, the recombinant microbial host cell/microbial cell is from Escherichia coli. In an embodiment, the recombinant microbial host cell/microbial cell is from Geobacillus sp. In another embodiment, the recombinant microbial host cell/microbial cell is from Geobacillus stearothermophilus. In an embodiment, the recombinant microbial host cell/microbial cell is from Lactobacillus sp. In another embodiment, the recombinant microbial host cell/microbial cell is from Lactobacillus fermentum. In an embodiment, the recombinant microbial host cell/microbial cell is from Lactococcus sp. In another embodiment, the recombinant microbial host cell/microbial cell is from Lactococcus lactis. In an embodiment, the recombinant microbial host cell/microbial cell is from Macrococcus sp. In another embodiment, the recombinant microbial host cell/microbial cell is from Macrococcus caseolyticus. In an embodiment, the recombinant microbial host cell/microbial cell is from Microbacterium sp. In another embodiment, the recombinant microbial host cell/microbial cell is from Microbacterium arborescens. In an embodiment, the recombinant microbial host cell/microbial cell is from Micrococcus sp. In another embodiment, the recombinant microbial host cell/microbial cell is from Micrococcus lysodeikticus. In an embodiment, the recombinant microbial host cell/microbial cell is from Priestia sp. In another embodiment, the recombinant microbial host cell/microbial cell is from Priestia flexa. In an embodiment, the recombinant microbial host cell/microbial cell is from Streptomyces sp. In another embodiment, the recombinant microbial host cell/microbial cell is from Streptomyces mobaraensis. In another embodiment, the recombinant microbial host cell/microbial cell is from Streptomyces murinus. In another embodiment, the recombinant microbial host cell/microbial cell is from Streptomyces olivaceus. In another embodiment, the recombinant microbial host cell/microbial cell is from Streptomyces olivochromogenes. In another embodiment, the recombinant microbial host cell/microbial cell is from Streptomyces rubiginosus. In another embodiment, the recombinant microbial host cell/microbial cell is from Streptomyces violaceoruber. In an embodiment, the recombinant microbial host cell/microbial cell is from Pseudomonas sp. In another embodiment, the recombinant microbial host cell/microbial cell is from Pseudomonas fluorescens. In an embodiment, the recombinant microbial host cell/microbial cell is from Weizmannia sp. In another embodiment, the recombinant microbial host cell/microbial cell is from Weizmannia coagulans.

In an embodiment, the recombinant host cell is obtained from a microbial cell which is a yeast. In some embodiments, the yeast is a budding yeast. In other embodiments, the yeast is methylotrophic (e.g., yeast able to utilize methanol as the sole carbon and energy source). Embodiments of methylotrophic yeasts include, but are not limited to Komagataella sp. and Ogataea sp. In some embodiments, the yeast is an oleaginous yeast (e.g., a yeast capable of accumulating more than 20% of its dry cell weight as lipids or triglycerides).

In an embodiment, the recombinant microbial host cell/microbial cell is from Blastobotrys sp. In another embodiment, the recombinant microbial host cell/microbial cell is from Blastobotrys adeninivorans (basonym Trichosporon adeninivorans). In an embodiment, the recombinant microbial host cell/microbial cell is from Candida sp. In another embodiment, the recombinant microbial host cell/microbial cell is from Candida albicans. In an embodiment, the recombinant microbial host cell/microbial cell is from Cyberlindnera sp. In another embodiment, the recombinant microbial host cell/microbial cell is from Cyberlindnera jadinii (basonym Saccharomyces jadinii). In an embodiment, the recombinant microbial host cell/microbial cell is from the Debaryomyces sp. In another embodiment, the recombinant microbial host cell/microbial cell is from Debaryomyces hansenii. In another embodiment, the recombinant microbial host cell/microbial cell is from Debaryomyces hansenii. In an embodiment, the recombinant microbial host cell/microbial cell is from Hanseniaspora sp. (also known as Kloeckera sp.). In another embodiment, the recombinant microbial host cell/microbial cell is from Hanseniaspora guilliermondii. In another embodiment, the recombinant microbial host cell/microbial cell is from Hanseniaspora pseudoguilliermondii. In an embodiment, the recombinant microbial host cell/microbial cell is from the Kazachstania sp. In another embodiment, the recombinant microbial host cell/microbial cell is from Kazachstania bulderi (basonym Saccharomyces bulderi). In another embodiment, the recombinant microbial host cell/microbial cell is from Kazachstania barnettii (basonym Saccharomyces barnettii). In another embodiment, the recombinant microbial host cell/microbial cell is from Kazachstania exigua (basonym Saccharomyces exiguus). In an embodiment, the recombinant microbial host cell/microbial cell is from Kluyveromyces sp. In another embodiment, the recombinant microbial host cell/microbial cell is from Kluyveromyces lactis (basonym Torulaspora lactis). In another embodiment, the recombinant microbial host cell/microbial cell is from Kluyveromyces marxianus also known as Kluyveromyces fragilis (basonym Saccharomyces marxianus). In an embodiment, the recombinant microbial host cell/microbial cell is from Komagataella sp. In another embodiment, the recombinant microbial host cell/microbial cell is from Komagataella phaffii. In an embodiment, the recombinant microbial host cell/microbial cell is from Limtongozyma sp. In another embodiment, the recombinant microbial host cell/microbial cell is from Limtongozyma cylindracea (basonym Candida cylindracea). In an embodiment, the recombinant microbial host cell/microbial cell is from Lipomyces sp. In an embodiment, the recombinant microbial host cell/microbial cell is from Metschnikowia sp. In another embodiment, the recombinant microbial host cell/microbial cell is from Metschnikowia sinensis. In another embodiment, the recombinant microbial host cell/microbial cell is from Metschnikowia fructicola. In another embodiment, the recombinant microbial host cell/microbial cell is from Metschnikowia pulcherrima. In another embodiment, the recombinant microbial host cell/microbial cell is from Metschnikowia zobellii. In another embodiment, the recombinant microbial host cell/microbial cell is from Metschnikowia shanxiensis. In an embodiment, the recombinant microbial host cell/microbial cell is from Ogataea sp. In another embodiment, the recombinant microbial host cell/microbial cell is from Ogataea polymorpha (basonym Hansenula polymorpha). In another embodiment, the recombinant microbial host cell/microbial cell is from Ogataea methanolica (basonym Pichia methanolica). In an embodiment, the recombinant microbial host cell/microbial cell is from Pichia sp. (also known as Hansenula sp.). In an embodiment, the recombinant microbial host cell/microbial cell is from Rasamsonia sp. In another embodiment, the recombinant microbial host cell/microbial cell is from Rasamsonia emersonii. In an embodiment, the recombinant microbial host cell/microbial cell is from Saccharomyces sp. In another embodiment, the recombinant microbial host cell/microbial cell is from Saccharomyces cerevisiae. In yet another embodiment, the recombinant microbial host cell/microbial cell is from Saccharomyces cerevisiae var. diastaticus. In another embodiment, the recombinant microbial host cell/microbial cell is from Saccharomyces uvarum. In another embodiment, the recombinant microbial host cell/microbial cell is from Saccharomyces boulardii. In an embodiment, the recombinant microbial host cell/microbial cell is from Scheffersomyces sp. In another embodiment, the recombinant microbial host cell/microbial cell is from Scheffersomyces stipitis (basonym Pichia stipitis). In an embodiment, the recombinant microbial host cell/microbial cell is from Schwanniomyces sp. In another embodiment, the recombinant microbial host cell/microbial cell is from Schwanniomyces polymorphus (basonym Pichia polymorpha). In another embodiment, the recombinant microbial host cell/microbial cell is from Schwanniomyces occidentalis. In an embodiment, the recombinant microbial host cell/microbial cell is from Wickerhamomyces sp. In another embodiment, the recombinant microbial host cell/microbial cell is from Wickerhamomyces anomalus. In an embodiment, the recombinant microbial host cell/microbial cell is from Yarrowia sp. In another embodiment, the recombinant microbial host cell/microbial cell is from Yarrowia lipolytica.

In an embodiment, the recombinant host cell is obtained from a microbial cell which is a fungus. In some embodiments, the fungus is an ascomycete fungus. In alternative embodiments, the fungus is a basidiomycete fungus. In a further embodiment, the fungus is an oleaginous fungus (e.g., a fungus capable of accumulating more than 20% of its dry cell weight as lipids or triglycerides). In an embodiment, the recombinant microbial host cell/microbial cell is from Aspergillus sp. In another embodiment, the recombinant microbial host cell/microbial cell is from Aspergillus acidus. In another embodiment, the recombinant microbial host cell/microbial cell is from Aspergillus fijiensis. In another embodiment, the recombinant microbial host cell/microbial cell is from Aspergillus japonicus. In another embodiment, the recombinant microbial host cell/microbial cell is from Aspergillus luchuensis. In another embodiment, the recombinant microbial host cell/microbial cell is from Aspergillus melleus. In another embodiment, the recombinant microbial host cell/microbial cell is from Aspergillus niger. In another embodiment, the recombinant microbial host cell/microbial cell is from Aspergillus oryzae. In an embodiment, the recombinant microbial host cell/microbial cell is from Blakeslea sp. In an embodiment, the recombinant microbial host cell/microbial cell is from Cunninghamella sp. In an embodiment, the recombinant microbial host cell/microbial cell is from Cryphonectria sp. In another embodiment, the recombinant microbial host cell/microbial cell is from Cryphonectria parasitica. In an embodiment, the recombinant microbial host cell/microbial cell is from Cryptococcus sp. In an embodiment, the recombinant microbial host cell/microbial cell is from Disporotrichum sp. In another embodiment, the recombinant microbial host cell/microbial cell is from Disporotrichum dimorphosporum. In an embodiment, the recombinant microbial host cell/microbial cell is from Fusarium sp. In another embodiment, the recombinant microbial host cell/microbial cell is from Fusarium venenatum. In an embodiment, the recombinant microbial host cell/microbial cell is from Humicola sp. In another embodiment, the recombinant microbial host cell/microbial cell is from Humicola insolens. In an embodiment, the recombinant microbial host cell/microbial cell is from Mortierella sp. In an embodiment, the recombinant microbial host cell/microbial cell is from Mucor sp. In another embodiment, the recombinant microbial host cell/microbial cell is from Mucor circinelloides. In an embodiment, the recombinant microbial host cell/microbial cell is from Mycothermus sp. In another embodiment, the recombinant microbial host cell/microbial cell is from Mycothermus thermophiloides. In an embodiment, the recombinant microbial host cell/microbial cell is from Penicillum sp. In another embodiment, the recombinant microbial host cell/microbial cell is from Penicillium camemberti. In another embodiment, the recombinant microbial host cell/microbial cell is from Penicillum chrysogenum. In another embodiment, the recombinant microbial host cell/microbial cell is from Penicillium rubens. In another embodiment, the recombinant microbial host cell/microbial cell is from Penicillium roquefortii. In an embodiment, the recombinant microbial host cell/microbial cell is from Phaffia sp. In another embodiment, the recombinant microbial host cell/microbial cell is from Phaffia rhodozyma. In an embodiment, the recombinant microbial host cell/microbial cell is from Phycomyces sp. In an embodiment, the recombinant microbial host cell/microbial cell is from Rhizomucor sp. In another embodiment, the recombinant microbial host cell/microbial cell is from Rhizomucor miehei. In another embodiment, the recombinant microbial host cell/microbial cell is from Rhizomucorpusillus. In an embodiment, the recombinant microbial host cell/microbial cell is from Rhizopus sp. In another embodiment, the recombinant microbial host cell/microbial cell is from Rhizopus arrhizus (also know as Rhizopus oryzae). In another embodiment, the recombinant microbial host cell/microbial cell is from Rhizopus delemar. In another embodiment, the recombinant microbial host cell/microbial cell is from Rhizopus niveus. In an embodiment, the recombinant microbial host cell/microbial cell is from Rhodotorula sp. (also known as Rhodosporidum sp.). In an embodiment, the recombinant microbial host cell/microbial cell is from Schizosaccharomyces sp. In another embodiment, the recombinant microbial host cell/microbial cell is from Schizosaccharomyces pombe. In an embodiment, the recombinant microbial host cell/microbial cell is from Talaromyces sp. In another embodiment, the recombinant microbial host cell/microbial cell is from Talaromyces funiculosus (also known as Penicillium funiculosum). In an embodiment, the recombinant microbial host cell/microbial cell is from Trichoderma sp. In another embodiment, the recombinant microbial host cell/microbial cell is from Trichoderma reesei. In an embodiment, the recombinant microbial host cell/microbial cell is from Trichosporon sp.

The present disclosure also provides a method of making the recombinant microbial host cell described herein. The method comprises introducing, in the recombinant microbial host cell, a heterologous nucleic acid molecule comprising one or more engineered promoters. In embodiments, the heterologous nucleic acid molecule can be provided as a vector or an expression cassette. The heterologous nucleic acid molecules can be introduced in the genome of the recombinant microbial host cell by any known genetic engineering methods, such as, for example, by homologous recombination, a double strand break mechanism, Cre-LoxP mediated recombination, delitto perfetto, meganuclease-mediated double strand break, MAD7, TALEN, and/or CRISPR/Cas9. The method can also include, in some embodiments, determining the presence and optionally the location of the integrated heterologous nucleic acid molecule. In some embodiments in which the heterologous nucleic acid molecule includes a selection marker, this can be achieved by applying a selective pressure to identify microbial host cell bearing and expressing the heterologous nucleic acid molecule. Alternatively, or in combination, in embodiments in which the heterologous nucleic acid molecule comprises the one or more engineered promoter operatively linked to a gene, the method can include determine the level of expression of the gene (and in some embodiments, the amount and/or activity of the polypeptide that may be encoded by the gene).

### Method of Expressing a Gene Using the Engineered Promoters

The present disclosure comprises a method for expressing a gene using the engineered promoters described herein. The method comprises a step of contacting the recombinant microbial host cell (which comprises one or more engineered promoters operatively linked to the gene intended to be expressed) with an inducer (a C2 carbon source like ethanol) or a non-C2 carbon source (like glucose, fructose, or glycerol for example). This step is usually conducted by adding the inducer (a C2 carbon source like ethanol)/non-C2 carbon source to the medium for culturing the recombinant microbial host cell. As it is known in the art, the amount of the inducer/non-C2 carbon source as well as the incubation conditions (time, temperature, etc.) with the inducer/non-C2 carbon source can be adjusted to optimize gene expression by the recombinant microbial host cell. In some embodiments, during the expression step, the C2 carbon source like ethanol can be provided as the sole source of metabolizable carbon for the recombinant microbial host cell. In other embodiments, during the expression step, the C2 carbon source like ethanol is provided with one or more additional sources of metabolizable carbon for the recombinant microbial host cell. In some embodiments, the expression step can include determining the level of expression of the gene prior to and/or after the addition of the inducer (a C2 carbon source like ethanol)/non-C2 carbon source (like glucose, fructose, or glycerol) to the medium. In embodiments in which the gene encodes a polypeptide, the method can include determining the amount/biological activity of the polypeptide encoded by the gene prior to and/or after the addition of the inducer (a C2 carbon source like ethanol)/non-C2 carbon source (like glucose, fructose, or glycerol) to the medium. The determination steps (expression, amount of polypeptide, activity associated with the polypeptide) can be used to determine if further additions of the inducer (a C2 carbon source like ethanol)/non-C2 carbon source (like glucose, fructose, or glycerol) are required or would be beneficial to further increase gene expression. In some embodiments, the expression step is performed as a continuous fermentation. In alternative embodiments, the expression step is performed as a batch fermentation. In yet further embodiments, the expression step is performed as a fed-batch fermentation. The expression step can be performed, at least in part, in aerobic conditions. In some embodiments, the expression step can be performed in aerobic conditions. The expression step can be performed, at least in part, in anaerobic conditions.

The method can include, prior to the step of expressing the gene, a step of propagating the recombinant microbial host cell. The goal of the propagation step is to increase the biomass associated with the recombinant microbial host cell prior to the expression step. In some embodiments, during the propagation step, the recombinant microbial host cell is placed in contact with a source of metabolizable carbon sources which can include glucose, fructose, glycerol, or a combination thereof. In an embodiment, the propagation step is performed using a medium comprising glucose, such as, for example, molasses. In a specific embodiment, during the propagation step, the recombinant microbial host cell is contacted with glucose as the sole source of metabolizable carbohydrate. In another specific embodiment, during the propagation step, the recombinant microbial host cell is contacted with glycerol as the sole source of metabolizable carbohydrate. In still another embodiment, during the propagation step, the recombinant microbial host cell is contacted with fructose as the sole source of metabolizable carbohydrate. In some embodiments, the propagation step is performed as a continuous fermentation. In alternative embodiments, the propagation step is performed as a batch fermentation. In embodiments in which a batch fermentation is used, especially when the recombinant microbial host cells are recombinant yeast host cells, a diauxic shift occurs in the last phase of a batch fermentation. In some embodiments, the method can comprise determining if the diauxic shift is happening or has happened during the batch fermentation and proceeding to the expression step only after the diauxic shift has been determined to have occurred in the cultures. In yet further embodiments, the propagation step is performed as a fed-batch fermentation. The expression step can be performed, at least in part, in aerobic conditions. In some embodiments, the expression step can be performed in aerobic conditions. The expression step can be performed, at least in part, in anaerobic conditions.

In an embodiment, the engineered promoter(s) is operatively linked to a gene encoding a polypeptide. As used in the context of the present application, a “polypeptide” refers to a polymer comprising at least two amino acid residues (and includes, without limitation, peptides, oligopeptides, as well as proteins). As such, in some embodiments, the method can be used to produce the polypeptide encoded by the gene. Consequently, the method can further include a step of purifying (at least in part) the polypeptide from the recombinant microbial host cell. The purifying step refers to a step of physically dissociating, at least in part, the expressed polypeptide from the components of the recombinant microbial host cell having expressed same. The expression “substantially purified form” refers to the fact that the expressed polypeptides have been physically dissociated from the majority of the components of the recombinant microbial host cells having expressed the polypeptides. In an embodiment, a composition comprising the expressed polypeptides in substantially purified form is at least 40%, 45%, 50%, 55%, 60%, 65%, 70%, 75%, 80%, 85%, 90%, 95%, 96%, 97%, 98%, or 99% pure. In some embodiments, the composition comprising the expressed polypeptides lacks a detectable amount of deoxyribonucleic acids from the recombinant microbial host cell used to express it. The purification step can include, without limitation, a filtrating step, a centrifugating step, a dialysing step, etc.

In embodiments in which the polypeptide is intended to be expressed intracellularly, the method can include a cell-lysing step (after the expression step). The person skilled in the art will recognize that are many ways of lysing recombinant microbial host cells. For example, the cells can be homogenized (for example using a bead-milling technique, a bead-beating, or a high-pressure homogenization technique) and, as such, the method can include a homogenizing step. In another example, the cells can be submitted to an enzyme treatment step (using autolysis or external enzymes or both) and, as such, the method can include an enzymatic lysis step.

The method can also include a drying step (before, after, or both before and after the purifying step). The drying step can include, for example, roller-drying, electrospray-drying, freeze-drying, spray-drying, lyophilization, and/or fluid-bed drying. The method can also include a washing step (before, after, or both before and after the purifying step).

The polypeptide encoded by the gene can be designed, for example, to be secreted, and in such embodiments, it may include a signal sequence (which is intended to be cleaved upon the secretion of the polypeptide). In some embodiments, the polypeptide is designed to be secreted in a free form (not intended to be physically associated with the recombinant microbial host cell) or in a cell-associated form (intended to remain physically associated with the recombinant microbial host cell). In some embodiments of the secreted and cell-associated polypeptide, the latter can be physically tethered to the external surface of the microbial host cell, and in some embodiment, the polypeptide can include a tethering moiety to locate it to the external surface of the microbial host cell.

In another specific embodiment, the polypeptide exhibits enzymatic activity (e.g., it is an enzyme). In yet a further embodiment, the enzyme is suitable in food, feed, and beverage applications. In still a further embodiment, the enzyme is suitable for biofuel applications. In yet another embodiment, the enzyme is suitable for plant health, like agricultural applications. In still another embodiment, the enzyme is suitable for animal health, like veterinary applications. In yet another embodiment, the enzyme is suitable for human health, like medical applications.

In some embodiments, the polypeptide exhibits therapeutic activity and can be used in plant, animal and/or human health.

In another specific embodiment, the polypeptide exhibits antimicrobial activity. For example, the polypeptide can exhibit antibacterial, antifungal and/or antiviral activity. The antimicrobial polypeptide can be used in agriculture, in animal health and/or in in human health applications.

In some embodiments, the polypeptide can be an oxidoreductase (E.C. 1). For example, the polypeptide can be an α-acetolactate decarboxylase. In yet another embodiment, the polypeptide can act on the CH—OH group of donors (E.C. 1.1). In another embodiment, the polypeptide can use oxygen as an acceptor (E.C. 1.1.3). In yet another embodiment, the polypeptide can be a glucose oxidase (1.1.3.4). In a specific embodiment, the glucose oxidase is obtained or derived from Aspergillus sp. and in a further embodiment, from Aspergillus niger. In yet another embodiment, the glucose oxidase is obtained or derived from Accession Number ACB30370.1. Variants of the Aspergillus niger glucose oxidase have been described in U.S. provisional patent application 63/582,640 (filed on Sep. 14, 2023) incorporated herewith in its entirety. In another embodiment, the polypeptide can be a hexose oxidase (E.C. 1.1.3.5). In yet another embodiment, the polypeptide can act on peroxide as a receptor (E.C. 1.11). In another embodiment, the polypeptide can be a peroxidase (E.C. 1.11.1). In yet another embodiment, the polypeptide can be a catalase (E.C. 1.11.1.6).

In some embodiments, the polypeptide can be a transferase (E.C. 2). For example, the polypeptide can be an acyltransferase (E.C. 2.3). In yet another embodiment, the polypeptide can be an aminoactyltransferase (E.C. 2.3.2). In another embodiment, the polypeptide can be a protein-glutamine gamma-glutamyltransferase (also known as a transglutaminase, E.C. 2.3.2.13).

In some embodiments, the polypeptide can be a hydrolase or a lytic enzyme (E.C. 3). In still another embodiment, the lytic enzyme can be a glycoside hydrolase or a glycosylase (E.C. 3.2). In the context of the present disclosure, the term “glycoside hydrolase” refers to an enzyme involved in carbohydrate digestion, metabolism, and/or hydrolysis. Glycoside hydrolases include, without limitation, glycosidases (like mannanases, E.C. 3.2.1), amylases (GH family 13 and/or corresponding to E.C. 3.2.1.1), arabinofuranosidases, asparaginases (E.C. 3.5), cellulases (E.C. 3.2.1.4), inulinases (E.C. 3.2.1.7), cellulolytic and amylolytic accessory enzymes, endoglucanases, esterases (E.C. 3.1), galactosidases, hemicellulases, lactases (E.C. 3.2.1.108), levanases, pectinases, peptidases (including aminopeptidases, carboxypeptidases, and endopeptidases), proteases (E.C. 3.4), asparaginases (E.C. 3.5.1.1.), fructan beta-fructosidase (e.g., invertase, E.C. 3.2.1.80), lysozymes (E.C. 3.2.1.17), trehalases (E.C. 3.2.1.28), pullalanases (E.C. 3.2.1.41), xylanases, and xylosidases. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases. Esterases include, but are not limited to, phytases, lipases, phospholipases A1, and phospholipases A2. In some embodiments, the lipase is from Fusarium sp., such as, for example, from Fusarium oxysporum and can be derived from, in additional embodiments, Accession Number KAH7177381.

Amylases can be, for example, from plant, fungal and/or bacterial origin. Amylases include, but are not limited to, alpha-amylases (E.C. 3.2.1.1, sometimes referred to fungal alpha-amylase), beta-amylases (E.C. 3.2.1.2), maltogenic alpha-amylases (E.C. 3.2.1.133), glucoamylases (E.C. 3.2.1.3), glucan 1,4-α-maltotetraohydrolase (E.C. 3.2.1.60), pullulanase (E.C. 3.2.1.41), iso-amylase (E.C. 3.2.1.68), and amylomaltase (E.C. 2.4.1.25). In an embodiment, the one or more amylolytic enzymes can be an alpha-amylase from Aspergillus oryzae, Saccharomycopsis fibuligera (GenBank Accession #CAA29233.1 for example), and Bacillus amyloliquefaciens (GenBank Accession #ABS72727 for example); an alpha-amylase from Geobacillus stearothermophilus (Uniprot P19531 for example) or derivatives thereof, for example, those described in PCT/IB2023/053276 and PCT/IB2023/052263 (which are both incorporated herein in their entirety); an archaeal alpha amylase obtained or derived from archaeal alpha-amylase comprises a polypeptide derived from Thermococcus sp., such as, for example, from Thermococcus hydrothermalis or derivatives thereof, for example those described in PCT/IB2023/052263 (which is incorporated herein in its entirety); an archaeal alpha amylase obtained or derived from archaeal alpha-amylase comprises a polypeptide derived from Pyrococcus sp., such as, for example, from Pyrococcus furiosus or derivatives thereof, for example those described in PCT/IB2023/052263 (which is incorporated herein in its entirety); a glucan 1,4-alpha-maltotetraohydrolase from Pseudomonas saccharophila; a pullulanase from Bacillus naganoensis; a pullulanase from Bacillus acidopullulyticus; an iso-amylase from Pseudomonas amyloderamosa; and/or an amylomaltase from Thermus thermophiles. In an embodiment, the trehalase can be from Aspergillus fumigatus (GenBank Accession #XP_748551) or Neurospora crassa (GenBank Accession #XP_960845.1).

A “cellulase” can be any enzyme involved in cellulose digestion, metabolism and/or hydrolysis, including an endoglucanase, glucosidase, cellobiohydrolase, glucanase, cellobiose phosphorylase, cellodextrin phosphorylase.

Cellulolytic and amylolytic accessory enzymes can include, for example, xylanase, xylosidase, xylan esterase, arabinofuranosidase, galactosidase, mannanase, mannosidase, xyloglucanase, endoxylanase, glucuronidase, acetylxylanesterase, arabinofuranohydrolase, swollenin, glucuronyl esterase, expansin, pectinase, and feruloyl esterase protein.

The polypeptide can have “hemicellulolytic activity”, an enzyme involved in hemicellulose digestion, metabolism and/or hydrolysis. The term “hemicellulase” refers to a class of enzymes that catalyze the hydrolysis of hemicellulose. Several different kinds of enzymes are known to have hemicellulolytic activity including, but not limited to, xylanases and mannanases and xylan esterases, endoxylanase, glucuronidase, acetylxyl transferease, arabinofura hydrolase, feruloyl esterase, galactanase, beta-glucanase.

The polypeptide can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous enzyme can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein.

The polypeptide can be a “pentose sugar utilizing enzyme” involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase. In an embodiment, the one or more xylanase enzymes can be a xylanase from Aspergillus niger (GenBank Accession #CAA03655.1)

The polypeptide can have “mannan-degrading activity”, an enzyme having the ability to hydrolyze the terminal, non-reducing β-D-mannose residues in β-D-mannosides. Mannanases are capable of breaking down hemicellulose, one of the major components of plant cell walls.

The polypeptide can be a “pectinase”, an enzyme, such as pectin lyase (E.C. 4.2.2.10), polygalacturonase, endopolygalacturonase (EPG), pectin methyl esterase (PME). These enzymes break down pectin, a polysaccharide substrate that is found in the cell walls of plants.

The polypeptide can have “phytolytic activity”, an enzyme catalyzing the conversion of phytic acid into inorganic phosphorus. Phytases (E.C. 3.2.3) can be belong to the histidine acid phosphatases, β-propeller phytases, purple acid phosphastases or protein tyrosine phosphatase-like phytases family. In an embodiment, the one or more phytase enzymes can be a phytase from Citrobacter braakii(GenBank Accession #AY471611.1).

The polypeptide can have “proteolytic activity”, an enzyme involved in protein digestion, metabolism and/or hydrolysis, including serine proteases, threonine proteases, cysteine proteases, aspartate proteases (e.g., proteases having aspartic activity), glutamic acid proteases, and metalloproteases. Proteases also include aminopeptidases, carboxypeptidases, and endopeptidases (such as, for example the aspartic endopeptidase chymosin E.C. 3.4.23., the cysteine endopeptidase ficin E.C. 3.4.22.4 as well as the serine endopeptidase trypsin E.C. 3.4.21.4). In an embodiment, the one or more protease enzymes can be a protease from Saccharomycopsis fibuligera (GenBank Accession #P22929) or Aspergillus fumigatus (GenBank Accession #P41748).

The polypeptide can have “hydrolase” activity, e.g., the ability to act on carbon-hydrogen bonds that are not peptide bonds (E.C. 3.5). In some embodiment, the polypeptide acts on linear amines (E.C. 3.5.1). In specific embodiments, the polypeptide can be a glutaminase (E.C. 3.5.1.2). In other specific embodiments, the polypeptide can be a urease (E.C. 3.5.1.5).

In some embodiments, the polypeptide can be a lyase (E.C. 4). For example, the lyase can cleave carbon-carbon bonds (E.C. 4.1), such as decarboxylases (E.C. 4.1.1), aldehyde lyases (E.C. 4.1.2), oxo acid lyases (E.C. 4.1.3), and others (E.C. 4.1.99). In another example, the lyase can cleave carbon-oxygen bonds (E.C. 4.2), such as dehydratases. In a further example, the lyase can cleave carbon-nitrogen bonds (E.C. 4.3), carbon-sulfur bonds (E.C. 4.4), carbon-halide bonds (E.C. 4.5), phosphorus-oxygen bonds (E.C. 4.6 includes lyases that cleave such as adenylyl cyclase and guanylyl cyclase), etc. (including E.C. 4.99, such as ferrochelatase).

In some embodiments, the polypeptide can be an isomerase (E.C. 5). In another embodiment, the polypeptide can have intramolecular oxidoreductase activity (E.C. 5.3). In additional embodiments, the polypeptide can be able to interconvert aldoses, ketoses and related products (E.C. 5.3.1.). In still a further embodiment, the polypeptide can be a glucose isomerase (E. C. 5.3.1.18).

The present invention will be more readily understood by referring to the following examples which are given to illustrate the invention rather than to limit its scope.

### Example

Table 2 provides the description of the various Komagataella phaffii strains that were engineered to express a reporter phospholipase. The genetically modified strains were all generated (directly or indirectly) from the K. phaffii strain NRRL Y-11430 obtained from ATCC (76273).

TABLE 2
Komagataella phaffii strains (M*) and isolates (T*) that were made. All strains
(except the wild-type strains) and isolates were engineered to express a reporter
gene (Fusarium oxysporum phospholipase (PLA)) having the amino acid sequence
of SEQ ID NO: 25 and encoded by the nucleic acid sequence of SEQ ID NO: 36. All
strains are derived (directly or indirectly) from the wild-type strain M17500.
Each strain/isolate comprises a single chromosomal copy of an expression cassette
comprising a promoter and the reporter gene, except strain M32702 which includes
two chromosomal copies of the expression cassette. Data obtained with isolates
(T*) refer to the average of several isolates following a transformation.

| | | | | Position of CSRE with respect to | | |
|---|---|---|---|---|---|---|
| Strain | | | | | start codon | |
| or | | SEQ | CSRE in | TATA | of reporter | |
| isolate | Promoter | ID NO: | epromoter? | box | gene | TSS |
| M17500 | N.A. - this is a wild-type strain | | | | | |
| M34673 | N.A. - this is a wild-type strain | | | | | |
| | (isolate obtained from M17500) | | | | | |
| M31676 | AOX1 | 1 | N.A. | N.A. | N.A. | N.A. |
| | promoter | | | | | |
| M32338 | KpADH2 | 2 | N.A. | N.A. | N.A. | N.A. |
| M32702 | promoter | | | | | |
| M32696 | KpSPI1 | 3 | N.A. | N.A. | N.A. | N.A. |
| | promoter | | | | | |
| M32716 | OpADH1 | 4 | N.A. | N.A. | N.A. | N.A. |
| | promoter | | | | | |
| M32347 | OpFMD | 5 | N.A. | N.A. | N.A. | N.A. |
| | promoter | | | | | |
| M32816 | eADH2-01 | 6 | CSRE#1 | −131/−132 | −214/−213 | N.A. |
| | promoter | | | | | |
| M32818 | eADH2-02 | 7 | CSRE#1 | −131/−132 | −214/−213 | N.A. |
| | promoter | | CSRE#2 | −200/−201 | −283/−282 | N.A. |
| M32820 | eADH2-03 | 8 | CSRE#1 | −131/−132 | −214/−213 | N.A. |
| | promoter | | CSRE#2 | −200/−201 | −283/−282 | N.A. |
| | | | CSRE #3 | −320/−321 | −403/−402 | N.A. |
| M33401 | eADH2-05 | 9 | CSRE#1 | −95/−96 | −178/−177 | N.A. |
| | promoter | | CSRE#2 | −113/−114 | −196/−195 | N.A. |
| | | | CSRE #3 | −131/−132 | −214/−213 | N.A. |
| | | | CSRE#4 | −200/−201 | −283/−282 | N.A. |
| | | | CSRE#5 | −320/−321 | −403/−402 | N.A. |
| M33403 | eADH2-10 | 10 | CSRE#1 | −96/−97 | −178/−177 | N.A. |
| | promoter | | CSRE#2 | −101/−102 | −184/−183 | N.A. |
| | | | CSRE #3 | −106/−107 | −189/−188 | N.A. |
| | | | CSRE#4 | −111/−112 | −194/−193 | N.A. |
| | | | CSRE#5 | −116/−117 | −199/−198 | N.A. |
| | | | CSRE#6 | −121/−122 | −204/−203 | N.A. |
| | | | CSRE#7 | −126/−127 | −209/−208 | N.A. |
| | | | CSRE#8 | −131/−132 | −214/−213 | N.A. |
| | | | CSRE#9 | −200/−201 | −283/−282 | N.A. |
| | | | CSRE#10 | −320/−321 | −403/−402 | N.A. |
| T13859 | eADH2p- | 11 | CSRE#3 | −131/−132 | −214/−213 | N.A. |
| M33399 | 03.1 | | | | | |
| | promoter | | | | | |
| T15016 | eADH2p- | 24 | CSRE#3 | −320/−321 | −403/−402 | N.A. |
| | 03.7 | | | | | |
| | promoter | | | | | |
| T15015 | eADH2p- | 23 | CSRE#3 | −241/−242 | −324/−323 | N.A. |
| | 03.6 | | | | | |
| | promoter | | | | | |
| T15014 | eADH2p- | 22 | CSRE#3 | −191/−192 | −274/−273 | N.A. |
| | 03.5 | | | | | |
| | promoter | | | | | |
| T15011 | eADH2p- | 19 | CSRE#3 | −78/−79 | −162/−161 | N.A. |
| | 03.2 | | | | | |
| | promoter | | | | | |
| T15012 | eADH2p- | 20 | CSRE#3 | −49/−50 | −132/−131 | N.A. |
| | 03.3 | | | | | |
| | promoter | | | | | |
| T15013 | eADH2p- | 21 | CSRE#3 | −9/−10 | −92/−91 | N.A. |
| | 03.4 | | | | | |
| | promoter | | | | | |
| T13863 | eSPI1p- | 13 | CSRE#1 | −46/−47 | −140/−139 | −50/−49 |
| M33406 | 03 | | CSRE#2 | −72/−73 | −166/−165 | −76/−75 |
| | promoter | | CSRE#3 | −78/−79 | −172/−171 | −82/−81 |
| M35140 | eSPI1p- | 18 | CSRE#3 | +16/+17 | −172/−171 | −82/−81 |
| | 07 | | | | | |
| | promoter | | | | | |
| M35141 | eSPI1p- | 14 | N.A. | N.A. | N.A. | N.A. |
| | 04 | | | | | |
| | promoter | | | | | |
| M35142 | eSPI1p- | 15 | N.A. | N.A. | N.A. | N.A. |
| | 05 | | | | | |
| | promoter | | | | | |
| M35143 | eSPI1p- | 16 | N.A. | N.A. | N.A. | N.A. |
| | 06 | | | | | |
| | promoter | | | | | |

Aerobic fermentation conditions. The different strains were inoculated from agar plates into growth medium containing dextrose for 20-24 hours, after which the biomass was transferred to growth medium containing ethanol or methanol (as indicated in the figure legends) for 48 hours. In assays conducted in 96-well plates, a concentration of 20 g/L of ethanol or methanol was used. In assays conducted in bioreactors, 3.6 mL/hour of ethanol or methanol was used.

Lipase enzymatic assay. The fermentation samples were incubated with the lipase-specific fluorogenic substrate DGGR (1,2-o-dilauryl-rac-glycero-3-glutaric acid-(6′-methylresorufin) ester) for 20 minutes at 25° C., with data recorded every 30 seconds (through excitation at 529 nm and emission reading at 600 nm). Lipase activity units were either provided as relative fluorescence units compared to a control or computed by comparing the emission data against a standard curve of a commercial lipase sample. Productivity was calculated by dividing the measured lipase activity by the optical density obtained at 600 nm or by the dry cell weight (in g).

The promoter of the adh2 gene of K. phaffii (KpADH2p) is an exemplary ethanol-responsive promoter. It was first investigated if its expression strength could be further enhanced in the presence of ethanol. As such, it was first tested if the addition of one or more CSREs could modulate the expression strength of the KpADH2 promoter. Various engineered KpADH2-based (eKpADH-2) promoters are depicted in FIGS. 1 and 6. The wild-type and engineered promoters were all cloned upstream of a reporter gene (see Table 2) and expressed by K. phaffii during aerobic fermentation in the presence of ethanol as the sole carbon source. As shown in FIG. 2, the addition of one (present in strain M32816), two (present in strain M32818), or three (present in strain M32820) CSRE(s) in engineered eADH2 promoters increased the expression of the reporter gene when compared to the wild-type KpADH2 promoter (present in strain M32338).

When ethanol was used as the sole carbon source, the strongest promoter tested in FIG. 2 was the eADH2p-03 promoter (present in strain M32820). It was then determined if similar results could be obtained with an engineered promoter comprising fewer CSREs. The expression strengths of two additional eADH2 promoters were compared. The eADH2p-03 promoter comprised 3 added CSREs, whereas the eADH2p-03.1 promoter had a single added CSRE, both promoters having a commonly added CSRE (see FIG. 1). As shown in FIG. 3, the CSRE engineered most proximally to the ADH2 core promoter region in eADH2p-03.1 (present in isolate T13859) accounts for 57% of the reporter enzyme activity increase compared to eADH2p-03 (present in strain M32820) relative to the native ADH2p promoter (present in strain M32338).

It was then determined how the engineered promoter eADH2p-03 compared to a well-known and currently used methanol-inducible promoter from the aox1 gene from K. phaffii (AOX1p). Strains M31676, M32338, and M32820 expressed the same reporter gene, and were engineered in the same manner, and respectively include the wild-type methanol-inducible promoters AOX1p, the wild-type ethanol-responsive promoter KpADH2p, or the eADH2p-03 promoter. As illustrated in FIG. 4 with strain M32338 (comprising the wild-type KpADH2p), K. phaffii growth on ethanol yielded comparable biomass production and higher reporter enzymatic activity titers when compared the methanol-inducible strain M31676 (comprising the wild-type AOX1p). As also shown on FIG. 4, strain M32820 (comprising the eADH2p-03) growth on ethanol yielded comparable biomass production and higher reporter enzymatic activity titers when compared to the methanol-inducible strain M31676 (comprising the wild-type AOX1p). As the rhombi show on FIG. 4, the amount of reporter enzymatic activity per unit of biomass produced is 1.5 times higher for strain M32820 than for strain M32338, suggesting that under control of the engineered eADH2p-03 promoter, the magnitude of the ethanol induction effect is higher for strain M32820.

It was also determined if adding further CSREs (outside the core section and outside any other known CSREs) could further increase the strength of the engineered promoters derived from KpADH2p in the presence of ethanol. The strength of the wild-type promoter KpADH2p (present in strain M32388) and engineered promoters eADH2p-01 (present in strain M32816), eADH2p-02 (present in strain M32818), and eADH2p-03 (present in strain M32820) was compared to engineered promoters comprising 5 added CSREs (eADH2p-05, present in strain M33401) or 10 added CSREs (eADH2p-10, present in strain M33403). As shown on FIG. 5, reporter gene expression driven under control of eADH2p-03, eADH2p-05, or eADH2p-10, respectively resulted in a 187%, 206%, or 213% increase in reporter enzyme activity relative to the wild-type KpADH2p (strains M32820, M33401, and M33403 compared to strain M32338). The growth and biomass production of the strains were substantially equivalent. As the squares depicted on the right hand-side y axis highlight on FIG. 5, the increase in measured reporter gene activity is the result of increased productivity of the yeast biomass (233%, 294%, and 339%, productivity values over the wild-type KpADH2p-driven expression for eADH2p-03, eADH2p-05, or eADH2p-10, respectively). These data support the notion that the increasing number of CSREs drives stronger gene expression in the presence of ethanol as the sole carbon source.

Promoters usually include a core section (e.g., a section that the RNA polymerase physically occupies when it transcribes the gene and which may include a TATA box) and it was further determined how adding CSREs closer to the core section, and even within the core section modulates gene expression. A further set of engineered promoters (as schematically shown on FIG. 6) have been designed and their ability to drive the expression of a reporter gene in the presence of ethanol as the sole carbon source was assessed. Under control of the eADH2p-03.7 promoter, where the engineered CSRE is most distal to the core promoter, measured secreted reporter enzyme activity and biomass productivity are statistically equivalent to that of the wild-type KpADH2p (T15016 versus M32338). As the engineered CSRE is integrated gradually more proximal to the core promoter (T15015, T15014, M33399, and T15011), the magnitude of reporter enzymatic activity increased correspondingly (24%, 61%, 121%, and 148% increase relative to the wild-type KpADH2p). Similarly, the reporter enzyme productivity per biomass also increased in a dose-dependent manner (28%, 70%, 134%, and 187% increase relative to the wild-type KpADH2p). Once the CSRE is integrated within the bounds of the core promoter (T15012 and T15013), the measured activity and productivity begin decreasing (140% and 81% over wild-type KpADH2p for activity, and 180% and 87% over the native KpADH2p for productivity). It is worth noting that the strength of all of the engineered promoters tested was still substantially higher than that of the wild-type KpADH2p.

The promoter of the spi1 gene of K. phaffii (KpSPI1p) is an exemplary constitutive promoter (data not shown). FIG. 8 compares how expression driven by the wild-type KpSPI1p promoter compared to that driven by the wild-type KpADH2p promoter, as well as the methanol-inducible AOX1 promoter. This figure highlights reporter enzymatic activity data in shake flasks, comparing secreted enzymatic activity between strains expressing the reporter gene under each of the promoters denoted in the graph. In this evaluation, the same reporter enzyme reached higher expression titers under the control of the wild-type KpSPI1p or the wild-type Ogataea polymorpha ADH1 promoter (OpADH1p) compared to the wild-type KpAOX1p or KpADH2p expression systems. In batch systems where cells reach stationary phase (such as shake flasks), the wild-type KpSPI1p drives higher reporter gene expression than wild-type KpADH2p (FIG. 8). However, as illustrated in the aerobic fed-batch fermentation depicted in FIG. 9 (where ethanol is the sole carbon source and biomass growth is controlled), the highest levels of secreted reporter enzyme activity were achieved under control of the wild-type KpADH2 promoter.

To confer ethanol responsiveness to the constitutive KpSPI1p, one or three CSREs was/were added upstream of the core KpSPI1 promoter (schematically depicted in FIG. 10). When ethanol is used as the sole carbon source, as shown on FIG. 11, the use of the eSPI1p-03 (included in strain M33046) or the eSPI1p-07 (included in strain M35140) respectively caused a 16% and 19% increase in secreted reporter enzyme activity relative to the wild-type KpSPI1p (included in strain M32696). As also shown on FIG. 11, the use of the eSPI1p-03 (included in strain M33046) or the eSPI1p-07 (included in strain M35140) respectively caused a 23% and 25% increase in productivity relative to the wild-type KpSPI1p (included in strain M32696).

Table 3 provides the description of the various Komagataella phaffii strains that were engineered to express a reporter glucose oxidase. The genetically modified strains were all generated (directly or indirectly) from the K. phaffii strain NRRL Y-11430 obtained from ATCC (76273).

TABLE 3
Komagataella phaffii strains (M*) that were made. All strains
(except the wild-type strains) were engineered to express a reporter
gene (Aspergillus niger glucose oxidase (GOx)) having the
amino acid sequence of SEQ ID NO: 38 and encoded by the nucleic
acid sequence of SEQ ID NO: 37. All strains are derived (directly
or indirectly) from the wild-type strain M17500. Each strain/isolate
comprises a single chromosomal copy of an expression cassette
comprising a promoter and the reporter gene.

| | | | CSRE | Position of CSRE with respect to | | |
|---|---|---|---|---|---|---|
| | | SEQ | in | | start codon | |
| | | ID | epro- | TATA | of reporter | |
| Strain | Promoter | NO: | moter? | box | gene | TSS |
| M17500 | N.A. - this is a wild-type strain | | | | | |
| M32685 | KpADH2 | 2 | N.A. | N.A. | N.A. | N.A. |
| | promoter | | | | | |
| M33193 | eADH2- | 8 | CSRE#1 | −131/−132 | −214/−213 | N.A. |
| | 03 | | CSRE#2 | −200/−201 | −283/−282 | N.A. |
| | promoter | | CSRE #3 | −320/−321 | −403/−402 | N.A. |

Glucose oxidase activity in 96 well culture plates was determined after 48 h of growth with ethanol as the carbon source. Deep well supernatants were diluted 1:50 in phosphate buffered saline and incubated with glucose, peroxidase and the chromogenic substrate mix (p-hydroxybenzoic acid and 4-aminoantipyrine). Enzymatic activity was monitored by absorbance at 510 nm at 25° C. and the value at 9 minutes is reported. As shown in FIG. 12, the use of the eADH2-03p (included in strain M33193) caused an increase in glucose oxidase activity and productivity compared to the strain M32685 (using the wild-type ADH2 promoter). Table 4 provides the description of the various Komagataella phaffii strains that were engineered to express a reporter alpha-amylase (AA). The genetically modified strains were all generated (directly or indirectly) from the K. phaffii strain NRRL Y-11430 obtained from ATCC (76273).

TABLE 4
Komagataella phaffii strains (M*) that were made. All strains
(except the wild-type strains) were engineered to express a reporter
gene (Aspergillus oryzae alpha-amylase (AA)) having the amino
acid sequence of SEQ ID NO: 40 and encoded by the nucleic acid
sequence of SEQ ID NO: 41. All strains are derived (directly
or indirectly) from the wild-type strain M17500. Each strain/isolate
comprises a single chromosomal copy of an expression cassette
comprising a promoter and the reporter gene.

| | | | CSRE | Position of CSRE with respect to | | |
|---|---|---|---|---|---|---|
| | | SEQ | in | | start codon | |
| | | ID | epro- | TATA | of reporter | |
| Strain | Promoter | NO: | moter? | box | gene | TSS |
| M17500 | N.A. - this is a wild-type strain | | | | | |
| M33232 | KpADH2 | 2 | N.A. | N.A. | N.A. | N.A. |
| | promoter | | | | | |
| M33328 | eADH2- | 8 | CSRE#1 | −131/−132 | −214/−213 | N.A. |
| | 03 | | CSRE#2 | −200/−201 | −283/−282 | N.A. |
| | promoter | | CSRE #3 | −320/−321 | −403/−402 | N.A. |

Alpha-amylase activity in 96 well culture plates was determined after 48 h of growth with ethanol as the carbon source. Deep well supernatants were diluted 1:100 in phosphate buffered saline and incubated with α-glucosidase and the chromogenic substrate (p-nitrophenyl maltoheptaoside) and incubated at 40° C. for 10 minutes. The reaction was stopped by addition of 1% tri-sodium phosphate and the activity was measured by absorbance at 400 nm. As shown in FIG. 13, the use of the eADH2-03p (included in strain M33328) caused an increase in alpha-amylase activity and productivity compared to the strain M33232 (using the wild-type ADH2 promoter).

What is claimed is:

1. An engineered promoter (i) derived from a parental promoter having a transcription start site and (ii) for expressing a gene,
wherein the engineered promoter has at least one external carbon source-responsive element (CSRE), wherein the at least one external CSRE has the nucleic acid sequence of formula (I):
N₁N₂CCN₃N₄TN₅N₆N₇CCGN₈ (I)
N₁ is any nucleic acid residue;
N₂ is any nucleic acid residue, preferably C or T;
N₃ is any nucleic acid residue, preferably A, G or T;
N₄ is any nucleic acid residue, preferably C or T;
N₅ is any nucleic acid residue, preferably A, C or G;
N₆ is any nucleic acid residue, preferably A or G;
N₇ is any nucleic acid residue, preferably G or T; and
N₈ is any nucleic acid residue, preferably A or G; and
wherein the at least one external CSRE comprises a first external CSRE located upstream of and being proximal to the transcription start site.

2. The engineered promoter of claim 1, wherein the gene comprises an open reading frame having a start codon and optionally a TATA box.

3. The engineered promoter of claim 2, wherein the first external CSRE is located at most 390 base pairs upstream (−390) of the start codon.

4. The engineered promoter of claim 1, wherein, in the presence of a C2 carbon source, the engineered promoter is capable of inducing transcription of the gene at a higher level than the parental promoter.

5. The engineered promoter of claim 1, wherein the at least one external CSRE comprises the nucleic acid sequence of any one of SEQ ID NO: 26 to 35.

6. The engineered promoter of claim 1 comprising at least two, three, four, five, six, seven, eight, nine, or ten external CSREs.

7. The engineered promoter of claim 1, wherein the parental promoter is an ethanol responsive promoter or a constitutive promoter.

8. The engineered promoter of claim 7, wherein the parental promoter is the promoter of the adh2 gene (adh2p).

9. The engineered promoter of claim 8 having the nucleic acid sequence of SEQ ID NO: 6, 7, 8, 9, 10, 11, 19, 20, 21, 22, or 23.

10. The engineered promoter of claim 7, wherein the parental promoter is the promoter of the sti1 gene (sti1p).

11. The engineered promoter of claim 10 having the nucleic acid sequence of SEQ ID NO: 12, 13, 14, 15, 16, 17, or 18.

12. A heterologous nucleic acid molecule having the engineered promoter of claim 1 operably associated with a gene.

13. A recombinant microbial host cell comprising the engineered promoter of claim 1.

14. The recombinant microbial host cell of claim 13 having native alcohol dehydrogenase activity.

15. The recombinant microbial host cell of claim 13 being a yeast.

16. The recombinant microbial host cell of claim 15 being from Komagataella sp. or from Komagataella phaffii.

17. A method for expressing a gene in the recombinant microbial host cell claim 13, the method comprises (i) contacting the recombinant microbial host cell with a C2 carbon source so as to allow the expression of the gene.

18. The method of claim 17 further comprising:
before the step (i), (i′) propagating the recombinant microbial host cell with an alternative carbon source different from the C2 carbon source; and/or
after step (i), (ii) substantially separating the polypeptide from the recombinant microbial host cell.

19. The method of claim 18, wherein the alternative carbon source comprises glucose, fructose and/or glycerol.

20. The method of claim 17, wherein the gene encodes a polypeptide.

21. The method of claim 20, wherein the polypeptide is an intracellular polypeptide or a secreted polypeptide.

22. The method of claim 20, wherein the polypeptide is an enzyme.


SEQUENCE LISTING (40 sequences)

SEQ ID NO: 1 - DNA, 939 bp, Komagataella phaffii
  source 1..939: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 2 - DNA, 1211 bp, Komagataella phaffii
  source 1..1211: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 3 - DNA, 853 bp, Komagataella phaffii
  source 1..853: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 4 - DNA, 623 bp, Ogataea polymorpha
  source 1..623: mol_type = other DNA
aatgtatctaaacgcaaactccgagctggaaaaatgttaccggcgatgcgcggacaatttagaggcggcgatcaagaaacacctgctgggcgagcagtctggagcacagtcttcgatgggcccgagatcccaccgcgttcctgggtaccgggacgtgaggcagcgcgacatccatcaaatataccaggcgccaaccgagtctctcggaaaacagcttctggatatcttccgctggcggcgcaacgacgaataatagtccctggaggtgacggaatatatatgtgtggagggtaaatctgacagggtgtagcaaaggtaatattttcctaaaacatgcaatcggctgccccgcaacgggaaaaagaatgactttggcactcttcaccagagtggggtgtcccgctcgtgtgtgcaaataggctcccactggtcaccccggattttgcagaaaaacagcaagttccggggtgtctcactggtgtccgccaataagaggagccggcaggcacggagtctacatcaagctgtctccgatacactcgactaccatccgggtctctcagagaggggaatggcactataaataccgcctccttgcgctctctgccttcatcaatcaaatc

SEQ ID NO: 5 - DNA, 623 bp, Ogataea polymorpha; identical to SEQ ID NO: 4
  source 1..623: mol_type = other DNA

SEQ ID NO: 6 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgttccgttcgtccggctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 7 - DNA, 1239 bp, synthetic construct
  source 1..1239: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgttccgttcgtccggctaatttcgttgattcccacccccctttccaactccacaaattgtccaatctcgttttccatttgggagaatctgcatgtcgactacataaagcgaccggtgtccgaaaagatctgtgtactccgctcagccgagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 8 - DNA, 1253 bp, synthetic construct
  source 1..1253: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgttccgttcgtccggctaatttcgttgattcccacccccctttccaactccacaaattgtccaatctcgttttccatttgggagaatctgcatgtcgactacataaagcgaccggtgtccgaaaagatctgtgtactccgctcagccgagttttcaacattttgtgctccccccgctgtttgaaaacgggggtgagcgctctccggggtgcgaattcgtcccgttggtccgatgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 9 - DNA, 1281 bp, synthetic construct
  source 1..1281: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgttccgttcgtccggctaatttcgttgattcccacccccctttccaactccacaaattgtccaatctcgttttccatttgggagaatctgcatgtcgactacataaagcgaccggtgtccgaaaagatctgtgtactccgctcagccgagttttcaacattttgtgctccccccgctgtttgaaaacgggggtgagcgctctccggggtgcgaattcgtcccgttggtccgatgcccaattcctttcaccacccgttcagccgactgcctattgtagacgtcctccattcggccgaaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 10 - DNA, 1351 bp, synthetic construct
  source 1..1351: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgttccgttcgtccggctaatttcgttgattcccacccccctttccaactccacaaattgtccaatctcgttttccatttgggagaatctgcatgtcgactacataaagcgaccggtgtccgaaaagatctgtgtactccgctcagccgagttttcaacattttgtgctccccccgctgtttgaaaacgggggtgagcgctctccggggtgcgaattcgtcccgttggtccgatgcccacccgttcagccgaaattcctccattcggccgactttctccctttaatccgaaccctgcccgttagtccgagcctattccattaagccggttgtacccctttcggccgggacgtatccattcatccggcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 11 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtcccgttggtccgatgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 12 - DNA, 1521 bp, synthetic construct
  source 1..1521: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 13 - DNA, 876 bp, synthetic construct
  source 1..876: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaattccgttcgtccgggtccagctccgctcagccgaattagggcagattttgagtttaaaattcccgttggtccgaaccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 14 - DNA, 834 bp, synthetic construct
  source 1..834: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 15 - DNA, 853 bp, synthetic construct
  source 1..853: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaattaaaaaatagatcaagataaccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 16 - DNA, 853 bp, synthetic construct
  source 1..853: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaatacaagaataagttaaataaaccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 17 - DNA, 895 bp, synthetic construct
  source 1..895: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttccgttcgtccggttatagctttctccgctcagccgatcgcctggtgtcccgttggtccgaccgtacggtataaatacatactctcctcccccccctggttctctttttcttttgttacttacattttaccgttccgtcactcgcttcactcaacaacaaaa

SEQ ID NO: 18 - DNA, 867 bp, synthetic construct
  source 1..867: mol_type = other DNA
gtccagccatttttttctttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgttcccgttggtccgattatagcttttcgcctggtgccgtacggtataaatacatactctcctcccccccctggttctctttttcttttgttacttacattttaccgttccgtcactcgcttcactcaacaacaaaa

SEQ ID NO: 19 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgctcccgttggtccgagaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 20 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccatcccgttggtccgaacaattggtccacccctccccaatctctaatattcacaattcacctcactataaatacccctgtcctgctcccaaattcttttttccttcttccatcagctactagcttttatcttatttactttacgaaa

SEQ ID NO: 21 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcccgttggtccgatcacctcactataaatacccctgtcctgctcccaaattcttttttccttcttccatcagctactagcttttatcttatttactttacgaaa

SEQ ID NO: 22 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcgactacataaagcgaccggtgtccgaaaagatctgtgtagttttcaactcccgttggtccgaattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 23 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatctgctaatttcgttgattcccacccccctttccaactccacaaattgtccaatctcgttttccatttgggagaatctgcatgtcccgttggtccgatcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 24 - DNA, 1225 bp, synthetic construct
  source 1..1225: mol_type = other DNA
gcagcgttttctgacggtactagaggactcttaggggaaggtagaatcaataaagatcatattaggtaagcaaattttggatggaataggagactaggtgtggatgcgcgatctcgccaaattgcacgaccagagtggatgccggatggtggtaaaccgtttcttcctttttaccacccaagtgcgagtgaaacaccccatggctgctctccgattgcccctctacaggcataagggtgtgactttgtgggcttgaattttacaccccctccaacttttctcgcatcaattgatcctgttaccaatattgcatgcccggaggagacttgccccctaatttcgcggcgtcgtcccggatcgcagggtgagactgtagagaccccacatagtgacaatgattatgtaagaagaggggggtgattcggccggctatcgaactctaacaactaggggggtgaacaatgcccagcagtcctccccactctttgacaaatcagtatcaccgattaacaccccaaatcttattctcaacggtccctcatccttgcacccctctttggacaaatggcagttagcattggtgcactgactgactgcccaaccttaaacccaaatttcttagaaggggcccatctagttagcgaggggtgaaaaattcctccatcggagatgtattgaccgtaagttgctgcttaaaaaaaatcagttcagatagcgagacttttttgatttcgcaacgggagtgcctgttccattcgattgcaattctcaccccttctgcccagtcctgccaattgcccatgaatcttcccgttggtccgagctaatttcgttgattcccacccccctttccaactccacaaattgtccaatctcgttttccatttgggagaatctgcatgtcgactacataaagcgaccggtgtccgaaaagatctgtgtagttttcaacattttgtgctccccccgctgtttgaaaacgggggtgagcgctctccggggtgcgaattcgtgcccaattcctttcaccctgcctattgtagacgtcaacccgcatctggtgcgaatatagcgcacccccaatgatcacaccaacaattggtccacccctccccaatctctaatattcacaattcacctcactataaatacccctgtcctgctcccaaattcttttttccttcttccatcagctactagcttttatcttatttactttacgaaa

SEQ ID NO: 25 - protein, 423 aa, Fusarium oxysporum
MRQVWFSWIVGLFLCFFNVSSAAPVNTTTEDETAQIPAEAVIGYSDLEGDFDVAVLPFSNSTNNGLLFINTTIASIAAKEEGVSLEKREAEASPVALDDYVNSLEERAVGVTTTDFSNFKFYIQHGAAAYCNSEAAAGSKITCSNNGCPTVQGNGATIVTSFVGSKTGIGGYVATDSARKEIVVSFRGSINIRNWLTNLDFGQEDCSLVSGCGVHSGFQRAWNEISSQATAAVASARKANPSFNVISTGHSLGGAVAVLAAANLRVGGTPVDIYTYGSPRVGNAQLSAFVSNQAGGEYRVTHADDPVPRLPPLIFGYRHTTPEFWLSGGGGDKVDYTISDVKVCEGAANLGCNGGTLGLDIAAHLHYFQATDACNAGGFSWRRYRSAESVDKRATMTDAELEKKLNSYVQMDKEYVKNNQARS

SEQ ID NO: 26 - DNA, 14 bp, synthetic construct
  source 1..14: mol_type = other DNA
ttccgttcgtccgg

SEQ ID NO: 27 - DNA, 14 bp, synthetic construct
  source 1..14: mol_type = other DNA
ctccgctcagccga

SEQ ID NO: 28 - DNA, 14 bp, synthetic construct
  source 1..14: mol_type = other DNA
tcccgttggtccga

SEQ ID NO: 29 - DNA, 14 bp, synthetic construct
  source 1..14: mol_type = other DNA
acccgttcagccga

SEQ ID NO: 30 - DNA, 14 bp, synthetic construct
  source 1..14: mol_type = other DNA
ctccattcggccga

SEQ ID NO: 31 - DNA, 14 bp, synthetic construct
  source 1..14: mol_type = other DNA
tccctttaatccga

SEQ ID NO: 32 - DNA, 14 bp, synthetic construct
  source 1..14: mol_type = other DNA
gcccgttagtccga

SEQ ID NO: 33 - DNA, 14 bp, synthetic construct
  source 1..14: mol_type = other DNA
ttccattaagccgg

SEQ ID NO: 34 - DNA, 14 bp, synthetic construct
  source 1..14: mol_type = other DNA
cccctttcggccgg

SEQ ID NO: 35 - DNA, 14 bp, synthetic construct
  source 1..14: mol_type = other DNA
atccattcatccgg

SEQ ID NO: 36 - DNA, 1023 bp, Fusarium oxysporum
  source 1..1023: mol_type = other DNA
atgagacaagtttggttctcttggatcgttggtttgttcttgtgtttcttcaacgtttcttctgctgctccagttaacactactactgaagacgaaactgctcaaatcccagctgaagctgttatcggttactctgacttggaaggtgacttcgacgttgctgttttgccattctctaactctactaacaacggtttgttgttcatcaacactactatcgcttctatcgctgctaaggaagaaggtgtttctttggaaaagagagaagctgaagcttctccagttgctttggatgattacgttaactctttggaagaaagagctgttggcgttactactactgatttctctaacttcaagttctacatccaacatggtgctgctgcttactgtaattctgaagctgctgctggttctaagattacctgttctaacaatggttgtccaaccgttcaaggtaatggtgctactatcgttacttcttttgttggttccaagactggtattggtggttatgttgctactgattccgctagaaaagaaatcgttgtttccttcagaggctccatcaatattagaaactggttgaccaatttggacttcggtcaagaagattgctctttggtttctggttgtggtgttcattctggttttcaaagagcttggaacgaaatttcatctcaagctactgctgctgttgcatctgcaagaaaagctaatccatccttcaacgttatttccaccggtcattctttaggtggtgctgttgctgttttggctgctgctaatttgagagttggtggtactccagttgatatctatacttatggttctccaagagttggtaacgctcaattgtctgctttcgtttctaatcaagctggtggtgagtatagagttactcatgctgatgatccagttccaagattgccaccattgatttttggttacagacataccactccagagttttggttgtcaggtggtggtggcgataaggttgattatacaatttccgatgtt

SEQ ID NO: 37 - DNA, 1818 bp, Aspergillus niger
  source 1..1818: mol_type = other DNA
atgcagaccttgttggtttcctctttggttgtttctttggctgctgctttgccacattacattagatccaatggtattgaggcttccttgttgactgatccaaaagatgtttctggtagaaccgtcgattacattattgctggtggtggtttgactggtttaactactgctgctagattgactgaaaacccaaacatctccgttttggttatcgaatctggttcctacgaatctgatagaggtccaattatcgaagatttgaacgcctacggtgatatctttggttcttctgttgatcatgcttacgaaaccgttgaattggctaccaacaatcaaactgccttgattagatctggtaacggtttaggtggttctactttggttaatggtggtacttggactagaccacataaggctcaagttgattcttgggaaactgtttttggtaacgaaggttggaattgggataatgttgctgcttattcattgcaagctgaaagggctagagcaccaaatgctaaacaaattgctgctggtcattacttcaatgcctcttgtcatggtgttaacggtactgttcatgctggtccaagagatactggtgatgattattctccaatcgttaaggctttgatgtccgctgttgaagatagaggtgttccaactaagaaagatttcggttgtggtgatccacatggtgtttctatgtttccaaataccttgcacgaagatcaagttagatctgatgctgcaagagaatggttgttgccaaattatcaaagaccaaacttgcaggtcttgactggtcaatatgttggtaaggttttgttgtcccaaaacggtactactccaagagctgttggtgttgaatttggtactcataagggtaacacccataacgtttacgccaaacatgaagttttgttagctgctggttctgctgtttctccaactattttggaatactccggtatcggtatgaagtccattttggaaccattgggtattgataccgttgttgatttgccagttggtttgaacttacaagatcaaactaccgctaccgtcagatctagaattacttctgctggtgctggtcaaggtcaagctgcttggtttgctacttttaacgaaactttcggtgactactctgaaaaggcccatgaattattgaacaccaaattggaacaatgggctgaagaggctgttgctagaggtggttttcataacactactgccttgttgatccagtacgaaaattacagagattggatcgttaaccacaacgttgcttactctgagttgtttttggatactgctggtgttgcttcttttgatgtatgggatttgttgccattcaccagaggttacgttcacattttggataaggatccatacttgcatcatttcgcttacgacccacaatactttttgaacgaattggacttgttgggtcaagccgctgctactcaattggctagaaacatttctaattccggtgctatgcaaacctatttcgctggtgaaactattccaggtgataacttggcttatgatgctgatttgtctgcttggactgagtacattccataccatttcagaccaaattaccacggtgttggtacttgttctatgatgccaaaagaaatgggtggtgttgttgataatgcagctagagtttacggtgttcaaggtttgagagttatcgatggttctattccaccaactcaaatgtcctctcatgttatgactgttttctacgctatggccttgaagatttccgatgcaattttggaagattacgcctccatgcaataa

SEQ ID NO: 38 - protein, 605 aa, Aspergillus niger
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 39 - DNA, 1497 bp, Aspergillus oryzae
  source 1..1497: mol_type = other DNA
atggttgcttggtggtctttgttcttgtacggtttacaagttgctgctccagctttggctgctactccagctgattggagatcacaatctatctactttttgttgaccgacagattcgctagaactgatggttctactactgctacttgtaataccgctgatagaaagtattgtggtggtacttggcaaggtatcatcgataagttggattacattcaaggtatgggtttcaccgctatttggattactccagttactgctcaattgccacaaactactgcttatggtgatgcttatcatggttattggcaacaggatatctactccttgaacgaaaattacggtactgccgatgatttgaaggctttgtcatctgctttacatgaacgtggtatgtacttgatggttgatgttgttgctaaccacatgggttatgatggtgctggttcttctgttgattactctgtttttaagcccttcagctcccaagattactttcatccattctgcttgatccaaaactacgaagatcaaactcaagtcgaagattgctggttgggtgataatactgtttctttgccagatttggataccaccaaggatgttgttaagaacgaatggtatgattgggtcggttctttggtttccaactactctattgatggtttgagaatcgataccgtcaagcacgttcaaaaagatttttggccaggttacaacaaagctgctggtgtttactgtattggtgaagttttagatggtgatccagcttacacttgtccataccaaaatgttatggatggcgttttgaactacccaatctactacccattattgaacgctttcaagtctacctctggttctatggatgacttgtacaacatgatcaacaccgttaagtctgattgtccagattctactttgttgggtactttcgttgaaaaccacgataatccaagattcgcttcttacaccaacgatattgctttggctaaaaacgttgccgccttcattattttgaacgatggtattccaattatctacgccggtcaagaacaacattatgctggtggtaatgatccagcaaatagagaagctacttggttgtctggttatccaactgattccgagttgtacaaattgattgcttccgctaacgccattagaaactacgctatttctaaggatactggcttcgttacttacaagaattggcctatctacaaggatgataccactattgctatgagaaagggtacagatggttctcaaatcgttaccatcttgtctaacaaaggtgcttctggtgattcctacactttgtctttgtctggtgcaggttatactgctggtcaacaattgactgaagttattggttgtactaccgttaccgttggttctgatggtaatgttcctgttccaatggctggtggtttgccaagagtcttgtatccaacagaaaaattggccggttccaagatctgttcttcttcttga

SEQ ID NO: 40 - protein, 498 aa, Aspergillus oryzae
MVAWWSLFLYGLQVAAPALAATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS